SEQUENCE LISTING (66 sequences)

SEQ ID NO: 1 - DNA, 40 bp, synthetic construct
  misc_feature 1..40: note = Description of Artificial Sequence: Synthetic oligonucleotide
  source 1..40: mol_type = other DNA
aaaaattgaaattttattttttttttttggaatataaata

SEQ ID NO: 2 - DNA, 31 bp, synthetic construct
  misc_feature 1..31: note = Description of Artificial Sequence: Synthetic oligonucleotide
  source 1..31: mol_type = other DNA
gaatttcattttgtttttttctatgctataa

SEQ ID NO: 3 - DNA, 987 bp, Homo sapiens
  source 1..987: mol_type = unassigned DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 4 - DNA, 762 bp, Homo sapiens
  source 1..762: mol_type = unassigned DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 5 - DNA, 597 bp, synthetic construct
  misc_feature 1..597: note = Description of Artificial Sequence: Synthetic polynucleotide
  source 1..597: mol_type = other DNA
gccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 6 - DNA, 30 bp, synthetic construct
  misc_feature 1..30: note = Description of Artificial Sequence: Synthetic oligonucleotide
  source 1..30: mol_type = other DNA
gttcctggagtaggggtacctggggtgggc

SEQ ID NO: 7 - DNA, 1080 bp, Homo sapiens
  source 1..1080: mol_type = unassigned DNA
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 8 - DNA, 720 bp, synthetic construct
  misc_feature 1..720: note = Description of Artificial Sequence: Synthetic polynucleotide
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 1611 bp, synthetic construct
  misc_feature 1..1611: note = Description of Artificial Sequence: Synthetic polynucleotide
  source 1..1611: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 10 - DNA, 3522 bp, synthetic construct
  misc_feature 1..3522: note = Description of Artificial Sequence: Synthetic polynucleotide
  source 1..3522: mol_type = other DNA
gaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 11 - DNA, 4758 bp, synthetic construct
  misc_feature 1..4758: note = Description of Artificial Sequence: Synthetic polynucleotide
  source 1..4758: mol_type = other DNA
gatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccat

SEQ ID NO: 12 - DNA, 7474 bp, synthetic construct
  misc_feature 1..7474: note = Description of Artificial Sequence: Synthetic polynucleotide
  source 1..7474: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgatatctagatgcattcgcgaggtaccgagctcgaattcgatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 13 - DNA, 51 bp, synthetic construct
  misc_feature 1..51: note = Description of Artificial Sequence: Synthetic primer
  source 1..51: mol_type = other DNA
ggggacaagtttgtacaaaaaagcaggctcgtagacgcggtgtttctatcc

SEQ ID NO: 14 - DNA, 50 bp, synthetic construct
  misc_feature 1..50: note = Description of Artificial Sequence: Synthetic primer
  source 1..50: mol_type = other DNA
ggggaccactttgtacaagaaagctgggtaaacgtaacaccgtaactgcc

SEQ ID NO: 15 - DNA, 31 bp, synthetic construct; identical to SEQ ID NO: 2
  misc_feature 1..31: note = Description of Artificial Sequence: Synthetic primer
  source 1..31: mol_type = other DNA

SEQ ID NO: 16 - DNA, 52 bp, synthetic construct
  misc_feature 1..52: note = Description of Artificial Sequence: Synthetic primer
  source 1..52: mol_type = other DNA
ggggacaactttattatacaaagttgtttaggaagcattcagatagctcatc

SEQ ID NO: 17 - skipped

SEQ ID NO: 18 - DNA, 702 bp, Homo sapiens
  source 1..702: mol_type = unassigned DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 19 - skipped

SEQ ID NO: 20 - DNA, 4267 bp, synthetic construct
  misc_feature 1..4267: note = Description of Artificial Sequence: Synthetic polynucleotide
  source 1..4267: mol_type = other DNA
gaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatgtctgaatttcattttgtttttttctatgctataaatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaagcttgaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 21 - DNA, 5503 bp, synthetic construct
  misc_feature 1..5503: note = Description of Artificial Sequence: Synthetic polynucleotide
  source 1..5503: mol_type = other DNA
gatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatgtctgaatttcattttgtttttttctatgctataaatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaagcttgaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccat

SEQ ID NO: 22 - DNA, 8219 bp, synthetic construct
  misc_feature 1..8219: note = Description of Artificial Sequence: Synthetic polynucleotide
  source 1..8219: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaggcctctgcagtcgacgggcccgggatccgatatctagatgcattcgcgaggtaccgagctcgaattcgatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagaatttcattttgtttttttctatgctataaatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatgtctgaatttcattttgtttttttctatgctataaatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaagcttgaaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 23 - DNA, 22 bp, synthetic construct
  misc_feature 1..22: note = Description of Artificial Sequence: Synthetic primer
  source 1..22: mol_type = other DNA
gaagaggacctgggagtagatg

SEQ ID NO: 24 - DNA, 355 bp, synthetic construct
  misc_feature 1..355: note = Description of Artificial Sequence: Synthetic polynucleotide
  source 1..355: mol_type = other DNA
tatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccag

SEQ ID NO: 25 - DNA, 4077 bp, synthetic construct
  misc_feature 1..4077: note = Description of Artificial Sequence: Synthetic polynucleotide
  source 1..4077: mol_type = other DNA
aaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatctcgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaagcttggactcctgttgatagatccagaaaaattgaaattttattttttttttttggaatataaataatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccagatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaggggacaactttgtataataaagttgctaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 26 - DNA, 5338 bp, synthetic construct
  misc_feature 1..5338: note = Description of Artificial Sequence: Synthetic polynucleotide
  source 1..5338: mol_type = other DNA
gatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagacaacttttctatacaaagttgccaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatctcgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaagcttggactcctgttgatagatccagaaaaattgaaattttattttttttttttggaatataaataatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccagatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaggggacaactttgtataataaagttgctaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccat

SEQ ID NO: 27 - DNA, 11039 bp, synthetic construct
  misc_feature 1..11039: note = Description of Artificial Sequence: Synthetic polynucleotide
  source 1..11039: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctatcaacaagtttgtacaaaaaagcaggctcgatggatatctttaatcatttaaatagcattaacccacgcacacgtttttgtttttctcctgtatccgttagctatgcattatctgtatgttgtacgggtaatgttccgtcggattacgtatcctccacagtcgttgtaaaaaacaaagtatacataaacgcgtttaaacagtcccccgtacgcggtacatcgtacgcacacttcactaacgatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaagacaacttttctatacaaagttgccaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatctcgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaagcttggactcctgttgatagatccagaaaaattgaaattttattttttttttttggaatataaataatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagtatgctagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctctagtttcactttccctagtttcactttccctagtttcactttccctagtttcactttccctagtttcactttcccctcgaggatatcaagatctggcctcggcggccagatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaggggacaactttgtataataaagttgctaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccattacccagctttcttgtacaaagtggttgatctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattga

SEQ ID NO: 28 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 30 - protein, 198 aa, synthetic construct
  REGION 1..198: note = Description of Artificial Sequence: Synthetic polypeptide
ARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 10 aa, synthetic construct
  REGION 1..10: note = Description of Artificial Sequence: Synthetic peptide
VPGVGVPGVG

SEQ ID NO: 32 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 33 - protein, 239 aa, synthetic construct
  REGION 1..239: note = Description of Artificial Sequence: Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 34 - protein, 536 aa, synthetic construct
  REGION 1..536: note = Description of Artificial Sequence: Synthetic polypeptide
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 36 - protein, 435 aa, synthetic construct
  REGION 1..435: note = Description of Artificial Sequence: Synthetic polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGMVSVPTASPSASSSSSQCRSSMCQSRYLLFLATLALLNHLSLARVIPVSGPARCLSQSRNLLKTTDDMVKTAREKLKHYSCTAEDIDHED

SEQ ID NO: 37 - protein, 335 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 38 - protein, 90 aa, synthetic construct
  REGION 1..90: note = Description of Artificial Sequence: Synthetic polypeptide
MVSVPTASPSASSSSSQCRSSMCQSRYLLFLATLALLNHLSLARVIPVSGPARCLSQSRNLLKTTDDMVKTAREKLKHYSCTAEDIDHED

SEQ ID NO: 39 - protein, 236 aa, Mus sp.
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 40 - protein, 354 aa, Mus sp.
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 41 - DNA, 675 bp, Homo sapiens
  source 1..675: mol_type = unassigned DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagatgagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtag

SEQ ID NO: 42 - DNA, 625 bp, synthetic construct
  misc_feature 1..625: note = Description of Artificial Sequence: Synthetic polynucleotide
  source 1..625: mol_type = other DNA
ctggcgaaagggggatgtgctgcaaggcgattaagttgggtaacgccagggttttcccagtcacgacgttgtaaaacgacggccagtgaattgtaatacgactcactatagggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagtttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgata

SEQ ID NO: 43 - protein, 224 aa, Homo sapiens
MSTESMIRDVELAEEALPKKTGGPQGSRRCLFLSLFSFLIVAGATTLFCLLHFGVIGPQREEFPRDLSLISPLAQADEPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 44 - protein, 250 aa, Homo sapiens
MKATIILLLLAQVSWAGPFQQRGLFDFMLEDEASGIGPEVPDDRDFEPSLGPVCPFRCQCHLRVVQCSDLELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 45 - protein, 212 aa, Homo sapiens
MKATIILLLLAQVSWAGPFQQRGLFDFMLEDEASGIGPEVPDDRDFEPSLGPVCPFRCQCHLRVVQCSDLGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 46 - protein, 172 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHVVYLHNNNISVVGSSDFCPPGHNTKKASYSGVSLFSNPVQYWEIQPSTFRCVYVRSAIQLGNYK

SEQ ID NO: 47 - protein, 75 aa, Homo sapiens
MKATIILLLLAQVSWAGPFQQRGLFDFMLEDEASGIGPEVPDDRDFEPSLGPVCPFRCQCHLRVVQCSDLGCLPS

SEQ ID NO: 48 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 49 - protein, 239 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 50 - protein, 215 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 51 - protein, 5 aa, synthetic construct
  REGION 1..5: note = Description of Artificial Sequence: Synthetic peptide
GGGGS

SEQ ID NO: 52 - protein, 4 aa, synthetic construct
  REGION 1..4: note = Description of Artificial Sequence: Synthetic peptide
GGGS

SEQ ID NO: 53 - skipped

SEQ ID NO: 54 - protein, 18 aa, synthetic construct
  REGION 1..18: note = Description of Artificial Sequence: Synthetic peptide
KESGSVSSEQLAQFRSLD

SEQ ID NO: 55 - protein, 14 aa, synthetic construct
  REGION 1..14: note = Description of Artificial Sequence: Synthetic peptide
EGKSSGSGSESKST

SEQ ID NO: 56 - protein, 12 aa, synthetic construct
  REGION 1..12: note = Description of Artificial Sequence: Synthetic peptide
GSAGSAAGSGEF

SEQ ID NO: 57 - protein, 5 aa, synthetic construct
  REGION 1..5: note = Description of Artificial Sequence: Synthetic peptide
EAAAK

SEQ ID NO: 58 - protein, 5 aa, synthetic construct
  REGION 1..5: note = Description of Artificial Sequence: Synthetic peptide
EAAAR

SEQ ID NO: 59 - protein, 5 aa, synthetic construct
  REGION 1..5: note = Description of Artificial Sequence: Synthetic peptide
PAPAP

SEQ ID NO: 60 - protein, 12 aa, synthetic construct
  REGION 1..12: note = Description of Artificial Sequence: Synthetic peptide
AEAAAKEAAAKA

SEQ ID NO: 61 - DNA, 39 bp, synthetic construct
  misc_feature 1..39: note = Description of Artificial Sequence: Synthetic oligonucleotide
  source 1..39: mol_type = other DNA
aaaattgaaattttattttttttttttggaatataaata

SEQ ID NO: 62 - DNA, 1080 bp, Homo sapiens
  source 1..1080: mol_type = unassigned DNA
atgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatctcgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaa

SEQ ID NO: 63 - DNA, 4294 bp, synthetic construct
  misc_feature 1..4294: note = Description of Artificial Sequence: Synthetic polynucleotide
  source 1..4294: mol_type = other DNA
aaaaattgaaattttattttttttttttggaatataaataatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaaaaaaattgaaattttattttttttttttggaatataaataatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttagctggcgaaagggggatgtgctgcaaggcgattaagttgggtaacgccagggttttcccagtcacgacgttgtaaaacgacggccagtgaattgtaatacgactcactatagggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagtttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaaaattgaaattttattttttttttttggaatataaataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 64 - protein, 581 aa, synthetic construct
  REGION 1..581: note = Description of Artificial Sequence: Synthetic polypeptide
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGMVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 65 - DNA, 593 bp, synthetic construct
  misc_feature 1..593: note = Description of Artificial Sequence: Synthetic polynucleotide
  source 1..593: mol_type = other DNA
gatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggtctagactttttgtttatggggaaactctaaga

SEQ ID NO: 66 - DNA, 644 bp, synthetic construct
  misc_feature 1..644: note = Description of Artificial Sequence: Synthetic polynucleotide
  source 1..644: mol_type = other DNA
tatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaggatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtacttttttggatacgtcgcacatgttgaggttgcgacgaggcaggcgagcacgatgataatataccgcgccat

(19) United States
(12) Patent Application Publication
(10) Pub. No.: US 2024/0092852 A1
(43) Pub. Date: Mar. 21, 2024

(54) MULTI-ARMED MYXOMA VIRUS

(71) Applicant: OncoMyx Therapeutics, Inc., Phoenix, AZ (US)

(72) Inventors: Leslie Lynne SHARP, Rancho Santa Fe, CA (US); Lina FRANCO ACHURY, Tempe, AZ (US); Lino TORRES-DOMINGUEZ, Tempe, AZ (US)

(21) Appl. No.: 18/459,209

(22) Filed: Aug. 31, 2023

Related U.S. Application Data
(63) Continuation of application No. PCT/US2022/018164, filed on Feb. 28, 2022.
(60) Provisional application No. 63/155,195, filed on Mar. 1, 2021.

(51) Int. Cl. C07K 14/54; C07K 14/005; C07K 14/47; C12N 15/86

(52) U.S. Cl. CPC C07K 14/5434; C07K 14/005; C07K 14/4702; C12N 15/86; C12N 2710/24022; C12N 2710/24043

(57) ABSTRACT

Disclosed herein, in certain embodiments, are recombinant myxoma viruses (MYXVs) and nucleic acid constructs encoding the recombinant oncolytic virus genomes and parts thereof. In some embodiments, the nucleic acid constructs include at least a portion of myxoma virus (MYXV) genome and a transgene (e.g., IL-12) driven by poxvirus P11 late promoter. The transgene is inserted at the MYXV genome to reduce or disrupt the expression of M153 gene of the MYXV genome.

### CROSS REFERENCE

This application is a continuation of International Patent Application No. PCT/US2022/018164 filed Feb. 28, 2022, which claims priority to and the benefit of U.S. Provisional Patent Application No. 63/155,195, filed Mar. 1, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Aug. 29, 2023, is named 55842_708_301_SL.xml and is 148,429 bytes in size.

### FIELD

Disclosed herein are recombinant oncolytic viruses, i.e., myxoma viruses (MYXVs), nucleic acid constructs useful for making recombinant oncolytic viruses, and methods of use thereof.

### BACKGROUND

Current treatments used to treat various types of cancer tend to work by poisoning or killing the cancerous cell, but treatments that are toxic to cancer cells typically tend to be toxic to healthy cells as well. Moreover, the heterogenous nature of tumors is one of the primary reasons that effective treatments for cancer remain elusive. Current mainstream therapies such as chemotherapy and radiotherapy tend to be used within a narrow therapeutic window of toxicity. These types of therapies have limited applicability due to the varying types of tumor cells and the limited window in which these treatments can be administered.

### SUMMARY

Disclosed herein, in some aspects, is a recombinant nucleic acid comprising: at least a portion of myxoma virus (MYXV) genome and a first nucleic acid encoding interleukin-12 subunit beta (IL-12β); wherein the first nucleic acid is inserted at the MYXV genome to reduce or disrupt the expression of M153 gene of the MYXV genome; and wherein expression of the IL-12β is driven by a first poxvirus P11 late promoter.

In some embodiments, the IL-12β is human IL-12β. In some embodiments, the recombinant nucleic acid further comprises a second nucleic acid encoding interleukin-12 subunit alpha (IL-12α). In some embodiments, the IL-12α is human IL-12α. In some embodiments, the 5′ end of the second nucleic acid is coupled to the 3′-end of the first nucleic acid. In some embodiments, the first and second nucleic acids are coupled via a third nucleic acid encoding an elastin linker. In some embodiments, the recombinant nucleic acid further comprises a fourth nucleic acid encoding decorin. In some embodiments, the decorin is human decorin. In some embodiments, expression of the decorin is driven by a first sE/L promoter. In some embodiments, the 5′ end of the fourth nucleic acid is coupled to the 3′-end of the second nucleic acid. In some embodiments, the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; and (f) the fourth nucleic acid encoding the decorin. In some embodiments, the recombinant nucleic acid comprises, consists essentially of, or consists of a vMyx-P11 late promoter-hIL-12β-elastin linker-hIL-12α-sE/L promoter-hdecorin expression cassette. In some embodiments, the recombinant nucleic acid comprises, consists essentially of, or consists of a nucleotide sequence with at least 80%, at least 85%, at least 90%, at least 95%, or at least 98% sequence identity to nucleotides 1-2762 of SEQ ID NO: 10. In some embodiments, the recombinant nucleic acid comprises, consists essentially of, or consists of a nucleotide sequence that is nucleotides 1-2762 of SEQ ID NO: 10. In some embodiments, the recombinant nucleic acid further comprises a fifth nucleic acid encoding a reporter tag. In some embodiments, the reporter tag comprises a green fluorescent protein (GFP). In some embodiments, expression of the reporter tag is driven by a second sE/L promoter. In some embodiments, the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; (f) the fourth nucleic acid encoding the decorin; (g) the second sE/L promoter; and (h) the fifth nucleic acid encoding the reporter tag. In some embodiments, the recombinant nucleic acid comprises, consists essentially of, or consists of a vMyx-P11 late promoter-hIL-12β-elastin linker-hIL-12α-sE/L promoter-hdecorin-sE/L promoter-GFP expression cassette. In some embodiments, the recombinant nucleic acid comprises, consists essentially of, or consists of a nucleotide sequence with at least 80%, at least 85%, at least 90%, at least 95%, or at least 98% sequence identity to SEQ ID NO: 10 or SEQ ID NO: 11. In some embodiments, the recombinant nucleic acid comprises, consists essentially of, or consists of a nucleotide sequence that is SEQ ID NO: 10 or SEQ ID NO: 11. In some embodiments, the recombinant nucleic acid further comprises a sixth nucleic acid encoding tumor necrosis factor alpha (TNF-α). In some embodiments, the TNF-α is human TNF-α. In some embodiments, the TNF-α is a soluble polypeptide. In some embodiments, expression of the TNF-α is driven by a second poxvirus P11 late promoter. In some embodiments, the sixth nucleic acid is located between the second nucleic acid encoding IL-12α and the fourth nucleic acid encoding decorin. In some embodiments, the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the second poxvirus P11 late promoter; (f) the sixth nucleic acid encoding TNF-α; (g) the first sE/L promoter; (h) the fourth nucleic acid encoding the decorin; (i) optionally, the second sE/L promoter; and (j) optionally, the fifth nucleic acid encoding the reporter tag. In some embodiments, the recombinant nucleic acid comprises, consists essentially of, or consists of a vMyx-P11 late promoter-hIL-12β-elastin linker-hIL-12α-P11 late promoter-TNF-α-sE/L promoter-hdecorin expression cassette. In some embodiments, the recombinant nucleic acid comprises, consists essentially of, or consists of a nucleotide sequence with at least 80%, at least 85%, at least 90%, at least 95%, or at least 98% sequence identity to nucleotides 1-3507 of SEQ ID NO: 20. In some embodiments, the recombinant nucleic acid comprises, consists essentially of, or consists of a nucleotide sequence that is nucleotides 1-3507 of SEQ ID NO: 20. In some embodiments, the recombinant nucleic acid comprises or consists of a vMyx-P11 late promoter-hIL-12β-elastin linker-hIL-12α-P11 late promoter-TNF-α-sE/L promoter-hdecorin-sE/L promoter-GFP expression cassette. In some embodiments, the recombinant nucleic acid comprises, consists essentially of, or consists of a nucleotide sequence with at least 80%, at least 85%, at least 90%, at least 95%, or at least 98% sequence identity to SEQ ID NO: 20 or SEQ ID NO: 21. In some embodiments, the recombinant nucleic acid comprises, consists essentially of, or consists of a nucleotide sequence that is SEQ ID NO: 20 or SEQ ID NO: 21.

Disclosed herein, in some aspects, is a recombinant nucleic acid comprising at least a portion of myxoma virus (MYXV) genome, and a nucleic acid expression cassette inserted at the MYXV genome to reduce or disrupt expression of M153 gene of the MYXV genome, wherein nucleic acid expression cassette comprises, from 5′ to 3′: sE/L promoter-hdecorin-sE/L promoter-hIL-12β-IRES-hIL-12α-sE/L promoter-GFP.

In some embodiments, the recombinant nucleic acid comprises, consists essentially of, or consists of a sequence with at least 80%, at least 85%, at least 90%, at least 95%, or at least 98% sequence identity to SEQ ID NO: 25, SEQ ID NO: 26, SEQ ID NO: 63, nucleotides 1-3288 of SEQ ID NO: 25, or nucleotides 1-3534 of SEQ ID NO: 63. In some embodiments, the recombinant nucleic acid comprises, consists essentially of, or consists of a nucleotide sequence that is SEQ ID NO: 25, SEQ ID NO: 26, SEQ ID NO: 63, nucleotides 1-3288 of SEQ ID NO: 25, or nucleotides 1-3534 of SEQ ID NO: 63.

Disclosed herein, in some aspects, is a genetically engineered MYXV having enhanced immune-modulatory or anti-tumor activity, wherein at least 80% of a nucleic acid encoding M153 protein in MYXV genome is knocked out, wherein the genetically engineered MYXV comprises the recombinant nucleic acid of any one of the preceding embodiments.

In some embodiments, expression of the IL-12β is reduced in a non-cancer cell infected by the genetically engineered MYXV as compared to a non-cancer cell infected with a corresponding control myxoma virus in which expression of the IL-12β is driven by a sE/L promoter. In some embodiments, expression of the IL-12β is reduced in a peripheral blood mononuclear cell (PBMC) infected by the genetically engineered MYXV as compared to a PBMC infected by a corresponding control myxoma virus in which expression of the IL-12β is driven by a sE/L promoter. In some embodiments, expression of the IL-12β by a cell infected by the genetically engineered MYXV is reduced at four hours post-infection as compared to a cell infected by a corresponding control myxoma virus in which expression of the IL-12β is driven by a sE/L promoter.

Disclosed herein, in some aspects, is a genetically engineered MYXV comprising a nucleic acid that encodes a cytokine, wherein expression of the cytokine is driven by a poxvirus p11 late promoter, wherein the MYXV is genetically engineered to attenuate expression or activity of M153.

In some embodiments, the cytokine comprises IL-12β, IL-12α, or a combination thereof. In some embodiments, the cytokine comprises TNF-α. In some embodiments, at least 80% of a nucleic acid encoding the M153 is deleted in a genome of the genetically engineered MYXV. In some embodiments, expression of the cytokine is reduced in a non-cancer cell infected by the genetically engineered MYXV as compared to a non-cancer cell infected by a corresponding control myxoma virus in which expression of the cytokine is driven by a sE/L promoter. In some embodiments, expression of the cytokine is reduced in a PBMC infected by the genetically engineered MYXV as compared to a PBMC infected by a corresponding control myxoma virus in which expression of the cytokine is driven by a sE/L promoter. In some embodiments, expression of the cytokine by a cell infected by the genetically engineered MYXV is reduced at four hours post-infection as compared to a cell infected by a corresponding control myxoma virus in which expression of the cytokine is driven by a sE/L promoter. In some embodiments, the MYXV comprises a nucleic acid sequence that comprises, consists essentially of, or consists of a nucleotide sequence with at least 80%, at least 85%, at least 90%, at least 95%, or at least 98% sequence identity to SEQ ID NO 10, SEQ ID NO 11, SEQ ID NO: 20, SEQ ID NO: 21, SEQ ID NO: 25, SEQ ID NO: 26, SEQ ID NO: 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63. In some embodiments, the MYXV comprises a nucleic acid sequence that comprises, consists essentially of, or consists of SEQ ID NO 10, SEQ ID NO 11, SEQ ID NO: 20, SEQ ID NO: 21, SEQ ID NO: 25, SEQ ID NO: 26, SEQ ID NO: 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63. In some embodiments, the MYXV is genetically engineered Lausanne strain MYXV. In some embodiments, the poxvirus p11 late promoter comprises, consists essentially of, or consists of a nucleotide sequence with at least 90% sequence identity to SEQ ID NO: 2. In some embodiments, the poxvirus p11 late promoter comprises, consists essentially of, or consists of the nucleotide sequence of SEQ ID NO: 2.

Disclosed herein, in some aspects, is a mammalian cell treated ex vivo with the recombinant nucleic acid or the genetically engineered MYXV of any one of the preceding embodiments.

In some embodiments, the mammalian cell is a tumor cell. In some embodiments, the mammalian cell is a peripheral blood mononuclear cell (PBMC) or a bone marrow (BM) cell.

Disclosed herein, in some aspects, is a composition comprising the recombinant nucleic acid, genetically engineered MYXV, or mammalian cell of any one of the preceding embodiments.

In some embodiments, the composition is formulated for systemic administration. In some embodiments, the composition is formulated for local administration.

Disclosed herein, in some aspects, is a method of increasing an immune response against a tumor in a subject in need thereof, comprising administering to the subject the composition of any one of the preceding embodiments.

In some embodiments, the subject has, is suspected of having the tumor. In some embodiments, the administration is systemic administration. In some embodiments, the administering is intravenous. In some embodiments, the administering is local. In some embodiments, the administering is intratumoral. In some embodiments, the tumor comprises a solid tumor. In some embodiments, the tumor is a lung cancer, colon cancer, gastric cancer, liver cancer, breast cancer, or melanoma. In some embodiments, the administration improves the subject's survival. In some embodiments, the administration reduces cancer cell viability, or activates immunogenic cell death in the cancer. In some embodiments, the administration is performed in a dose and a schedule effective to increase expression of at least two cytokines in the tumor of the subject. In some embodiments, the administration is performed in a dose and a schedule effective to reduce volume of the tumor at least 10%. In some embodiments, the administration is performed in a dose and a schedule effective to reduce the growth of the tumor at least 10%. In some embodiments, the subject survives at least 10% longer than a subject administered a ten-fold higher dose of a corresponding control myxoma virus that expresses M153, lacks the recombinant nucleic acid, or a combination thereof.

### BRIEF DESCRIPTION OF THE DRAWINGS

The novel features of certain embodiments of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a schematic diagram showing a recombinant nucleic acid that can be used to generate a recombinant myxoma virus (HV11) disclosed herein.

FIG. 1B is a schematic diagram showing a recombinant nucleic acid and generation of a recombinant myxoma virus (HV11) comprising the recombinant nucleic acid.

FIG. 2A is a schematic diagram showing a recombinant nucleic acid that can be used to generate a recombinant myxoma virus (HV14) disclosed herein.

FIG. 2B is a schematic diagram showing a recombinant nucleic acid and generation of a recombinant myxoma virus (HV14) comprising the recombinant nucleic acid.

FIG. 3A is a schematic diagram showing a recombinant nucleic acid that can be used to generate a recombinant myxoma virus (HV12) disclosed herein.

FIG. 3B is a schematic diagram showing a recombinant nucleic acid and generation of a myxoma virus (HV12) comprising the recombinant nucleic acid.

FIG. 4A is a schematic diagram showing a recombinant nucleic acid that can be used to generate a recombinant myxoma virus (MV2) disclosed herein.

FIG. 4B is a schematic diagram showing a recombinant nucleic acid that can be used to generate a recombinant myxoma virus (MV4) disclosed herein.

FIG. 4C is a schematic diagram showing a recombinant nucleic acid that can be used to generate a recombinant myxoma virus (MV1) disclosed herein.

FIG. 4D is a schematic diagram showing a recombinant nucleic acid that can be used to generate a recombinant myxoma virus (MV3) disclosed herein.

FIG. 4E is a schematic diagram showing a recombinant nucleic acid that can be used to generate a recombinant myxoma virus (HV13) disclosed herein.

FIG. 4F is a schematic diagram showing a recombinant nucleic acid that can be used to generate a recombinant myxoma virus (MV5) disclosed herein.

FIG. 5A is a graph showing IL-12 release from Vero cells infected by HV11, HV12, HV13, or HV14.

FIG. 5B is a graph showing decorin release from Vero cells infected by HV11, HV12, HV13, or HV14.

FIG. 5C is a graph showing TNF-α release from Vero cells infected by HV13 or HV14.

FIG. 6A is a graph showing TNF-α release from Vero cells infected by HV11, HV12, HV13, or HV14 in dose (MOI) responsive manner.

FIG. 6B is a graph showing IL-12 release from Vero cells infected by HV11, HV12, HV13, or HV14 in dose (MOI) responsive manner.

FIG. 6C is a graph showing decorin release from Vero cells infected by HV11, HV12, HV13, or HV14 in dose (MOI) responsive manner.

FIG. 7A is a graph showing IL-12 release from Vero cells infected by HV11, HV12, HV13, or HV14 in time responsive manner.

FIG. 7B is a graph showing decorin release from Vero cells infected by HV11, HV12, HV13, or HV14 in time responsive manner.

FIG. 7C is a graph showing TNF-α release from Vero cells infected by HV11, HV12, HV13, or HV14 in time responsive manner.

FIG. 8 is a graph showing expression level of bifunctional IL-12 by Vero cells infected by HV11, HV12, HV13, or HV14 as measured by a reporter cell line.

FIG. 9A is a graph showing IL-12 detected in serum samples of immunodeficient A549 tumor-bearing mice infected by HV11 or HV12 via intravenous (IV) or intratumoral (IT) injection.

FIG. 9B is a graph showing IL-12 detected in tumor samples of immunodeficient A549 tumor-bearing mice infected by HV11 or HV12 via intravenous (IV) or intratumoral (IT) injection.

FIG. 10A is a graph showing TNF-α release from Vero cells infected by MV1, MV2, MV3, or MV4 in dose (MOI) responsive manner.

FIG. 10B is a graph showing IL-12 release from Vero cells infected by MV1, MV2, MV3, or MV4 in dose (MOI) responsive manner.

FIG. 10C is a graph showing decorin release from Vero cells infected by MV1, MV2, MV3, or MV4 in dose (MOI) responsive manner.

FIG. 11A is a graph showing IL-12 release from Vero cells infected by MV1, MV2, MV3, or MV4 in time responsive manner.

FIG. 11B is a graph showing decorin release from Vero cells infected by MV1, MV2, MV3, or MV4 in time responsive manner.

FIG. 11C is a graph showing TNF-α release from Vero cells infected by MV3 or MV4 in time responsive manner.

FIG. 12 is a graph showing levels of bifunctional IL-12 produced Vero cells infected by MV1, MV2, MV3, or MV4 as determined by a reporter cell assay.

FIG. 13A is a graph showing tumor volume changes in an EMT-6 breast carcinoma mouse model upon treatment with MV1 or MV3.

FIG. 13B is a survival plot of an EMT-6 breast carcinoma mouse model upon treatment with MV1 or MV3.

FIG. 13C is a graph showing tumor volume changes in EMT-6 mouse breast carcinoma upon re-challenge 59 days after initial treatment with the indicated myxoma virus.

FIG. 14A is a graph showing tumor volume changes in a B16-F10 mouse melanoma model upon treatment with MV1, MV2, MV3, or MV4 by intratumoral injection.

FIG. 14B is a survival plot of a B16-F10 mouse melanoma model upon treatment with MV1, MV2, MV3, or MV4 by intratumoral injection.

FIG. 14C is a graph showing tumor volume changes in B16-F10 mouse melanoma upon treatment with MV1, MV2, MV3, or MV4 by intravenous injection.

FIG. 14D is a survival plot of B16-F10 mouse melanoma animals upon treatment with MV1, MV2, MV3, or MV4 by intravenous injection.

FIG. 15A is a graph showing tumor volume changes in B16-F10 mouse melanoma upon treatment with MV1 by intratumoral injection.

FIG. 15B is a survival plot of B16-F10 mouse melanoma animals upon treatment with MV1 by intratumoral injection.

FIG. 15C is a graph showing tumor volume changes in B16-F10 mouse melanoma upon treatment with MV1 by intravenous injection.

FIG. 15D is a survival plot of B16-F10 mouse melanoma animals upon treatment with MV1 by intravenous injection.

FIG. 16A is a graph showing tumor volume changes in a B16-F10-Luc disseminated melanoma mouse model upon treatment with MV1, MV2, MV3, or MV4 by intravenous injection.

FIG. 16B is a survival plot of a B16-F10-Luc disseminated melanoma mouse model upon treatment with MV1, MV2, MV3, or MV4 by intravenous injection.

FIG. 17A is a graph showing tumor volume changes in B16-F10-Luc disseminated melanoma mouse model upon treatment with MV1 or MV2 by intravenous injection.

FIG. 17B is a survival plot of B16-F10-Luc disseminated melanoma mouse model upon treatment with MV1 or MV2 by intravenous injection.

FIG. 18A is a survival plot of K7M2-Luc disseminated osteosarcoma mouse model upon treatment with MV1 or MV2 by intravenous injection.

FIG. 18B is a survival plot of K7M2-Luc disseminated osteosarcoma mouse model upon treatment with MV1, MV2, MV3, or MV4 by intravenous injection.

FIG. 19A is a graph showing IL-12 release from Vero cells infected by MV1, MV2, MV5, or HV11 in a dose (MOI) responsive manner.

FIG. 19B is a graph showing IL-12 release from B16-F10 cells infected by MV1, MV2, MV5, or HV11 in a dose (MOI) responsive manner.

FIG. 19C is a graph showing decorin release from Vero cells infected by MV1, MV2, MV5, or HV11 in a dose (MOI) responsive manner.

FIG. 19D is a graph showing decorin release from B16-F10 cells infected by MV1, MV2, MV5, or HV11 in a dose (MOI) responsive manner.

FIG. 20A is a graph showing IL-12 release from Vero cells infected by MV1, MV2, MV5, or HV11 in a time responsive manner.

FIG. 20B is a graph showing IL-12 release from B16-F10 cells infected by MV1, MV2, MV5, or HV11 in a time responsive manner.

FIG. 20C is a graph showing decorin release from Vero cells infected by MV1, MV2, MV5, or HV11 in a time responsive manner.

FIG. 20D is a graph showing decorin release from B16-F10 cells infected by MV1, MV2, MV5, or HV11 in a time responsive manner.

FIG. 21A is a graph plotting the % maximum growth inhibition versus EC50 for human solid tumor cell lines infected with HV11.

FIG. 21B is a graph plotting the % maximum growth inhibition versus EC50 for human solid tumor cell lines infected with HV12.

FIG. 21C is a graph plotting the % maximum growth inhibition versus EC50 for human solid tumor cell lines infected with HV13.

FIG. 21D is a graph plotting the % maximum growth inhibition versus EC50 for human solid tumor cell lines infected with HV14.

FIG. 22A is a graph plotting the % maximum growth inhibition versus EC50 for human multiple myeloma cell lines infected with HV11 at 24 hours post-infection.

FIG. 22B is a graph plotting the % maximum growth inhibition versus EC50 for human multiple myeloma cell lines infected with HV11 at 72 hours post-infection.

FIG. 23A is a graph showing decorin production by human solid tumor cell lines 24 hours after infection with MYXV-GFP, HV11, HV12, HV13, or HV14.

FIG. 23B is a graph showing IL-12 production by human solid tumor cell lines 24 hours after infection with MYXV-GFP, HV11, HV12, HV13, or HV14

FIG. 23C is a graph showing TNF-α production by human solid tumor cell lines 24 hours after infection with MYXV-GFP, HV13, or HV14.

FIG. 24A is a graph showing production of decorin and IL-12 by human solid tumor cell lines 24 hours after infection with HV11.

FIG. 24B is a graph showing production of decorin and IL-12 by human solid tumor cell lines 24 hours after infection with HV12.

FIG. 24C is a graph showing production of decorin and IL-12 by human solid tumor cell lines 24 hours after infection with HV13.

FIG. 24D is a graph showing production of decorin and IL-12 by human solid tumor cell lines 24 hours after infection with HV14.

FIG. 24E is a graph showing production of decorin and TNF-α by human solid tumor cell lines 24 hours after infection with HV13.

FIG. 24F is a graph showing production of decorin and TNF-α by human solid tumor cell lines 24 hours after infection with HV14.

FIG. 25A is a graph showing decorin production by human multiple myeloma cell lines 24 hours after infection with MYXV-GFP or HV11.

FIG. 25B is a graph showing IL-12 production by human multiple myeloma cell lines 24 hours after infection with MYXV-GFP or HV11.

### DETAILED DESCRIPTION

Described herein are oncolytic viruses, specifically oncolytic poxviruses such as engineered oncolytic myxoma viruses. Myxoma viruses can be referred to herein as MYXV or vMyx.

Some embodiments relate to double or triple transgene-armed oncolytic viruses such as MYXVs, and methods of their use for treatment of cancers, such as solid and/or metastatic cancers. Some embodiments include a recombinant MYXV construct that expresses 2 human transgenes: a human IL-12 (hIL-12) that can amplify anti-tumor immune responses, and a human Decorin (hDecorin) that blocks TGF-beta signaling within tumor beds, or three human transgenes: a human cytokine (hTNF) that improves the efficacy of the treatment of cancers that metastasize to the lung or other parts of the body, hIL-12, and hDecorin.

In some embodiments, the MYXV is genetically engineered to inactivate, disrupt, or attenuate expression of an M153 gene or protein, for example, genetically engineered to attenuate an activity or expression level of the M153 gene or protein. The modification to the myxoma virus as described herein has unexpectedly improved the oncolytic activity of the MYXV when compared with unmodified MYXV, MYXV that contain an intact wild type M153 gene, or MYXV with modification at another gene locus. In addition to modification at the M153 locus, the MYXV can also include one or more transgenes that encode non-viral molecules, such as a TNF-α, IL-12, and/or decorin to further enhance the oncolytic activity, increase an anti-tumor immune response, or decrease adverse side effects of the MYXV.

Some embodiments relate to recombinant nucleic acid constructs such as virus double-transgene or triple-transgene constructs that encode the transgenes and can be integrated into the MYXV genome, e.g., to the M153 locus. In some embodiments, the transgenes and other modifications to the MYXV improve cancer therapy efficacy.

### Definitions

The section headings used herein are for organizational purposes only and are not to be construed as limiting the subject matter described.

The following explanations of terms are provided for the purpose of describing particular embodiments and examples only and are not intended to be limiting.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

An “effective amount” or “therapeutically effective amount” refers to an amount of a compound or composition of the disclosure that is sufficient to produce a desired effect, which can be a therapeutic and/or beneficial effect.

A “subject in need thereof” or “a subject in need of” is a subject known to have, or that is suspected of having a disease, or condition, such as a cancer.

As used herein, the term “inhibiting” or “treating” a disease refers to inhibiting the full development or progression of a disease or condition. “Treatment” refers to a therapeutic intervention that ameliorates a sign or symptom of a disease or pathological condition after it has begun to develop. The term “ameliorating,” with reference to a disease or pathological condition, refers to any observable or detectable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease, for example, compared to a control subject or cohort of subjects, or compared to before the treating. A “prophylactic” treatment is a treatment administered to a subject who does not exhibit signs of a disease or exhibits only early signs for the purpose of decreasing the risk of developing pathology or disease progression, for example metastatic cancer.

MYXV may infect cells that have a deficient innate anti-viral response. Having “a deficient innate anti-viral response” as used herein refers to a cell that, when exposed to a virus or when invaded by a virus, does not induce, substantially does not induce, or exhibits reduced anti-viral defense mechanisms, which can include inhibition of viral replication, production of interferon, induction of the interferon response pathway, and apoptosis. The term includes a cell, such as a cancer cell, that has a reduced or defective innate anti-viral response upon exposure to or infection by a virus as compared to a normal cell, for example, a non-infected or non-cancer cell. This includes a cell that is non-responsive to interferon and a cell that has a reduced or defective apoptotic response or induction of the apoptotic pathway. The deficiency may be due to various causes, including infection, genetic or epigenetic defect, or environmental stress. It will however be understood that when the deficiency is caused by a pre-existing infection, superinfection by MYXV may be excluded and a skilled person can readily identify such instances. A skilled person can readily determine without undue experimentation whether any given cell type has a deficient innate anti-viral response and therefore is susceptible to infection by MYXV. Thus, in certain embodiments, the MYXV is capable of infecting cells that have a deficient innate anti-viral response. In certain embodiments, the cells are non-responsive to interferon. In specific embodiments, the cell is a mammalian cancer cell. In certain embodiments, the cell is a human cancer cell including a human solid tumor cell. In certain embodiments, the cells that have a deficient innate anti-viral response comprise cancer cells.

### Engineered Myxoma Viruses

Disclosed herein, in certain embodiments, are myxoma viruses (MYXVs). The MYXV may comprise a wild-type strain of MYXV or it may comprise a genetically modified strain of MYXV. In some embodiments, the MYXV comprises a Lausanne strain. In some embodiments, the MYXV comprises or is engineered from a Lausanne strain, such as ATCC VR-1829; GenBank: GCF_000843685.1, or GenBank Accession Number AF170726.2, published on Jul. 11, 2019. Wild type Lausanne strain has a genome of a size of 161.8 kb with 171 genes in the genome in both directions (main and complementary strand). From these 171 genes, 159 genes have been found to have a predictive open reading frame (ORF). All ORFs have been assigned a designation with a letter R or L, depending on the direction of the transcription.

In some instances, the MYXV comprises a South American MYXV strain that circulates in Sylvilagus brasiliensis. In some instances, the MYXV comprises a Californian MYXV strain that circulates in Sylvilagus bachmani. In some instances, the MYXV comprises 6918, an attenuated Spanish field strain that comprises modifications in genes M009L, M036L, M135R, and M148R (for example, GenBank Accession number EU552530, published on Jul. 11, 2019). In some instances, the MYXV comprises 6918VP60-T2 (GenBank Accession Number EU552531, published on Jul. 11, 2019). In some instances, the MYXV comprises a Standard laboratory Strain (SLS). In some embodiments, the MYXV comprises a nucleic acid construct or MYXV genome as described herein.

In some instances, the MYXV is not a South American MYXV strain that circulates in Sylvilagus brasiliensis, or is not a derivative thereof. In some instances, the MYXV is not a Californian MYXV strain that circulates in Sylvilagus bachmani, or is not a derivative thereof. In some instances, the MYXV is not 6918, an attenuated Spanish field strain that comprises modifications in genes MOO9L, M036L, M135R, and M148R (for example, GenBank Accession number EU552530, published on Jul. 11, 2019), or is not a derivative thereof. In some instances, the MYXV is not 6918VP60-T2 (GenBank Accession Number EU552531, published on Jul. 11, 2019), or is not a derivative thereof. In some instances, the MYXV is not a Standard laboratory Strain (SLS) or a derivative thereof. In some embodiments, the MYXV is not an SG33 strain, a CNCM I-1594 strain, a Toulouse 1 strain, or a derivative thereof.

In some embodiments, a MYXV comprises an intact or functional M001 gene. In some embodiments, a MYXV comprises an intact or functional M151 gene. In some embodiments, a MYXV comprises an intact or functional M152 gene. In some embodiments, a MYXV comprises an intact or functional M153 gene. In some embodiments, a MYXV comprises an intact or functional M154 gene. In some embodiments, a MYXV comprises an intact or functional M156 gene. In some embodiments, a MYXV comprises two intact or functional copies of M008.1 gene. In some embodiments, a MYXV comprises two intact or functional copies of M008 gene. In some embodiments, a MYXV comprises two intact or functional copies of M007 gene. In some embodiments, a MYXV comprises two intact or functional copies of M006 gene. In some embodiments, a MYXV comprises two intact or functional copies of M005 gene. In some embodiments, a MYXV comprises two intact or functional copies of M004.1 gene. In some embodiments, a MYXV comprises two intact or functional copies of M004 gene. In some embodiments, a MYXV comprises two intact or functional copies of M003.2 gene. In some embodiments, a MYXV comprises two intact or functional copies of M003.1 gene. In some embodiments, a MYXV comprises two intact or functional copies of M002 gene. In some embodiments, a MYXV comprises an intact or functional M11L gene.

In some instances, the MYXV or a parental strain of an engineered MYXV disclosed herein comprises at least 70%, at least 75%, at least 80%, at least 85%, at least 90%, at least 95%, at least 96%, at least 97%, at least 98%, or at least 99%, such as between 95% and 98%, 95% and 99%, including 90%, 91%, 92%, 93%, 94%, 95%, 96%, 97%, 98%, 99% nucleic acid sequence identity to a sequence disclosed in Cameron, et al., “The complete DNA sequence of Myxoma Virus,” Virology 264: 298-318 (1999), which is incorporated by reference for such disclosure. In some cases, the MYXV comprises the sequence disclosed in Cameron, et al., “The complete DNA sequence of Myxoma Virus,” Virology 264: 298-318 (1999).

The degree of sequence identity between two sequences as disclosed herein can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A Needleman and Wunsch global alignment algorithm can be used to align two sequences over their entire length, maximizing the number of matches and minimizes the number of gaps. Default settings can be used.

In some embodiments, a MYXV is engineered to inactivate or attenuate an activity or expression level of a viral gene or protein. In some embodiments, the viral gene or protein is M153. In some embodiments, the inactivated or attenuated activity or expression level of the viral gene or protein results in the MYXV exhibiting enhanced anti-cancer activity in relation to a wild-type MYXV, or in relation to a MYXV not having the inactivated or attenuated activity or expression level of the viral gene or protein, for example, a MYXV that comprises a wild type M153 gene and/or expresses a wild type (e.g., functional) M153 protein. In some embodiments, the MYXV is engineered to inactivate or attenuate an activity or expression level of more than one viral gene or protein.

In some embodiments, the MYXV comprises a recombinant nucleic acid that encodes a non-viral molecule, for example, a transgene that encodes a protein not native to the MYXV, such as a cytokine or an extracellular matrix protein. In some embodiments, the MYXV includes a transgene such as a transgene described herein. In some embodiments, the transgene encodes a tumor necrosis factor (TNF, e.g., TNF-α), an interleukin-12 (IL-12), or a decorin. In some embodiments, the MYXV includes two, three, four, five, or more transgenes. In some embodiments, two or more transgenes are knocked in to a MYXV genome. In some embodiments, a transgene disrupts a gene in the MYXV genome, for example, a transgene inserted within or replaces part or all of the gene in the MYXV genome, thereby disrupting expression of the gene and/or the protein it encodes. Such a disruption can be referred to as a knockout (KO). In some embodiments, two or more transgenes are tandemly arrayed. Transgenes can be present in an expression cassette disclosed herein.

The MYXV may be modified to produce any non-viral molecule (e.g., modified to carry any transgene) that enhances the anticancer effect of the MYXV. Such a non-viral molecule can be involved in triggering apoptosis, or in targeting the infected cell for immune destruction, such as a non-viral molecule that stimulates a response to interferon (e.g., repairs a lack of response to interferon), or that results in the expression of a cell surface marker that stimulates an antibody response, such as a pathogen-associated molecular pattern, for example, a bacterial cell surface antigen. The MYXV can also be modified to produce a non-viral molecule involved in shutting off the neoplastic or cancer cell's proliferation and growth, thereby preventing the cells from dividing. In some embodiments, the MYXV is modified to produce therapeutic non-viral molecules, such as molecules involved in the synthesis of chemotherapeutic agents, or it can be modified to have increased replication levels in cells of the particular species from which the cells to be inhibited or killed are derived, for example, human cells.

In some embodiments, the MYXV includes a recombinant construct that encodes or expresses two or three separate non-viral molecules, for example, human transgenes (e.g., human TNF, human Decorin and/or human IL-12), and/or non-human mammalian transgenes (e.g., mouse TNF, mouse Decorin, and/or mouse IL-12). In some embodiments, the recombinant construct further encodes or expresses one or more reporter tags, for example, fluorescent proteins such as eGFP and dsRed.

In some embodiments, the MYXV is genetically engineered to attenuate an activity or expression level of its M153 gene and/or protein, for example, comprises a disruption of the viral M153 gene (M153-knockout, M153KO). In some embodiments, attenuating the activity or expression level of M153 improves the WIC-dependent anti-tumor immune responses to virus-infected cancer cells, for example, improves CD4+ T cell and/or CD8+ T cell responses to virus-infected cancer cells. In some embodiments, the MYXV is an oncolytic virus for use in treating cancer. Some embodiments combine a M153KO backbone with the immune-enhancing properties of transgenes disclosed herein to enhance the oncolytic properties of the MYXV.

In some embodiments, the MYXV encodes a TNF (e.g., TNF-α) transgene, an IL-12 transgene, a decorin transgene, or any combination of two or more of those. In some embodiments, the MYXV includes a TNF (e.g., TNF-α) transgene, an IL-12 transgene, and a decorin transgene. In some embodiments, the MYXV includes a TNF-α transgene and an IL-12 transgene. In some embodiments, the MYXV includes a TNF-α transgene and a decorin transgene. In some embodiments, the MYXV includes an IL-12 transgene and a decorin transgene.

In some embodiments, upon administration of a MYXV to a subject that expresses TNF, the TNF activates and jump-starts the innate and adaptive arms of the anti-tumor immune system and promotes cancer cell death in a by-stander paracrine-like manner. In some embodiments, the IL-12 amplifies the resulting anti-cancer innate and adaptive immune responses. In some embodiments, the decorin interrupts local immunosuppressive actions mediated by TGF-β, thus enhancing the actions of both TNF and IL-12 and promoting the anti-cancer immune response. In some embodiments, the synergistic actions of the three transgenes plus the effects of MYXV in the tumor microenvironment (TME) increase the immunotherapeutic potential of oncolytic MYXV vectors. In some embodiments, the addition of the human transgenes that encode non-viral molecules (hTNF, hIL-12, and/or hDecorin) to the MYXV genome improves the MYXV's capacity to trigger robust anti-tumor immune responses in the tumor microenvironment (TME).

In some embodiments, the MYXV is modified to enhance the ease of detection of the virus or cells infected by the virus. For example, the MYXV may be genetically modified to express a marker, such as a reporter tag, that can be readily detected by phase contrast microscopy, fluorescence microscopy, or by radioimaging. The marker can be an expressed fluorescent protein or an expressed enzyme that is involved in a colorimetric or radiolabeling reaction. In some embodiments, the marker includes a gene product that interrupts or inhibits a particular function of the cells being tested.

In some embodiments, the engineered MYXV comprises a fluorescent protein. Illustrative fluorescent proteins include blue/UV proteins such as TagBFP, Azurite, Sims, or Sapphire; cyan proteins such as ECFP, cerulean, or mTurquoise; green proteins such as green fluorescent protein (GFP), Emerald, mUKG, mWasabi, or Clover; yellow proteins such as EYFP, citrine, venus, or SYFP2; orange proteins such as monomeric Kusabira-Orange, mKO2, or mOrange; red proteins such as dsRed, mRaspberrym mCherry, mStrawberry, mTangerine, tdTomato, mApple, or mRuby; photoactivatible proteins such as PA-GFP, PAmCherry 1, or PATagRFP; and photoswitchable proteins such as Dropna. In some embodiments, the MYXV includes more than one fluorescent protein. In some embodiments the engineered MYXV does not encode a fluorescent protein.

In some embodiments, the MYXV comprises transgenes encoding decorin, IL-12, and optionally GFP, wherein one or more of the transgenes are inserted at the M153 locus (e.g., such that M153 is disrupted or knocked out). In some embodiments, the MYXV comprises transgenes encoding TNF-α, decorin, IL-12, and optionally GFP, wherein one or more of the transgenes are inserted at the M153 locus (e.g., such that M153 is disrupted or knocked out). In some embodiments, a recombinant nucleic acid disclosed herein that comprises the TNF-α, decorin, IL-12, and/or GFP is introduced into the M153 locus to generate a MYXV of the disclosure (e.g., such that M153 is disrupted or knocked out).

In some embodiments, the MYXV comprises a modification at or adjacent to one or more genes associated with rabbit cell tropism. In some instances, the one or more genes associated with rabbit cell tropism comprises M11L, M063, M135R, M136R, M-T2, M-T4, M-T5, or M-T7. In some instances, the one or more genes associated with rabbit cell tropism comprise M135R, M136R, or a combination thereof.

The MYXV may be prepared using standard techniques known in the art. For example, the virus may be prepared by infecting cultured rabbit cells, or immortalized permissive human or primate cells, with the MYXV strain that is to be used, allowing the infection to progress such that the virus replicates in the cultured cells and can be released by standard methods known in the art for disrupting the cell surface and thereby releasing the virus particles for harvesting. Once harvested, the virus titer may be determined by infecting a confluent lawn of permissive (e.g., rabbit) cells and performing a plaque assay.

### M153 Modification

The MYXV M153 gene product is an E3-Ubiquitin ligase that may participate in the down-regulation of diverse cellular receptors and proteins, for example, degradation of MHC Class I and CD4 in human cells. In some embodiments, a MYXV of the disclosure has an attenuated activity and/or expression level of M153 protein. In some embodiments, an attenuated activity and/or expression level of M153 protein can enhance presentation of immune epitopes, for example, MHC-dependent presentation of viral and/or cancer immune peptides. Enhanced presentation of immune epitopes by infected cancer cells can elicit stronger immune responses, including anti-cancer T cell responses, such as anti-cancer CD8+ T cell responses. In some embodiments, an attenuated activity and/or expression level of M153 protein increases direct antigen presentation from M153KO virus-infected tumor cells by MHC-I, and enhances immune activation mediated by the MYXV. In some embodiments, an attenuated activity and/or expression level of M153 protein increases CD4 expression or activity, thereby enhancing T cell activation and an anti-cancer immune response.

In some embodiments, the MYXV comprises a modification of an M153 gene. In some instances, the modification is a mutation that attenuates an activity or expression level of a protein encoded by the M153 gene (e.g., impairs the function of the protein encoded by the M153 gene).

In some instances, the mutation is a deletion, for example, a deletion that attenuates an activity or expression level of a protein encoded by the M153 gene. In some embodiments, the mutation is a deletion of at least about 1%, at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, at least about 97%, or at least about 99%, of the nucleic acid sequence of the M153 gene. In some embodiments, the mutation is a deletion of the entire M153 gene. In some cases, the modification is a partial deletion, for example, a deletion of about 10%, about 20%, about 30%, about 40%, about 50%, about 60%, about 70%, about 80%, about 90%, or about 95% of the nucleic acid sequence of the M153 gene. In some embodiments, the deletion is a deletion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 750, at least 500, at least 550, or at least 600 nucleic acids. In some embodiments, the deletion disrupts a promoter (e.g., a promoter that drives expression of M153 in a wild type MYXV). In some embodiments, the deletion introduces a stop codon into the M153 gene sequence, for example, a premature stop codon that prevents expression of a full length M153 transcript and/or protein.

In some instances, the mutation is an insertion, for example, an insertion that attenuates an activity or expression level of a protein encoded by the M153 gene. In some embodiments, the insertion comprises a transgene that encodes a non-viral molecule, for example, a transgene that encodes TNF, decorin, IL-12, a reporter tag, or a combination thereof. In some embodiments, the insertion comprises two transgenes. In some embodiments, the insertion comprises three transgenes. In some embodiments, the insertion comprises four transgenes. In some embodiments, the insertion comprises five transgenes. The transgene(s) can disrupt (e.g., interrupt) the viral M153 gene and attenuate an activity or expression level of a M153 transcript and/or protein. In some embodiments, the insertion comprises a transgene that encodes TNF. In some embodiments, the insertion comprises a transgene that encodes IL-12 and a transgene that encodes decorin. In some embodiments, the insertion comprises a transgene that encodes TNF and a transgene that encodes IL-12. In some embodiments, the insertion comprises a transgene that encodes TNF and a transgene that encodes decorin. In some embodiments, the insertion comprises a transgene that encodes TNF, a transgene that encodes IL-12, and a transgene that encodes decorin. In some embodiments, the insertion comprises one or more promoter(s) that drive expression of the one or more transgene(s). In some embodiments, the insertion comprises one or more promoters, e.g., a p11 promoter and/or an sE/L promoter. In some embodiments, the insertion disrupts a promoter (e.g., a promoter that drives expression of M153 in a wild type MYXV). In some embodiments, combining M153 gene disruption with transgene expression improves the anti-tumor properties of the resulting recombinant virus.

In some embodiments, the insertion is an insertion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 500, at least 600, at least 700, at least 800, at least 900, at least 1000, at least 1500, or at least 2000 nucleic acids.

In some embodiments, the mutation comprises an insertion and a deletion, for example, a deletion of one or more nucleotides of M153 and an insertion of one or more transgenes disclosed herein.

In some embodiments, the insertion introduces a stop codon into the M153 gene sequence, for example, a premature stop codon that prevents expression of a full length M153 transcript and/or protein. In some embodiments, the insertion alters the reading frame of the M153 gene sequence, thereby disrupting expression of the M153 transcript and/or protein.

In some instances, the mutation is a substitution, for example, a substitution that attenuates an activity or expression level of a protein encoded by the M153 gene. In some embodiments, at least 1, at least 2, at least 3, at least 4, at least 5, at least 7, at least 10, at least 20, at least 30 nucleic acids are substituted. In some embodiments, the substitution introduces a stop codon into the M153 gene sequence, for example, a premature stop codon that prevents expression of a full length M153 transcript and/or protein. In some embodiments, the substitution disrupts a promoter (e.g., a promoter that drives expression of M153 in a wild type MYXV).

In some embodiments, a modification or mutation disclosed herein attenuates the activity level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a corresponding MYXV that encodes a functional wild type M153.

In some embodiments, a modification or mutation disclosed herein attenuates the expression level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a corresponding MYXV that encodes a functional wild type M153.

In some embodiments, a MYXV disclosed herein has an activity level of the M153 protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a corresponding MYXV that encodes a functional wild type M153.

In some embodiments, a MYXV disclosed herein has an expression level of the M153 gene and/or protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a corresponding MYXV that encodes a functional wild type M153.

In some embodiments, an attenuated activity and/or expression level of M153 gene and/or protein increases activation of T cells in response to cells infected by a MYXV (e.g., activation of CD4+ or CD8+ T cells specific for a viral or cancer antigen) by at least about 5%, at least about 10%, at least about 20%, at least about 30%, at least about 40%, at least about 50%, at least about 60%, at least about 70%, at least about 80%, at least about 90%, at least about 95%, at least about 2-fold, at least about 5-fold, at least about 10-fold, or at least about 100-fold, for example, as determined by a flow cytometry assay measuring T cell proliferation or activation marker expression.

### TNF

In some embodiments, the MYXV comprises a transgene that encodes tumor necrosis factor (TNF) protein. In some embodiments, the TNF protein is a TNF-α protein. In some embodiments, the TNF-α protein is a human TNF-α protein. In some embodiments, the TNF-α protein is soluble. In some embodiments, the TNF-α protein is membrane- or surface-bound. In some embodiments, the TNF-α protein enhances the anti-cancer activity of the MYXV by activating anti-tumor immune cells or inducing cancer cell death.

TNF is a cytokine that is part of the innate inflammatory immune response. In some embodiments, TNF participates in amplifying acquired (e.g., adaptive) immune responses. TNF can be expressed as a cell surface immune ligand and it can also be secreted as a cleaved soluble trimeric cytokine when produced in specific cells that express the converting proteolytic enzymes (such as TACE) that catalyze cleavage and release of the soluble ligand, for example that are expressed at high levels in cells of the myeloid lineage. One TNF effector pathway is the induction of cellular death through the TNF Receptor-1 (TNFR1) pathway. In some embodiments, induction of the TNFR1 pathway by TNF leads to apoptosis or necroptosis. In some embodiments, TNF activates the innate and adaptive immune responses, for example, by activating anti-tumor CD8+ T cells and NK cells.

Despite the early hope that systemic administration of soluble TNF may function in humans as a potent anti-tumor drug, some clinical trials showed that the secreted cytokine caused severe systemic toxicities in patients treated systemically with the soluble ligand. Additionally, the systemic TNF treatment did not induce the dramatic anti-tumor effects in patients that was reported preclinically. TNF expressed by cells infected with a MYXV disclosed herein, e.g., a secreted or cell surface membrane form of TNF, may improve local cancer cell death by eliciting a greater degree of bystander cell killing in the tumor microenvironment, and also stimulate anti-cancer activity of various classes of immune cells residing within the same tumor beds, while minimizing systemic TNF-mediated adverse toxic effects.

In some embodiments, the TNF-α is encoded by a gene that replaces or is adjacent to an M135R gene of the MYXV genome. In some embodiments, the TNF-α gene is inserted between an M135R gene and an M136R gene of the MYXV genome. In some embodiments, the TNF-α gene is inserted in the intergenic region between an M135R gene and an M136R gene of the MYXV genome. In some embodiments, the TNF-α is encoded by a gene that is inserted between the M152 and M154 genes of the MYXV genome. In some embodiments, the TNF-α is encoded by a gene that replaces or disrupts an M153 gene of the MYXV genome. In some embodiments, the TNF-α gene replaces or disrupts an M153 gene of the MYXV genome, e.g., as part of an insertion of a recombinant nucleic acid disclosed herein.

In some embodiments, expression of the TNF-α gene is driven by a promoter such as a poxvirus synthetic early/late (sE/L) promoter. In some embodiments, expression of the TNF-α gene is driven by an internal ribosome entry site (IRES).

In some embodiments, expression of the TNF-α gene is driven by a P11 promoter (e.g., poxvirus P11 late promoter). In some embodiments, the use of the late promoter p11 limits or substantially limits the expression of TNF-α to cancer cells, which are permissive to the virus, and reduces expression of TNF-α in abortive infections of the virus in other cell types, such as peripheral blood mononuclear cells. In some embodiments, the use of the late promoter p11 limits toxicity associated with TNF-α expression from other promoters due to reduced expression in non-cancer cells, for example, at early time points after infection. A level of TNF-α expression can be as determined by an example disclosed herein.

In some embodiments, a MYXV of the disclosure comprises a recombinant nucleic acid that facilitates expression of TNF-α at a desired stage of cellular infection. In some embodiments, a MYXV of the disclosure comprises a recombinant nucleic acid that facilitates expression of TNF-α at an early stage of cellular infection, for example, a measurable level of TNF-α, or a level that is at least 100, at least 500, at least 1000, at least 5000, or at least 10000 pg/mL in the culture supernatant of infected cells in less than 18, less than 12, less than 6, less than 4, or less than 2 hours post-infection. The cells can be plated at approximately 1-1.5×10⁵ cells per replicate and/or infected at approximately 70% confluence or at least 70% confluence.

In some embodiments, a recombinant nucleic acid facilitates expression of TNF-α at a late stage of cellular infection by a MYXV that comprises the recombinant nucleic acid, for example, to produce a measurable level of TNF-α (e.g., above a limit of detection), or a level that is at least 100, at least 500, at least 1000, at least 5000, or at least 10000 pg/mL in the culture supernatant of infected cells (e.g., cancer cells or cells with a deficient innate anti-viral response) at about 6, about 12, about 18, about 20, about 24, about 30, about 36, or about 48 hours post-infection. In some embodiments, a recombinant nucleic acid facilitates expression of at least 100, at least 500, at least 1000, at least 5000, or at least 10000 pg/mL of TNF-α in the culture supernatant of infected cells (e.g., cancer cells or cells with a deficient innate anti-viral response) at about 6 hours post-infection. In some embodiments, a recombinant nucleic acid facilitates expression of at least 100, at least 500, at least 1000, at least 5000, or at least 10000 pg/mL of TNF-α in the culture supernatant of infected cells at about 12 hours post-infection. In some embodiments, a recombinant nucleic acid facilitates expression of at least 100, at least 500, at least 1000, at least 5000, or at least 10000 pg/mL of TNF-α in the culture supernatant of infected cells at about 18 hours post-infection. In some embodiments, a recombinant nucleic acid facilitates expression of at least 100, at least 500, at least 1000, at least 5000, or at least 10000 pg/mL of TNF-α in the culture supernatant of infected cells at about 24 hours post-infection. In some embodiments, a recombinant nucleic acid facilitates expression of at least 100, at least 500, at least 1000, at least 5000, or at least 10000 pg/mL of TNF-α in the culture supernatant of infected cells at about 32 hours post-infection. In some embodiments, a recombinant nucleic acid facilitates expression of at least 100, at least 500, at least 1000, at least 5000, or at least 10000 pg/mL of TNF-α in the culture supernatant of infected cells at about 48 hours post-infection. In some embodiments, TNF-α is not expressed at a level of at least 100, at least 500, at least 1000, at least 5000, or at least 10000 pg/mL until at least about 6 hours post-infection. In some embodiments, TNF-α is not expressed at a level of at least 100, at least 500, at least 1000, at least 5000, or at least 10000 pg/mL until at least about 12 hours post-infection. In some embodiments, TNF-α is not expressed at a level of at least 100, at least 500, at least 1000, at least 5000, or at least 10000 pg/mL until at least about 18 hours post-infection. In some embodiments, TNF-α is not expressed at a level of at least 100, at least 500, at least 1000, at least 5000, or at least 10000 pg/mL until at least about 24 hours post-infection. In some embodiments, TNF-α is not expressed at a level of at least 100, at least 500, at least 1000, at least 5000, or at least 10000 pg/mL until at least about 32 hours post-infection. In some embodiments, TNF-α is not expressed at a level of at least 100, at least 500, at least 1000, at least 5000, or at least 10000 pg/mL until at least about 48 hours post-infection. In some embodiments, the level of TNF-α is below a limit of detection at the recited time point. The infected cells can be cancer cells, for example, solid tumor cells, hematologic cancer cells, lung cancer cells, colorectal cancer cells, melanoma cells, multiple myeloma cells, NCI-N87 (gastric carcinoma), SK-MEL-1 (melanoma), COLO205 (colon cancer), LoVo (colorectal cancer), HCC1806 (acantholytic squamous cell carcinoma/breast cancer), HCC1599 (breast cancer), HT1080 (fibrosarcoma), SW620 (colorectal cancer), HEP3B (hepatocellular carcinoma), MKN-45 (metastatic gastric adenocarcinoma), SJSA-1 (osteosarcoma), HUH-7 (hepatocellular carcinoma), A673 (Ewing sarcoma), MDA-MB-435 (metastatic melanoma), H1975 (lung adenocarcinoma/non-small cell lung cancer), SK-MEL-28 (melanoma), HT-29 (colorectal adenocarcinoma), A204 (Rhabdomyosarcoma), A549 (lung adenocarcinoma), DLD-1 (colorectal adenocarcinoma), A375 (melanoma), MDA-MB-231 (metastatic breast adenocarcinoma), SK-MES-1 (lung squamous cell carcinoma), H358 (Bronchioalveolar carcinoma/non-small cell lung cancer), HEP-G2 (hepatoblastoma/hepatocellular carcinoma), MDA-MB-157 (metastatic breast carcinoma), KMS-34(r), LP-1, RMPI-8226, L363, NCI-H929, MM1.s, U266, KMS-34, or ANBL-6 cells. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, a myxoma virus disclosed herein elicits less than 100, less than 500, less than 1000, less than 5000, or less than 10000 pg/mL of TNF-α by non-cancer cells (e.g., PBMCs) at 3 hours post-infection. In some embodiments, a myxoma virus disclosed herein elicits less than 100, less than 500, less than 1000, less than 5000, or less than 10000 pg/mL of TNF-α by non-cancer cells (e.g., PBMCs) at 6 hours post-infection. In some embodiments, a myxoma virus disclosed herein elicits less than 100, less than 500, less than 1000, less than 5000, or less than 10000 pg/mL of TNF-α by non-cancer cells (e.g., PBMCs) at 12 hours post-infection. In some embodiments, a myxoma virus disclosed herein elicits less than 100, less than 500, less than 1000, less than 5000, or less than 10000 pg/mL of TNF-α by non-cancer cells (e.g., PBMCs) at 18 hours post-infection. In some embodiments, a myxoma virus disclosed herein elicits less than 100, less than 500, less than 1000, less than 5000, or less than 10000 pg/mL of TNF-α by non-cancer cells (e.g., PBMCs) at 24 hours post-infection. In some embodiments, a myxoma virus disclosed herein elicits less than 100, less than 500, less than 1000, less than 5000, or less than 10000 pg/mL of TNF-α by non-cancer cells (e.g., PBMCs) at 36 hours post-infection. In some embodiments, the level of TNF-α is below a limit of detection at the recited time point. The cells can be infected by treatment with the MYXV at a multiplicity of infection of 1. The cells can be plated at approximately 1-1.5×10⁵ cells per replicate and/or infected at approximately 70% confluence or at least 70% confluence. In some embodiments the level of TNF-α elicited is below a limit of detection.

In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells or cells with a deficient innate anti-viral response disclosed herein that are exposed to or infected with the same virus, for example, when evaluated at 6 hours post-infection. In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells disclosed herein that are exposed to or infected with the same virus, for example, when evaluated at 12 hours post-infection. In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells disclosed herein that are exposed to or infected with the same virus, for example, when evaluated at 18 hours post-infection. In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells disclosed herein that are exposed to or infected with the same virus, for example, when evaluated at 24 hours post-infection. In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells disclosed herein that are exposed to or infected with the same virus, for example, when evaluated at 36 hours post-infection. In some embodiments, a myxoma virus disclosed herein elicits a level TNF-α production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of TNF-α produced by a population of cancer cells disclosed herein that are exposed to or infected with the same virus, for example, when evaluated at 48 hours post-infection. In some embodiments, expression of TNF-α is below a limit of detection for the non-cancer cells (e.g., PBMCs) and is above a limit of detection for the cancer cells. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 6 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 12 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 18 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 24 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 36 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses TNF-α under regulatory control of an sE/L promoter at 48 hours post-infection. In some embodiments, the level of TNF-α produced under regulatory control of the p11 promoter is below a limit of detection at the recited time point and is above a limit of detection if driven by the sE/L promoter. The cells can be plated at approximately 1-1.5×10⁵ cells per replicate and/or infected at approximately 70% confluence or at least 70% confluence.

In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 6 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 12 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 18 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 24 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 36 hours post-infection. In some embodiments, upon infection of a population of cells (e.g., a population of non-cancer, PBMC, or cancer cells disclosed herein) with a MYXV that expresses TNF-α under regulatory control of a p11 promoter, the population of infected cells expresses a level TNF-α that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses TNF-α under regulatory control of an sE/L promoter at 48 hours post-infection. The cells can be plated at approximately 1-1.5×10⁵ cells per replicate and/or infected at approximately 70% confluence or at least 70% confluence.

In some instances, the TNF protein comprises at least 80%, at least 90%, at least 91%, at least 92%, at least 93%, at least 94%, at least 95%, at least 96%, at least 97%, at least 98%, or at least 99% sequence identity to the sequence illustrated in UniProtKB-P01375, published on Jul. 3, 2019 (Entry version 247). In some instances, the TNF protein comprises between 95% and 98%, or 95% and 99% sequence identity to the sequence illustrated in UniProtKB-P01375, published on Jul. 3, 2019 (Entry version 247). In some instances, the TNF protein comprises about 90%, about 91%, about 92%, about 93%, about 94%, about 95%, about 96%, about 97%, about 98%, or about 99% sequence identity to the sequence illustrated in UniProtKB-P01375, published on Jul. 3, 2019 (Entry version 247). In some embodiments, the TNF protein comprises the sequence illustrated in UniProtKB-P01375, published on Jul. 3, 2019 (Entry version 247).

In some instances, the TNF protein comprises at least 90%, at least 95%, at least 96%, at least 97%, at least 98%, or at least 99% sequence identity to residues 77-233 of UniProtKB-P01375. In some instances, the TNF protein comprises between 95% and 98%, or 95% and 99% sequence identity to residues 77-233 of UniProtKB-P01375. In some instances, the TNF protein comprises about 90%, about 91%, about 92%, about 93%, about 94%, about 95%, about 96%, about 97%, about 98%, or about 99% sequence identity to residues 77-233 of UniProtKB-P01375. In some embodiments, the TNF protein comprises residues 77-233 of UniProtKB-P01375.

In some instances, the TNF protein is encoded by a gene comprising at least 80%, at least 90%, at least 91%, at least 92%, at least 93%, at least 94%, at least 95%, at least 96%, at least 97%, at least 98%, or at least 99% sequence identity to SEQ ID NO: 18 or SEQ ID NO: 41. In some instances, the TNF protein is encoded by a gene comprising between 95% and 98%, or 95% and 99% sequence identity to SEQ ID NO: 18 or SEQ ID NO: 41. In some instances, the TNF protein is encoded by a gene comprising about 90%, about 91%, about 92%, about 93%, about 94%, about 95%, about 96%, about 97%, about 98%, or about 99% sequence identity to SEQ ID NO: 18 or SEQ ID NO: 41. In some embodiments, the TNF protein is encoded by a gene comprising, consisting essentially of, or consisting of SEQ ID NO: 18 or SEQ ID NO: 41. In some embodiments, the TNF is encoded by a gene comprising the sequence of SEQ ID NO: 18 or SEQ ID NO: 41. In some embodiments, the gene encoding the TNF comprises a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to that of SEQ ID NO: 18 or SEQ ID NO: 41.

In some instances, the TNF protein encoded by a MYXV or recombinant nucleic acid of the disclosure comprises, consists essentially of, or consists of at least 80%, at least 90%, at least 91%, at least 92%, at least 93%, at least 94%, at least 95%, at least 96%, at least 97%, at least 98%, or at least 99% sequence identity to SEQ ID NO: 35, residues 77-233 of SEQ ID NO: 35, or SEQ ID NO: 43. In some instances, the TNF protein comprises, consists essentially of, or consists of between 95% and 98%, or 95% and 99% sequence identity to SEQ ID NO: 35, residues 77-233 of SEQ ID NO: 35, or SEQ ID NO: 43. In some instances, the TNF protein comprises, consists essentially of, or consists of about 90%, about 91%, about 92%, about 93%, about 94%, about 95%, about 96%, about 97%, about 98%, or about 99% sequence identity to SEQ ID NO: 35, residues 77-233 of SEQ ID NO: 35, or SEQ ID NO: 43. In some embodiments, the TNF protein comprises, consists essentially of, or consists of SEQ ID NO: 35, residues 77-233 of SEQ ID NO: 35, or SEQ ID NO: 43.

In some embodiments, a MYXV does not encode a tumor necrosis factor (TNF) protein.

### IL-12

In some embodiments, the MYXV comprises (e.g., encodes) a non-viral molecule, for example, comprises one or more transgenes that encode(s) interleukin-12 (IL-12) protein. In some embodiments, the IL-12 protein is a human IL-12 protein. In some embodiments, the IL-12 protein is soluble. In some embodiments, the IL-12 protein is membrane- or surface-bound. In some embodiments, the IL-12 protein further enhances the anti-cancer activity of the MYXV by promoting immune cell differentiation or eliciting immune cell cytotoxicity.

IL-12 is a cytokine. In some embodiments, IL-12 promotes T helper type 1 (Th1) differentiation and enhances the cytotoxicity of natural killer (NK) cells and cytotoxic T lymphocytes (CTLs). In some embodiments, the actions of this IL-12 create an improved interconnection between the elements of innate and adaptive immunity to promote an anti-cancer immune response. In some embodiments, due to this bridging the innate and adaptive immunity, IL-12 enhances the anti-tumor effects of the MYXV. In some embodiments, IL-12 potently stimulates production of IFN-γ (a cytokine that coordinates mechanisms of anticancer defense), thereby enhancing the anti-tumor effects of the MYXV.

Clinical trials of systemic delivery of recombinant IL-12 cytokine therapy have not induced satisfactory outcomes in cancer patients due to toxicity events, the transient nature of systemically administered IL-12, and tumor-induced immunosuppression. Nevertheless, viruses expressing IL-12 locally within the tumor microenvironment (TME) may result in potent antitumor efficacy, for example, with IL-12 expression driven by an appropriate promoter. In some embodiments, expression of IL-12 from an oncolytic virus that is restricted to tumor beds, such that the transgenes are expressed locally within the TME, reduces the toxic effects associated with the systemic delivery of this cytokine. Thus, in some embodiments, the co-expression of the two subunits of IL-12 by a MYXV improves the anti-tumor immunity induced by an armed-MYXV against one or more type of cancers.

In some embodiments, IL-12 comprises an IL-12α (p35) subunit. In some embodiments, the IL-12α subunit is encoded by an IL-12α gene. In some embodiments, the IL-12α gene is a human IL-12a gene. In some embodiments, the IL-12α gene is driven by an IRES. In some embodiments, the IL-12α gene is driven by a promoter such as an sE/L promoter. In some embodiments, expression of the IL-12α gene is driven by a promoter such as a P11 promoter (e.g., poxvirus P11 late promoter, vaccinia virus late promoter P11). In some embodiments, the use of late promoter P11 limits or substantially limits the expression of IL-12α to cancer cells or cells with a deficient innate anti-viral response, which are permissive to the virus, and reduces expression of IL-12α in abortive infections of the virus in other cell types, such as peripheral blood mononuclear cells. In some embodiments, the use of late promoter P11 limits or reduces toxicity associated with IL-12α expression from other promoters (e.g., early promoter or sE/L promoter).

In some embodiments, IL-12α gene is between the M152 and M154 genes in the MYXV genome, e.g., in a MYXV with a deletion or disruption of M153. In some embodiments, IL-12α gene replaces or disrupts the M153 gene or a part thereof. In some embodiments, IL-12α gene is inserted in the intergenic region between an M135R gene and an M136R gene of the MYXV genome.

In some embodiments, IL-12 comprises an IL-12β(p40) subunit. In some embodiments, the IL-12β subunit is encoded by an IL-12β gene. In some embodiment, the IL-12β gene is a human IL-12β gene. In some embodiments, expression of the IL-12β gene is driven by an IRES. In some embodiments, expression of the IL-12β gene is driven by a promoter such as an sE/L promoter. In some embodiments, expression of the IL-12β gene is driven by a P11 promoter (e.g., poxvirus P11 late promoter, vaccinia virus late promoter P11). In some embodiments, the use of late promoter P11 limits or substantially limits the expression of IL-12β to cancer cells or cells with a deficient innate anti-viral response, which are permissive to the virus, and reduces expression of IL-12β in abortive infections of the virus in other cell types, such as peripheral blood mononuclear cells. In some embodiments, the use of late promoter p11 limits or reduces toxicity associated with IL-12β expression from other promoters.

In some embodiments, IL-12β gene is between the M152 and M154 genes in the MYXV genome, e.g., in a MYXV with a deletion or disruption of M153. In some embodiments, IL-12β gene replaces or disrupts a MYXV M153 gene. In some embodiments, IL-12β gene is inserted in the intergenic region between an M135R gene and an M136R gene of the MYXV genome.

In some embodiments, IL-12 comprises an IL-12α subunit and an IL-12β subunit. In some embodiments the IL-12α subunit and the IL-12β subunit are covalently linked. In some embodiments the IL-12α subunit and the IL-12β subunit are not covalently linked. In some embodiments the IL-12a subunit and the IL-12β subunit are expressed as one transcript. In some embodiments the IL-12α subunit and the IL-12β subunit are expressed as different transcripts, e.g., driven by separate promoters. In some embodiments the IL-12α subunit and the IL-12β subunit are expressed as one polypeptide, for example, with a peptide linker joining the two subunits. A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. A linker can be at least 1, at least 2, at least 3, at least 4, at least 5, at least 6, at least 7, at least 8, at least 9, or at least 10 amino acid residues in length. A linker can be at most 4, at most 5, at most 6, at most 7, at most 8, at most 9, at most 10, at most 15, at most 20, at most 25, at most 30, at most 40, or at most 50 amino acid residues in length. A flexible linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. A linker can comprise any of the sequences of SEQ ID NOs: 31 or 51-60 or repeats thereof. SEQ ID NOs: 51-56 provide examples flexible linker sequences. SEQ ID NOs: 57-60 provide examples of rigid linker sequences. A linker can be an elastin or elastin-like linker, for example, the linker provided in SEQ ID NO: 31 (encoded by, e.g., SEQ ID NO: 6), or a linker with 1, 2, 3, 4, or 5 amino acid insertions, deletions, or substitutions relative to SEQ ID NO: 31. A linker can be a self-cleaving linker, for example, a 2A peptide linker, e.g., to facilitate production of an appropriate ratio of IL-12 subunits.

In some embodiments, the MYXV expresses a relatively low level of IL-12. Relatively lower expression of IL-12 can be achieved, for example, by use of an IRES sequence between the sequences that encode the IL-12 subunits. In some embodiments, the MYXV expresses a relatively high level of IL-12. Relatively higher expression of IL-12 can be achieved, for example, by use of a suitable linker that joins the subunits of IL-12 in a single polypeptide, for example, an elastin linker, such as the linker of SEQ ID NO: 31.

In some embodiments, a level of IL-12 expression can be as determined by an example disclosed herein, e.g., the assay of example 2. For example, Vero cells can be infected with a MYXV of the disclosure at an MOI of 1, supernatant can be harvested at 24 hours post-infection, and the amount of IL-12 can be measured by ELISA. In some embodiments, a low level of IL-12 expression is less than 500, less than 400, less than 300, less than 200, less than 100, less than 50, less than 40, less than 30, less than 20, less than 10, or less than 5 ng/mL of IL-12 as determined by the ELISA assay of example 2. In some embodiments, a high level of IL-12 expression is more than 20, more than 30, more than 40, more than 50, more than 60, more than 70, more than 80, more than 90, more than 100, more than 150, more than 200, more than 250, more than 300, more than 400, or more than 500 ng/mL of IL-12 as determined by the assay of example 2. In some embodiments, a high level of IL-12 expression is more than 150 ng/mL of IL-12, and a low level of IL-12 expression is less than 150 ng/mL of IL-12. The cells can be plated at approximately 1-1.5×10⁵ cells per replicate and/or infected at approximately 70% confluence or at least 70% confluence.

In some embodiments, a MYXV of the disclosure comprises a recombinant nucleic acid that facilitates expression of IL-12 at a desired stage of cellular infection. In some embodiments, a MYXV of the disclosure comprises a recombinant nucleic acid that facilitates expression of IL-12 at an early stage of cellular infection, for example, to produce a measurable level of IL-12 (e.g., above a limit of detection), or a level that is at least 100, at least 500, at least 1000, at least 5000, or 10000 pg/mL in the culture supernatant of infected cells in less than 18, less than 12, less than 6, less than 4, or less than 2 hours post-infection. The cells can be plated at approximately 1-1.5×10⁵ cells per replicate and/or infected at approximately 70% confluence or at least 70% confluence.

In some embodiments, a recombinant nucleic acid facilitates expression of IL-12 at a late stage of cellular infection by a MYXV that comprises the recombinant nucleic acid, for example, to produce a measurable level of IL-12 (e.g., above a limit of detection), or a level that is at least 100, at least 500, at least 1000, at least 5000, at least 10,000, at least 50,000, at least 100,000, at least 500,000, or at least 1,000,000 pg/mL in the culture supernatant of infected cells (e.g., cancer cells or cells with a deficient innate anti-viral response) at about 6, about 12, about 18, about 20, about 24, about 30, about 36, or about 48 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells (e.g., cancer cells or cells with a deficient innate anti-viral response) at about 6 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells at about 12 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells at about 18 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells at about 24 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells at about 32 hours post-infection. In some embodiments, a recombinant nucleic acid facilitates expression of at least 100, at least 500, at least 1000, at least 5000, at least 10,000, at least 50,000, at least 100,000, at least 500,000, or at least 1,000,000 pg/mL of IL-12 in the culture supernatant of infected cells at about 48 hours post-infection. The cells can be plated at approximately 1-1.5×10⁵ cells per replicate and/or infected at approximately 70% confluence or at least 70% confluence.

In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 6 hours post-infection. In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 12 hours post-infection. In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 18 hours post-infection. In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 24 hours post-infection. In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 32 hours post-infection. In some embodiments, IL-12 is not expressed at a level of at least 100, at least 500, at least 1000, at least 5000, at least 10,000, at least 50,000, at least 100,000, at least 500,000, or at least 1,000,000 pg/mL until at least about 48 hours post-infection. In some embodiments, the IL-12 is below a limit of detection at the recited time point. The infected cells can be cancer cells, for example, solid tumor cells, hematological cancer cells, lung cancer cells, colorectal cancer cells, melanoma cells, multiple myeloma cells, NCI-N87 (gastric carcinoma), SK-MEL-1 (melanoma), COLO205 (colon cancer), LoVo (colorectal cancer), HCC1806 (acantholytic squamous cell carcinoma/breast cancer), HCC1599 (breast cancer), HT1080 (fibrosarcoma), SW620 (colorectal cancer), HEP3B (hepatocellular carcinoma), MKN-45 (metastatic gastric adenocarcinoma), SJSA-1 (osteosarcoma), HUH-7 (hepatocellular carcinoma), A673 (Ewing sarcoma), MDA-MB-435 (metastatic melanoma), H1975 (lung adenocarcinoma/non-small cell lung cancer), SK-MEL-28 (melanoma), HT-29 (colorectal adenocarcinoma), A204 (Rhabdomyosarcoma), A549 (lung adenocarcinoma), DLD-1 (colorectal adenocarcinoma), A375 (melanoma), MDA-MB-231 (metastatic breast adenocarcinoma), SK-MES-1 (lung squamous cell carcinoma), H358 (Bronchioalveolar carcinoma/non-small cell lung cancer), HEP-G2 (hepatoblastoma/hepatocellular carcinoma), MDA-MB-157 (metastatic breast carcinoma), KMS-34(r), LP-1, RMPI-8226, L363, NCI-H929, MM1.s, U266, KMS-34, or ANBL-6 cells. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, a myxoma virus disclosed herein elicits less than 100, less than 500, less than 1000, less than 5000, less than 10,000, less than 50,000, less than 100,000, less than 500,000, or less than 1,000,000 pg/mL of IL-12 by cells (e.g., non-cancer cells, PBMCs) at 6 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of IL-12 by cells (e.g., non-cancer cells, PBMCs) at 12 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of IL-12 by cells (e.g., non-cancer cells, PBMCs) at 18 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of IL-12 by cells (e.g., non-cancer cells, PBMCs) at 24 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of IL-12 by cells (e.g., non-cancer cells, PBMCs) at 36 hours post-infection. In some embodiments, the IL-12 is below a limit of detection. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 6 hours post-infection. In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 12 hours post-infection. In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 18 hours post-infection. In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 24 hours post-infection. In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 36 hours post-infection. In some embodiments, a myxoma virus disclosed herein elicits a level IL-12 production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of IL-12 produced by a population of cancer cells disclosed herein that have been infected with or exposed to the same virus, for example, when evaluated at 48 hours post-infection. In some embodiments the level of IL-12 produced by the non-cancer cells (e.g., PBMCs) is below a limit of detection. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 6 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 12 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 18 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 24 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 36 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a population of cells infected with a corresponding MYXV that expresses IL-12 under regulatory control of an sE/L promoter at 48 hours post-infection. In some embodiments, the level of IL-12 produced under regulatory control of the p11 promoter is below a limit of detection at the recited time point and is above a limit of detection if driven by the sE/L promoter. The cells can be plated at approximately 1-1.5×10⁵ cells per replicate and/or infected at approximately 70% confluence or at least 70% confluence.

In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 6 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 12 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 18 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 24 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 36 hours post-infection. In some embodiments, upon infection of a population of cells (e.g., a population of non-cancer, PBMC, or cancer cells disclosed herein) with a MYXV that expresses IL-12 under regulatory control of a p11 promoter, the population of infected cells expresses a level IL-12 that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold higher than a population of cells infected with a corresponding MYXV that expresses IL-12 under regulatory control of an sE/L promoter at 48 hours post-infection. The cells can be plated at approximately 1-1.5×10⁵ cells per replicate and/or infected at approximately 70% confluence or at least 70% confluence.

In some embodiments, one or both of the IL-12 subunits can be truncated. An example of an IL-12 with a truncated subunit is provided in SEQ ID NO: 36, which comprises mouse IL-12β(SEQ ID NO: 37), an elastin linker (SEQ ID NO: 31), and a truncated mouse IL-12α (SEQ ID NO: 38).

In some instances, the IL-12α subunit comprises, consists essentially of, or consists of an amino acid sequence with at least 85%, at least 90%, at least 91%, at least 92%, at least 93%, at least 94%, at least 95%, at least 96%, at least 97%, at least 98%, or at least 99% sequence identity to SEQ ID NO: 29, residues 35-253 of SEQ ID NO: 29, residues 57-253 of SEQ ID NO: 29, SEQ ID NO: 30, SEQ ID NO: 38, SEQ ID NO: 39, SEQ ID NO: 48, SEQ ID NO: 49, or SEQ ID NO: 50.

In some instances, the IL-12α subunit comprises, consists essentially of, or consists of an amino acid sequence with between 95% and 98%, or 95% and 99% sequence identity to SEQ ID NO: 29, residues 35-253 of SEQ ID NO: 29, residues 57-253 of SEQ ID NO: 29, SEQ ID NO: 30, SEQ ID NO: 38, SEQ ID NO: 39, SEQ ID NO: 48, SEQ ID NO: 49, or SEQ ID NO: 50. In some instances, the IL-12α subunit comprises about 90%, about 91%, about 92%, about 93%, about 94%, about 95%, about 96%, about 97%, about 98%, or about 99% sequence identity to SEQ ID NO: 29, residues 35-253 of SEQ ID NO: 29, residues 57-253 of SEQ ID NO: 29, SEQ ID NO: 30, SEQ ID NO: 38, SEQ ID NO: 39, SEQ ID NO: 48, SEQ ID NO: 49, or SEQ ID NO: 50. In some embodiments, the IL-12α subunit comprises, consists essentially of, or consists of SEQ ID NO: 29, residues 35-253 of SEQ ID NO: 29, residues 57-253 of SEQ ID NO: 29, SEQ ID NO: 30, SEQ ID NO: 38, SEQ ID NO: 39, SEQ ID NO: 48, SEQ ID NO: 49, or SEQ ID NO: 50.

In some instances, the IL-12β subunit comprises, consists essentially of, or consists of an amino acid sequence with at least 85%, at least 90%, at least 91%, at least 92%, at least 93%, at least 94%, at least 95%, at least 96%, at least 97%, at least 98%, or at least 99% sequence identity to SEQ ID NO: 28, residues 23-328 of SEQ ID NO: 28, or SEQ ID NO: 37. In some instances, the IL-12β subunit comprises, consists essentially of, or consists of an amino acid sequence with between 95% and 98%, or 95% and 99% sequence identity to SEQ ID NO: 28, residues 23-328 of SEQ ID NO: 28, or SEQ ID NO: 37. In some instances, the IL-12β subunit comprises about 90%, about 91%, about 92%, about 93%, about 94%, about 95%, about 96%, about 97%, about 98%, or about 99% sequence identity to SEQ ID NO: 28, residues 23-328 of SEQ ID NO: 28, or SEQ ID NO: 37. In some embodiments, the IL-12β subunit comprises, consists essentially of, or consists of SEQ ID NO: 28, residues 23-328 of SEQ ID NO: 28, or SEQ ID NO: 37.

In some instances, the IL-12 comprises at least 85%, at least 90%, at least 91%, at least 92%, at least 93%, at least 94%, at least 95%, at least 96%, at least 97%, at least 98%, or at least 99% sequence identity to SEQ ID NO: 34. In some instances, the IL-12 comprises between 95% and 98%, or 95% and 99% sequence identity to SEQ ID NO: 34. In some instances, the IL-12 comprises about 90%, about 91%, about 92%, about 93%, about 94%, about 95%, about 96%, about 97%, about 98%, or about 99% sequence identity to SEQ ID NO: 34. In some embodiments, the IL-12 comprises, consists essentially of, or consists of SEQ ID NO: 34.

In some instances, the IL-12α subunit is encoded by a gene comprising at least 90%, at least 91%, at least 92%, at least 93%, at least 94%, at least 95%, at least 96%, at least 97%, at least 98%, or at least 99% sequence identity to SEQ ID NO: 4 or SEQ ID NO: 5. In some instances, the IL-12α subunit is encoded by a gene comprising between 95% and 98%, or 95% and 99% sequence identity to SEQ ID NO: 4 or SEQ ID NO: 5. In some instances, the IL-12α subunit is encoded by a gene comprising about 90%, about 91%, about 92%, about 93%, about 94%, about 95%, about 96%, about 97%, about 98%, or about 99% sequence identity to SEQ ID NO: 4 or SEQ ID NO: 5. In some embodiments, the IL-12α subunit is encoded by a gene comprising, consisting essentially of, or consisting of SEQ ID NO: 4 or SEQ ID NO: 5. In some embodiments, the IL-12α subunit is encoded by a gene comprising the sequence of SEQ ID NO: 4 or SEQ ID NO: 5. In some embodiments, the gene encoding the IL-12α subunit comprises a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to that of SEQ ID NO: 4 or SEQ ID NO: 5.

In some instances, the IL-12β subunit is encoded by a gene comprising at least 90%, at least 91%, at least 92%, at least 93%, at least 94%, at least 95%, at least 96%, at least 97%, at least 98%, or at least 99% sequence identity to SEQ ID NO: 3. In some instances, the IL-12β subunit is encoded by a gene comprising between 95% and 98%, or 95% and 99% sequence identity to SEQ ID NO: 3. In some instances, the IL-12β subunit is encoded by a gene comprising about 90%, about 91%, about 92%, about 93%, about 94%, about 95%, about 96%, about 97%, about 98%, or about 99% sequence identity to SEQ ID NO: 3. In some embodiments, the IL-12β subunit is encoded by a gene comprising, consisting essentially of, or consisting of SEQ ID NO: 3. In some embodiments, the IL-12β subunit is encoded by a gene comprising the sequence of SEQ ID NO: 3. In some embodiments, the gene encoding the IL-12β subunit comprises a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to that of SEQ ID NO: 3.

In some instances, the IL-12 is encoded by a gene comprising at least 90%, at least 91%, at least 92%, at least 93%, at least 94%, at least 95%, at least 96%, at least 97%, at least 98%, or at least 99% sequence identity to SEQ ID NO: 9. In some instances, the IL-12 is encoded by a gene comprising between 95% and 98%, or 95% and 99% sequence identity to SEQ ID NO: 9. In some instances, the IL-12 is encoded by a gene comprising about 90%, about 91%, about 92%, about 93%, about 94%, about 95%, about 96%, about 97%, about 98%, or about 99% sequence identity to SEQ ID NO: 9. In some embodiments, the IL-12 is encoded by a gene comprising, consisting essentially of, or consisting of SEQ ID NO: 9. In some embodiments, the IL-12 is encoded by a gene comprising the sequence of SEQ ID NO: 9. In some embodiments, the gene encoding the IL-12 comprises a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to that of SEQ ID NO: 9.

### Decorin

In some embodiments, the MYXV comprises a transgene that encodes decorin. In some embodiments, the decorin protein is a human decorin protein. In some embodiments, the decorin protein is soluble. In some embodiments, the decorin protein is membrane- or surface-bound. In some embodiments, the decorin protein enhances the anti-cancer activity of the MYXV by blocking or decreasing TGF-β signaling.

Decorin is a member of the extracellular matrix proteoglycans family that exists and functions within stromal tissues and epithelial cells. In some embodiments, decorin affects the biology of different types of cancer by directly or indirectly targeting signaling molecules involved in cell growth, survival, metastasis and/or angiogenesis. In some embodiments, decorin blocks TGF-β-induced signaling. In some embodiments, TGF-β is a cytokine that contributes to immune suppression in some tumor microenvironments (TMEs). In some cases, TGF-β converts effector T-cells, which may otherwise recognize and attack cancer cells, into regulatory (suppressor) T-cells, which instead turn off or reduce the innate inflammatory reactions and acquired immune pathways needed to recognize and eliminate the cancer cells. In multiple type of cancers, parts of the TGF-β signaling pathways are mutated, and this cytokine no longer controls at least some of the cell targets. These cancer cells may proliferate and increase their endogenous production of TGF-β, which may act on the surrounding stromal cells, immune cells, endothelial and smooth-muscle, causing local immunosuppression within the cancer tissue and tumor bed angiogenesis, which makes the cancer even more invasive. Hence, in some embodiments, an oncolytic MYXV vector expressing decorin blocks TGF-β directly within the TME and thereby induces a stronger anti-tumor immune response than a MYXV not expressing the decorin.

Additionally, decorin can inhibit tumor cell growth and proliferation. Viral delivery of decorin into various solid tumors may directly counteract tumorigenesis. In some embodiments, decorin is used as an anti-cancer target for at least some types of cancer that are protected by the local over-expression of TGF-0.

In some embodiments, the decorin protein is encoded by a decorin gene. In some embodiments, the decorin gene is a human decorin gene. In some embodiments, the decorin gene is driven by an IRES. In some embodiments, the decorin gene is driven by a promoter such as an sE/L promoter, e.g., for expression in multiple stages of the infectious cycle. In some embodiments, expression of the decorin gene is driven by a promoter such as a P11 promoter (e.g., poxvirus P11 late promoter, vaccinia virus late promoter P11). In some embodiments, the use of late promoter P11 limits or substantially limits the expression of decorin to cancer cells, which are permissive to the virus, and reduces expression of decorin in abortive infections of the virus in other cell types, such as peripheral blood mononuclear cells. In some embodiments, the use of late promoter P11 limits toxicity associated with decorin expression from other promoters.

In some embodiments, a MYXV of the disclosure comprises a recombinant nucleic acid that facilitates expression of decorin at a desired stage of cellular infection. In some embodiments, a MYXV of the disclosure comprises a recombinant nucleic acid that facilitates expression of decorin at an early stage of cellular infection, for example, to produce a measurable level of decorin, or a level that is at least 10, at least 100, at least 500, at least 1000, at least 5000, at least 10,000, at least 50,000, at least 100,000, at least 500,000, or at least 1,000,000 pg/mL in the culture supernatant of infected cells in less than 18, less than 12, less than 6, less than 4, or less than 2 hours post-infection. The cells can be plated at approximately 1-1.5×10⁵ cells per replicate and/or infected at approximately 70% confluence or at least 70% confluence.

In some embodiments, a recombinant nucleic acid facilitates expression of decorin at a late stage of cellular infection by a MYXV that comprises the recombinant nucleic acid, for example, to produce a measurable level of decorin (e.g., above a limit of detection), or a level that is at least 100, at least 500, at least 1000, at least 5000, at least 10,000, at least 50,000, at least 100,000, at least 500,000, or at least 1,000,000 pg/mL in the culture supernatant of infected cells (e.g., cancer cells) at about 6, about 12, about 18, about 20, about 24, about 30, about 36, or about 48 hours post-infection. The cells can be plated at approximately 1-1.5×10⁵ cells per replicate and/or infected at approximately 70% confluence or at least 70% confluence.

In some embodiments, decorin is not expressed at a level of at least 100, at least 500, at least 1000, at least 5000, at least 10,000, at least 50,000, at least 100,000, at least 500,000, or at least 1,000,000 pg/mL until at least about 6, at least about 12, at least about 18, at least about 24, at least about 26, or at least about 48 hours post-infection. The infected cells can be cancer cells, for example, solid tumor cells, hematological cancer cells, lung cancer cells, colorectal cancer cells, melanoma cells, multiple myeloma cells, or another cell type disclosed herein. The cells can be plated at approximately 1-1.5×10⁵ cells per replicate and/or infected at approximately 70% confluence or at least 70% confluence.

In some embodiments, a myxoma virus disclosed herein elicits at least 100, at least 500, at least 1000, at least 5000, at least 10,000, at least 50,000, at least 100,000, at least 500,000, or at least 1,000,000 pg/mL of decorin by cells (e.g., cancer cells, non-cancer cells, PBMCs) at 6 hours post-infection. In some embodiments, a myxoma virus disclosed herein elicits at least 100, at least 500, at least 1000, at least 5000, at least 10,000, at least 50,000, at least 100,000, at least 500,000, or at least 1,000,000 pg/mL of decorin by cells (e.g., cancer cells, non-cancer cells, PBMCs) at 12 hours post-infection. In some embodiments, a myxoma virus disclosed herein elicits at least 100, at least 500, at least 1000, at least 5000, at least 10,000, at least 50,000, at least 100,000, at least 500,000, or at least 1,000,000 pg/mL of decorin by cells (e.g., cancer cells, non-cancer cells, PBMCs) at 18 hours post-infection. In some embodiments, a myxoma virus disclosed herein elicits at least 100, at least 500, at least 1000, at least 5000, at least 10,000, at least 50,000, at least 100,000, at least 500,000, or at least 1,000,000 pg/mL of decorin by cells (e.g., cancer cells, non-cancer cells, PBMCs) at 24 hours post-infection. In some embodiments, a myxoma virus disclosed herein elicits at least 100, at least 500, at least 1000, at least 5000, at least 10,000, at least 50,000, at least 100,000, at least 500,000, or at least 1,000,000 pg/mL of decorin by cells (e.g., cancer cells, non-cancer cells, PBMCs) at 36 hours post-infection. The cells can be plated at approximately 1-1.5×10⁵ cells per replicate and/or infected at approximately 70% confluence or at least 70% confluence. The cells can be plated at approximately 1-1.5×10⁵ cells per replicate and/or infected at approximately 70% confluence or at least 70% confluence.

In some embodiments, a myxoma virus disclosed herein elicits less than 100, less than 500, less than 1000, less than 5000, less than 10,000, less than 50,000, less than 100,000, less than 500,000, or less than 1,000,000 pg/mL of decorin by cells (e.g., non-cancer cells, PBMCs) at 6 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of decorin by cells (e.g., non-cancer cells, PBMCs) at 12 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of decorin by cells (e.g., non-cancer cells, PBMCs) at 18 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of decorin by cells (e.g., non-cancer cells, PBMCs) at 24 hours post-infection. In some embodiments, a myxoma virus disclosed herein elicits less than 100, less than 500, less than 1000, less than 5000, less than 10,000, less than 50,000, less than 100,000, less than 500,000, or less than 1,000,000 pg/mL of decorin by cells (e.g., non-cancer cells, PBMCs) at 36 hours post-infection. In some embodiments, the level of decorin is below a limit of detection at the recited time point. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 6 hours post-infection. In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 12 hours post-infection. In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 18 hours post-infection. In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 24 hours post-infection. In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 36 hours post-infection. In some embodiments, a myxoma virus disclosed herein elicits a level of decorin production by a population of non-cancer cells (e.g., PBMCs) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold lower than a level of decorin produced by a population of cancer cells disclosed herein that is infected with or exposed to the same virus, for example, when evaluated at 48 hours post-infection. In some embodiments, the level of decorin production is below a limit of detection for the non-cancer cells (e.g., PBMCs) and is above a limit of detection for the cancer cells. The cells can be infected by treatment with the MYXV at a multiplicity of infection of 1. The cells can be plated at approximately 1-1.5×10⁵ cells per replicate and/or infected at approximately 70% confluence or at least 70% confluence.

In some embodiments, the decorin gene is between the M152 and M154 genes in the MYXV genome, e.g., in a MYXV with a deletion or disruption of M153. In some embodiments, the decorin gene replaces or disrupts an M153 gene. In some embodiments, the decorin gene is inserted in the intergenic region between an M135R gene and an M136R gene of the MYXV genome.

In some embodiments, the decorin is encoded by a gene comprising, consisting essentially of, or consisting of the sequence of SEQ ID NO: 7. In some embodiments, the gene encoding the decorin comprises a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to that of SEQ ID NO: 7. In some instances, the decorin is encoded by a gene comprising at least 85, at least 90%, at least 91%, at least 92%, at least 93%, at least 94%, at least 95%, at least 96%, at least 97%, at least 98%, or at least 99% sequence identity to SEQ ID NO: 7. In some instances, the decorin is encoded by a gene comprising between 95% and 98%, or 95% and 99% sequence identity to SEQ ID NO: 7. In some instances, the decorin is encoded by a gene comprising about 90%, about 91%, about 92%, about 93%, about 94%, about 95%, about 96%, about 97%, about 98%, or about 99% sequence identity to SEQ ID NO: 7.

In some instances, the decorin protein comprises at least 85, at least 90%, at least 91%, at least 92%, at least 93%, at least 94%, at least 95%, at least 96%, at least 97%, at least 98%, or at least 99% sequence identity to SEQ ID NO: 32, residues 31-359 of SEQ ID NO: 32, or any one of SEQ ID NOs: 40 or 44-47. In some instances, the decorin protein comprises between 95% and 98%, or 95% and 99% sequence identity to SEQ ID NO: 32, residues 31-359 of SEQ ID NO: 32, or any one of SEQ ID NOs: 40 or 44-47. In some instances, the decorin protein comprises about 90%, about 91%, about 92%, about 93%, about 94%, about 95%, about 96%, about 97%, about 98%, or about 99% sequence identity to SEQ ID NO: 32, residues 31-359 of SEQ ID NO: 32, or any one of SEQ ID NOs: 40 or 44-47. In some embodiments, the decorin protein comprises residues SEQ ID NO: 32, residues 31-359 of SEQ ID NO: 32, or any one of SEQ ID NOs: 40 or 44-47.

### Recombinant Nucleic Acids

Disclosed herein, in certain embodiments, are recombinant nucleic acids. Some embodiments relate to a recombinant nucleic acid comprising at least a portion of a MYXV genome. In some embodiments, the recombinant nucleic acid comprises DNA. In some embodiments, the MYXV genome or the portion of the MYXV genome is modified to reduce expression of the M153 gene. In some embodiments, the M153 gene is modified to delete or knock out at least a portion of the M153 gene in the MYXV genome.

In some embodiments, the recombinant nucleic acid is engineered to introduce a mutation to the M153 gene. The mutation can comprise, for example, an insertion, deletion, substation, or a combination thereof. In some embodiments, the recombinant nucleic acid comprises a gene knock-in where the M153 gene is disrupted.

In some embodiments, the recombinant nucleic acid comprises a nucleic acid that encodes a non-viral molecule. In some embodiments, the recombinant nucleic acid comprises 1, 2, 3, 4, 5, 6, 7, 8, 9, 10, or more nucleic acid that each encode a non-viral molecule or component thereof, for example, transgenes that encode proteins.

In some embodiments, the recombinant nucleic acid comprises a nucleic acid that encodes tumor necrosis factor alpha (TNF-α). In some embodiments, the TNF-α is a human TNF-α. In some embodiments, the nucleic acid that encodes the TNF-α replaces or is adjacent to an M135R gene of the MYXV genome. In some embodiments, the nucleic acid that encodes the TNF-α is inserted between an M135R gene and an M136R gene of the MYXV genome. In some embodiments, expression of TNF-α is driven by a poxvirus synthetic early/late (sE/L) promoter. In some embodiments, expression of the TNF-α is driven by a promoter such as a P11 promoter (e.g., poxvirus P11 late promoter). In some embodiments, the nucleic acid that encodes the TNF-α disrupts, replaces, or is adjacent to an M153 gene of the MYXV genome, and/or is between an M152 and M154 gene in the MYXV genome.

In some embodiments, the recombinant nucleic acid comprises a nucleic acid that encodes an interleukin-12 subunit alpha (IL-12α). In some embodiments, the IL-12α is a human IL-12α. In some embodiments, expression of the IL-12α is driven by an internal ribosome entry site (IRES). In some embodiments, expression of the IL-12α is driven by an sE/L promoter. In some embodiments, expression of the IL-12α is driven by a promoter such as a P11 promoter (e.g., poxvirus P11 late promoter). In some embodiments, the nucleic acid that encodes IL-12α disrupts expression of an M153 gene of the MYXV genome, and/or is between an M152 and M154 gene in the MYXV genome.

In some embodiments, the recombinant nucleic acid comprises a nucleic acid that encodes an interleukin-12 subunit beta (IL-12β). In some embodiments, the IL-12β is a human IL-12β gene. In some embodiments, expression of the IL-12β is driven by an sE/L promoter. In some embodiments, expression of the IL-12β is driven by a promoter such as a P11 promoter (e.g., poxvirus P11 late promoter). In some embodiments, expression of the IL-12β is driven by an internal ribosome entry site (IRES). In some embodiments, the nucleic acid that encodes IL-12β disrupts expression of an M153 gene of the MYXV genome, and/or is between an M152 and M154 gene in the MYXV genome. In some embodiments, the nucleic acid that encodes IL-12β and the nucleic acid that encodes IL-12α both disrupt expression of an M153 gene of the MYXV genome, and/or are between an M152 and M154 gene in the MYXV genome.

In some embodiments, the recombinant nucleic acid comprises a nucleic acid that encodes decorin. In some embodiments, the decorin is a human decorin. In some embodiments, expression of the decorin is driven by an sE/L promoter. In some embodiments, expression of decorin is driven by a promoter such as a P11 promoter (e.g., poxvirus P11 late promoter). In some embodiments, expression of decorin is driven by an internal ribosome entry site (IRES). In some embodiments, the recombinant nucleic acid comprises a nucleic acid that encodes decorin disrupts expression of an M153 gene of the MYXV genome, and/or is between an M152 and M154 gene in the MYXV genome.

In some embodiments, the recombinant nucleic acid comprises a nucleic acid that encodes a reporter tag, for example, a fluorescent protein. In some embodiments, the reporter tag comprises a green fluorescent protein (GFP). In some embodiments, expression of the reporter tag is driven by an sE/L promoter. In some embodiments, the recombinant nucleic acid further comprises a nucleic acid that encodes a second reporter tag. In some embodiments, the second reporter tag comprises a red fluorescent protein (RFP), e.g., dsRed. In some embodiments, expression of the second reporter tag is driven by a poxvirus P11 late promoter. In some embodiments, the nucleic acid that encodes the second reporter tag disrupts expression of an M153 gene of the MYXV genome, and/or is between an M152 and M154 gene in the MYXV genome.

In some embodiments, use of a p11 promoter to drive expression of a first transgene (e.g., IL-12) in a recombinant nucleic acid disclosed herein results in a surprising and unexpected effect, for example, an altered and beneficial production profile of a second transgene (e.g., decorin) independent of the promoter that drives expression of the second transgene.

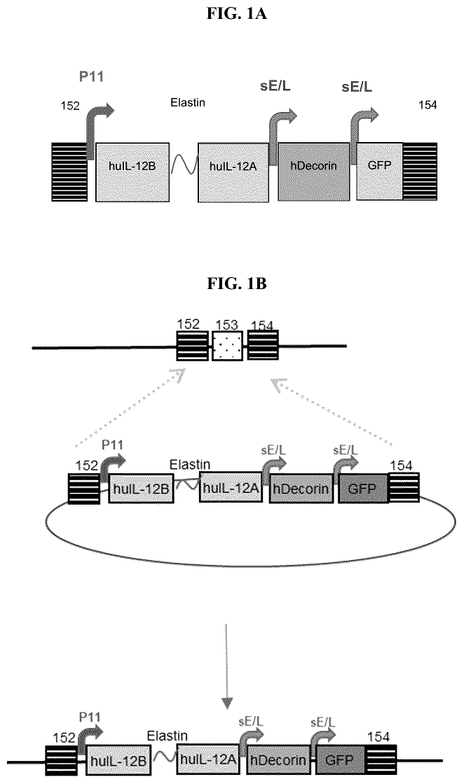

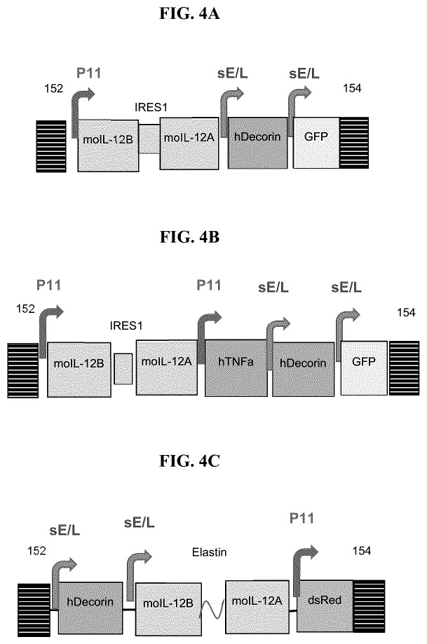

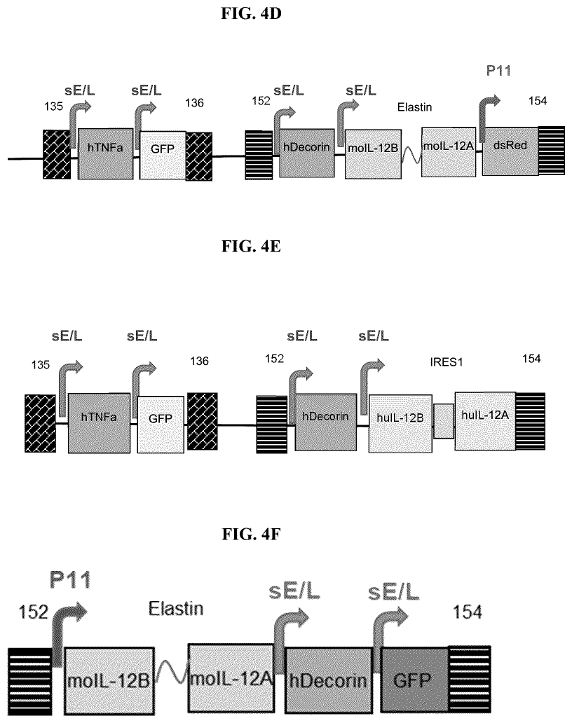

In some embodiments, the recombinant nucleic acid comprises, consists essentially of, or consists of, from 5′ to 3′, (i) a p11 promoter operatively linked to an IL-12 transgene comprising an IL-12β subunit, a linker (e.g., an elastin linker or another linker of the disclosure), and an IL-12α subunit, (ii) an sE/L promoter operatively linked to a decorin transgene, and optionally (iii) a sE/L promoter operatively linked to a reporter transgene (e.g., GFP). A non-limiting example of such a recombinant nucleic acid is provided in FIG. 1A and SEQ ID NO: 10. An additional non-limiting example of is provided in FIG. 4F. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (ii) or (iii), e.g., as provided in SEQ ID NO: 11.

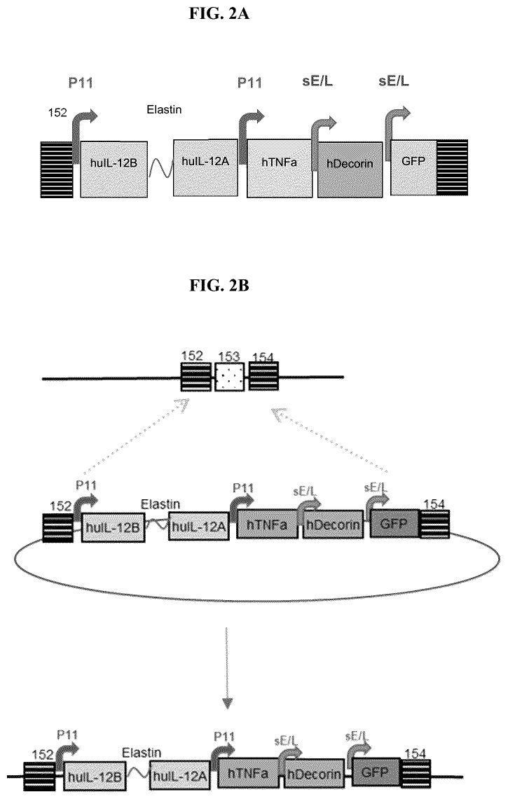

In some embodiments, the recombinant nucleic acid comprises, consists essentially of, or consists of, from 5′ to 3′, (i) a p11 promoter operatively linked to an IL-12 transgene comprising an IL-12β subunit, a linker (e.g., an elastin linker or another linker of the disclosure), and an IL-12α subunit, (ii) a p11 promoter operatively linked to a TNF-α transgene, (iii) an sE/L promoter operatively linked to a decorin transgene, and optionally (iv) a sE/L promoter operatively linked to a reporter transgene (e.g., GFP). A non-limiting example of such a recombinant nucleic acid is provided in FIG. 2A and SEQ ID NO: 20. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (iii) or (iv), e.g., as provided in SEQ ID NO: 21.

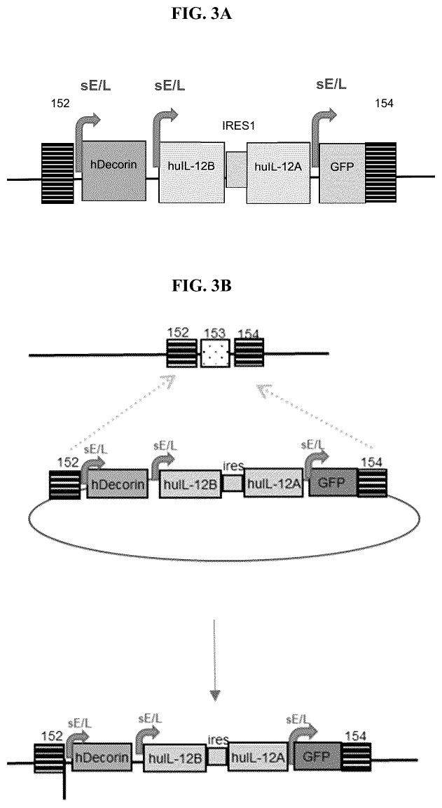

In some embodiments, the recombinant nucleic acid comprises, consists essentially of, or consists of, from 5′ to 3′, (i) an sE/L promoter operatively linked to a decorin transgene, (ii) an sE/L promoter operatively linked to an IL-12 transgene comprising an IL-12β subunit, an IRES, and an IL-12α subunit, and optionally (iii) a sE/L promoter operatively linked to a reporter transgene (e.g., GFP). A non-limiting example of such a recombinant nucleic acid is provided in FIG. 3A and SEQ ID NO: 25. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (ii) or (iii), e.g., as provided in SEQ ID NO: 26.

In some embodiments, the recombinant nucleic acid comprises, consists essentially of, or consists of, from 5′ to 3′, (i) p11 promoter operatively linked to an IL-12 transgene comprising an IL-12β subunit, an IRES, and an IL-12α subunit, (ii) an sE/L promoter operatively linked to a decorin transgene, and optionally (iii) an sE/L promoter operatively linked to a reporter transgene (e.g., GFP). A non-limiting example of such a recombinant nucleic acid is provided in FIG. 4A. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (ii) or (iii).

In some embodiments, the recombinant nucleic acid comprises, consists essentially of, or consists of, from 5′ to 3′, (i) p11 promoter operatively linked to an IL-12 transgene comprising an IL-12β subunit, an IRES, and an IL-12α subunit, (ii) a p11 promoter operatively linked to a TNF-α transgene, (iii) an sE/L promoter operatively linked to a decorin transgene, and optionally (iv) an sE/L promoter operatively linked to a reporter transgene (e.g., GFP). A non-limiting example of such a recombinant nucleic acid is provided in FIG. 4B. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (iii) or (iv).

In some embodiments, the recombinant nucleic acid comprises, consists essentially of, or consists of, from 5′ to 3′, (i) an sE/L promoter operatively linked to a decorin transgene, (ii) an sE/L promoter operatively linked to an IL-12 transgene comprising an IL-12β subunit, a linker (such as an elastin linker or another linker of the disclosure), and an IL-12α subunit, and optionally (iii) a sE/L or p11 promoter operatively linked to a reporter transgene (e.g., dsRed). A non-limiting example of such a recombinant nucleic acid is provided in FIG. 4C. The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (ii) or (iii).

In some embodiments, a recombinant nucleic acid comprises, consists essentially of, or consists of, from 5′ to 3′, (i) a sE/L promoter operatively linked to a TNF-α transgene, and (ii) optionally a sE/L promoter operatively linked to a reporter transgene (e.g., GFP). The recombinant nucleic acid can optionally contain recombination arms that are homologous to regions of the myxoma virus genome to target integration into the myxoma virus genome and/or deletion of a portion of the myxoma virus genome, for example, further comprising a 5′ recombination arm to the 5′ end of (i) and further comprising a 3′ recombination arm to the 3′ end of (i) or (i), (e.g., an intergenic region between M135 and M136, as shown in FIG. 4D and FIG. 4E).

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to any one of SEQ ID NOs: 10, 11, 20, 21, 25, 26, 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63. In some instances, the recombinant nucleic acid comprises, consists essentially of, or consists of a sequence with between 95% and 98%, or 95% and 99% sequence identity to any one of SEQ ID NOs: 10, 11, 20, 21, 25, 26, 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63. In some instances, the recombinant nucleic acid comprises, consists essentially of, or consists of a sequence with about 90%, about 91%, about 92%, about 93%, about 94%, about 95%, about 96%, about 97%, about 98%, or about 99% sequence identity to any one of SEQ ID NOs: 10, 11, 20, 21, 25, 26, 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63. In some embodiments, the recombinant nucleic acid comprises, consists essentially of, or consists of a sequence that is SEQ ID NO: 10, 11, 20, 21, 25, or 26. In some embodiments, the recombinant nucleic acid comprises, consists essentially of, or consists of a sequence that is 70%, 75%, 80%, 85%, 90%, 91%, 92%, 93%, 94%, 95%, 96%, 97%, 98%, 99%, or 100%, or a range of percentages defined by any two of the aforementioned percentages, identical to any one of SEQ ID NOs: 10, 11, 20, 21, 25, 26, 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 10. In some embodiments, the recombinant nucleic acid comprises, consists essentially of, or consists of a sequence that is SEQ ID NO: 10.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to nucleotides 1-2762 of SEQ ID NO: 10. In some embodiments, the recombinant nucleic acid comprises, consists essentially of, or consists of a sequence that is nucleotides 1-2762 of SEQ ID NO: 10.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 11. In some embodiments, the recombinant nucleic acid comprises, consists essentially of, or consists of a sequence that is SEQ ID NO: 11.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 20. In some embodiments, the recombinant nucleic acid comprises, consists essentially of, or consists of a sequence that is SEQ ID NO: 20.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to nucleotides 1-3507 of SEQ ID NO: 20. In some embodiments, the recombinant nucleic acid comprises, consists essentially of, or consists of a sequence that is nucleotides 1-3507 of SEQ ID NO: 20.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 21. In some embodiments, the recombinant nucleic acid comprises, consists essentially of, or consists of a sequence that is SEQ ID NO: 21.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 25. In some embodiments, the recombinant nucleic acid comprises, consists essentially of, or consists of a sequence that is SEQ ID NO: 25.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to nucleotides 1-3288 of SEQ ID NO: 25. In some embodiments, the recombinant nucleic acid comprises, consists essentially of, or consists of a sequence that is nucleotides 1-3288 of SEQ ID NO: 25.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 26. In some embodiments, the recombinant nucleic acid comprises, consists essentially of, or consists of a sequence that is SEQ ID NO: 26.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to SEQ ID NO: 63. In some embodiments, the recombinant nucleic acid comprises, consists essentially of, or consists of a sequence that is SEQ ID NO: 63.

In some instances, the recombinant nucleic acid comprises, consists essentially of, or consists of a sequence with at least 90%, at least 91%, at least 92%, at least 93%, at least 94%, at least 95%, at least 96%, at least 97%, at least 98%, or at least 99% sequence identity to nucleotides 1-3534 of SEQ ID NO: 63. In some embodiments, the recombinant nucleic acid comprises, consists essentially of, or consists of a sequence that is nucleotides 1-3534 of SEQ ID NO: 63.

A recombinant nucleic acid can contain recombination arms (e.g., one or two recombination arms) that are homologous to regions of the myxoma virus genome to target integration and/or deletion of a portion of the myxoma virus genome, for example, by homologous recombination. In some embodiments, a recombinant nucleic acid comprises a 5′ recombination arm. In some embodiments, a recombinant nucleic acid comprises a 3′ recombination arm. In some embodiments, a recombinant nucleic acid comprises a 5′ recombination arm and a 3′ recombination arm. The recombination arm nucleotide sequences can remain present in the genome of a MYXV after integration of the recombinant nucleic acid.

A 5′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 65. A 5′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 200 consecutive nucleotides of SEQ ID NO: 65. A 5′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 300 consecutive nucleotides of SEQ ID NO: 65. A 5′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 400 consecutive nucleotides of SEQ ID NO: 65. A 5′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 500 consecutive nucleotides of SEQ ID NO: 65. A 5′ recombination arm can comprise at least 50 consecutive nucleotides of SEQ ID NO: 65. A 5′ recombination arm can comprise at least 100 consecutive nucleotides of SEQ ID NO: 65. A 5′ recombination arm can comprise at least 150 consecutive nucleotides of SEQ ID NO: 65. A 5′ recombination arm can comprise at least 200 consecutive nucleotides of SEQ ID NO: 65. A 5′ recombination arm can comprise at least 300 consecutive nucleotides of SEQ ID NO: 65. A 5′ recombination arm can comprise at least 400 consecutive nucleotides of SEQ ID NO: 65. A 5′ recombination arm can comprise at least 500 consecutive nucleotides of SEQ ID NO: 65. A 5′ recombination arm can comprise, consist essentially of, or consist of the nucleotide sequence of SEQ ID NO: 65.

A 3′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to SEQ ID NO: 66. A 3′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 200 consecutive nucleotides of SEQ ID NO: 66. A 3′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 300 consecutive nucleotides of SEQ ID NO: 66. A 3′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 400 consecutive nucleotides of SEQ ID NO: 66. A 3′ recombination arm can comprise, consist essentially of, or consist of a nucleotide sequence with at least 80%, at least 85%, at least 90%, at least 91%, at least 92%, at least 93%, at least 94%, at least 95%, at least 96%, at least 97%, at least 98%, or at least 99% sequence identity to at least 500 consecutive nucleotides of SEQ ID NO: 66. A 3′ recombination arm can comprise at least 50 consecutive nucleotides of SEQ ID NO: 66. A 3′ recombination arm can comprise at least 100 consecutive nucleotides of SEQ ID NO: 66. A 3′ recombination arm can comprise at least 150 consecutive nucleotides of SEQ ID NO: 66. A 3′ recombination arm can comprise at least 200 consecutive nucleotides of SEQ ID NO: 66. A 3′ recombination arm can comprise at least 300 consecutive nucleotides of SEQ ID NO: 66. A 3′ recombination arm can comprise at least 400 consecutive nucleotides of SEQ ID NO: 66. A 3′ recombination arm can comprise at least 500 consecutive nucleotides of SEQ ID NO: 66. A 3′ recombination arm can comprise, consist essentially of, or consist of the nucleotide sequence of SEQ ID NO: 66.

In certain embodiments, a recombinant nucleic acid, transgene, or protein of the disclosure comprises one or more substitutions, deletions or insertions relative to any one of the sequences provided in SEQ ID NOs: 1-66. In some embodiments, the recombinant nucleic acid, transgene, or protein comprises from 1, 2, 3, 4, 5, 6, 7, 8, 9, 10 or more up to about 100, 90, 80, 70, 60, 50, 45, 40, 35, 30, 25, 20, 15 substitutions, deletions, or insertions. In some embodiments, the recombinant nucleic acid, transgene, or protein comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 substitutions, deletions, or insertions. In some embodiments, the recombinant nucleic acid, transgene, or protein comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 substitutions, deletions, or insertions. In some embodiments, the recombinant nucleic acid, transgene, or protein comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, or 20-25 substitutions, deletions, or insertions. In some embodiments, the recombinant nucleic acid, transgene, or protein comprises 1, 2, 3, 4, 5, 6, 7, 8, 9, 10, 11, 12, 13, 14, 15, 16, 17, 18, 19, or 20 substitutions, deletions, or insertions. A substitution can be a conservative or a non-conservative substitution. The one or more substitutions, deletions, or insertions can be at the N-terminus, the C-terminus, the 5′ end, the 3′ end, within the sequence, or a combination thereof. The substitutions, deletions, or insertions can be contiguous, non-contiguous, or a combination thereof.

In some embodiments, a recombinant nucleic acid, transgene, or a protein encoded therefrom comprises or encodes a signal sequence. In some embodiments, a recombinant nucleic acid, transgene, or a protein encoded therefrom lacks or does not encode a signal sequence, e.g., has a signal sequence removed relative to a sequence provided herein. In some embodiments, a recombinant nucleic acid, transgene, or a protein encoded therefrom comprises a different signal sequence to a signal sequence disclosed herein.

### Composition and Administration

Disclosed herein, in certain embodiments, are compositions comprising a MYXV as described herein. In some embodiments, the composition is or comprises a pharmaceutical composition. In some embodiments, the composition comprises a pharmaceutically acceptable carrier or excipient.

In some embodiments, the pharmaceutically acceptable carrier comprises an injectable fluid such as water, physiological saline, balanced salt solutions, aqueous dextrose, glycerol or the like. In some embodiments, the composition comprises a solid composition such as a powder, pill, tablet, or capsule. In some embodiments such as those including solid compositions, the pharmaceutically acceptable carrier comprises mannitol, lactose, starch, or magnesium stearate. In some embodiments, the pharmaceutically acceptable carrier comprises a biologically-neutral carrier. In some embodiments, the composition comprises wetting or emulsifying agents, preservatives, and pH buffering agents and the like, for example sodium acetate or sorbitan monolaurate.

In some embodiments, the identity or proportion of the pharmaceutically acceptable carrier or excipient is determined based on a route of administration, compatibility with a live virus, or standard pharmaceutical practice. In some embodiments, the pharmaceutical composition is formulated with components that do not significantly impair the biological properties of the MYXV. The pharmaceutical composition can be prepared by known methods for the preparation of pharmaceutically acceptable compositions suitable for administration to subjects, such that an effective quantity of the active substance or substances is combined in a mixture with a pharmaceutically acceptable vehicle. In some embodiments, the composition includes solutions of the MYXV in association with one or more pharmaceutically acceptable excipient, vehicles, or diluents, and contained in buffer solutions with a suitable pH and iso-osmotic with physiological fluids.

In some embodiments, the pharmaceutical composition is formulated for administration to a subject. The pharmaceutical composition may be administered to a subject in a variety of forms depending on the selected route of administration, as will be understood by those skilled in the art. In some instances, the pharmaceutical composition is administered systemically, or formulated for systemic administration. In some embodiments, the pharmaceutical composition is administered locally, or formulated for local administration.

In some embodiments, the pharmaceutical composition is administered parenterally, or formulated for parenteral administration. Examples of parenteral administration include intravenous, intratumoral, intraperitoneal, subcutaneous, intramuscular, transepithelial, nasal, intrapulmonary, intrathecal, rectal and topical modes of administration. Parenteral administration may be by continuous infusion over a selected period of time. Parenteral administration may be by bolus injection.

In some embodiments, the pharmaceutical composition is administered orally, or formulated for oral administration. The pharmaceutical composition may be administered orally, for example, with an inert diluent or with a carrier, or it may be enclosed in hard or soft shell gelatin capsules, or it may be compressed into tablets. For oral therapeutic administration, the MYXV may be incorporated with an excipient and be used in the form of ingestible tablets, buccal tablets, troches, capsules, elixirs, suspensions, syrups, wafers and the like.

Solutions of MYXV may be prepared in a physiologically suitable buffer. In some embodiments, under ordinary conditions of storage and use, these preparations contain a preservative to prevent the growth of microorganisms, but that will not inactivate the live virus. In some embodiments, a dose of the pharmaceutical composition to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. In certain embodiments, the therapeutic virus may be freeze dried for storage at room temperature.

The pharmaceutical compositions may additionally contain additional therapeutic agents, such as additional anti-cancer agents. In some embodiments, the compositions include a chemotherapeutic agent. The chemotherapeutic agent, for example, may be substantially any agent, which exhibits an oncolytic effect against cancer cells or neoplastic cells of the subject and that does not inhibit or diminish the tumor killing effect of the MYXV. For example, the chemotherapeutic agent may be, without limitation, an anthracycline, an alkylating agent, an alkyl sulfonate, an aziridine, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. Preferably, the chemotherapeutic agent is one that is known to be effective against the particular cell type that is cancerous or neoplastic. In some cases, the additional therapeutic agent comprises an immune checkpoint modulator.

In some embodiments, the composition comprises peripheral blood mononuclear cells (PBMCs), bone marrow (BM) cells, or a combination thereof treated ex vivo by an MYXV as described herein. In some embodiments, the PBMCs, BM cells, or a combination thereof comprise autologous cells. In some embodiments, the PBMCs, BM cells, or a combination thereof are obtained from an allogeneic donor. In some embodiments, the PBMCs, BM cells, or a combination thereof are obtained from heterologous donors.

### Methods of Use

Disclosed herein, in certain embodiments, are methods of inhibiting, alleviating, treating, reducing, or preventing a cancer in a subject in need thereof, comprising administering to the subject a composition or pharmaceutical composition as described herein. In certain embodiments, the method includes administering to a subject, such as a human subject, a MYXV as described herein, thereby treating and/or inhibiting the cancer in the subject in need thereof.

Some embodiments include prophylactic treatment with the MYXV. In some embodiments, the subject has, is suspected of having, or is at risk of having the cancer. Some embodiments include selecting the subject suspected of having the cancer. Some embodiments include selecting the subject at risk of having the cancer. In some embodiments, the subject has the cancer. In some embodiments, the methods include selecting the subject with the cancer.

In some embodiments, the subject is a human. In some embodiments, the subject is a patient. In some embodiments, the subject is an animal or nonhuman animal. Examples of nonhuman animals include vertebrates such as mammals and non-mammals. Some examples of mammals include nonhuman primates, sheep, dog, cat, horse, cow, and rodents such as mice and rats.

In some embodiments, the cancer is a solid tumor. Examples of solid tumors such as sarcomas and carcinomas include but are not limited to fibrosarcoma, myxosarcoma, liposarcoma, chondrosarcoma, osteosarcoma, osteogenic sarcoma, and other sarcomas, synovioma, mesothelioma, Ewing's sarcoma, leiomyosarcoma, rhabdomyosarcoma, colon carcinoma, lymphoid malignancy, pancreatic cancer, breast cancer, metastatic breast carcinoma/adenocarcinoma, lung cancers, non-small cell lung cancer, lung adenocarcinoma, lung squamous cell carcinoma, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, hepatoblastoma, bile duct carcinoma, choriocarcinoma, Wilms' tumor, cervical cancer, testicular tumor, bladder carcinoma, Merkel cell carcinoma, head and neck squamous cell carcinoma (HNSCC), colorectal cancer, colorectal adenocarcinoma, gastric cancer, gastric adenocarcinoma, gastrointestinal cancer, adenoid cystic carcinoma, neuroendocrine tumors, acantholytic squamous cell carcinoma, acantholytic squamous cell carcinoma, bronchioalveolar carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma). In some embodiments, the cancer comprises an osteosarcoma, triple negative breast cancer, or melanoma.

In some embodiments, the cancer has metastasized to a location in the subject. In some embodiments, the location comprises a lung, a brain, a liver and/or a lymph node of the subject.

In some embodiments, the cancer comprises a hematologic cancer. Non-limiting examples of hematologic cancers include Hodgkin's lymphoma, non-Hodgkin's lymphoma, B-cell or T-cell hematologic cancers, acute lymphocytic leukemia (ALL), acute myelogenous leukemia (AML), chronic myelogenous leukemia (CML), multiple myeloma, mixed phenotype leukemia, myelofibrosis, high risk myelodysplastic syndrome, very high risk myelodysplastic syndrome.

In some embodiments, the composition reduces cancer cell viability, and/or activates immunogenic cell death in the cancer. In some embodiments, the cancer is inhibited, alleviated, or prevented upon administration of the composition. In some embodiments, the administration improves the subject's survival.

MYXV or the composition comprising the MYXV can be administered to the subject using standard methods of administration. In some embodiments, the virus or the composition comprising the virus is administered systemically (e.g., IV injection). In some embodiments, the virus or the composition comprising the virus is administered by injection at the disease site (e.g., intratumorally). In some embodiments, the virus or the composition comprising the virus is administered orally or parenterally, or by any standard method known in the art. In certain embodiments, the MYXV or the composition comprising the MYXV is administered at a site of a tumor and/or metastasis.

The MYXV can be administered initially in a suitable amount that may be adjusted as required, depending on the clinical response of the subject. The effective amount of virus can be determined empirically and depends on the maximal amount of the MYXV that can be administered safely, and the minimal amount of the virus that produces the desired result.

The concentration of virus to be administered may vary depending on the virulence of the particular strain of MYXV that is to be administered and on the nature of the cells that are being targeted. In one embodiment, a dose of less than about 3×10¹⁰ focus forming units (“ffu”), also called “infectious units”, is administered to a human subject, in various embodiments, between about 10² to about 10⁹ pfu, between about 10² to about 10⁷ pfu, between about 10³ to about 10⁶ pfu, or between about 10⁴ to about 10⁵ pfu may be administered in a single dose.

In some embodiments, the MYXV is administered at a dose and schedule effective to increase expression of a cytokine by immune cells (e.g., PBMCs) in the subject. The expression of a cytokine by immune cells can be increased, for example, by at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold. In some embodiments, expression of the cytokine is increased from below a limit of detection to a detectable level. In some embodiments, the MYXV is administered at a dose and schedule effective to increase expression of two, three, four, five, six, or more cytokines by immune cells in the subject. In some embodiments, the MYXV is administered at a dose and schedule effective to increase expression of at least one, at least two, at least three, at least four, at least five, at least six, or more cytokines by immune cells in the subject. The cytokines can comprise, for example, IFN-γ, IL-2, IL-6, IL-10, IL-12, TNF-α, or any combination thereof. In some embodiments, expression of TNF-α is increased. In some embodiments, expression of IL-12 is increased. In some embodiments, expression of decorin is increased. In some embodiments, expression of IFN-γ is increased.

In some embodiments, the MYXV is administered at a dose and schedule effective to increase expression of a cytokine by cancer cells in the subject. The expression of a cytokine by cancer cells can be increased, for example, by at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 50-fold, at least about 100-fold, at least about 1000-fold, or at least about 5000-fold. In some embodiments, expression of the cytokine is increased from below a limit of detection to a detectable level. In some embodiments, the MYXV is administered at a dose and schedule effective to increase expression of two, three, four, five, six, or more cytokines by cancer cells in the subject. In some embodiments, the MYXV is administered at a dose and schedule effective to increase expression of at least one, at least two, at least three, at least four, at least five, at least six, or more cytokines by cancer cells in the subject. The cytokines can comprise, for example, IFN-γ, IL-2, IL-6, IL-10, IL-12, TNF-α, or any combination thereof. In some embodiments, expression of TNF-α is increased. In some embodiments, expression of IL-12 is increased. In some embodiments, expression of decorin is increased. In some embodiments, expression of IFN-γ is increased.

Myxoma viruses disclosed herein can exhibit advantageous properties compared to control myxoma viruses that, for example, express a functional M153 protein, lack one or more transgenes, contain a different recombinant nucleic acid, and/or utilize different promoters for transgene expression.

In some embodiments, a MYXV with reduced activity or expression of M153 that comprises a recombinant nucleic acid disclosed herein exhibits an EC50 for killing or growth inhibition of a cancer (e.g., cancer cell line) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold lower than an EC50 exhibited by a control myxoma virus that expresses a functional M153 protein, for example, according to an in vitro assay disclosed herein. The assay can be conducted, for example, with cells that are approximately 70% confluent or at least 70% confluent.

In some embodiments, a MYXV that comprises a recombinant nucleic acid disclosed herein and expresses a transgene exhibits an EC50 for killing or growth inhibition of a cancer (e.g., cancer cell line) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold lower than an EC50 exhibited by a control myxoma virus that lacks the transgene, for example, according to an in vitro assay disclosed herein. The assay can be conducted, for example, with cells that are approximately 70% confluent or at least 70% confluent.

In some embodiments, a MYXV that comprises a recombinant nucleic acid disclosed herein and expresses a transgene (e.g., IL-12, TNF-α, or decorin) from a p11 promoter exhibits an EC50 for killing or growth inhibition of a cancer (e.g., cancer cell line) that is at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold lower than an EC50 exhibited by a corresponding control myxoma virus that expresses the transgene from a different promoter, for example, a sE/L promoter.

EC50 can be calculated as 50% of the maximum response inhibition compared to control, e.g., determined from the luminescence signals in a cell titer glow viability assays at 72 hours post-infection. The surviving fraction of cells can be determined by dividing the mean luminescence values of the test agents by the mean luminescence values of untreated control. The effective concentration value for the test agent and control can be estimated using Prism 8 software (GraphPad Software, Inc.) by curve-fitting the normalized response data using the non-linear regression analysis.

Myxoma viruses disclosed herein can exhibit advantageous properties in the treatment of cancer compared to control myxoma viruses that, for example, express a functional M153 protein, lack one or more transgenes, contain a different recombinant nucleic acid, and/or utilize different promoters for transgene expression.

In some embodiments, a MYXV disclosed herein with reduced activity or expression of M153 reduces tumor volume at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold more than a control myxoma virus that expresses a functional M153 protein, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene reduces tumor volume at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold more than a control myxoma virus that lacks the transgene, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene (e.g., IL-12, TNF-α, or decorin) from a p11 promoter reduces tumor volume at least about 10%, at least about 20%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold more than a corresponding control myxoma virus that expresses the transgene from a different promoter, for example, an sE/L promoter. In some embodiments, the effect is achieved even compared to a higher dose of the control myxoma virus, for example, a two-fold, five-fold, or ten-fold higher dose.

In some embodiments, a MYXV disclosed herein with reduced activity or expression of M153 improves a rate of survival of subjects with a cancer at least about 5%, at least about 10%, at least about 15%, at least about 20%, at least about 25%, at least about 30%, at least about 40%, at least about 50%, at least about 60%, at least about 70%, at least about 80%, or at least about 90% compared to a control myxoma virus that expresses a functional M153 protein, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene improves a rate of survival of subjects with a cancer at least about 5%, at least about 10%, at least about 15%, at least about 20%, at least about 25%, at least about 30%, at least about 40%, at least about 50%, at least about 60%, at least about 70%, at least about 80%, or at least about 90% compared to a control myxoma virus that lacks the transgene, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene (e.g., IL-12, TNF-α, or decorin) from a p11 promoter improves a rate of survival of subjects with a cancer at least about 5%, at least about 10%, at least about 15%, at least about 20%, at least about 25%, at least about 30%, at least about 40%, at least about 50%, at least about 60%, at least about 70%, at least about 80%, or at least about 90% compared to a corresponding control myxoma virus that expresses the transgene from a different promoter, for example, an sE/L promoter. In some embodiments, the effect is achieved even compared to a higher dose of the control myxoma virus, for example, a two-fold, five-fold, or ten-fold higher dose.

In some embodiments, a MYXV disclosed herein with reduced activity or expression of M153 extends a mean survival time at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold compared to a control myxoma virus that expresses a functional M153 protein, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene extends a mean survival time at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold more than a control myxoma virus that lacks the transgene, for example, according to an assay disclosed herein. In some embodiments, the effect is achieved even compared to a higher dose of the control myxoma virus, for example, a two-fold, five-fold, or ten-fold higher dose.

In some embodiments, a MYXV that comprises a recombinant nucleic acid disclosed herein and expresses a transgene (e.g., IL-12, TNF-α, or decorin) from a p11 promoter extends a mean survival time at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, at least about 10-fold, at least about 25-fold, at least about 50-fold, at least about 100-fold, or at least about 1000-fold more than a corresponding control myxoma virus that expresses the transgene from a different promoter, for example, an sE/L promoter. In some embodiments, the effect is achieved even compared to a higher dose of the control myxoma virus, for example, a two-fold, five-fold, or ten-fold higher dose.

In some embodiments, the MYXV is administered at a dose and schedule effective to reduce the volume of a tumor in the subject. The volume of the tumor can be reduced, for example, by at least about 5%, at least about 10%, at least about 20%, at least about 30%, at least about 40%, at least about 50%, at least about 60%, at least about 70%, at least about 80%, or at least about 90%, e.g., relative to before the administering, relative to untreated subjects, or relative to subjects administered a control MYXV.

In some embodiments, the MYXV is administered at a dose and schedule effective to reduce the rate of tumor or cancer cell growth in the subject. The rate of tumor or cancer cell growth can be reduced, for example, by at least about 5%, at least about 10%, at least about 20%, at least about 30%, at least about 40%, at least about 50%, at least about 60%, at least about 70%, at least about 80%, or at least about 90%, e.g., relative to before the administering, relative to untreated subjects, or relative to subjects treated with a control MYXV.

In some embodiments, the MYXV is administered at a dose and schedule effective to increase the rate of survival of subjects with cancer that are treated with the MYXV. The rate of survival can be increased, for example, by at least about 5%, at least about 10%, at least about 15%, at least about 20%, at least about 25%, at least about 30%, at least about 40%, at least about 50%, at least about 60%, at least about 70%, at least about 80%, or at least about 90%, e.g., relative to subjects that are not treated or that are treated with a control MYXV.

In some embodiments, the MYXV is administered at a dose and schedule effective to increase the time of survival (e.g., mean time to death) of subjects with cancer. The time of survival can be increased, for example, by at least about 5%, at least about 10%, at least about 15%, at least about 20%, at least about 25%, at least about 30%, at least about 40%, at least about 50%, at least about 60%, at least about 70%, at least about 80%, at least about 90%, at least about 2-fold, at least about 5-fold, or at least about 10-fold, e.g., compared to subjects that are not treated or subjects that are treated with a control MYXV.

In some embodiments, a myxoma virus comprising a recombinant nucleic acid of the disclosure that encodes IL-12 and decorin exhibits surprisingly and unexpectedly enhanced anti-tumor efficacy compared a corresponding virus that further expresses TNF-α, for example, achieving a larger reduction of tumor volume, an increased rate of survival, or an extended time of survival (e.g., mean time to death) for subjects administered the MYXV that comprises the recombinant nucleic acid and expresses IL-12 and decorin compared to a corresponding control MYXV that further expresses TNF-α.

The MYXV can be administered as a sole therapy or may be administered in combination with other therapies, including chemotherapy, immunotherapy and/or radiation therapy. For example, the MYXV can be administered either prior to or following surgical removal of a primary tumor or prior to, concurrently with or following treatment such as administration of radiotherapy or conventional chemotherapeutic drugs. In some embodiments, the MYXV can be administered at least 1 day, 2 days, 3 days, 4 days, 5 days, 6 days, 7 days, 1.5 weeks, 2 weeks, or 3 weeks before the other therapy. In some embodiments, the MYXV can be administered at least 1 day, 2 days, 3 days, 4 days, 5 days, 6 days, 7 days, 1.5 weeks, 2 weeks, or 3 weeks after the other therapy. In some embodiments, the MYXV can be administered within 1 day, 2 days, 3 days, 4 days, 5 days, 6 days, or 7 days of the other therapy. In some embodiments, the MYXV can be administered concurrently with the other therapy.

Some embodiments further comprise administering to the subject an additional therapeutic agent. In some embodiments, the additional therapeutic agent is an immune checkpoint modulator. In some embodiments, the additional therapeutic agent is administered to the subject before administering the composition. In some embodiments, the additional therapeutic agent is administered to the subject after administering the composition. In some embodiments, the additional therapeutic agent is administered to the subject as a combination with the composition.

In some embodiments, the additional therapeutic agent comprises an immune modulator, for example, an immune activation modulator, an immune checkpoint modulator, or an immune checkpoint inhibitor. Exemplary immune checkpoint modulators include, but are not limited to, PD-L1 inhibitors or activation modulators such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; PD-L2 inhibitors or activation modulators such as GlaxoSmithKline's AMP-224 (Amplimmune), and rHIgM12B7; PD-1 inhibitors or activation modulators such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 inhibitors or activation modulators such as ipilimumab (also known as Yervoy®, MDX-010, BMS-734016 and MDX-101) from Bristol Meyers Squibb, tremelimumab (CP-675,206, ticilimumab) from Pfizer, or AGEN 1884 from Agenus; LAG3 inhibitors or activation modulators such as BMS-986016 from Bristol-Myers Squibb, IMP701 from Novartis Pharmaceuticals, LAG525 from Novartis Pharmaceuticals, or REGN3767 from Regeneron Pharmaceuticals; B7-H3 inhibitors or activation modulators such as enoblituzumab (MGA271) from MacroGenics; KIR inhibitors or activation modulators such as Lirilumab (IPH2101; BMS-986015) from Innate Pharma; CD137 activation modulators such as urelumab (BMS-663513, Bristol-Myers Squibb), PF-05082566 (anti-4-1BB, PF-2566, Pfizer), or XmAb-5592 (Xencor); PS inhibitors or activation modulators such as Bavituximab; and immune activation modulators such as an antibody or fragments (e.g., a monoclonal antibody, a human, humanized, or chimeric antibody) thereof, RNAi molecules, or small molecules that target, modulate, inhibit, activate, or bind to TIM3, CD40, CD52, CD30, CD20, CD33, CD27, OX40, GITR, ICOS, BTLA (CD272), CD160, 2B4, LAIR1, TIGHT, LIGHT, DR3, CD226, CD2, or SLAM.

Further disclosed is a delivery strategy where the therapeutic MYXV virus is first adsorbed ex vivo to cells prior to infusion of the cells into the subject. In this strategy, MYXV can be delivered to cancer sites (e.g., primary and/or metastatic sites) via migration of the cells contacted with virus ex vivo. This systemic delivery method is sometimes called “ex vivo virotherapy”, or EVV (aka EV2), because the virus is first delivered to isolated cells prior to infusion into the subject. The MYXV construct and this delivery strategy may significantly reduce tumor burden and increase survival in a subject in need thereof.

In some embodiments, the cells are leukocytes. In some embodiments, the cells are peripheral blood mononuclear cells (PBMCs). In some embodiments, the cells are bone marrow-derived cells. In some embodiments, the cells are primary cells. In some embodiments, the cells are not primary cells, e.g., are a cell line. In some embodiments, the cells are engineered cells, e.g., cells engineered to express or overexpress an immune receptor, such as a chimeric antigen receptor (CAR), T cell receptor, cytokine receptor, chemokine receptor, or NK receptor. In some embodiments, the cells are stem cells. In some embodiments, the cells are hematopoietic stem cells to be administered as part of an autologous or allogeneic hematopoietic stem cell transplant. In some embodiments, the cells are induced pluripotent stem cells (iPSCs). In some embodiments, the cells are mesenchymal stem cells (MSCs). In some embodiments, the cells are partially-differentiated or terminally-differentiated stem cells.

In some embodiments, the cells are adsorbed with MYXV constructs for one hour ex vivo, and then the MYXV-loaded cells are infused back into the recipient. In some embodiments, the cells are adsorbed with MYXV constructs for at least or about 30 minutes, one hour, two hours, three hours, four hours, six hours, or more ex vivo, and then the MYXV-loaded cells are infused back into the recipient.

In certain embodiments, the cells are obtained from the subject, for example as autologous cells. In some embodiments, the cells are obtained from one or more allogeneic donors, for example, a donor that is matched to the recipient for at least 1, at least 2, at least 3, at least 4, at least 5, at least 6, at least 7, or at least 8 HLA alleles (such as one or both copies of HLA-A, HLA-B, HLA-A, and/or HLA-DR alleles). HLA alleles can be types, for example, using DNA-based methods. In some embodiments, the mononuclear peripheral blood cells and/or bone marrow cells are obtained from one or more haploidentical donors.

### EMBODIMENTS

Embodiment 1. A recombinant nucleic acid comprising: at least a portion of myxoma virus (MYXV) genome and a first nucleic acid encoding interleukin-12 subunit beta (IL-12β); wherein the first nucleic acid is inserted at the MYXV genome to reduce or disrupt the expression of M153 gene of the MYXV genome; and wherein expression of the IL-12β is driven by a first poxvirus P11 late promoter.

Embodiment 2. The recombinant nucleic acid of embodiment 1, wherein the IL-12β is human IL-12β.

Embodiment 3. The recombinant nucleic acid of embodiment 1 or embodiment 2, further comprising a second nucleic acid encoding interleukin-12 subunit alpha (IL-12α).

Embodiment 4. The recombinant nucleic acid of embodiment 3, wherein the IL-12α is human IL-12α.

Embodiment 5. The recombinant nucleic acid of embodiment 3 or 4, wherein the 5′ end of the second nucleic acid is coupled to the 3′-end of the first nucleic acid.

Embodiment 6. The recombinant nucleic acid of any one of embodiments 3-5, wherein the first and second nucleic acids are coupled via a third nucleic acid encoding an elastin linker.

Embodiment 7. The recombinant nucleic acid of any one of the preceding embodiments, further comprising a fourth nucleic acid encoding decorin.

Embodiment 8. The recombinant nucleic acid of embodiment 7, wherein the decorin is human decorin.

Embodiment 9. The recombinant nucleic acid of embodiment 7 or embodiment 8, wherein expression of the decorin is driven by a first sE/L promoter.

Embodiment 10. The recombinant nucleic acid of any one of embodiments 7-9, wherein the 5′ end of the fourth nucleic acid is coupled to the 3′-end of the second nucleic acid.

Embodiment 11. The recombinant nucleic acid of embodiment 9 or embodiment 10, wherein the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; and (f) the fourth nucleic acid encoding the decorin.

Embodiment 12. The recombinant nucleic acid of any one of the preceding embodiments, wherein the recombinant nucleic acid comprises, consists essentially of, or consists of a vMyx-P11 late promoter-hIL-12β-elastin linker-hIL-12α-sE/L promoter-hdecorin expression cassette.

Embodiment 13. The recombinant nucleic acid of one of the any preceding embodiments, wherein the recombinant nucleic acid comprises, consists essentially of, or consists of a nucleotide sequence with at least 80%, at least 85%, at least 90%, at least 95%, or at least 98% sequence identity to nucleotides 1-2762 of SEQ ID NO: 10.

Embodiment 14. The recombinant nucleic acid of any one of the preceding embodiments, wherein the recombinant nucleic acid comprises, consists essentially of, or consists of a nucleotide sequence that is nucleotides 1-2762 of SEQ ID NO: 10.

Embodiment 15. The recombinant nucleic acid of any one of the preceding embodiments, further comprising a fifth nucleic acid encoding a reporter tag.

Embodiment 16. The recombinant nucleic acid of embodiment 15, wherein the reporter tag comprises a green fluorescent protein (GFP).

Embodiment 17. The recombinant nucleic acid of embodiment 15 or embodiment 16, wherein expression of the reporter tag is driven by a second sE/L promoter.

Embodiment 18. The recombinant nucleic acid of any one of embodiments 15-17, wherein the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; (f) the fourth nucleic acid encoding the decorin; (g) the second sE/L promoter; and (h) the fifth nucleic acid encoding the reporter tag.

Embodiment 19. The recombinant nucleic acid of any one of embodiments 15-17, wherein the recombinant nucleic acid comprises, consists essentially of, or consists of a vMyx-P11 late promoter-hIL-12β-elastin linker-hIL-12α-sE/L promoter-hdecorin-sE/L promoter-GFP expression cassette.

Embodiment 20. The recombinant nucleic acid of any one of the preceding embodiments, wherein the recombinant nucleic acid comprises, consists essentially of, or consists of a nucleotide sequence with at least 80%, at least 85%, at least 90%, at least 95%, or at least 98% sequence identity to SEQ ID NO: 10 or SEQ ID NO: 11.

Embodiment 21. The recombinant nucleic acid of any one of the preceding embodiments, wherein the recombinant nucleic acid comprises, consists essentially of, or consists of a nucleotide sequence that is SEQ ID NO: 10 or SEQ ID NO: 11.

Embodiment 22. The recombinant nucleic acid of any one of embodiments 1-21, further comprising a sixth nucleic acid encoding tumor necrosis factor alpha (TNF-α).

Embodiment 23. The recombinant nucleic acid of embodiment 22, wherein the TNF-α is human TNF-α.

Embodiment 24. The recombinant nucleic acid of embodiment 22 or embodiment 23, wherein the TNF-α is a soluble polypeptide.

Embodiment 25. The recombinant nucleic acid of any one of embodiments 22-24, wherein expression of the TNF-α is driven by a second poxvirus P11 late promoter.

Embodiment 26. The recombinant nucleic acid of any one of embodiments 22-25, wherein the sixth nucleic acid is located between the second nucleic acid encoding IL-12α and the fourth nucleic acid encoding decorin.

Embodiment 27. The recombinant nucleic acid of any one of embodiments 22-26, wherein the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the second poxvirus P11 late promoter; (f) the sixth nucleic acid encoding TNF-α; (g) the first sE/L promoter; (h) the fourth nucleic acid encoding the decorin; (i) optionally, the second sE/L promoter; and (j) optionally, the fifth nucleic acid encoding the reporter tag.

Embodiment 28. The recombinant nucleic acid of any one of embodiments 22-27, wherein the recombinant nucleic acid comprises, consists essentially of, or consists of a vMyx-P11 late promoter-hIL-12β-elastin linker-hIL-12α-P11 late promoter-TNF-α-sE/L promoter-hdecorin expression cassette.

Embodiment 29. The recombinant nucleic acid of any one of embodiments 22-28, wherein the recombinant nucleic acid comprises, consists essentially of, or consists of a nucleotide sequence with at least 80%, at least 85%, at least 90%, at least 95%, or at least 98% sequence identity to nucleotides 1-3507 of SEQ ID NO: 20.

Embodiment 30. The recombinant nucleic acid of any one of embodiments 22-28, wherein the recombinant nucleic acid comprises, consists essentially of, or consists of a nucleotide sequence that is nucleotides 1-3507 of SEQ ID NO: 20.

Embodiment 31. The recombinant nucleic acid of any one of embodiments 22-28, wherein the recombinant nucleic acid comprises or consists of a vMyx-P11 late promoter-hIL-12β-elastin linker-hIL-12α-P11 late promoter-TNF-α-sE/L promoter-hdecorin-sE/L promoter-GFP expression cassette.

Embodiment 32. The recombinant nucleic acid of any one of embodiments 22-28, wherein the recombinant nucleic acid comprises, consists essentially of, or consists of a nucleotide sequence with at least 80%, at least 85%, at least 90%, at least 95%, or at least 98% sequence identity to SEQ ID NO: 20 or SEQ ID NO: 21.

Embodiment 33. The recombinant nucleic acid of any one of embodiments 22-28, wherein the recombinant nucleic acid comprises, consists essentially of, or consists of a nucleotide sequence that is SEQ ID NO: 20 or SEQ ID NO: 21.

Embodiment 34. A recombinant nucleic acid comprising at least a portion of myxoma virus (MYXV) genome, and a nucleic acid expression cassette inserted at the MYXV genome to reduce or disrupt expression of M153 gene of the MYXV genome, wherein nucleic acid expression cassette comprises, from 5′ to 3′: sE/L promoter-hdecorin-sE/L promoter-hIL-12β-IRES-hIL-12α-sE/L promoter-GFP.

Embodiment 35. The recombinant nucleic acid of embodiment 34, wherein the recombinant nucleic acid comprises, consists essentially of, or consists of a sequence with at least 80%, at least 85%, at least 90%, at least 95%, or at least 98% sequence identity to SEQ ID NO: 25, SEQ ID NO: 26, SEQ ID NO: 63, nucleotides 1-3288 of SEQ ID NO: 25, or nucleotides 1-3534 of SEQ ID NO: 63.

Embodiment 36. The recombinant nucleic acid of embodiment 34, wherein the recombinant nucleic acid comprises, consists essentially of, or consists of a nucleotide sequence that is SEQ ID NO: 25, SEQ ID NO: 26, SEQ ID NO: 63, nucleotides 1-3288 of SEQ ID NO: 25, or nucleotides 1-3534 of SEQ ID NO: 63.

Embodiment 37. A genetically engineered MYXV having enhanced immune-modulatory or anti-tumor activity, wherein at least 80% of a nucleic acid encoding M153 protein in MYXV genome is knocked out, wherein the genetically engineered MYXV comprises the recombinant nucleic acid of any one of embodiments 1-36.

Embodiment 38. The genetically engineered MYXV of embodiment 37, wherein expression of the IL-12β is reduced in a non-cancer cell infected by the genetically engineered MYXV as compared to a non-cancer cell infected with a corresponding control myxoma virus in which expression of the IL-12β is driven by a sE/L promoter.

Embodiment 39. The genetically engineered MYXV of embodiment 37 or embodiment 38, wherein expression of the IL-12β is reduced in a peripheral blood mononuclear cell (PBMC) infected by the genetically engineered MYXV as compared to a PBMC infected by a corresponding control myxoma virus in which expression of the IL-12β is driven by a sE/L promoter.

Embodiment 40. The genetically engineered MYXV of embodiment 37, wherein expression of the IL-12β by a cell infected by the genetically engineered MYXV is reduced at four hours post-infection as compared to a cell infected by a corresponding control myxoma virus in which expression of the IL-12β is driven by a sE/L promoter.

Embodiment 41. A genetically engineered MYXV comprising a nucleic acid that encodes a cytokine, wherein expression of the cytokine is driven by a poxvirus p11 late promoter, wherein the MYXV is genetically engineered to attenuate expression or activity of M153.

Embodiment 42. The genetically engineered MYXV of embodiment 41, wherein the cytokine comprises IL-12β, IL-12α, or a combination thereof.

Embodiment 43. The genetically engineered MYXV of embodiment 41 or embodiment 42, wherein the cytokine comprises TNF-α.

Embodiment 44. The genetically engineered MYXV of any one of embodiments 41-43, wherein at least 80% of a nucleic acid encoding the M153 is deleted in a genome of the genetically engineered MYXV.

Embodiment 45. The genetically engineered MYXV of any one of embodiments 41-44, wherein expression of the cytokine is reduced in a non-cancer cell infected by the genetically engineered MYXV as compared to a non-cancer cell infected by a corresponding control myxoma virus in which expression of the cytokine is driven by a sE/L promoter.

Embodiment 46. The genetically engineered MYXV of any one of embodiments 41-44, wherein expression of the cytokine is reduced in a PBMC infected by the genetically engineered MYXV as compared to a PBMC infected by a corresponding control myxoma virus in which expression of the cytokine is driven by a sE/L promoter.

Embodiment 47. The genetically engineered MYXV of any one of embodiments 41-44, wherein expression of the cytokine by a cell infected by the genetically engineered MYXV is reduced at four hours post-infection as compared to a cell infected by a corresponding control myxoma virus in which expression of the cytokine is driven by a sE/L promoter.

Embodiment 48. The genetically engineered MYXV of any one of embodiments 41-47, wherein the MYXV comprises a nucleic acid sequence that comprises, consists essentially of, or consists of a nucleotide sequence with at least 80%, at least 85%, at least 90%, at least 95%, or at least 98% sequence identity to SEQ ID NO 10, SEQ ID NO 11, SEQ ID NO: 20, SEQ ID NO: 21, SEQ ID NO: 25, SEQ ID NO: 26, SEQ ID NO: 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63.

Embodiment 49. The genetically engineered MYXV of any one of embodiments 41-47, wherein the MYXV comprises a nucleic acid sequence that comprises, consists essentially of, or consists of SEQ ID NO 10, SEQ ID NO 11, SEQ ID NO: 20, SEQ ID NO: 21, SEQ ID NO: 25, SEQ ID NO: 26, SEQ ID NO: 63, nucleotides 1-2762 of SEQ ID NO: 10, nucleotides 1-3507 of SEQ ID NO: 20, nucleotides 1-3288 of SEQ ID NO: 25, or nucleotides 1-3534 of SEQ ID NO: 63.

Embodiment 50. The genetically engineered MYXV of any one of embodiments 37-49, wherein the MYXV is genetically engineered Lausanne strain MYXV.

Embodiment 51. The genetically engineered MYXV of any one of embodiments 37-50, wherein the p11 promoter comprises, consists essentially of, or consists of a nucleotide sequence with at least 90% sequence identity to SEQ ID NO: 2.

Embodiment 52. The genetically engineered MYXV of any one of embodiments 37-50, wherein the p11 promoter comprises, consists essentially of, or consists of the nucleotide sequence of SEQ ID NO: 2.

Embodiment 53. A mammalian cell treated ex vivo with the recombinant nucleic acid of any one of embodiments 1-36 or the genetically engineered MYXV of any one of embodiments 37-52.

Embodiment 54. The mammalian cell of embodiment 53, wherein the mammalian cell is a tumor cell.

Embodiment 55. The mammalian cell of embodiment 53, wherein the mammalian cell is a peripheral blood mononuclear cell (PBMC) or a bone marrow (BM) cell.

Embodiment 56. A composition comprising the recombinant nucleic acid of any one of embodiments 1-36, the genetically engineered MYXV of any one of embodiments 37-52, or the mammalian cell of any one of embodiments 53-55.

Embodiment 57. The composition of embodiment 56, formulated for systemic administration.

Embodiment 58. The composition of embodiment 56, formulated for local administration.

Embodiment 59. A method of increasing an immune response against a tumor in a subject in need thereof, comprising administering to the subject the composition of any one of embodiments 56-58.

Embodiment 60. The method of embodiment 59, wherein the subject has, is suspected of having the tumor.

Embodiment 61. The method of embodiment 59 or embodiment 60, wherein the administration is systemic administration.

Embodiment 62. The method of any one of embodiments 59-61, wherein the administering is intravenous.

Embodiment 63. The method of embodiment 59 or embodiment 60, wherein the administering is local.

Embodiment 64. The method of any one of embodiments 59, 60, and 63, wherein the administering is intratumoral.

Embodiment 65. The method of any one of the embodiments 59-64, wherein the tumor comprises a solid tumor.

Embodiment 66. The method of any one of the embodiments 59-65, wherein the tumor is a lung cancer, colon cancer, gastric cancer, liver cancer, breast cancer, or melanoma.

Embodiment 67. The method of any one of the embodiments 59-66, wherein the administration improves the subject's survival.

Embodiment 68. The method of any one of the embodiments 59-67, wherein the administration reduces cancer cell viability, or activates immunogenic cell death in the cancer.

Embodiment 69. The method of any one of the embodiments 59-68, wherein the administration is performed in a dose and a schedule effective to increase expression of at least two cytokines in the tumor of the subject.

Embodiment 70. The method of any one of the embodiments 59-69, wherein the administration is performed in a dose and a schedule effective to reduce volume of the tumor at least 10%.

Embodiment 71. The method of any one of the embodiments 59-70, wherein the administration is performed in a dose and a schedule effective to reduce the growth of the tumor at least 10%.

Embodiment 72. The method of any one of embodiments 59-71, wherein the subject survives at least 10% longer than a subject administered a ten-fold higher dose of a corresponding control myxoma virus that expresses M153, lacks the recombinant nucleic acid, or a combination thereof.

### EXAMPLES

These examples are provided for illustrative purposes only and not to limit the scope of the claims provided herein.

### Example 1—Virus Construction

This example describes the design and generation of novel engineered Myxoma viruses with M153 knocked out, and with transgenes encoding IL-12, decorin, TNF-α, GFP, and/or dsRed introduced into the viral genome. The Myxoma virus Lausanne strain (ATCC VR-1829; GenBank: GCF 000843685.1) was the parental virus used for generation of these engineered viruses.

HV11 Myxoma Virus

An oncolytic myxoma virus was constructed to contain IL-12, decorin, and GFP transgenes at the M153 locus, with knockout of M153. As shown in FIG. 1A, a p11 promoter drives expression of human IL-12A and IL-12B, which are joined by an elastin linker; a synthetic early/late (sE/L) promoter drives expression of human decorin; and a sE/L promoter drives expression of GFP as a reporter.

To generate the new recombinant virus with the desired transgenes and promoters inserted in the M153 locus, a recombination plasmid vector was designed. The recombination plasmid included the insert sequence, and 0.5-1 kb flanking recombination arms containing sequences homologous to regions upstream and downstream of M153, as shown in FIG. 1B.

The sE/L promoter used was SEQ ID NO: 1. The p11 promoter used was SEQ ID NO: 2. The IL-12 contained, from 5′ to 3′, human IL-12B excluding the stop codon (nucleotides 1-984 of SEQ ID NO: 3), elastin linker (SEQ ID NO: 6), and human IL-12A lacking the signal peptide (SEQ ID NO: 5). The sequence encoding the IL-12B-elastin-IL-12A fusion protein is provided in SEQ ID NO: 9. The decorin gene had the sequence of SEQ ID NO: 7. The GFP gene had the sequence of SEQ ID NO: 8. The combined insert sequence containing the promoters and transgenes is provided in SEQ ID NO: 10. The insert sequence including the upstream and downstream flanking sequences to direct recombination at the M153 locus of the myxoma virus of the genome is shown in SEQ ID NO: 11. The full recombination plasmid sequence is provided in SEQ ID NO: 12.

A monolayer of Vero cells was infected with parental Myxoma virus Lausanne strain at a multiplicity of infection (MOI) of 1. One hour after adding the virus, the recombination plasmid of SEQ ID NO: 12 was transfected into the Vero cells. Foci of recombinant virus were identified based on expression of GFP, and four rounds of clonal selection were done to isolate recombinant Myxoma virus containing the insertion sequence. Insertion was confirmed by PCR with primers targeting sequences upstream and downstream of M153, resulting in a band of approximately 0.7 kb for the parental virus, and 4.5 kb for recombinant virus with the insert (primers of SEQ ID NO: 13 and SEQ ID NO: 14).

Clones were tested for expression of IL-12 and decorin via ELISA of infected cell culture supernatants. A clone confirmed to express IL-12 and decorin was selected for subsequent use.

The presence of the p11 promoter upstream of IL-12 was confirmed by PCR with a p11-specific forward primer (SEQ ID NO: 15) and an IL-12-specific reverse primer (SEQ ID NO: 16), and by sequencing. Master stocks of HV11 were generated for use in subsequent experiments.

### HV14 Myxoma Virus

An oncolytic myxoma virus was constructed to contain IL-12, TNF-α, decorin, and GFP transgenes at the M153 locus, with knockout of M153. As shown in FIG. 2A, a p11 promoter drives expression of human IL-12A and IL-12B, which are joined by an elastin linker; a p11 promoter drives expression of human TNF-α; a synthetic early/late (sE/L) promoter drives expression of human decorin; and a sE/L promoter drives expression of GFP as a reporter.

To generate the new recombinant virus with the desired transgenes and promoters inserted in the M153 locus, a recombination plasmid vector was designed. The recombination plasmid includes the insert sequence, and 0.5-1 kb flanking recombination arms containing sequences homologous to regions upstream and downstream of M153, as shown in FIG. 2B.

The sE/L promoter used was SEQ ID NO: 1. The p11 promoter used was SEQ ID NO: 2. The IL-12 contained, from 5′ to 3′, human IL-12B excluding the stop codon (nucleotides 1-984 of SEQ ID NO: 3), elastin linker (SEQ ID NO: 6), and human IL-12A lacking the signal peptide (SEQ ID NO: 5). The sequence encoding the IL-12B-elastin-IL-12A fusion protein is provided in SEQ ID NO: 9. A six base pair spacer was inserted between the IL-12A gene and the p11 promoter that drives expression of TNF-α (SEQ ID NO: 17). The TNF-α gene had the sequence of SEQ ID NO: 18. A six base pair spacer (SEQ ID NO: 19) was inserted between the TNF-α gene and the sE/L promoter that drives expression of decorin. The decorin gene had the sequence of SEQ ID NO: 7. The GFP gene had the sequence of SEQ ID NO: 8. The combined insert sequence containing the promoters and transgenes is provided in SEQ ID NO: 20. The insert sequence including the upstream and downstream flanking sequences to direct recombination at the M153 locus of the myxoma virus of the genome is shown in SEQ ID NO: 21. The full recombination plasmid sequence is provided in SEQ ID NO: 22.

A monolayer of Vero cells was infected with parental Myxoma virus Lausanne strain at a multiplicity of infection (MOI) of 1. One hour after adding the virus, the recombination plasmid of SEQ ID NO: 22 was transfected into the Vero cells. Foci of recombinant virus were identified based on expression of GFP, and four rounds of clonal selection were done to isolate recombinant Myxoma virus containing the insertion sequence. Insertion was confirmed by PCR with primers targeting sequences upstream and downstream of M153, resulting in a band of approximately 0.7 kb for the parental virus, and 5.5 kb for recombinant virus with the insert (primers of SEQ ID NO: 13 and SEQ ID NO: 14).

Clones were tested for expression of IL-12, TNF-α, and decorin via ELISA of infected cell culture supernatants. A clone confirmed to express IL-12, TNF-α, and decorin was selected for subsequent use.

The presence of the p11 promoter upstream of IL-12 and TNF-α was confirmed by PCR with a p11-specific forward primer (SEQ ID NO: 15) and an IL-12-specific reverse primer (SEQ ID NO: 16), or a TNF-α specific reverse primer (SEQ ID NO: 23), and by sequencing. Master stocks of HV14 were generated for use in subsequent experiments.

HV12 Myxoma Virus

An oncolytic myxoma virus was constructed to contain decorin, IL-12, and GFP transgenes at the M153 locus, with knockout of M153. As shown in FIG. 3A, a synthetic early/late (sE/L) promoter drives expression of each of the transgenes.

To generate the new recombinant virus with the desired transgenes and promoters inserted in the M153 locus, a recombination plasmid vector was designed. The recombination plasmid includes the insert sequence, and 0.5-1 kb flanking recombination arms containing sequences homologous to regions upstream and downstream of M153, as shown in FIG. 3B.

The sE/L promoter used was SEQ ID NO: 1 or SEQ ID NO: 61. The decorin gene had the sequence of SEQ ID NO: 7 or SEQ ID NO: 62. The IL-12 contained, from 5′ to 3′, human IL-12B (SEQ ID NO: 3), an internal ribosome entry site (IRES; SEQ ID NO: 24 or SEQ ID NO: 42), and human IL-12A (SEQ ID NO: 4). The GFP gene had the sequence of SEQ ID NO: 8. The combined insert sequence containing the promoters and transgenes is provided in SEQ ID NO: 25; an alternative sequence is provided in SEQ ID NO: 63. The insert sequence including the upstream and downstream flanking sequences to direct recombination at the M153 locus of the myxoma virus of the genome is shown in SEQ ID NO: 26. The full recombination plasmid sequence is provided in SEQ ID NO: 27.

A monolayer of Vero cells was infected with parental Myxoma virus Lausanne strain at a multiplicity of infection (MOI) of 1. One hour after adding the virus, the recombination plasmid of SEQ ID NO: 27 was transfected into the Vero cells. Foci of recombinant virus were identified based on expression of GFP, and five rounds of clonal selection were done to isolate recombinant Myxoma virus containing the insertion sequence. Insertion was confirmed by PCR with primers targeting sequences upstream and downstream of M153, resulting in a band of approximately 0.7 kb for the parental virus, and 4.5 kb for recombinant virus with the insert (primers of SEQ ID NO: 13 and SEQ ID NO: 14).

Clones were tested for expression of IL-12 and decorin via ELISA of infected cell culture supernatants. A clone confirmed to express IL-12 and decorin was selected for subsequent use.

Master stocks of HV12 were generated for use in subsequent experiments.

MV1, MV2, MV3, MV4, and HV13 Myxoma Viruses

Similar techniques were used to generate additional myxoma viruses with transgene insertions as follows.

The MV2 virus was constructed to contain IL-12, decorin, and GFP transgenes at the M153 locus, with knockout of M153. As shown in FIG. 4A, a p11 promoter drives expression of murine IL-12A and IL-12B, which are separated by an IRES; a sE/L promoter drives expression of human decorin; and a sE/L promoter drives expression of GFP as a reporter.

The MV4 virus was constructed to contain IL-12, TNF-α, decorin, and GFP transgenes at the M153 locus, with knockout of M153. As shown in FIG. 4B, a p11 promoter drives expression of murine IL-12A and IL-12B, which are separated by an IRES; a p11 promoter drives expression human TNF-α; a sE/L promoter drives expression of human decorin; and a sE/L promoter drives expression of GFP as a reporter.

The MV1 virus was constructed to contain IL-12, decorin, and dsRed transgenes at the M153 locus, with knockout of M153. As shown in FIG. 4C, a sE/L promoter drives expression of human decorin; a sE/L promoter drives expression of mouse IL-12A and IL-12B, which are joined by an elastin linker, and a p11 promoter drives expression of dsRed as a reporter.

The MV3 virus was constructed to contain IL-12, decorin, and dsRed transgenes at the M153 locus, with knockout of M153, and TNF-α and GFP transgenes present in an intergenic region between M135 and M136. As shown in FIG. 4D, a sE/L promoter drives expression of human decorin; a sE/L promoter drives expression of mouse IL-12A and IL-12B, which are joined by an elastin linker; a p11 promoter drives expression of dsRed as a reporter; a sE/L promoter drives expression of human TNF-α, and a sE/L promoter drives expression of GFP.

The HV13 virus was constructed to contain IL-12 and decorin transgenes at the M153 locus, with knockout of M153, and TNF-α and GFP transgenes present in an intergenic region between M135 and M136. As shown in FIG. 4E, a sE/L promoter drives expression of human decorin; a sE/L promoter drives expression of human IL-12B and IL-12A, which are separated by an IRES, a sE/L promoter drives expression of TNF-α, and a sE/L promoter drives expression of GFP.

The MV5 virus was constructed to contain IL-12, decorin, and GFP transgenes at the M153 locus, with knockout of M153. As shown in FIG. 4F, a p11 promoter drives expression of mouse IL-12A and IL-12B, which are joined by an elastin linker; a sE/L promoter drives expression of human decorin; and a sE/L promoter drives expression of GFP.

### Example 2—Transgene Expression by Infected Cells

This example demonstrates that cells infected with myxoma viruses of the disclosure secrete TNF, Decorin, and/or IL-12.

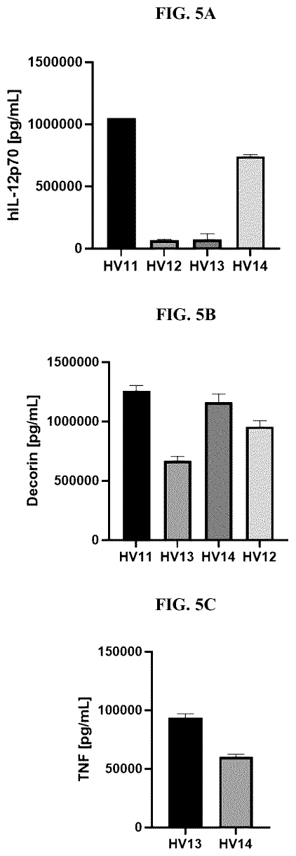

Vero cells were plated at approximately 1.5×10⁵ cells/well in 24 well plates and allowed to adhere overnight. The cells (at least 70% confluent) were infected with the HV11, HV12, HV13, and HV14 myxoma viruses at a multiplicity of infection (MOI) of 1. After 24 hours, cell culture supernatant was harvested, and subjected to ELISA to measure the production of IL-12, decorin, and TNF-α. IL-12 and decorin were detected in the supernatant for all of the engineered viruses as shown in FIG. 5A and FIG. 5B, respectively, indicating the viruses are capable of inducing expression and secretion of IL-12 and decorin by infected cells. Relatively higher levels of IL-12 were detected for the HV11 and HV14 viruses, which have an elastin linker joining the IL-12A and IL-12B subunits. TNF-α was also detected in the supernatant of the HV13 and HV14-infected Vero cells, as shown in FIG. 5C.

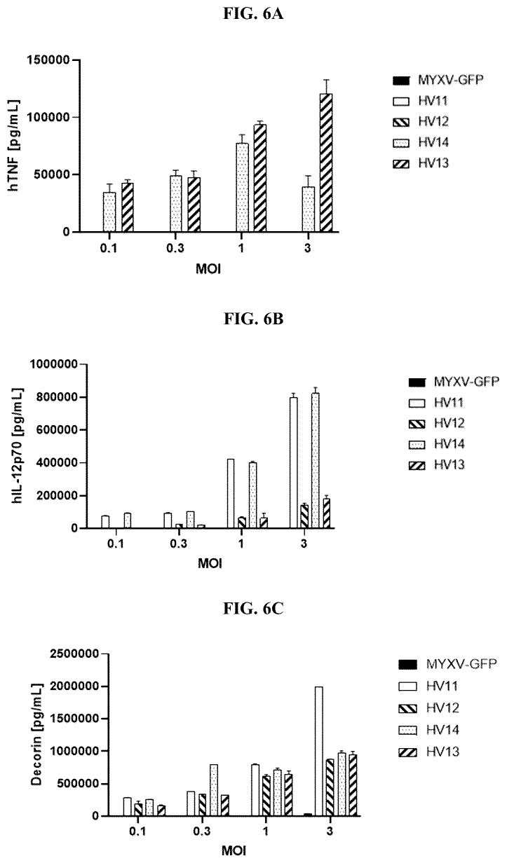

When the experiment was repeated with different MOI conditions from 0.1 to 3, an MOI-dependent effect on transgene expression was observed, with higher production of TNF-α, IL-12, and decorin detected for cultures infected with a higher concentration of virus as shown in FIG. 6A, FIG. 6B, and FIG. 6C, respectively. TNF-α, IL-12, and decorin were not detected in cultures infected with an “empty” Myxoma virus that lacked the TNF-α, IL-12, and decorin transgenes (MYXV-GFP), and which contains an intact M153 gene.

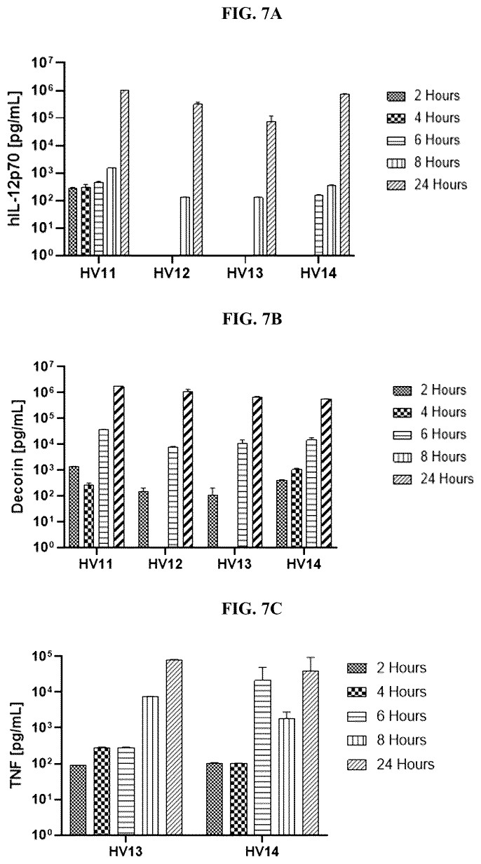

A time course experiment was done to measure production of the cytokines and decorin by infected Vero cells at 2, 4, 6, 8 and 24 hours post-infection. A time-dependent effect was observed, with the highest concentrations of IL-12, decorin, and TNF-α detected at 24 hours, as shown in FIG. 7A, FIG. 7B, and FIG. 7C, respectively. IL-12 was detected at earlier time points for cultures infected with HV11 and HV14, which have an elastin linker joining the IL-12A and IL-12B subunits, than the other engineered viruses (FIG. 7A).

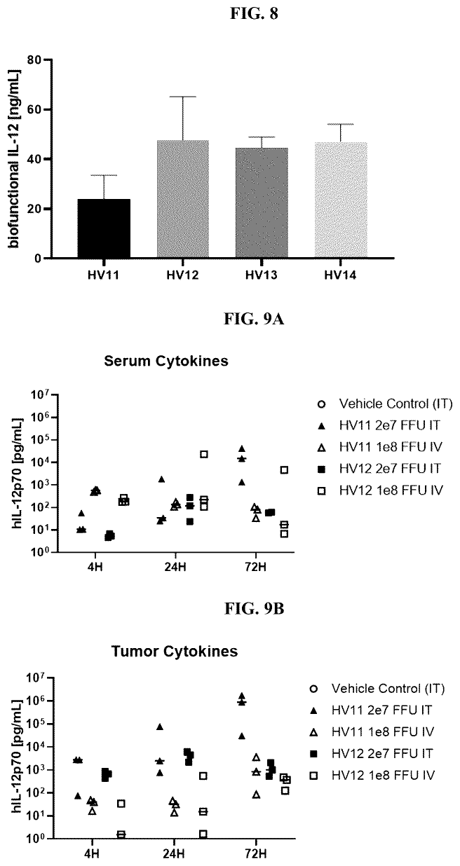

An assay was conducted to measure the biological functionality of IL-12 produced by Vero cells infected with HV11, HV12, HV13, and HV14. Supernatants of Vero cell cultures infected with the engineered viruses were collected at 24 hours post-infection, and the functional IL-12 activity of the supernatant measured using an IL-12 responsive reporter cell line (e.g., HEK-Blue IL-12 cells that produce alkaline phosphatase in response to IL-12 signaling, which can subsequently be measured to quantify IL-12 activity). Biologically active IL-12 was detected in supernatants originating from cultures infected with all four engineered Myxoma viruses, as shown in FIG. 8.

Production of IL-12, decorin, and TNF was evaluated for human cancer cell lines. A549 (lung carcinoma) and HeLa (cervical adenocarcinoma) cells were infected with HV11, HV12, HV13, and HV14 engineered myxoma viruses, each at an MOI of 1. Culture supernatants were harvested at 24 hours post-infection, and transgene production evaluated by ELISA. TABLE 1 shows the detected concentrations of the proteins in pg/mL. These results show that the engineered myxoma viruses elicit production of the cytokine and decorin transgenes in human cancer cells.

TABLE 1

| | hIL-12 | | | | hDecorin | | | | hTNF | |
|---|---|---|---|---|---|---|---|---|---|---|
| Cell line | HV11 | HV12 | HV13 | HV14 | HV11 | HV12 | HV13 | HV14 | HV13 | HV14 |
| A549 | 133786 | 6281 | 9249 | 68247 | 272442 | 378194 | 329307 | 331043 | 14858 | 19806 |
| HeLa | 154280 | 67377 | 0 | 199847 | 348917 | 387028 | 278565 | 300694 | 7393 | 11265 |

The MV1, MV2, MV3, and MV4 engineered viruses were also tested for the ability to elicit production of the encoded transgenes by infected cells.

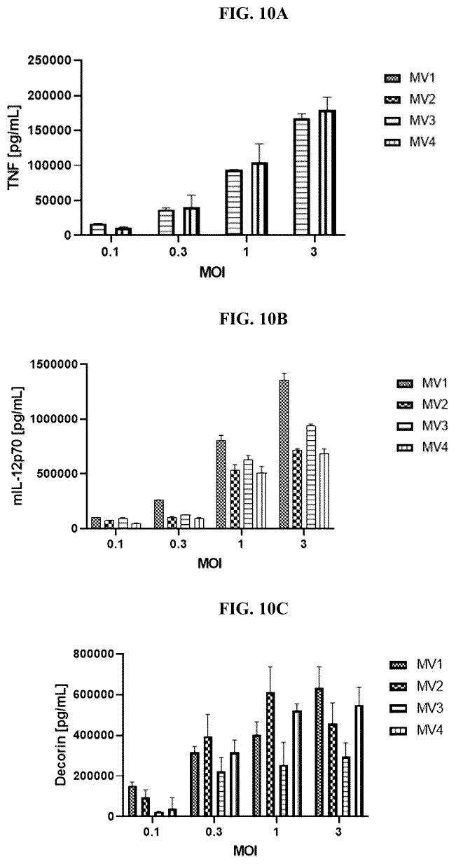

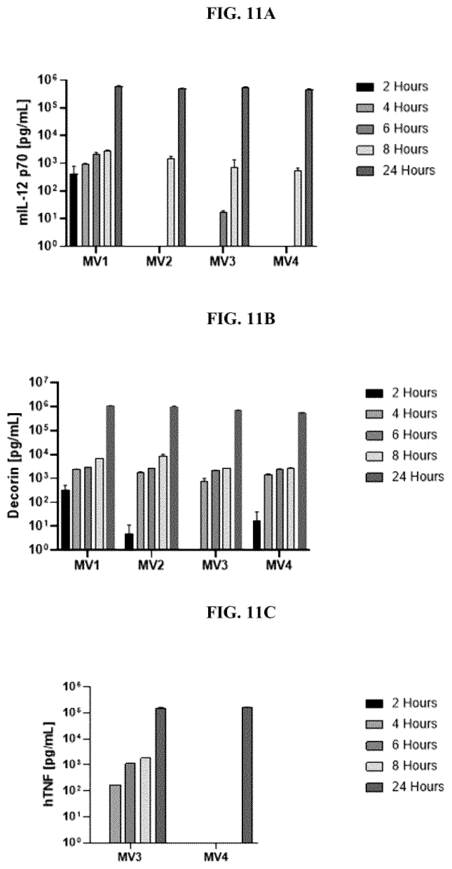

Vero cells were plated at approximately 1.5×10⁵ cells/well in 24 well plates and allowed to adhere overnight. The cells (at least 70% confluent) were infected with the MV1, MV2, MV3, and MV4 myxoma viruses at multiplicities of infection of 0.1, 0.3, 1, or 3. After 24 hours, cell culture supernatant was harvested and subjected to ELISA to measure the production of IL-12, decorin, and TNF-α. An MOI-dependent effect on transgene expression was observed, with higher production of TNF-α, IL-12, and decorin detected for cultures infected with a higher concentration of virus as shown in FIG. 10A, FIG. 10B, and FIG. 10C, respectively.

A time course experiment was done to measure production of TNF-α, IL-12, and decorin by infected Vero cells at 2, 4, 6, 8 and 24 hours post-infection. A time-dependent effect was observed, with the highest concentrations of IL-12, decorin, and TNF-α detected at 24 hours, as shown in FIG. 11A, FIG. 11B, and FIG. 11C, respectively. TNF-α was detected at earlier timepoints for MV3, for which TNF-α expression is driven by the sE/L promoter, compared to MV4, for which TNF-α expression is driven by the p11 promoter (FIG. 11C); IL-12 was also detected at earlier time points for cultures infected with MV1 and MV3, for which IL-12 expression is driven by the sE/L promoter, and which have an elastin linker joining the IL-12A and IL-12B subunits (FIG. 11A).

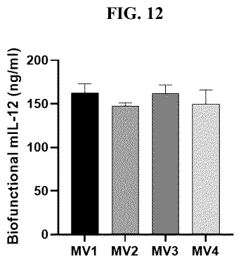

An assay was conducted to measure the biological functionality of IL-12 produced by Vero cells infected with MV1, MV2, MV3, and MV4. Supernatants of Vero cell cultures infected with the engineered viruses were collected at 24 hours post-infection, and the functional IL-12 activity of the supernatant measured using an IL-12 responsive reporter cell. Biologically active IL-12 was detected in supernatants originating from cultures infected with all four engineered Myxoma viruses, as shown in FIG. 12.

Production of IL-12, decorin, and TNF was evaluated for infected cancer cell lines. B16-F10 (melanoma), K7M2 (metastatic osteosarcoma), and CT-26 (colon carcinoma) cells were infected with MV1, MV2, MV3, and MV4 engineered myxoma viruses, each at an MOI of 1. Culture supernatants were harvested at 24 hours post-infection, and transgene production evaluated by ELISA. TABLE 2 shows the detected concentrations of the proteins in pg/mL. These results show that the engineered myxoma viruses elicit production of the cytokine and decorin transgenes in mouse cancer cells.

TABLE 2

| | mIL-12 | | | | hDecorin | | | | hTNF | |
|---|---|---|---|---|---|---|---|---|---|---|
| Cell line | MV1 | MV2 | MV3 | MV4 | MV1 | MV2 | MV3 | MV4 | MV3 | MV4 |
| B16-F10 | 496265 | 236311 | 105888 | 149017 | 606288 | 666382 | 268845 | 693668 | 13737 | 76533 |
| K7M2 | 82366 | 5000 | 3956 | 10132 | 133835 | 93828 | 24879 | 151672 | 2000 | 2250 |
| CT-26 | 72042 | 5000 | 5000 | 5000 | 93588 | 47454 | 34550 | 113429 | 2062 | 1432 |

### Example 3—Transgene Expression by Infected Cells In Vivo

This example demonstrates that the HV11 and HV12 engineered myxoma viruses elicit production of IL-12 in an in vivo cancer model.

Immunodeficient mice were implanted with 5×10⁶ A549 human non-small cell lung cancer cells subcutaneously on the flank. Tumor bearing animals were randomized when the mean tumor volume was 100-150 mm³ and were treated via intratumoral (IT) injection of 2×10⁷ focus forming units (FFU)/dose or intravenous (IV) injection of 1×10⁸ FFU/dose on Day 1 (n=3 animals per group). Serum and tumor samples were collected at 4-, 24-, or 72-hours post viral injection, and processed for cytokine quantification. Cytokine analysis was performed using MesoScale Discovery (MSD) U-Plex 6-assay 96-Well SECTOR plates. Symbols represent individual animals, line represents mean. The results showed that the HV11 and HV12 viruses elicited production of IL-12 in the sera (FIG. 9A) and tumors (FIG. 9B) of treated tumor-bearing mice.

### Example 4—Inhibition of Growth of Cancer Cell Lines In Vitro by Recombinant Myxoma Virus

To further characterize the ability of HV11, HV12, HV13, and HV14 to inhibit growth of cancer cell lines in vitro, 14 human cancer cell lines were infected at 9 different multiplicities of infection (MOI=0.01, 0.03, 0.1, 0.3, 1, 3, 10, 30, 100), and growth inhibition was determined using cell viability assays. Adherent cells were infected at approximately 70% confluence. TABLE 3 shows the EC50 values calculated for the cell lines. For cell lines that showed less than 50% total growth inhibition but exhibited a growth inhibition plateau at ≤50% relative to control, the value in parenthesis is the maximum growth inhibition observed for each virus. The data show that in many instances, HV11, HV12, HV13, and HV14 achieve growth inhibition at lower MOI than a myxoma virus that lacks the transgenes (MYXV-GFP). The data also provide examples of myxoma viruses disclosed herein that exhibit particularly strong inhibition of cancer cells, which can be dependent on, for example, the combination of transgenes, which promoter(s) drive expression of the transgene(s), the presence/absence of a linker between IL-12A and IL12B subunits, transgene orientation, and/or the cancer cell type.

TABLE 3
EC₅₀ results for human cell lines

| | | | | |
|---|---|---|---|---|
| MYXV-GFP | HV11 | HV12 | HV13 | HV14 |

Lung

| | | | | | |
|---|---|---|---|---|---|
| A549 | 51.6 | 52.24 | 51.94 | 52.31 | 6.937 (49.11) |
| H358 | 1.06 (34.035) | 2.693 (39.45) | 9.845 (38.14) | 4.2445 (32.89) | 1.498 (45.34) |
| H1975 | 53.02 | 10.175 (51.275) | 10.856 (49.795) | 10.7 | 7.3 |
| SK-MES-1 | 1.422 (33.7) | 2.113 (34.98) | 2.3745 (28.855) | 1.715 (31.9) | 1.336 (45.395) |

Colon, Gastric & Liver

| | | | | | |
|---|---|---|---|---|---|
| DLD-1 | 15.28 | 20.53 | 20.7 (51.54) | 59.11 | 57.15 |
| COLO205 | 33.9 | 63.53 | 7.06 | 5.75 | 3.52 |
| MKN-45 | 55.475 (58.085) | 113.725 (63.745) | 52.45 | 50.56 | 18.62 |
| HEP-G2 | >100 | 0.893 (1.4585) | 32.66 (24.55) | 29.03 (24.5) | 5.63 (38.55) |
| NCI-N87 | 13.03 | 11.24 | 0.8 | 0.99 | 1.51 |
| Hep3B | 58.94 | 58.25 | 14.85 | 21.23 | 7.43 |

Melanoma

| | | | | | |
|---|---|---|---|---|---|
| A375 | 11.66 (46.38) | 10.02 (47.78) | 54.56 | 55.9 | 17.15 (34.115) |
| MDA-MB-435 | 1.48 | 1.57 | 1.69 | 1.19 | 0.9 |

Breast

| | | | | | |
|---|---|---|---|---|---|
| MDA-MB-157 | 22.28 (23.29) | 12.19 (25.47) | 370.47 (66.82) | 34.39 (16.6) | 46.13 (56.47) |
| MDA-MB-231 | 20.31 (36.8) | 33.1 (47.1) | 18.29 (47.81) | 24.76 (54.9) | 7.75 |

### Example 5—Anti-Cancer Activity of Recombinant Myxoma Virus in Mouse Breast Carcinoma Model

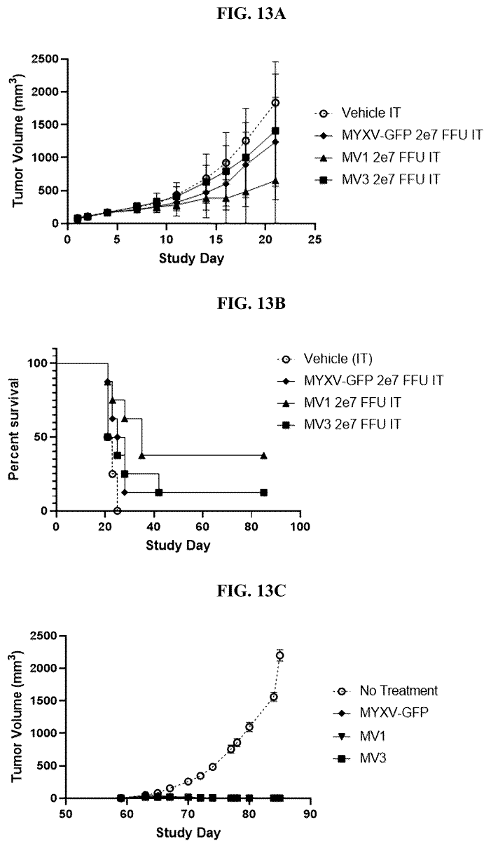

The anti-tumor efficacy of MV1 and MV3 was tested in a mouse breast carcinoma model. Balb/c mice were implanted subcutaneously with of 1×10⁶ EMT-6 cells in the right flank. Tumor bearing animals were randomized into treatment groups of 8 animals per group with an average tumor volume of 79 mm³ (range 64-99) mm³. Animals were treated via intratumoral (IT) injection of 2×10⁷ FFU/dose once every four days for four doses post-randomization with MV1, MV3, or with myxoma virus lacking the TNF-α, IL-12, and decorin transgenes (MYXV-GFP). As shown in FIG. 13A, myxoma virus treatment led to reduced tumor burden, with the lowest tumor volume observed for MV1-treated animals. Survival of the animals over time was monitored; survival endpoints were met when tumor volume was ≥1500 mm³ for an individual animal, or when IACUC guidelines for terminal sacrifice were met. As shown in FIG. 13B, treatment with myxoma virus increased the rate of survival, with the highest survival for the group treated with MV1. Animals that had survived to day 59 after initial myxoma virus dosing were re-challenged with 1×10⁶ EMT-6 cells implanted subcutaneously on the left flank, and tumor volume measurements were recorded three times per week. Animals previously treated with the myxoma viruses were resistant to tumor re-challenge, as shown in FIG. 13C.

### Example 6—Anti-Cancer Activity of Recombinant Myxoma Virus in Mouse Melanoma Model

The anti-tumor efficacy of MV1, MV2, MV3, and MV4 was tested in a mouse melanoma model. C57BL/6 mice were implanted subcutaneously with 1×10⁶ B16-F10 melanoma cells. Tumor bearing animals were randomized into treatment groups of 8 animals per group with an average tumor volume of 75-100 mm³.

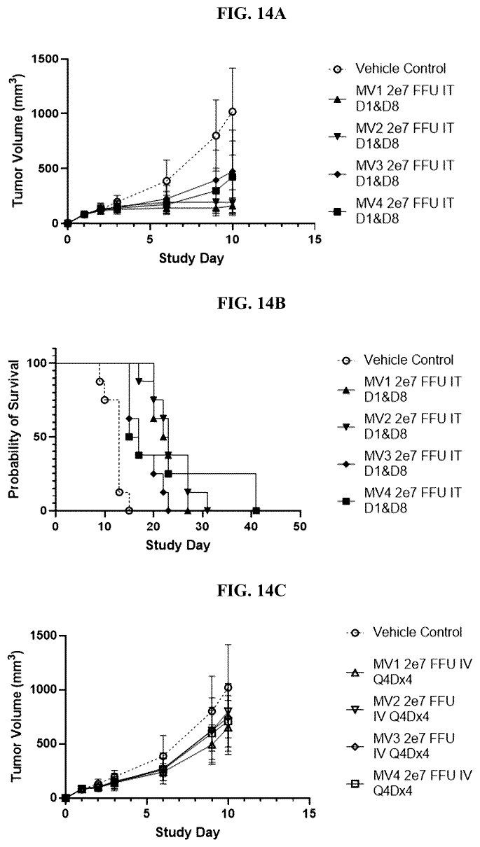

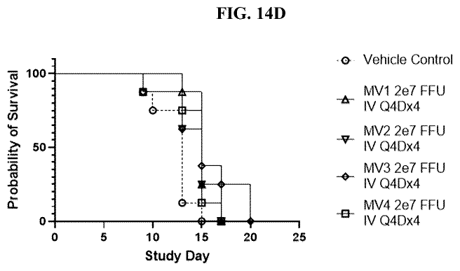

In a first experiment, animals were treated via intratumoral (IT) injection of 2×10⁷ FFU/dose on Day 1 and Day 8 post-randomization with MV1, MV2, MV3, or MV4. As shown in FIG. 14A, myxoma virus treatment led to reduced tumor burden compared to vehicle-treated control animals. Survival of the animals over time was monitored; survival endpoints were met when tumor volume was ≥1500 mm³ for an individual animal, or when IACUC guidelines for terminal sacrifice were met. As shown in FIG. 14B, treatment with myxoma virus increased the average survival time.

In a second experiment, animals were treated via intravenous (IV) injection of 2×10⁷ FFU/dose once every 4 days for 4 doses with MV1, MV2, MV3, or MV4. Tumor volume over time is plotted in FIG. 14C, and survival data is plotted in FIG. 14D. Treatment with myxoma viruses led to reduced tumor volume and increased average length of survival.

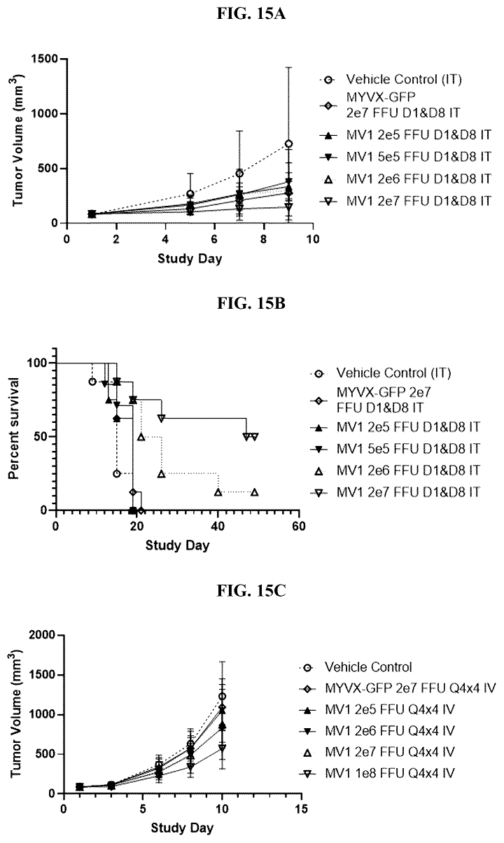

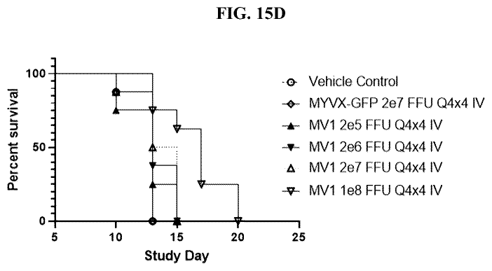

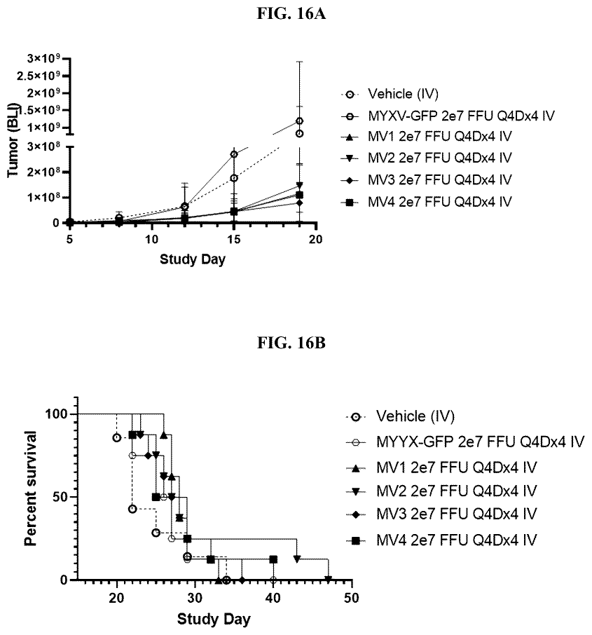

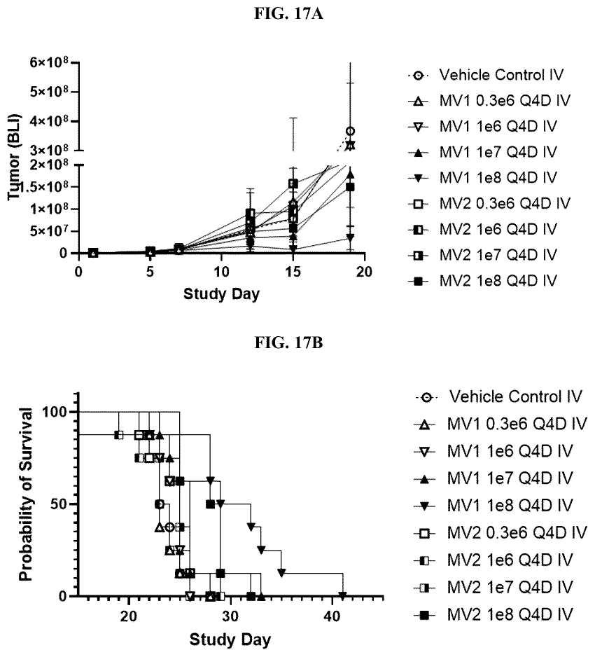

In a third experiment, animals were treated via IT injection of the indicated doses (2×10⁵, 5×10⁵, 2×10⁶, or 2×10⁷ FFU/dose) of MV1 on day 1 and day 8 post-randomization. As shown in FIG. 15A and FIG. 15B, does-dependent improvements in tumor burden and survival were observed for MV1-treated mice, and improved survival (e.g., survival rate, and/or mean survival time) was achieved compared to mice treated with myxoma virus lacking the decorin and IL-12 transgenes, even at a lower dose of oncolytic virus.

In a fourth experiment, animals were treated via IV injection of the indicated doses (2×10⁵, 2×10⁶, 2×10⁷, or 1×10⁸ FFU/dose) of MV1 once every four days for four doses post-randomization. As shown in FIG. 15C and FIG. 15D, does-dependent improvements in tumor burden and survival were observed for MV1-treated mice.

### Example 7—Anti-Cancer Activity of Recombinant Myxoma Virus in Mouse Metastatic Melanoma Model

The anti-tumor efficacy of MV1, MV2, MV3, and MV4 was tested in a mouse metastatic melanoma model. Albino C57BL/6 mice were implanted with 0.33×10⁶ B16-F10-Luc melanoma cells via intravenous injection in the tail vein.

In a first experiment, animals were treated via intravenous (IV) injection of 2×10⁷ FFU/dose of MV1, MV2, MV3, or MV4, once every four days for four doses, beginning day 3 after tumor cell injection. Luciferase bioluminescence intensity signal (BLI) was measured as an indicator of melanoma burden. As shown in FIG. 16A, melanoma burden was reduced in animals the received the MV1, MV2, MV3, or MV4 myxoma virus compared to vehicle-treated animals and compared to animals treated with a myxoma virus that lacks the IL-12, decorin, and/or TNF-α transgenes (MYXV-GFP). Survival of the animals over time was monitored; survival endpoints were met when IACUC guidelines for terminal sacrifice were met. As shown in FIG. 16B, treatment with myxoma virus increased the mean time to death or time of survival for some animals/groups.

In a second experiment, animals were treated via intravenous (IV) injection of the indicated dose (0.3×10⁶, 1×10⁶, 1×10⁷, or 1×10⁸) FFU/dose of MV1 or MV2, once every four days for four doses, beginning day 3 after tumor cell injection. As shown in FIG. 17A and FIG. 17B, reduced melanoma burden and increased survival were observed for some groups treated with MV1 or MV2, particularly those that received higher doses.

### Example 8—Anti-Cancer Activity of Recombinant Myxoma Virus in Mouse Metastatic Osteosarcoma Model

The anti-tumor efficacy of MV1, MV2, MV3, and MV4 was tested in a mouse metastatic osteosarcoma model. Balb/c mice were implanted with 2×10⁶ K7M2-Luc osteosarcoma cells via intravenous injection in the tail vein. Survival of the animals over time was monitored; survival endpoints were met when IACUC guidelines for terminal sacrifice were met.

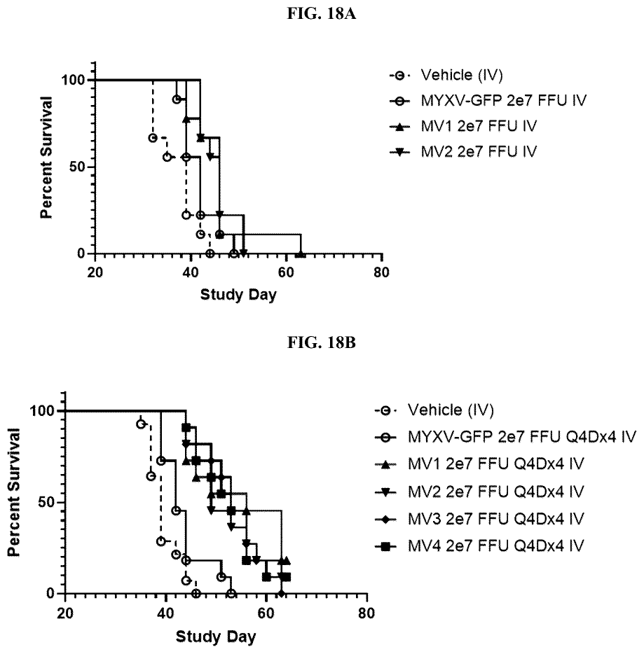

In a first experiment, animals were treated via a single intravenous (IV) injection of 2×10⁷ FFU of MV1 or MV2 on day 3 after tumor cell injection. As shown in FIG. 18A, time to death was delayed for animals treated with MV1 or MV2.

In a second experiment, animals were treated via IV injection of 2×10⁷ FFU/dose of MV1, MV2, MV3, or MV4 once every four days for four doses, beginning on day 3 after tumor cell injection. As shown in FIG. 18B, the four-dose regimen increased survival time for mice treated with MV1, MV2, MV3, or MV4 compared to vehicle-treated animals, and compared to animals treated with myxoma virus that lacks the IL-12, decorin, and/or TNF-α transgenes (MYXV-GFP).

### Example 9—Transgene Expression by Infected Cells

This example demonstrates that cells infected with myxoma viruses of the disclosure secrete Decorin and IL-12, and that the time course of transgene production can be modulated based on which promoter is utilized.

Vero cells or B16-F10 melanoma cells were plated at approximately 1.5×10⁵ cells/well in 24 well plates and allowed to adhere overnight. The cells (at least 70% confluent) were infected with the MV1, MV2, MV5, or HV11 myxoma virus at a multiplicity of infection (MOI) of 0.1, 0.3, 1, or 3. At 4 hours and 24 hours post-infection, cell culture supernatant was harvested and subjected to ELISA to measure the production of IL-12 and decorin.

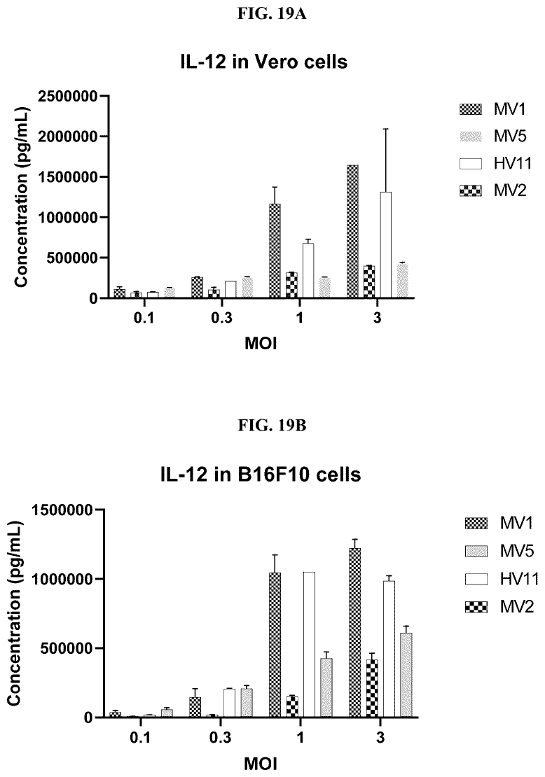

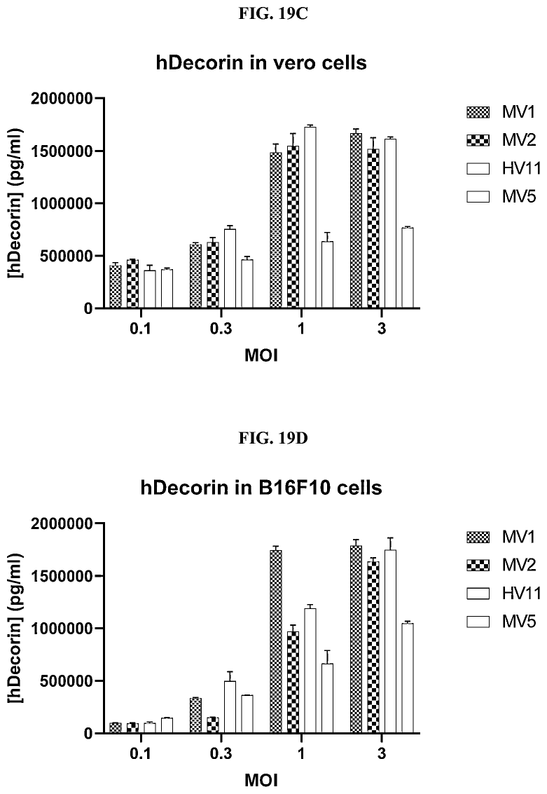

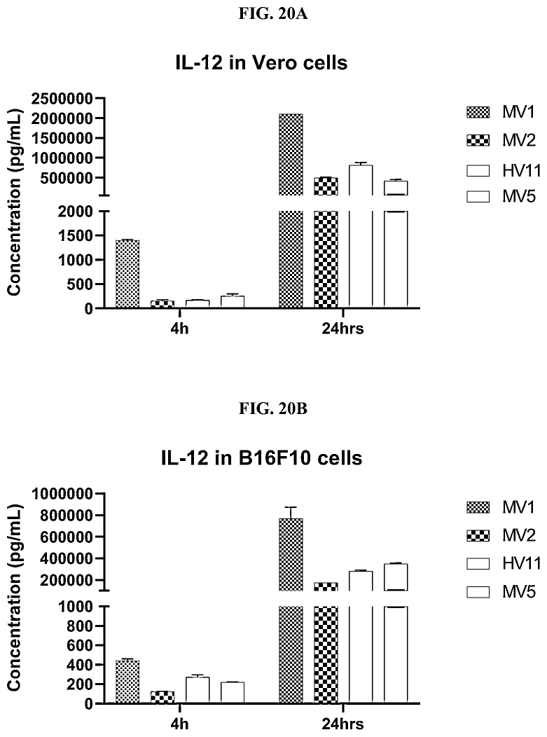

At 24 hours post-infection, an MOI-dependent effect on transgene expression was observed, with higher production of IL-12 and decorin detected for cultures infected with a higher concentration of virus (FIG. 19A—IL-12 production by Vero cells; FIG. 19B—IL-12 production by B16-F10 cells; FIG. 19C—decorin production by Vero cells; FIG. 19D—decorin production by B16-F10 cells). Relatively higher levels of IL-12 were generally detected for viruses expressing the IL-12 with an elastin linker joining the IL-12A and IL-12B subunits.

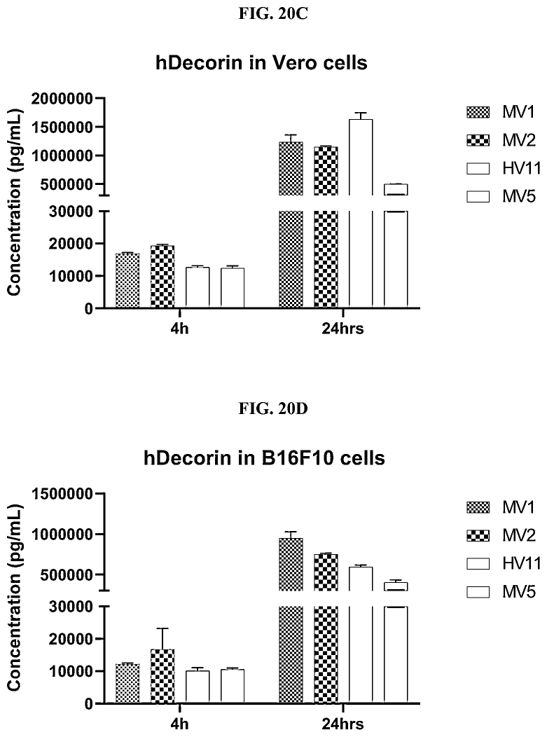

For cells infected at an MOI of 1, a time-dependent effect was observed, with higher concentrations of IL-12 and decorin detected at 24 hours compared to 4 hours post-infection (FIG. 20A—IL-12 production by Vero cells; FIG. 20B—IL-12 production by B16-F10 cells; FIG. 20C—decorin production by Vero cells; FIG. 20D—decorin production by B16-F10 cells).

At the early time point (4h) after infection, increased concentrations of IL-12 were observed for the MV1 virus, which expresses elastin-linked IL-12 from the sE/L promoter, compared to the MV5 and HV11 viruses, which each express elastin-linked IL-12 from the p11 promoter. These data demonstrate that the time course of transgene production can be modulated based on which promoter is utilized. For example, a p11 promoter can be utilized to reduce production of a transgene early in infection and/or restrict transgene expression to cancer cells in which higher viral replication occurs, which in some embodiments reduces toxicity associated with higher transgene expression from an alternative promoter, such as a sE/L promoter.

### Example 10—Inhibition of Growth of Solid Tumor Cell Lines In Vitro by Recombinant Myxoma Viruses

To further characterize the ability of HV11, HV12, HV13, and HV14 to inhibit growth of human cancers, human solid tumor cell lines were infected at 9 different multiplicities of infection (MOI=0.01, 0.03, 0.1, 0.3, 1, 3, 10, 30, 100), and growth inhibition was determined using cell titer glow viability assays at 72 hours post-infection. Adherent cell lines were infected at approximately 70% confluence.

The cell lines tested included NCI-N87 (gastric carcinoma), SK-MEL-1 (melanoma), COLO205 (colon cancer), LoVo (colorectal cancer), HCC1806 (acantholytic squamous cell carcinoma/breast cancer), HCC1599 (breast cancer), HT1080 (fibrosarcoma), SW620 (colorectal cancer), HEP3B (hepatocellular carcinoma), MKN-45 (metastatic gastric adenocarcinoma), SJSA-1 (osteosarcoma), HUH-7 (hepatocellular carcinoma), A673 (Ewing sarcoma), MDA-MB-435 (metastatic melanoma), H1975 (lung adenocarcinoma/non-small cell lung cancer), SK-MEL-28 (melanoma), HT-29 (colorectal adenocarcinoma), A204 (Rhabdomyosarcoma), A549 (lung adenocarcinoma), DLD-1 (colorectal adenocarcinoma), A375 (melanoma), MDA-MB-231 (metastatic breast adenocarcinoma), SK-MES-1 (lung squamous cell carcinoma), H358 (Bronchioalveolar carcinoma/non-small cell lung cancer), HEP-G2 (hepatoblastoma/hepatocellular carcinoma), and MDA-MB-157 (metastatic breast carcinoma).

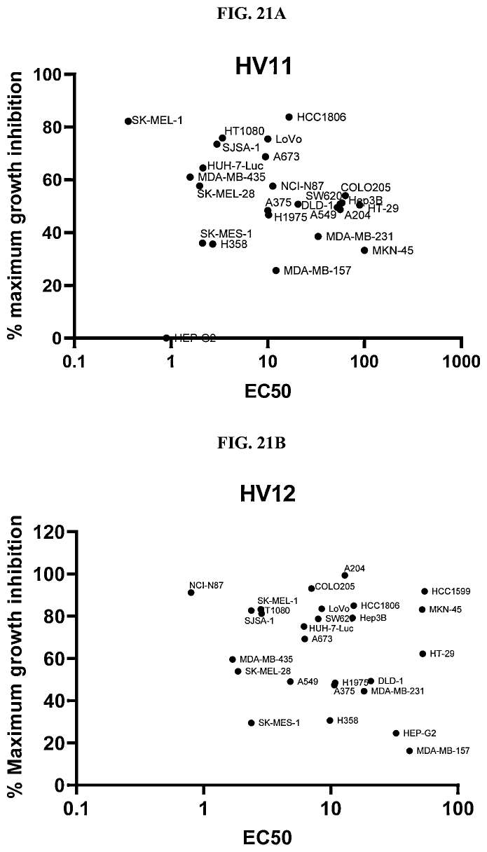

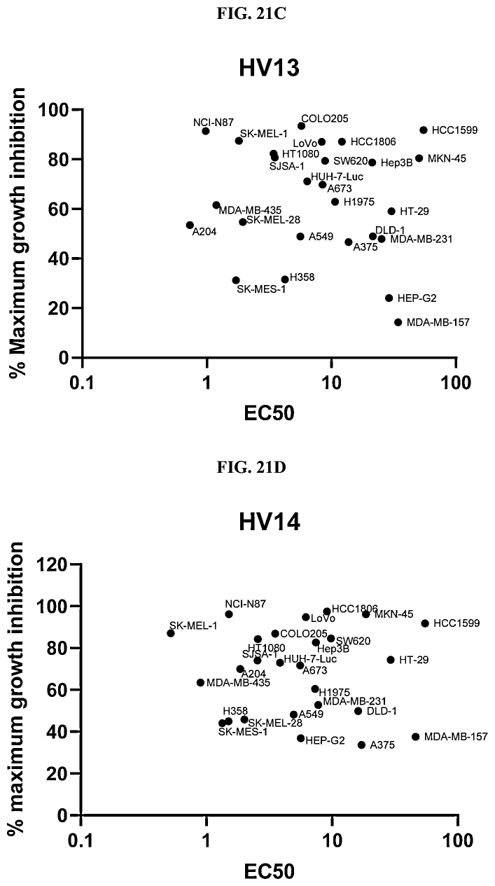

EC50 values were calculated and plotted against the percent of maximum growth inhibition, allowing visualization of how potently each virus could inhibit growth of the cancer cell lines (FIG. 21A—HV11; FIG. 21B—HV12; FIG. 21C—HV13; FIG. 21D—HV14). EC50 values were calculated as the 50% of the maximum response inhibition compared to control determined from the luminescence signals. The surviving fraction of cells was determined by dividing the mean luminescence values of the test agents by the mean luminescence values of untreated control. The effective concentration value for the test agent and control were estimated using Prism 8 software (GraphPad Software, Inc.) by curve-fitting the normalized response data using the non-linear regression analysis.

These data also provide examples of myxoma viruses disclosed herein that exhibit strong inhibition of cancer cells, which can be dependent on, for example, the combination of transgenes, which promoter(s) drive expression of the transgene(s), the presence/absence of a linker between IL-12A and IL-12B subunits, transgene orientation, cancer cell type, cancer cell characteristics, or a combination thereof.

### Example 11—Inhibition of Multiple Myeloma Cell Lines In Vitro by HV11

To further characterize the ability of HV11 to inhibit growth of multiple myeloma, cell lines were plated at approximately 1×10⁵ cells per well, infected at 9 different multiplicities of infection (MOI=0.01, 0.03, 0.1, 0.3, 1, 3, 10, 30, 100), and growth inhibition was determined at 24 and 72 hours post-infection using cell titer glow viability assays.

The cell lines tested included KMS-34(r), LP-1, RMPI-8226, L363, NCI-H929, MM1.s, U266, KMS-34, and ANBL-6.

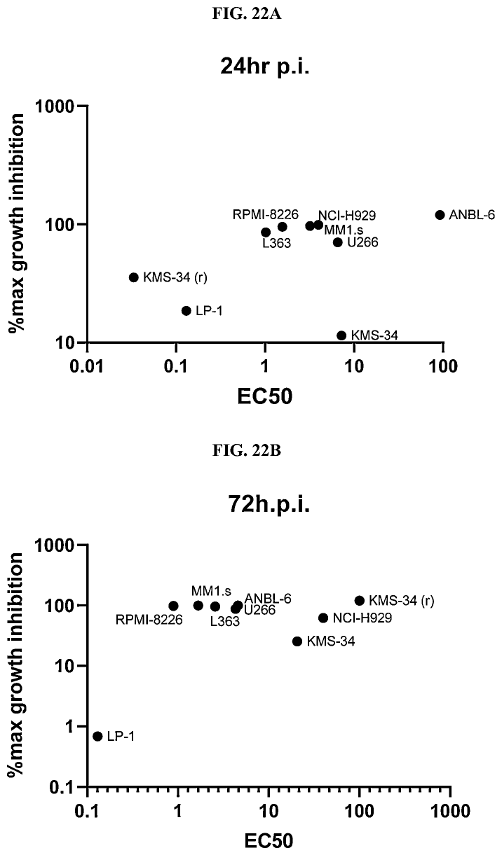

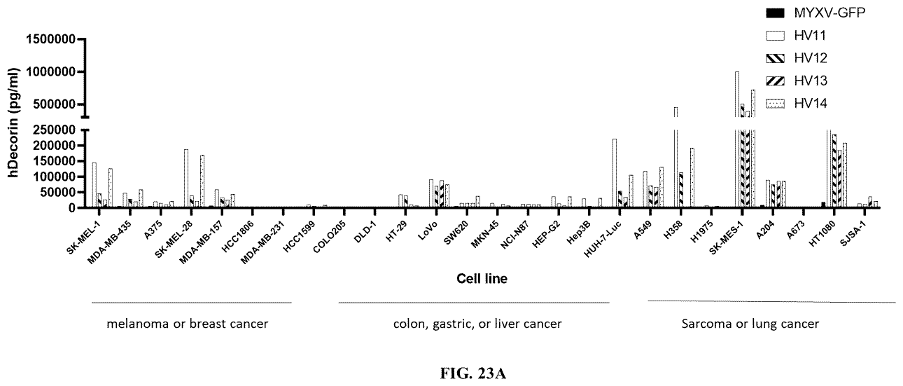

EC50 values were calculated and plotted against the percent of maximum growth inhibition, allowing visualization of how potently each virus inhibited growth of the multiple myeloma cell lines (FIG. 22A—24 hours; FIG. 22B—72 hours). EC50 values were calculated as the 50% of the maximum response inhibition compared to control determined from the luminescence signals. The surviving fraction of cells was determined by dividing the mean luminescence values of the test agents by the mean luminescence values of untreated control. The effective concentration value for the test agent and control were estimated using Prism 8 software (GraphPad Software, Inc.) by curve-fitting the normalized response data using the non-linear regression analysis.

### Example 12—Decorin, IL-12, and TNF-α Production by Solid Tumor Cell Lines Infected with Recombinant Myxoma Viruses In Vitro

To further characterize the ability of myxoma viruses disclosed herein to elicit production of decorin, IL-12, and/or TNF-α upon infection of cancer cells, human solid tumor cell lines were infected with HV11, HV12, HV13, or HV14 at a multiplicity of infection of 1, and the concentration of each protein quantified in supernatant at 24 hours post-infection. Adherent cells were infected at approximately 70% confluence. As a control, the cells were infected with an “empty” Myxoma virus (MYXV-GFP) that does not encode the decorin, IL-12, or TNF-α, and which contains an intact M153 gene.

The cell lines tested included NCI-N87 (gastric carcinoma), SK-MEL-1 (melanoma), COLO205 (colon cancer), LoVo (colorectal cancer), HCC1806 (acantholytic squamous cell carcinoma/breast cancer), HCC1599 (breast cancer), HT1080 (fibrosarcoma), SW620 (colorectal cancer), HEP3B (hepatocellular carcinoma), MKN-45 (metastatic gastric adenocarcinoma), SJSA-1 (osteosarcoma), HUH-7 (hepatocellular carcinoma), A673 (Ewing sarcoma), MDA-MB-435 (metastatic melanoma), H1975 (lung adenocarcinoma/non-small cell lung cancer), SK-MEL-28 (melanoma), HT-29 (colorectal adenocarcinoma), A204 (Rhabdomyosarcoma), A549 (lung adenocarcinoma), DLD-1 (colorectal adenocarcinoma), A375 (melanoma), MDA-MB-231 (metastatic breast adenocarcinoma), SK-MES-1 (lung squamous cell carcinoma), H358 (Bronchioalveolar carcinoma/non-small cell lung cancer), HEP-G2 (hepatoblastoma/hepatocellular carcinoma), and MDA-MB-157 (metastatic breast carcinoma).

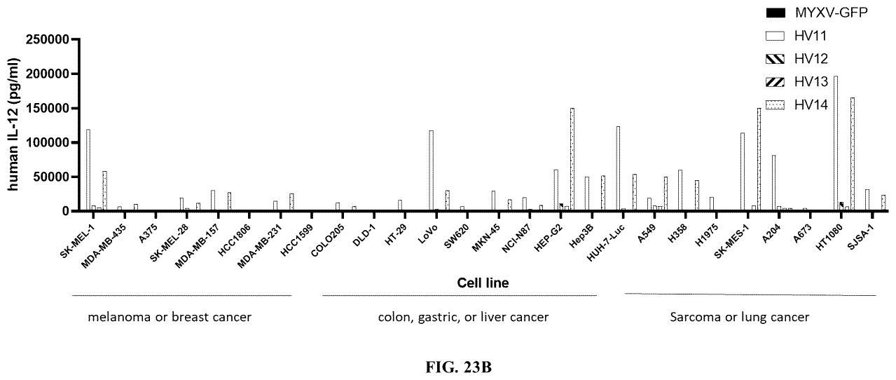

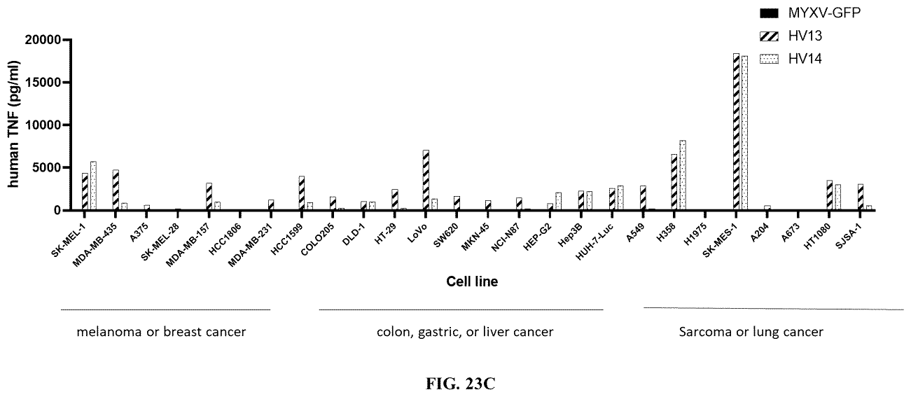

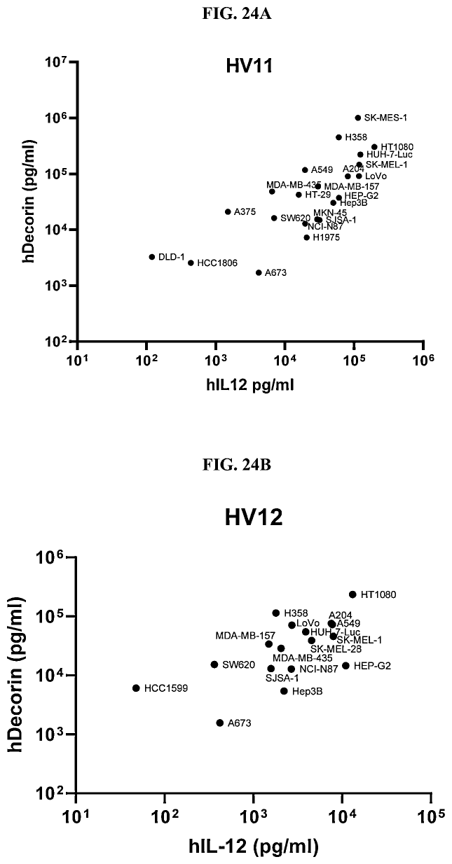

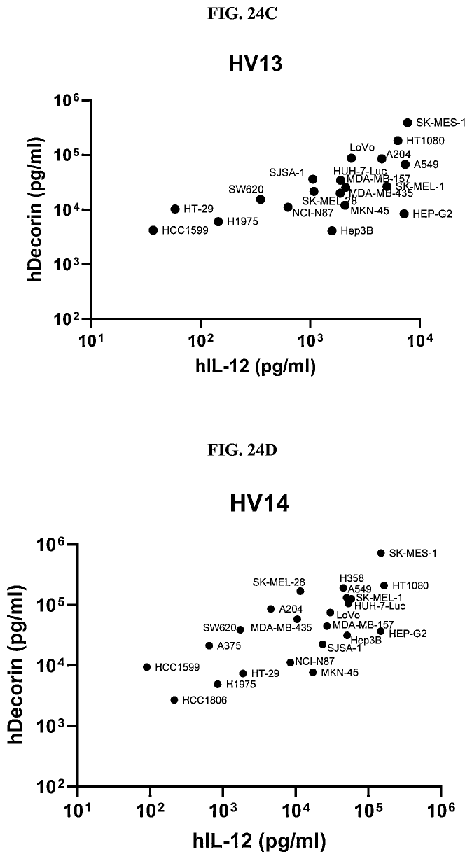

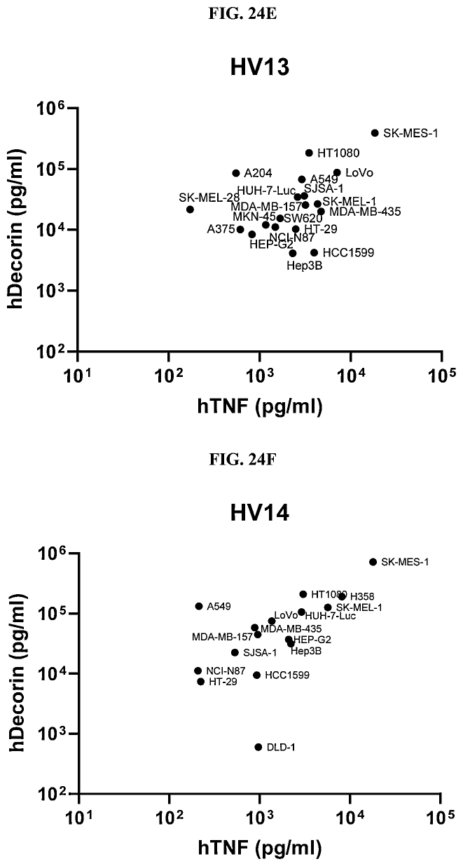

HV11, HV12, HV13, and HV14 elicited production of decorin by solid tumor cells (FIGS. 23A and 24A-F), whereas MYXV-GFP elicited less decorin, no decorin, or substantially no decorin (FIG. 23A). In a number of cases, higher decorin was observed in response to HV11 and HV14 (FIG. 23A), despite decorin expression being driven by the sE/L promoter in all the viruses.

HV11, HV12, HV13, and HV14 elicited production of IL-12 by solid tumor cells (FIGS. 23B and 24A-D), whereas MYXV-GFP elicited no or substantially no IL-12 (FIG. 23B). Higher IL-12 was produced by cells infected with HV11 or HV14 (FIG. 23B).

HV13 and HV14 elicited production of TNF-α by solid tumor cells (FIGS. 23C and 24E-F), whereas MYXV-GFP elicited less TNF-α, no TNF-α, or substantially no TNF-α (FIG. 23C). In a number of cases, higher TNF-α was produced by cells infected with HV13 (FIG. 23C), in which TNF-α is driven by an sE/L promoter rather than a p11 promoter.

### Example 13—Decorin and IL-12 Production by Multiple Myeloma Cell Lines Infected with HV11 In Vitro

To further characterize the ability of myxoma viruses disclosed herein to elicit production of decorin and IL-12 upon infection of cancer cells, human multiple myeloma cell lines were plated at approximately 1×10⁵ cells per well, infected with HV11 at a multiplicity of infection of 1, and the concentrations of decorin and IL-12 quantified at 24 hours post-infection. As a control, the cells were infected with an “empty” Myxoma virus (MYXV-GFP) that does not encode the decorin or IL-12, and which contains an intact M153 gene.

The cell lines tested included KMS-34(r), LP-1, RMPI-8226, L363, NCI-H929, MM1.s, U266, KMS-34, and ANBL-6.

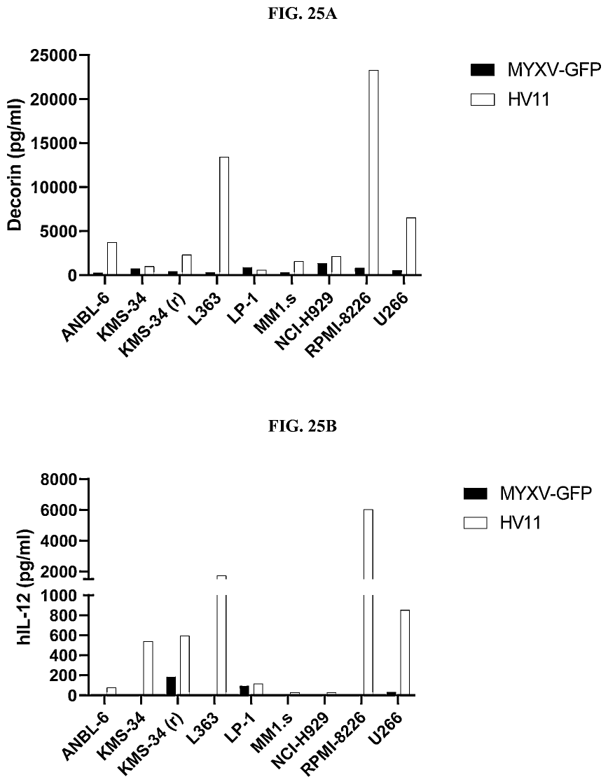

In a number of the cell lines tested, HV11 elicited IL-12 and decorin, whereas MYXV-GFP elicited less or substantially no decorin (FIG. 25A) and/or IL-12 (FIG. 25B).

### Additional Sequences

Exemplary sequences corresponding to the compositions and methods described herein are shown in Table 4.

TABLE 4

| SEQ | | |
|---|---|---|
| ID NO | Name | Sequence |
| 1 | Synthetic early/late | AAAAATTGAAATTTTATTTTTTTTTTTTGGAATATAAA |
| | promoter | TA |
| 2 | P11 (promoter) | GAATTTCATTTTGTTTTTTTCTATGCTATAA |
| 3 | hu IL-12B (p40) | ATGTGTCACCAGCAGTTGGTCATCTCTTGGTTTTCCCT |
| | | GGTTTTTCTGGCATCTCCCCTCGTGGCCATATGGGAAC |
| | | TGAAGAAAGATGTTTATGTCGTAGAATTGGATTGGTA |
| | | TCCGGATGCCCCTGGAGAAATGGTGGTCCTCACCTGT |
| | | GACACCCCTGAAGAAGATGGTATCACCTGGACCTTGG |
| | | ACCAGAGCAGTGAGGTCTTAGGCTCTGGCAAAACCCT |
| | | GACCATCCAAGTCAAAGAGTTTGGAGATGCTGGCCAG |
| | | TACACCTGTCACAAAGGAGGCGAGGTTCTAAGCCATT |
| | | CGCTCCTGCTGCTTCACAAAAAGGAAGATGGAATTTG |
| | | GTCCACTGATATTTTAAAGGACCAGAAAGAACCCAAA |
| | | AATAAGACCTTTCTAAGATGCGAGGCCAAGAATTATT |
| | | CTGGACGTTTCACCTGCTGGTGGCTGACGACAATCAGT |
| | | ACTGATTTGACATTCAGTGTCAAAAGCAGCAGAGGCT |
| | | CTTCTGACCCCCAAGGGGTGACGTGCGGAGCTGCTAC |
| | | ACTCTCTGCAGAGAGAGTCAGAGGGGACAACAAGGA |
| | | GTATGAGTACTCAGTGGAGTGCCAGGAGGACAGTGCC |
| | | TGCCCAGCTGCTGAGGAGAGTCTGCCCATTGAGGTCA |
| | | TGGTGGATGCCGTTCACAAGCTCAAGTATGAAAACTA |
| | | CACCAGCAGCTTCTTCATCAGGGACATCATCAAACCT |
| | | GACCCACCCAAGAACTTGCAGCTGAAGCCATTAAAGA |
| | | ATTCTCGGCAGGTGGAGGTCAGCTGGGAGTACCCTGA |
| | | CACCTGGAGTACTCCACATTCCTACTTCTCCCTGACAT |
| | | TCTGCGTTCAGGTCCAGGGCAAGAGCAAGAGAGAAAA |
| | | GAAAGATAGAGTCTTCACGGACAAGACCTCAGCCACG |
| | | GTCATCTGCCGCAAAAATGCCAGCATTAGCGTGCGGG |
| | | CCCAGGACCGCTACTATAGCTCATCTTGGAGCGAATG |
| | | GGCATCTGTGCCCTGCAGTTAG |
| 4 | hu IL-12A (p35) | ATGTGGCCCCCTGGGTCAGCCTCCCAGCCACCGCCCTC |
| | | ACCTGCCGCGGCCACAGGTCTGCATCCAGCGGCTCGC |
| | | CCTGTGTCCCTGCAGTGCCGGCTCAGCATGTGTCCAGC |
| | | GCGCAGCCTCCTCCTTGTGGCTACCCTGGTCCTCCTGG |
| | | ACCACCTCAGTTTGGCCAGAAACCTCCCCGTGGCCACT |
| | | CCAGACCCAGGAATGTTCCCATGCCTTCACCACTCCCA |
| | | AAACCTGCTGAGGGCCGTCAGCAACATGCTCCAGAAG |
| | | GCCAGACAAACTCTAGAATTTTACCCTTGCACTTCTGA |
| | | AGAGATTGATCATGAAGATATCACAAAAGATAAAACC |
| | | AGCACAGTGGAGGCCTGTTTACCATTGGAATTAACCA |
| | | AGAATGAGAGTTGCCTAAATTCCAGAGAGACCTCTTT |
| | | CATAACTAATGGGAGTTGCCTGGCCTCCAGAAAGACC |
| | | TCTTTTATGATGGCCCTGTGCCTTAGTAGTATTTATGA |
| | | AGACTTGAAGATGTACCAGGTGGAGTTCAAGACCATG |
| | | AATGCAAAGCTTCTGATGGATCCTAAGAGGCAGATCT |
| | | TTCTAGATCAAAACATGCTGGCAGTTATTGATGAGCTG |
| | | ATGCAGGCCCTGAATTTCAACAGTGAGACTGTGCCAC |
| | | AAAAATCCTCCCTTGAAGAACCGGATTTTTATAAAACT |
| | | AAAATCAAGCTCTGCATACTTCTTCATGCTTTCAGAAT |
| | | TCGGGCAGTGACTATTGATAGAGTGATGAGCTATCTG |
| | | AATGCTTCCTAA |
| 5 | hu IL-12A (p35)- | GCCAGAAACCTCCCCGTGGCCACTCCAGACCCAGGAA |
| | truncated, lacking | TGTTCCCATGCCTTCACCACTCCCAAAACCTGCTGAGG |
| | signal peptide | GCCGTCAGCAACATGCTCCAGAAGGCCAGACAAACTC |
| | | TAGAATTTTACCCTTGCACTTCTGAAGAGATTGATCAT |
| | | GAAGATATCACAAAAGATAAAACCAGCACAGTGGAG |
| | | GCCTGTTTACCATTGGAATTAACCAAGAATGAGAGTT |
| | | GCCTAAATTCCAGAGAGACCTCTTTCATAACTAATGG |
| | | GAGTTGCCTGGCCTCCAGAAAGACCTCTTTTATGATGG |
| | | CCCTGTGCCTTAGTAGTATTTATGAAGACTTGAAGATG |
| | | TACCAGGTGGAGTTCAAGACCATGAATGCAAAGCTGC |
| | | TGATGGATCCTAAGAGGCAGATCTTTCTAGATCAAAA |
| | | CATGCTGGCAGTTATTGATGAGCTGATGCAGGCCCTG |
| | | AATTTCAACAGTGAGACTGTGCCACAAAAATCCTCCC |
| | | TTGAAGAACCGGATTTTTATAAAACTAAAATCAAGCT |
| | | CTGCATACTTCTTCATGCTTTCAGAATTCGGGCAGTGA |
| | | CTATTGATAGAGTGATGAGCTATCTGAATGCTTCCTAA |
| 6 | Elastin linker | GTTCCTGGAGTAGGGGTACCTGGGGTGGGC |
| 7 | Hu Decorin | ATGAAGGCCACTATCATCCTCCTTCTGCTTGCACAAGT |
| | | TTCCTGGGCTGGACCGTTTCAACAGAGAGGCTTATTTG |
| | | ACTTTATGCTAGAAGATGAGGCTTCTGGGATAGGCCC |
| | | AGAAGTTCCTGATGACCGCGACTTCGAGCCCTCCCTA |
| | | GGCCCAGTGTGCCCCTTCCGCTGTCAATGCCATCTTCG |
| | | AGTGGTCCAGTGTTCTGATTTGGGTCTGGACAAAGTGC |
| | | CAAAGGATCTTCCCCCTGACACAACTCTGCTAGACCTG |
| | | CAAAACAACAAAATAACCGAAATCAAAGATGGAGAC |
| | | TTTAAGAACCTGAAGAACCTTCACGCATTGATTCTTGT |
| | | CAACAATAAAATTAGCAAAGTTAGTCCTGGAGCATTT |
| | | ACACCTTTGGTGAAGTTGGAACGACTTTATCTGTCCAA |
| | | GAATCAGCTGAAGGAATTGCCAGAAAAAATGCCCAAA |
| | | ACTCTTCAGGAGCTGCGTGCCCATGAGAATGAGATCA |
| | | CCAAAGTGCGAAAAGTTACTTTCAATGGACTGAACCA |
| | | GATGATTGTCATAGAACTGGGCACCAATCCGCTGAAG |
| | | AGCTCAGGAATTGAAAATGGGGCTTTCCAGGGAATGA |
| | | AGAAGCTCTCCTACATCCGCATTGCTGATACCAATATC |
| | | ACCAGCATTCCTCAAGGTCTTCCTCCTTCCCTTACGGA |
| | | ATTACATCTTGATGGCAACAAAATCAGCAGAGTTGAT |
| | | GCAGCTAGCCTGAAAGGACTGAATAATTTGGCTAAGT |
| | | TGGGATTGAGTTTCAACAGCATCTCTGCTGTTGACAAT |
| | | GGCTCTCTGGCCAACACGCCTCATCTGAGGGAGCTTC |
| | | ACTTGGACAACAACAAGCTTACCAGAGTACCTGGTGG |
| | | GCTGGCAGAGCATAAGTACATCCAGGTTGTCTACCTTC |
| | | ATAACAACAATATCTCTGTAGTTGGATCAAGTGACTTC |
| | | TGCCCACCTGGACACAACACCAAAAAGGCTTCTTATT |
| | | CGGGTGTGAGTCTTTTCAGCAACCCGGTCCAGTACTGG |
| | | GAGATACAGCCATCCACCTTCAGATGTGTCTACGTGC |
| | | GCTCTGCCATTCAACTCGGAAACTATAAGTAA |
| 8 | GFP | ATGGTGAGCAAGGGCGAGGAGCTGTTCACCGGGGTGG |
| | | TGCCCATCCTGGTCGAGCTGGACGGCGACGTAAACGG |
| | | CCACAAGTTCAGCGTGTCCGGCGAGGGCGAGGGCGAT |
| | | GCCACCTACGGCAAGCTGACCCTGAAGTTCATCTGCA |
| | | CCACCGGCAAGCTGCCCGTGCCCTGGCCCACCCTCGT |
| | | GACCACCCTGACCTACGGCGTGCAGTGCTTCAGCCGC |
| | | TACCCCGACCACATGAAGCAGCACGACTTCTTCAAGT |
| | | CCGCCATGCCCGAAGGCTACGTCCAGGAGCGCACCAT |
| | | CTTCTTCAAGGACGACGGCAACTACAAGACCCGCGCC |
| | | GAGGTGAAGTTCGAGGGCGACACCCTGGTGAACCGCA |
| | | TCGAGCTGAAGGGCATCGACTTCAAGGAGGACGGCAA |
| | | CATCCTGGGGCACAAGCTGGAGTACAACTACAACAGC |
| | | CACAACGTCTATATCATGGCCGACAAGCAGAAGAACG |
| | | GCATCAAGGTGAACTTCAAGATCCGCCACAACATCGA |
| | | GGACGGCAGCGTGCAGCTCGCCGACCACTACCAGCAG |
| | | AACACCCCCATCGGCGACGGCCCCGTGCTGCTGCCCG |
| | | ACAACCACTACCTGAGCACCCAGTCCGCCCTGAGCAA |
| | | AGACCCCAACGAGAAGCGCGATCACATGGTCCTGCTG |
| | | GAGTTCGTGACCGCCGCCGGGATCACTCTCGGCATGG |
| | | ACGAGCTGTACAAGTAA |
| 9 | IL-12B-elastin-IL- | ATGTGTCACCAGCAGTTGGTCATCTCTTGGTTTTCCCT |
| | 12A | GGTTTTTCTGGCATCTCCCCTCGTGGCCATATGGGAAC |
| | | TGAAGAAAGATGTTTATGTCGTAGAATTGGATTGGTA |
| | | TCCGGATGCCCCTGGAGAAATGGTGGTCCTCACCTGT |
| | | GACACCCCTGAAGAAGATGGTATCACCTGGACCTTGG |
| | | ACCAGAGCAGTGAGGTCTTAGGCTCTGGCAAAACCCT |
| | | GACCATCCAAGTCAAAGAGTTTGGAGATGCTGGCCAG |
| | | TACACCTGTCACAAAGGAGGCGAGGTTCTAAGCCATT |
| | | CGCTCCTGCTGCTTCACAAAAAGGAAGATGGAATTTG |
| | | GTCCACTGATATTTTAAAGGACCAGAAAGAACCCAAA |
| | | AATAAGACCTTTCTAAGATGCGAGGCCAAGAATTATT |
| | | CTGGACGTTTCACCTGCTGGTGGCTGACGACAATCAGT |
| | | ACTGATTTGACATTCAGTGTCAAAAGCAGCAGAGGCT |
| | | CTTCTGACCCCCAAGGGGTGACGTGCGGAGCTGCTAC |
| | | ACTCTCTGCAGAGAGAGTCAGAGGGGACAACAAGGA |
| | | GTATGAGTACTCAGTGGAGTGCCAGGAGGACAGTGCC |
| | | TGCCCAGCTGCTGAGGAGAGTCTGCCCATTGAGGTCA |
| | | TGGTGGATGCCGTTCACAAGCTCAAGTATGAAAACTA |
| | | CACCAGCAGCTTCTTCATCAGGGACATCATCAAACCT |
| | | GACCCACCCAAGAACTTGCAGCTGAAGCCATTAAAGA |
| | | ATTCTCGGCAGGTGGAGGTCAGCTGGGAGTACCCTGA |
| | | CACCTGGAGTACTCCACATTCCTACTTCTCCCTGACAT |
| | | TCTGCGTTCAGGTCCAGGGCAAGAGCAAGAGAGAAAA |
| | | GAAAGATAGAGTCTTCACGGACAAGACCTCAGCCACG |
| | | GTCATCTGCCGCAAAAATGCCAGCATTAGCGTGCGGG |
| | | CCCAGGACCGCTACTATAGCTCATCTTGGAGCGAATG |
| | | GGCATCTGTGCCCTGCAGTGTTCCTGGAGTAGGGGTA |
| | | CCTGGGGTGGGCGCCAGAAACCTCCCCGTGGCCACTC |
| | | CAGACCCAGGAATGTTCCCATGCCTTCACCACTCCCAA |
| | | AACCTGCTGAGGGCCGTCAGCAACATGCTCCAGAAGG |
| | | CCAGACAAACTCTAGAATTTTACCCTTGCACTTCTGAA |
| | | GAGATTGATCATGAAGATATCACAAAAGATAAAACCA |
| | | GCACAGTGGAGGCCTGTTTACCATTGGAATTAACCAA |
| | | GAATGAGAGTTGCCTAAATTCCAGAGAGACCTCTTTC |
| | | ATAACTAATGGGAGTTGCCTGGCCTCCAGAAAGACCT |
| | | CTTTTATGATGGCCCTGTGCCTTAGTAGTATTTATGAA |
| | | GACTTGAAGATGTACCAGGTGGAGTTCAAGACCATGA |
| | | ATGCAAAGCTGCTGATGGATCCTAAGAGGCAGATCTT |
| | | TCTAGATCAAAACATGCTGGCAGTTATTGATGAGCTG |
| | | ATGCAGGCCCTGAATTTCAACAGTGAGACTGTGCCAC |
| | | AAAAATCCTCCCTTGAAGAACCGGATTTTTATAAAACT |
| | | AAAATCAAGCTCTGCATACTTCTTCATGCTTTCAGAAT |
| | | TCGGGCAGTGACTATTGATAGAGTGATGAGCTATCTG |
| | | AATGCTTCCTAA |
| 10 | HV11 insert | GAATTTCATTTTGTTTTTTTCTATGCTATAAATGTGTCA |
| | | CCAGCAGTTGGTCATCTCTTGGTTTTCCCTGGTTTTTCT |
| | | GGCATCTCCCCTCGTGGCCATATGGGAACTGAAGAAA |
| | | GATGTTTATGTCGTAGAATTGGATTGGTATCCGGATGC |
| | | CCCTGGAGAAATGGTGGTCCTCACCTGTGACACCCCT |
| | | GAAGAAGATGGTATCACCTGGACCTTGGACCAGAGCA |
| | | GTGAGGTCTTAGGCTCTGGCAAAACCCTGACCATCCA |
| | | AGTCAAAGAGTTTGGAGATGCTGGCCAGTACACCTGT |
| | | CACAAAGGAGGCGAGGTTCTAAGCCATTCGCTCCTGC |
| | | TGCTTCACAAAAAGGAAGATGGAATTTGGTCCACTGA |
| | | TATTTTAAAGGACCAGAAAGAACCCAAAAATAAGACC |
| | | TTTCTAAGATGCGAGGCCAAGAATTATTCTGGACGTTT |
| | | CACCTGCTGGTGGCTGACGACAATCAGTACTGATTTG |
| | | ACATTCAGTGTCAAAAGCAGCAGAGGCTCTTCTGACC |
| | | CCCAAGGGGTGACGTGCGGAGCTGCTACACTCTCTGC |
| | | AGAGAGAGTCAGAGGGGACAACAAGGAGTATGAGTA |
| | | CTCAGTGGAGTGCCAGGAGGACAGTGCCTGCCCAGCT |
| | | GCTGAGGAGAGTCTGCCCATTGAGGTCATGGTGGATG |
| | | CCGTTCACAAGCTCAAGTATGAAAACTACACCAGCAG |
| | | CTTCTTCATCAGGGACATCATCAAACCTGACCCACCCA |
| | | AGAACTTGCAGCTGAAGCCATTAAAGAATTCTCGGCA |
| | | GGTGGAGGTCAGCTGGGAGTACCCTGACACCTGGAGT |
| | | ACTCCACATTCCTACTTCTCCCTGACATTCTGCGTTCA |
| | | GGTCCAGGGCAAGAGCAAGAGAGAAAAGAAAGATAG |
| | | AGTCTTCACGGACAAGACCTCAGCCACGGTCATCTGC |
| | | CGCAAAAATGCCAGCATTAGCGTGCGGGCCCAGGACC |
| | | GCTACTATAGCTCATCTTGGAGCGAATGGGCATCTGTG |
| | | CCCTGCAGTGTTCCTGGAGTAGGGGTACCTGGGGTGG |
| | | GCGCCAGAAACCTCCCCGTGGCCACTCCAGACCCAGG |
| | | AATGTTCCCATGCCTTCACCACTCCCAAAACCTGCTGA |
| | | GGGCCGTCAGCAACATGCTCCAGAAGGCCAGACAAAC |
| | | TCTAGAATTTTACCCTTGCACTTCTGAAGAGATTGATC |
| | | ATGAAGATATCACAAAAGATAAAACCAGCACAGTGG |
| | | AGGCCTGTTTACCATTGGAATTAACCAAGAATGAGAG |
| | | TTGCCTAAATTCCAGAGAGACCTCTTTCATAACTAATG |
| | | GGAGTTGCCTGGCCTCCAGAAAGACCTCTTTTATGATG |
| | | GCCCTGTGCCTTAGTAGTATTTATGAAGACTTGAAGAT |
| | | GTACCAGGTGGAGTTCAAGACCATGAATGCAAAGCTG |
| | | CTGATGGATCCTAAGAGGCAGATCTTTCTAGATCAAA |
| | | ACATGCTGGCAGTTATTGATGAGCTGATGCAGGCCCT |
| | | GAATTTCAACAGTGAGACTGTGCCACAAAAATCCTCC |
| | | CTTGAAGAACCGGATTTTTATAAAACTAAAATCAAGC |
| | | TCTGCATACTTCTTCATGCTTTCAGAATTCGGGCAGTG |
| | | ACTATTGATAGAGTGATGAGCTATCTGAATGCTTCCTA |
| | | AAAAAATTGAAATTTTATTTTTTTTTTTTGGAATATAA |
| | | ATAATGAAGGCCACTATCATCCTCCTTCTGCTTGCACA |
| | | AGTTTCCTGGGCTGGACCGTTTCAACAGAGAGGCTTAT |
| | | TTGACTTTATGCTAGAAGATGAGGCTTCTGGGATAGG |
| | | CCCAGAAGTTCCTGATGACCGCGACTTCGAGCCCTCCC |
| | | TAGGCCCAGTGTGCCCCTTCCGCTGTCAATGCCATCTT |
| | | CGAGTGGTCCAGTGTTCTGATTTGGGTCTGGACAAAGT |
| | | GCCAAAGGATCTTCCCCCTGACACAACTCTGCTAGAC |
| | | CTGCAAAACAACAAAATAACCGAAATCAAAGATGGA |
| | | GACTTTAAGAACCTGAAGAACCTTCACGCATTGATTCT |
| | | TGTCAACAATAAAATTAGCAAAGTTAGTCCTGGAGCA |
| | | TTTACACCTTTGGTGAAGTTGGAACGACTTTATCTGTC |
| | | CAAGAATCAGCTGAAGGAATTGCCAGAAAAAATGCCC |
| | | AAAACTCTTCAGGAGCTGCGTGCCCATGAGAATGAGA |
| | | TCACCAAAGTGCGAAAAGTTACTTTCAATGGACTGAA |
| | | CCAGATGATTGTCATAGAACTGGGCACCAATCCGCTG |
| | | AAGAGCTCAGGAATTGAAAATGGGGCTTTCCAGGGAA |
| | | TGAAGAAGCTCTCCTACATCCGCATTGCTGATACCAAT |
| | | ATCACCAGCATTCCTCAAGGTCTTCCTCCTTCCCTTAC |
| | | GGAATTACATCTTGATGGCAACAAAATCAGCAGAGTT |
| | | GATGCAGCTAGCCTGAAAGGACTGAATAATTTGGCTA |
| | | AGTTGGGATTGAGTTTCAACAGCATCTCTGCTGTTGAC |
| | | AATGGCTCTCTGGCCAACACGCCTCATCTGAGGGAGC |
| | | TTCACTTGGACAACAACAAGCTTACCAGAGTACCTGG |
| | | TGGGCTGGCAGAGCATAAGTACATCCAGGTTGTCTAC |
| | | CTTCATAACAACAATATCTCTGTAGTTGGATCAAGTGA |
| | | CTTCTGCCCACCTGGACACAACACCAAAAAGGCTTCTT |
| | | ATTCGGGTGTGAGTCTTTTCAGCAACCCGGTCCAGTAC |
| | | TGGGAGATACAGCCATCCACCTTCAGATGTGTCTACGT |
| | | GCGCTCTGCCATTCAACTCGGAAACTATAAGTAAAAA |
| | | AATTGAAATTTTATTTTTTTTTTTTGGAATATAAATAAT |
| | | GGTGAGCAAGGGCGAGGAGCTGTTCACCGGGGTGGTG |
| | | CCCATCCTGGTCGAGCTGGACGGCGACGTAAACGGCC |
| | | ACAAGTTCAGCGTGTCCGGCGAGGGCGAGGGCGATGC |
| | | CACCTACGGCAAGCTGACCCTGAAGTTCATCTGCACC |
| | | ACCGGCAAGCTGCCCGTGCCCTGGCCCACCCTCGTGA |
| | | CCACCCTGACCTACGGCGTGCAGTGCTTCAGCCGCTAC |
| | | CCCGACCACATGAAGCAGCACGACTTCTTCAAGTCCG |
| | | CCATGCCCGAAGGCTACGTCCAGGAGCGCACCATCTT |
| | | CTTCAAGGACGACGGCAACTACAAGACCCGCGCCGAG |
| | | GTGAAGTTCGAGGGCGACACCCTGGTGAACCGCATCG |
| | | AGCTGAAGGGCATCGACTTCAAGGAGGACGGCAACAT |
| | | CCTGGGGCACAAGCTGGAGTACAACTACAACAGCCAC |
| | | AACGTCTATATCATGGCCGACAAGCAGAAGAACGGCA |
| | | TCAAGGTGAACTTCAAGATCCGCCACAACATCGAGGA |
| | | CGGCAGCGTGCAGCTCGCCGACCACTACCAGCAGAAC |
| | | ACCCCCATCGGCGACGGCCCCGTGCTGCTGCCCGACA |
| | | ACCACTACCTGAGCACCCAGTCCGCCCTGAGCAAAGA |
| | | CCCCAACGAGAAGCGCGATCACATGGTCCTGCTGGAG |
| | | TTCGTGACCGCCGCCGGGATCACTCTCGGCATGGACG |
| | | AGCTGTACAAGTAA |
| 11 | HV11 insert | GATGTCGTACATCGATTACACAAAGAAGTAGAGTCAT |
| | including | ACGACGTACGTTTCCCTATAAAATCGGTAAACCTAGA |
| | recombination arms | CGCGGTGTTTCTATCCATAAACGTAACACGTGTACGTC |
| | | TACGTTGGAAGATACCCTTGACCGAACACAATCCTTAT |
| | | CAGACGGCCTACGGATGTTCTAACGACAGATTATACA |
| | | GCTACAACGAGTACGCTTTTTCTCATTTAAAACAAGAC |
| | | CGTGTAAAGATCATAGAACTCCCATGTGACGACGATT |
| | | ACAGCGTCGTGTTAATCACACACGATAGCCGTTCGAC |
| | | TATTACACCGGATAAAGTGACCGGGTGGCTGCGCACG |
| | | ACCCGTCTACGTTACGTAAACGTATCCCTACCCAAGG |
| | | GTTCCACGGAAACGGGACACAACGTAACGTGTCTAAC |
| | | TCCCACACACGTCAATCTATGTCATCGTTGTCGTATAA |
| | | CGATTACCAAAACGGGCGTGGACGCAACCGCGTTCTC |
| | | ATGCGTCGACGGCGATACATGCACCGAACACGACACG |
| | | ACCGCGTCAACGTGTACGATTATTATAAAAACGACGG |
| | | GTCTAGACTTTTTGTTTATGGGGAAACTCTAAGAATTT |
| | | CATTTTGTTTTTTTCTATGCTATAAATGTGTCACCAGCA |
| | | GTTGGTCATCTCTTGGTTTTCCCTGGTTTTTCTGGCATC |
| | | TCCCCTCGTGGCCATATGGGAACTGAAGAAAGATGTT |
| | | TATGTCGTAGAATTGGATTGGTATCCGGATGCCCCTGG |
| | | AGAAATGGTGGTCCTCACCTGTGACACCCCTGAAGAA |
| | | GATGGTATCACCTGGACCTTGGACCAGAGCAGTGAGG |
| | | TCTTAGGCTCTGGCAAAACCCTGACCATCCAAGTCAA |
| | | AGAGTTTGGAGATGCTGGCCAGTACACCTGTCACAAA |
| | | GGAGGCGAGGTTCTAAGCCATTCGCTCCTGCTGCTTCA |
| | | CAAAAAGGAAGATGGAATTTGGTCCACTGATATTTTA |
| | | AAGGACCAGAAAGAACCCAAAAATAAGACCTTTCTAA |
| | | GATGCGAGGCCAAGAATTATTCTGGACGTTTCACCTG |
| | | CTGGTGGCTGACGACAATCAGTACTGATTTGACATTCA |
| | | GTGTCAAAAGCAGCAGAGGCTCTTCTGACCCCCAAGG |
| | | GGTGACGTGCGGAGCTGCTACACTCTCTGCAGAGAGA |
| | | GTCAGAGGGGACAACAAGGAGTATGAGTACTCAGTGG |
| | | AGTGCCAGGAGGACAGTGCCTGCCCAGCTGCTGAGGA |
| | | GAGTCTGCCCATTGAGGTCATGGTGGATGCCGTTCAC |
| | | AAGCTCAAGTATGAAAACTACACCAGCAGCTTCTTCA |
| | | TCAGGGACATCATCAAACCTGACCCACCCAAGAACTT |
| | | GCAGCTGAAGCCATTAAAGAATTCTCGGCAGGTGGAG |
| | | GTCAGCTGGGAGTACCCTGACACCTGGAGTACTCCAC |
| | | ATTCCTACTTCTCCCTGACATTCTGCGTTCAGGTCCAG |
| | | GGCAAGAGCAAGAGAGAAAAGAAAGATAGAGTCTTC |
| | | ACGGACAAGACCTCAGCCACGGTCATCTGCCGCAAAA |
| | | ATGCCAGCATTAGCGTGCGGGCCCAGGACCGCTACTA |
| | | TAGCTCATCTTGGAGCGAATGGGCATCTGTGCCCTGCA |
| | | GTGTTCCTGGAGTAGGGGTACCTGGGGTGGGCGCCAG |
| | | AAACCTCCCCGTGGCCACTCCAGACCCAGGAATGTTC |
| | | CCATGCCTTCACCACTCCCAAAACCTGCTGAGGGCCGT |
| | | CAGCAACATGCTCCAGAAGGCCAGACAAACTCTAGAA |
| | | TTTTACCCTTGCACTTCTGAAGAGATTGATCATGAAGA |
| | | TATCACAAAAGATAAAACCAGCACAGTGGAGGCCTGT |
| | | TTACCATTGGAATTAACCAAGAATGAGAGTTGCCTAA |
| | | ATTCCAGAGAGACCTCTTTCATAACTAATGGGAGTTGC |
| | | CTGGCCTCCAGAAAGACCTCTTTTATGATGGCCCTGTG |
| | | CCTTAGTAGTATTTATGAAGACTTGAAGATGTACCAG |
| | | GTGGAGTTCAAGACCATGAATGCAAAGCTGCTGATGG |
| | | ATCCTAAGAGGCAGATCTTTCTAGATCAAAACATGCT |
| | | GGCAGTTATTGATGAGCTGATGCAGGCCCTGAATTTC |
| | | AACAGTGAGACTGTGCCACAAAAATCCTCCCTTGAAG |
| | | AACCGGATTTTTATAAAACTAAAATCAAGCTCTGCAT |
| | | ACTTCTTCATGCTTTCAGAATTCGGGCAGTGACTATTG |
| | | ATAGAGTGATGAGCTATCTGAATGCTTCCTAAAAAAA |
| | | TTGAAATTTTATTTTTTTTTTTTGGAATATAAATAATGA |
| | | AGGCCACTATCATCCTCCTTCTGCTTGCACAAGTTTCC |
| | | TGGGCTGGACCGTTTCAACAGAGAGGCTTATTTGACTT |
| | | TATGCTAGAAGATGAGGCTTCTGGGATAGGCCCAGAA |
| | | GTTCCTGATGACCGCGACTTCGAGCCCTCCCTAGGCCC |
| | | AGTGTGCCCCTTCCGCTGTCAATGCCATCTTCGAGTGG |
| | | TCCAGTGTTCTGATTTGGGTCTGGACAAAGTGCCAAA |
| | | GGATCTTCCCCCTGACACAACTCTGCTAGACCTGCAAA |
| | | ACAACAAAATAACCGAAATCAAAGATGGAGACTTTAA |
| | | GAACCTGAAGAACCTTCACGCATTGATTCTTGTCAACA |
| | | ATAAAATTAGCAAAGTTAGTCCTGGAGCATTTACACC |
| | | TTTGGTGAAGTTGGAACGACTTTATCTGTCCAAGAATC |
| | | AGCTGAAGGAATTGCCAGAAAAAATGCCCAAAACTCT |
| | | TCAGGAGCTGCGTGCCCATGAGAATGAGATCACCAAA |
| | | GTGCGAAAAGTTACTTTCAATGGACTGAACCAGATGA |
| | | TTGTCATAGAACTGGGCACCAATCCGCTGAAGAGCTC |
| | | AGGAATTGAAAATGGGGCTTTCCAGGGAATGAAGAAG |
| | | CTCTCCTACATCCGCATTGCTGATACCAATATCACCAG |
| | | CATTCCTCAAGGTCTTCCTCCTTCCCTTACGGAATTAC |
| | | ATCTTGATGGCAACAAAATCAGCAGAGTTGATGCAGC |
| | | TAGCCTGAAAGGACTGAATAATTTGGCTAAGTTGGGA |
| | | TTGAGTTTCAACAGCATCTCTGCTGTTGACAATGGCTC |
| | | TCTGGCCAACACGCCTCATCTGAGGGAGCTTCACTTGG |
| | | ACAACAACAAGCTTACCAGAGTACCTGGTGGGCTGGC |
| | | AGAGCATAAGTACATCCAGGTTGTCTACCTTCATAAC |
| | | AACAATATCTCTGTAGTTGGATCAAGTGACTTCTGCCC |
| | | ACCTGGACACAACACCAAAAAGGCTTCTTATTCGGGT |
| | | GTGAGTCTTTTCAGCAACCCGGTCCAGTACTGGGAGA |
| | | TACAGCCATCCACCTTCAGATGTGTCTACGTGCGCTCT |
| | | GCCATTCAACTCGGAAACTATAAGTAAAAAAATTGAA |
| | | ATTTTATTTTTTTTTTTTGGAATATAAATAATGGTGAGC |
| | | AAGGGCGAGGAGCTGTTCACCGGGGTGGTGCCCATCC |
| | | TGGTCGAGCTGGACGGCGACGTAAACGGCCACAAGTT |
| | | CAGCGTGTCCGGCGAGGGCGAGGGCGATGCCACCTAC |
| | | GGCAAGCTGACCCTGAAGTTCATCTGCACCACCGGCA |
| | | AGCTGCCCGTGCCCTGGCCCACCCTCGTGACCACCCTG |
| | | ACCTACGGCGTGCAGTGCTTCAGCCGCTACCCCGACC |
| | | ACATGAAGCAGCACGACTTCTTCAAGTCCGCCATGCC |
| | | CGAAGGCTACGTCCAGGAGCGCACCATCTTCTTCAAG |
| | | GACGACGGCAACTACAAGACCCGCGCCGAGGTGAAGT |
| | | TCGAGGGCGACACCCTGGTGAACCGCATCGAGCTGAA |
| | | GGGCATCGACTTCAAGGAGGACGGCAACATCCTGGGG |
| | | CACAAGCTGGAGTACAACTACAACAGCCACAACGTCT |
| | | ATATCATGGCCGACAAGCAGAAGAACGGCATCAAGGT |
| | | GAACTTCAAGATCCGCCACAACATCGAGGACGGCAGC |
| | | GTGCAGCTCGCCGACCACTACCAGCAGAACACCCCCA |
| | | TCGGCGACGGCCCCGTGCTGCTGCCCGACAACCACTA |
| | | CCTGAGCACCCAGTCCGCCCTGAGCAAAGACCCCAAC |
| | | GAGAAGCGCGATCACATGGTCCTGCTGGAGTTCGTGA |
| | | CCGCCGCCGGGATCACTCTCGGCATGGACGAGCTGTA |
| | | CAAGTAATTATATAAGGTATCTCGTTTGTCTATAACAA |
| | | AGATCGTAACTGACCTTTTTTATATCGAGAAAACATAC |
| | | GTTTAGTTCATCCTCAAACGTAACACCGTAACTGCCTC |
| | | GGACATCCTCCTTGTTGTCGTACACAAACATACTAATC |
| | | GGATGCGTGAAATGAGGATTCACTTTAATCGGATTGG |
| | | TTTCTAGGTTAACACATGTTACACAGGATCCTAAGATG |
| | | GTTATGGACACATCCTTGTTGTGATGTAACGAGTCGGG |
| | | AAGTTGATTGCCGTAGTTGCCCACGTCGCCCTCCGGTT |
| | | CCAGACACGTAATGGTTAGGTATATATCCGAATACTTC |
| | | GTCAACGGATGAGTCGTAAATAACATGATGGATAGCT |
| | | TGTTCCCATCTCCTGCACCAGCACTGGCCGCCACAAAT |
| | | CGTTGTACCACGTTAGTAATCGTAATGTTTATCATAAG |
| | | CCCGTACCCGGTTAATATGAGCGTGGACGTTTTATGAT |
| | | CGTATCGTTCCTTCATGTGACATTCTCCCATAACCGTT |
| | | TCGACGTACCGATTTAACCCGATGGTTAGCTCGGCGG |
| | | CTAAGTGCCAGTACTTTTTTGGATACGTCGCACATGTT |
| | | GAGGTTGCGACGAGGCAGGCGAGCACGATGATAATAT |
| | | ACCGCGCCAT |
| 12 | Full recombination | GACGAAAGGGCCTCGTGATACGCCTATTTTTATAGGTT |
| | plasmid sequence | AATGTCATGATAATAATGGTTTCTTAGACGTCAGGTGG |
| | for generation of | CACTTTTCGGGGAAATGTGCGCGGAACCCCTATTTGTT |
| | HV11 | TATTTTTCTAAATACATTCAAATATGTATCCGCTCATG |
| | | AGACAATAACCCTGATAAATGCTTCAATAATATTGAA |
| | | AAAGGAAGAGTATGAGTATTCAACATTTCCGTGTCGC |
| | | CCTTATTCCCTTTTTTGCGGCATTTTGCCTTCCTGTTTT |
| | | TGCTCACCCAGAAACGCTGGTGAAAGTAAAAGATGCT |
| | | GAAGATCAGTTGGGTGCACGAGTGGGTTACATCGAAC |
| | | TGGATCTCAACAGCGGTAAGATCCTTGAGAGTTTTCGC |
| | | CCCGAAGAACGTTTTCCAATGATGAGCACTTTTAAAGT |
| | | TCTGCTATGTGGCGCGGTATTATCCCGTATTGACGCCG |
| | | GGCAAGAGCAACTCGGTCGCCGCATACACTATTCTCA |
| | | GAATGACTTGGTTGAGTACTCACCAGTCACAGAAAAG |
| | | CATCTTACGGATGGCATGACAGTAAGAGAATTATGCA |
| | | GTGCTGCCATAACCATGAGTGATAACACTGCGGCCAA |
| | | CTTACTTCTGACAACGATCGGAGGACCGAAGGAGCTA |
| | | ACCGCTTTTTTGCACAACATGGGGGATCATGTAACTCG |
| | | CCTTGATCGTTGGGAACCGGAGCTGAATGAAGCCATA |
| | | CCAAACGACGAGCGTGACACCACGATGCCTGTAGCAA |
| | | TGGCAACAACGTTGCGCAAACTATTAACTGGCGAACT |
| | | ACTTACTCTAGCTTCCCGGCAACAATTAATAGACTGGA |
| | | TGGAGGCGGATAAAGTTGCAGGACCACTTCTGCGCTC |
| | | GGCCCTTCCGGCTGGCTGGTTTATTGCTGATAAATCTG |
| | | GAGCCGGTGAGCGTGGGTCTCGCGGTATCATTGCAGC |
| | | ACTGGGGCCAGATGGTAAGCCCTCCCGTATCGTAGTT |
| | | ATCTACACGACGGGGAGTCAGGCAACTATGGATGAAC |
| | | GAAATAGACAGATCGCTGAGATAGGTGCCTCACTGAT |
| | | TAAGCATTGGTAACTGTCAGACCAAGTTTACTCATATA |
| | | TACTTTAGATTGATTTAAAACTTCATTTTTAATTTAAA |
| | | AGGATCTAGGTGAAGATCCTTTTTGATAATCTCATGAC |
| | | CAAAATCCCTTAACGTGAGTTTTCGTTCCACTGAGCGT |
| | | CAGACCCCGTAGAAAAGATCAAAGGATCTTCTTGAGA |
| | | TCCTTTTTTTCTGCGCGTAATCTGCTGCTTGCAAACAA |
| | | AAAAACCACCGCTACCAGCGGTGGTTTGTTTGCCGGA |
| | | TCAAGAGCTACCAACTCTTTTTCCGAAGGTAACTGGCT |
| | | TCAGCAGAGCGCAGATACCAAATACTGTTCTTCTAGT |
| | | GTAGCCGTAGTTAGGCCACCACTTCAAGAACTCTGTA |
| | | GCACCGCCTACATACCTCGCTCTGCTAATCCTGTTACC |
| | | AGTGGCTGCTGCCAGTGGCGATAAGTCGTGTCTTACC |
| | | GGGTTGGACTCAAGACGATAGTTACCGGATAAGGCGC |
| | | AGCGGTCGGGCTGAACGGGGGGTTCGTGCACACAGCC |
| | | CAGCTTGGAGCGAACGACCTACACCGAACTGAGATAC |
| | | CTACAGCGTGAGCTATGAGAAAGCGCCACGCTTCCCG |
| | | AAGGGAGAAAGGCGGACAGGTATCCGGTAAGCGGCA |
| | | GGGTCGGAACAGGAGAGCGCACGAGGGAGCTTCCAG |
| | | GGGGAAACGCCTGGTATCTTTATAGTCCTGTCGGGTTT |
| | | CGCCACCTCTGACTTGAGCGTCGATTTTTGTGATGCTC |
| | | GTCAGGGGGGCGGAGCCTATGGAAAAACGCCAGCAA |
| | | CGCGGCCTTTTTACGGTTCCTGGCCTTTTGCTGGCCTTT |
| | | TGCTCACATGTTCTTTCCTGCGTTATCCCCTGATTCTGT |
| | | GGATAACCGTATTACCGCCTTTGAGTGAGCTGATACC |
| | | GCTCGCCGCAGCCGAACGACCGAGCGCAGCGAGTCAG |
| | | TGAGCGAGGAAGCGGAAGAGCGCCCAATACGCAAAC |
| | | CGCCTCTCCCCGCGCGTTGGCCGATTCATTAATGCAGC |
| | | TGGCACGACAGGTTTCCCGACTGGAAAGCGGGCAGTG |
| | | AGCGCAACGCAATTAATGTGAGTTAGCTCACTCATTA |
| | | GGCACCCCAGGCTTTACACTTTATGCTTCCGGCTCGTA |
| | | TGTTGTGTGGAATTGTGAGCGGATAACAATTTCACAC |
| | | AGGAAACAGCTATGACCATGATTACGCCAAGCTTGCA |
| | | TGCAGGCCTCTGCAGTCGACGGGCCCGGGATCCGATA |
| | | TCTAGATGCATTCGCGAGGTACCGAGCTCGAATTCGA |
| | | TGTCGTACATCGATTACACAAAGAAGTAGAGTCATAC |
| | | GACGTACGTTTCCCTATAAAATCGGTAAACCTAGACG |
| | | CGGTGTTTCTATCCATAAACGTAACACGTGTACGTCTA |
| | | CGTTGGAAGATACCCTTGACCGAACACAATCCTTATC |
| | | AGACGGCCTACGGATGTTCTAACGACAGATTATACAG |
| | | CTACAACGAGTACGCTTTTTCTCATTTAAAACAAGACC |
| | | GTGTAAAGATCATAGAACTCCCATGTGACGACGATTA |
| | | CAGCGTCGTGTTAATCACACACGATAGCCGTTCGACT |
| | | ATTACACCGGATAAAGTGACCGGGTGGCTGCGCACGA |
| | | CCCGTCTACGTTACGTAAACGTATCCCTACCCAAGGGT |
| | | TCCACGGAAACGGGACACAACGTAACGTGTCTAACTC |
| | | CCACACACGTCAATCTATGTCATCGTTGTCGTATAACG |
| | | ATTACCAAAACGGGCGTGGACGCAACCGCGTTCTCAT |
| | | GCGTCGACGGCGATACATGCACCGAACACGACACGAC |
| | | CGCGTCAACGTGTACGATTATTATAAAAACGACGGGT |
| | | CTAGACTTTTTGTTTATGGGGAAACTCTAAGAATTTCA |
| | | TTTTGTTTTTTTCTATGCTATAAATGTGTCACCAGCAGT |
| | | TGGTCATCTCTTGGTTTTCCCTGGTTTTTCTGGCATCTC |
| | | CCCTCGTGGCCATATGGGAACTGAAGAAAGATGTTTA |
| | | TGTCGTAGAATTGGATTGGTATCCGGATGCCCCTGGA |
| | | GAAATGGTGGTCCTCACCTGTGACACCCCTGAAGAAG |
| | | ATGGTATCACCTGGACCTTGGACCAGAGCAGTGAGGT |
| | | CTTAGGCTCTGGCAAAACCCTGACCATCCAAGTCAAA |
| | | GAGTTTGGAGATGCTGGCCAGTACACCTGTCACAAAG |
| | | GAGGCGAGGTTCTAAGCCATTCGCTCCTGCTGCTTCAC |
| | | AAAAAGGAAGATGGAATTTGGTCCACTGATATTTTAA |
| | | AGGACCAGAAAGAACCCAAAAATAAGACCTTTCTAAG |
| | | ATGCGAGGCCAAGAATTATTCTGGACGTTTCACCTGCT |
| | | GGTGGCTGACGACAATCAGTACTGATTTGACATTCAG |
| | | TGTCAAAAGCAGCAGAGGCTCTTCTGACCCCCAAGGG |
| | | GTGACGTGCGGAGCTGCTACACTCTCTGCAGAGAGAG |
| | | TCAGAGGGGACAACAAGGAGTATGAGTACTCAGTGGA |
| | | GTGCCAGGAGGACAGTGCCTGCCCAGCTGCTGAGGAG |
| | | AGTCTGCCCATTGAGGTCATGGTGGATGCCGTTCACA |
| | | AGCTCAAGTATGAAAACTACACCAGCAGCTTCTTCAT |
| | | CAGGGACATCATCAAACCTGACCCACCCAAGAACTTG |
| | | CAGCTGAAGCCATTAAAGAATTCTCGGCAGGTGGAGG |
| | | TCAGCTGGGAGTACCCTGACACCTGGAGTACTCCACA |
| | | TTCCTACTTCTCCCTGACATTCTGCGTTCAGGTCCAGG |
| | | GCAAGAGCAAGAGAGAAAAGAAAGATAGAGTCTTCA |
| | | CGGACAAGACCTCAGCCACGGTCATCTGCCGCAAAAA |
| | | TGCCAGCATTAGCGTGCGGGCCCAGGACCGCTACTAT |
| | | AGCTCATCTTGGAGCGAATGGGCATCTGTGCCCTGCA |
| | | GTGTTCCTGGAGTAGGGGTACCTGGGGTGGGCGCCAG |
| | | AAACCTCCCCGTGGCCACTCCAGACCCAGGAATGTTC |
| | | CCATGCCTTCACCACTCCCAAAACCTGCTGAGGGCCGT |
| | | CAGCAACATGCTCCAGAAGGCCAGACAAACTCTAGAA |
| | | TTTTACCCTTGCACTTCTGAAGAGATTGATCATGAAGA |
| | | TATCACAAAAGATAAAACCAGCACAGTGGAGGCCTGT |
| | | TTACCATTGGAATTAACCAAGAATGAGAGTTGCCTAA |
| | | ATTCCAGAGAGACCTCTTTCATAACTAATGGGAGTTGC |
| | | CTGGCCTCCAGAAAGACCTCTTTTATGATGGCCCTGTG |
| | | CCTTAGTAGTATTTATGAAGACTTGAAGATGTACCAG |
| | | GTGGAGTTCAAGACCATGAATGCAAAGCTGCTGATGG |
| | | ATCCTAAGAGGCAGATCTTTCTAGATCAAAACATGCT |
| | | GGCAGTTATTGATGAGCTGATGCAGGCCCTGAATTTC |
| | | AACAGTGAGACTGTGCCACAAAAATCCTCCCTTGAAG |
| | | AACCGGATTTTTATAAAACTAAAATCAAGCTCTGCAT |
| | | ACTTCTTCATGCTTTCAGAATTCGGGCAGTGACTATTG |
| | | ATAGAGTGATGAGCTATCTGAATGCTTCCTAAAAAAA |
| | | TTGAAATTTTATTTTTTTTTTTTGGAATATAAATAATGA |
| | | AGGCCACTATCATCCTCCTTCTGCTTGCACAAGTTTCC |
| | | TGGGCTGGACCGTTTCAACAGAGAGGCTTATTTGACTT |
| | | TATGCTAGAAGATGAGGCTTCTGGGATAGGCCCAGAA |
| | | GTTCCTGATGACCGCGACTTCGAGCCCTCCCTAGGCCC |
| | | AGTGTGCCCCTTCCGCTGTCAATGCCATCTTCGAGTGG |
| | | TCCAGTGTTCTGATTTGGGTCTGGACAAAGTGCCAAA |
| | | GGATCTTCCCCCTGACACAACTCTGCTAGACCTGCAAA |
| | | ACAACAAAATAACCGAAATCAAAGATGGAGACTTTAA |
| | | GAACCTGAAGAACCTTCACGCATTGATTCTTGTCAACA |
| | | ATAAAATTAGCAAAGTTAGTCCTGGAGCATTTACACC |
| | | TTTGGTGAAGTTGGAACGACTTTATCTGTCCAAGAATC |
| | | AGCTGAAGGAATTGCCAGAAAAAATGCCCAAAACTCT |
| | | TCAGGAGCTGCGTGCCCATGAGAATGAGATCACCAAA |
| | | GTGCGAAAAGTTACTTTCAATGGACTGAACCAGATGA |
| | | TTGTCATAGAACTGGGCACCAATCCGCTGAAGAGCTC |
| | | AGGAATTGAAAATGGGGCTTTCCAGGGAATGAAGAAG |
| | | CTCTCCTACATCCGCATTGCTGATACCAATATCACCAG |
| | | CATTCCTCAAGGTCTTCCTCCTTCCCTTACGGAATTAC |
| | | ATCTTGATGGCAACAAAATCAGCAGAGTTGATGCAGC |
| | | TAGCCTGAAAGGACTGAATAATTTGGCTAAGTTGGGA |
| | | TTGAGTTTCAACAGCATCTCTGCTGTTGACAATGGCTC |
| | | TCTGGCCAACACGCCTCATCTGAGGGAGCTTCACTTGG |
| | | ACAACAACAAGCTTACCAGAGTACCTGGTGGGCTGGC |
| | | AGAGCATAAGTACATCCAGGTTGTCTACCTTCATAAC |
| | | AACAATATCTCTGTAGTTGGATCAAGTGACTTCTGCCC |
| | | ACCTGGACACAACACCAAAAAGGCTTCTTATTCGGGT |
| | | GTGAGTCTTTTCAGCAACCCGGTCCAGTACTGGGAGA |
| | | TACAGCCATCCACCTTCAGATGTGTCTACGTGCGCTCT |
| | | GCCATTCAACTCGGAAACTATAAGTAAAAAAATTGAA |
| | | ATTTTATTTTTTTTTTTTGGAATATAAATAATGGTGAGC |
| | | AAGGGCGAGGAGCTGTTCACCGGGGTGGTGCCCATCC |
| | | TGGTCGAGCTGGACGGCGACGTAAACGGCCACAAGTT |
| | | CAGCGTGTCCGGCGAGGGCGAGGGCGATGCCACCTAC |
| | | GGCAAGCTGACCCTGAAGTTCATCTGCACCACCGGCA |
| | | AGCTGCCCGTGCCCTGGCCCACCCTCGTGACCACCCTG |
| | | ACCTACGGCGTGCAGTGCTTCAGCCGCTACCCCGACC |
| | | ACATGAAGCAGCACGACTTCTTCAAGTCCGCCATGCC |
| | | CGAAGGCTACGTCCAGGAGCGCACCATCTTCTTCAAG |
| | | GACGACGGCAACTACAAGACCCGCGCCGAGGTGAAGT |
| | | TCGAGGGCGACACCCTGGTGAACCGCATCGAGCTGAA |
| | | GGGCATCGACTTCAAGGAGGACGGCAACATCCTGGGG |
| | | CACAAGCTGGAGTACAACTACAACAGCCACAACGTCT |
| | | ATATCATGGCCGACAAGCAGAAGAACGGCATCAAGGT |
| | | GAACTTCAAGATCCGCCACAACATCGAGGACGGCAGC |
| | | GTGCAGCTCGCCGACCACTACCAGCAGAACACCCCCA |
| | | TCGGCGACGGCCCCGTGCTGCTGCCCGACAACCACTA |
| | | CCTGAGCACCCAGTCCGCCCTGAGCAAAGACCCCAAC |
| | | GAGAAGCGCGATCACATGGTCCTGCTGGAGTTCGTGA |
| | | CCGCCGCCGGGATCACTCTCGGCATGGACGAGCTGTA |
| | | CAAGTAATTATATAAGGTATCTCGTTTGTCTATAACAA |
| | | AGATCGTAACTGACCTTTTTTATATCGAGAAAACATAC |
| | | GTTTAGTTCATCCTCAAACGTAACACCGTAACTGCCTC |
| | | GGACATCCTCCTTGTTGTCGTACACAAACATACTAATC |
| | | GGATGCGTGAAATGAGGATTCACTTTAATCGGATTGG |
| | | TTTCTAGGTTAACACATGTTACACAGGATCCTAAGATG |
| | | GTTATGGACACATCCTTGTTGTGATGTAACGAGTCGGG |
| | | AAGTTGATTGCCGTAGTTGCCCACGTCGCCCTCCGGTT |
| | | CCAGACACGTAATGGTTAGGTATATATCCGAATACTTC |
| | | GTCAACGGATGAGTCGTAAATAACATGATGGATAGCT |
| | | TGTTCCCATCTCCTGCACCAGCACTGGCCGCCACAAAT |
| | | CGTTGTACCACGTTAGTAATCGTAATGTTTATCATAAG |
| | | CCCGTACCCGGTTAATATGAGCGTGGACGTTTTATGAT |
| | | CGTATCGTTCCTTCATGTGACATTCTCCCATAACCGTT |
| | | TCGACGTACCGATTTAACCCGATGGTTAGCTCGGCGG |
| | | CTAAGTGCCAGTACTTTTTTGGATACGTCGCACATGTT |
| | | GAGGTTGCGACGAGGCAGGCGAGCACGATGATAATAT |
| | | ACCGCGCCATGAATTCACTGGCCGTCGTTTTACAACGT |
| | | CGTGACTGGGAAAACCCTGGCGTTACCCAACTTAATC |
| | | GCCTTGCAGCACATCCCCCTTTCGCCAGCTGGCGTAAT |
| | | AGCGAAGAGGCCCGCACCGATCGCCCTTCCCAACAGT |
| | | TGCGCAGCCTGAATGGCGAATGGCGCCTGATGCGGTA |
| | | TTTTCTCCTTACGCATCTGTGCGGTATTTCACACCGCA |
| | | TATGGTGCACTCTCAGTACAATCTGCTCTGATGCCGCA |
| | | TAGTTAAGCCAGCCCCGACACCCGCCAACACCCGCTG |
| | | ACGCGCCCTGACGGGCTTGTCTGCTCCCGGCATCCGCT |
| | | TACAGACAAGCTGTGACCGTCTCCGGGAGCTGCATGT |
| | | GTCAGAGGTTTTCACCGTCATCACCGAAACGCGCGA |
| 13 | Insertion screening | GGGGACAAGTTTGTACAAAAAAGCAGGCTCGTAGACG |
| | primer | CGGTGTTTCTATCC |
| 14 | Insertion screening | GGGGACCACTTTGTACAAGAAAGCTGGGTAAACGTAA |
| | primer | CACCGTAACTGCC |
| 15 | P11 forward primer | GAATTTCATTTTGTTTTTTTCTATGCTATAA |
| 16 | IL-12 reverse | GGGGACAACTTTATTATACAAAGTTGTTTAGGAAGCA |
| | primer | TTCAGATAGCTCATC |
| 17 | Spacer | ATGTCT |
| 18 | Human TNF-α | ATGAGCACTGAAAGCATGATCCGGGACGTGGAGCTGG |
| | | CCGAGGAGGCGCTCCCCAAGAAGACAGGGGGGCCCC |
| | | AGGGCTCCAGGCGGTGCTTGTTCCTCAGCCTCTTCTCC |
| | | TTCCTGATCGTGGCAGGCGCCACCACGCTCTTCTGCCT |
| | | GCTGCACTTTGGAGTGATCGGCCCCCAGAGGGAAGAG |
| | | TTCCCCAGGGACCTCTCTCTAATCAGCCCTCTGGCCCA |
| | | GGCAGTCAGATCATCTTCTCGAACCCCGAGTGACAAG |
| | | CCTGTAGCCCATGTTGTAGCAAACCCTCAAGCTGAGG |
| | | GGCAGCTCCAGTGGCTGAACCGCCGGGCCAATGCCCT |
| | | CCTGGCCAATGGCGTGGAGCTGAGAGATAACCAGCTG |
| | | GTGGTGCCATCAGAGGGCCTGTACCTCATCTACTCCCA |
| | | GGTCCTCTTCAAGGGCCAAGGCTGCCCCTCCACCCATG |
| | | TGCTCCTCACCCACACCATCAGCCGCATCGCCGTCTCC |
| | | TACCAGACCAAGGTCAACCTCCTCTCTGCCATCAAGA |
| | | GCCCCTGCCAGAGGGAGACCCCAGAGGGGGCTGAGG |
| | | CCAAGCCCTGGTATGAGCCCATCTATCTGGGAGGGGT |
| | | CTTCCAGCTGGAGAAGGGTGACCGACTCAGCGCTGAG |
| | | ATCAATCGGCCCGACTATCTCGACTTTGCCGAGTCTGG |
| | | GCAGGTCTACTTTGGGATCATTGCCCTGTGA |
| 19 | Spacer | AGCTTG |
| 20 | HV14 insert | GAATTTCATTTTGTTTTTTTCTATGCTATAAATGTGTCA |
| | | CCAGCAGTTGGTCATCTCTTGGTTTTCCCTGGTTTTTCT |
| | | GGCATCTCCCCTCGTGGCCATATGGGAACTGAAGAAA |
| | | GATGTTTATGTCGTAGAATTGGATTGGTATCCGGATGC |
| | | CCCTGGAGAAATGGTGGTCCTCACCTGTGACACCCCT |
| | | GAAGAAGATGGTATCACCTGGACCTTGGACCAGAGCA |
| | | GTGAGGTCTTAGGCTCTGGCAAAACCCTGACCATCCA |
| | | AGTCAAAGAGTTTGGAGATGCTGGCCAGTACACCTGT |
| | | CACAAAGGAGGCGAGGTTCTAAGCCATTCGCTCCTGC |
| | | TGCTTCACAAAAAGGAAGATGGAATTTGGTCCACTGA |
| | | TATTTTAAAGGACCAGAAAGAACCCAAAAATAAGACC |
| | | TTTCTAAGATGCGAGGCCAAGAATTATTCTGGACGTTT |
| | | CACCTGCTGGTGGCTGACGACAATCAGTACTGATTTG |
| | | ACATTCAGTGTCAAAAGCAGCAGAGGCTCTTCTGACC |
| | | CCCAAGGGGTGACGTGCGGAGCTGCTACACTCTCTGC |
| | | AGAGAGAGTCAGAGGGGACAACAAGGAGTATGAGTA |
| | | CTCAGTGGAGTGCCAGGAGGACAGTGCCTGCCCAGCT |
| | | GCTGAGGAGAGTCTGCCCATTGAGGTCATGGTGGATG |
| | | CCGTTCACAAGCTCAAGTATGAAAACTACACCAGCAG |
| | | CTTCTTCATCAGGGACATCATCAAACCTGACCCACCCA |
| | | AGAACTTGCAGCTGAAGCCATTAAAGAATTCTCGGCA |
| | | GGTGGAGGTCAGCTGGGAGTACCCTGACACCTGGAGT |
| | | ACTCCACATTCCTACTTCTCCCTGACATTCTGCGTTCA |
| | | GGTCCAGGGCAAGAGCAAGAGAGAAAAGAAAGATAG |
| | | AGTCTTCACGGACAAGACCTCAGCCACGGTCATCTGC |
| | | CGCAAAAATGCCAGCATTAGCGTGCGGGCCCAGGACC |
| | | GCTACTATAGCTCATCTTGGAGCGAATGGGCATCTGTG |
| | | CCCTGCAGTGTTCCTGGAGTAGGGGTACCTGGGGTGG |
| | | GCGCCAGAAACCTCCCCGTGGCCACTCCAGACCCAGG |
| | | AATGTTCCCATGCCTTCACCACTCCCAAAACCTGCTGA |
| | | GGGCCGTCAGCAACATGCTCCAGAAGGCCAGACAAAC |
| | | TCTAGAATTTTACCCTTGCACTTCTGAAGAGATTGATC |
| | | ATGAAGATATCACAAAAGATAAAACCAGCACAGTGG |
| | | AGGCCTGTTTACCATTGGAATTAACCAAGAATGAGAG |
| | | TTGCCTAAATTCCAGAGAGACCTCTTTCATAACTAATG |
| | | GGAGTTGCCTGGCCTCCAGAAAGACCTCTTTTATGATG |
| | | GCCCTGTGCCTTAGTAGTATTTATGAAGACTTGAAGAT |
| | | GTACCAGGTGGAGTTCAAGACCATGAATGCAAAGCTG |
| | | CTGATGGATCCTAAGAGGCAGATCTTTCTAGATCAAA |
| | | ACATGCTGGCAGTTATTGATGAGCTGATGCAGGCCCT |
| | | GAATTTCAACAGTGAGACTGTGCCACAAAAATCCTCC |
| | | CTTGAAGAACCGGATTTTTATAAAACTAAAATCAAGC |
| | | TCTGCATACTTCTTCATGCTTTCAGAATTCGGGCAGTG |
| | | ACTATTGATAGAGTGATGAGCTATCTGAATGCTTCCTA |
| | | AATGTCTGAATTTCATTTTGTTTTTTTCTATGCTATAAA |
| | | TGAGCACTGAAAGCATGATCCGGGACGTGGAGCTGGC |
| | | CGAGGAGGCGCTCCCCAAGAAGACAGGGGGGCCCCA |
| | | GGGCTCCAGGCGGTGCTTGTTCCTCAGCCTCTTCTCCT |
| | | TCCTGATCGTGGCAGGCGCCACCACGCTCTTCTGCCTG |
| | | CTGCACTTTGGAGTGATCGGCCCCCAGAGGGAAGAGT |
| | | TCCCCAGGGACCTCTCTCTAATCAGCCCTCTGGCCCAG |
| | | GCAGTCAGATCATCTTCTCGAACCCCGAGTGACAAGC |
| | | CTGTAGCCCATGTTGTAGCAAACCCTCAAGCTGAGGG |
| | | GCAGCTCCAGTGGCTGAACCGCCGGGCCAATGCCCTC |
| | | CTGGCCAATGGCGTGGAGCTGAGAGATAACCAGCTGG |
| | | TGGTGCCATCAGAGGGCCTGTACCTCATCTACTCCCAG |
| | | GTCCTCTTCAAGGGCCAAGGCTGCCCCTCCACCCATGT |
| | | GCTCCTCACCCACACCATCAGCCGCATCGCCGTCTCCT |
| | | ACCAGACCAAGGTCAACCTCCTCTCTGCCATCAAGAG |
| | | CCCCTGCCAGAGGGAGACCCCAGAGGGGGCTGAGGCC |
| | | AAGCCCTGGTATGAGCCCATCTATCTGGGAGGGGTCT |
| | | TCCAGCTGGAGAAGGGTGACCGACTCAGCGCTGAGAT |
| | | CAATCGGCCCGACTATCTCGACTTTGCCGAGTCTGGGC |
| | | AGGTCTACTTTGGGATCATTGCCCTGTGAAGCTTGAAA |
| | | AATTGAAATTTTATTTTTTTTTTTTGGAATATAAATAAT |
| | | GAAGGCCACTATCATCCTCCTTCTGCTTGCACAAGTTT |
| | | CCTGGGCTGGACCGTTTCAACAGAGAGGCTTATTTGA |
| | | CTTTATGCTAGAAGATGAGGCTTCTGGGATAGGCCCA |
| | | GAAGTTCCTGATGACCGCGACTTCGAGCCCTCCCTAG |
| | | GCCCAGTGTGCCCCTTCCGCTGTCAATGCCATCTTCGA |
| | | GTGGTCCAGTGTTCTGATTTGGGTCTGGACAAAGTGCC |
| | | AAAGGATCTTCCCCCTGACACAACTCTGCTAGACCTGC |
| | | AAAACAACAAAATAACCGAAATCAAAGATGGAGACT |
| | | TTAAGAACCTGAAGAACCTTCACGCATTGATTCTTGTC |
| | | AACAATAAAATTAGCAAAGTTAGTCCTGGAGCATTTA |
| | | CACCTTTGGTGAAGTTGGAACGACTTTATCTGTCCAAG |
| | | AATCAGCTGAAGGAATTGCCAGAAAAAATGCCCAAAA |
| | | CTCTTCAGGAGCTGCGTGCCCATGAGAATGAGATCAC |
| | | CAAAGTGCGAAAAGTTACTTTCAATGGACTGAACCAG |
| | | ATGATTGTCATAGAACTGGGCACCAATCCGCTGAAGA |
| | | GCTCAGGAATTGAAAATGGGGCTTTCCAGGGAATGAA |
| | | GAAGCTCTCCTACATCCGCATTGCTGATACCAATATCA |
| | | CCAGCATTCCTCAAGGTCTTCCTCCTTCCCTTACGGAA |
| | | TTACATCTTGATGGCAACAAAATCAGCAGAGTTGATG |
| | | CAGCTAGCCTGAAAGGACTGAATAATTTGGCTAAGTT |
| | | GGGATTGAGTTTCAACAGCATCTCTGCTGTTGACAATG |
| | | GCTCTCTGGCCAACACGCCTCATCTGAGGGAGCTTCAC |
| | | TTGGACAACAACAAGCTTACCAGAGTACCTGGTGGGC |
| | | TGGCAGAGCATAAGTACATCCAGGTTGTCTACCTTCAT |
| | | AACAACAATATCTCTGTAGTTGGATCAAGTGACTTCTG |
| | | CCCACCTGGACACAACACCAAAAAGGCTTCTTATTCG |
| | | GGTGTGAGTCTTTTCAGCAACCCGGTCCAGTACTGGG |
| | | AGATACAGCCATCCACCTTCAGATGTGTCTACGTGCGC |
| | | TCTGCCATTCAACTCGGAAACTATAAGTAAAAAAATT |
| | | GAAATTTTATTTTTTTTTTTTGGAATATAAATAATGGT |
| | | GAGCAAGGGCGAGGAGCTGTTCACCGGGGTGGTGCCC |
| | | ATCCTGGTCGAGCTGGACGGCGACGTAAACGGCCACA |
| | | AGTTCAGCGTGTCCGGCGAGGGCGAGGGCGATGCCAC |
| | | CTACGGCAAGCTGACCCTGAAGTTCATCTGCACCACC |
| | | GGCAAGCTGCCCGTGCCCTGGCCCACCCTCGTGACCA |
| | | CCCTGACCTACGGCGTGCAGTGCTTCAGCCGCTACCCC |
| | | GACCACATGAAGCAGCACGACTTCTTCAAGTCCGCCA |
| | | TGCCCGAAGGCTACGTCCAGGAGCGCACCATCTTCTTC |
| | | AAGGACGACGGCAACTACAAGACCCGCGCCGAGGTG |
| | | AAGTTCGAGGGCGACACCCTGGTGAACCGCATCGAGC |
| | | TGAAGGGCATCGACTTCAAGGAGGACGGCAACATCCT |
| | | GGGGCACAAGCTGGAGTACAACTACAACAGCCACAAC |
| | | GTCTATATCATGGCCGACAAGCAGAAGAACGGCATCA |
| | | AGGTGAACTTCAAGATCCGCCACAACATCGAGGACGG |
| | | CAGCGTGCAGCTCGCCGACCACTACCAGCAGAACACC |
| | | CCCATCGGCGACGGCCCCGTGCTGCTGCCCGACAACC |
| | | ACTACCTGAGCACCCAGTCCGCCCTGAGCAAAGACCC |
| | | CAACGAGAAGCGCGATCACATGGTCCTGCTGGAGTTC |
| | | GTGACCGCCGCCGGGATCACTCTCGGCATGGACGAGC |
| | | TGTACAAGTAA |
| 21 | HV14 insert plus | GATGTCGTACATCGATTACACAAAGAAGTAGAGTCAT |
| | recombination arms | ACGACGTACGTTTCCCTATAAAATCGGTAAACCTAGA |
| | | CGCGGTGTTTCTATCCATAAACGTAACACGTGTACGTC |
| | | TACGTTGGAAGATACCCTTGACCGAACACAATCCTTAT |
| | | CAGACGGCCTACGGATGTTCTAACGACAGATTATACA |
| | | GCTACAACGAGTACGCTTTTTCTCATTTAAAACAAGAC |
| | | CGTGTAAAGATCATAGAACTCCCATGTGACGACGATT |
| | | ACAGCGTCGTGTTAATCACACACGATAGCCGTTCGAC |
| | | TATTACACCGGATAAAGTGACCGGGTGGCTGCGCACG |
| | | ACCCGTCTACGTTACGTAAACGTATCCCTACCCAAGG |
| | | GTTCCACGGAAACGGGACACAACGTAACGTGTCTAAC |
| | | TCCCACACACGTCAATCTATGTCATCGTTGTCGTATAA |
| | | CGATTACCAAAACGGGCGTGGACGCAACCGCGTTCTC |
| | | ATGCGTCGACGGCGATACATGCACCGAACACGACACG |
| | | ACCGCGTCAACGTGTACGATTATTATAAAAACGACGG |
| | | GTCTAGACTTTTTGTTTATGGGGAAACTCTAAGAATTT |
| | | CATTTTGTTTTTTTCTATGCTATAAATGTGTCACCAGCA |
| | | GTTGGTCATCTCTTGGTTTTCCCTGGTTTTTCTGGCATC |
| | | TCCCCTCGTGGCCATATGGGAACTGAAGAAAGATGTT |
| | | TATGTCGTAGAATTGGATTGGTATCCGGATGCCCCTGG |
| | | AGAAATGGTGGTCCTCACCTGTGACACCCCTGAAGAA |
| | | GATGGTATCACCTGGACCTTGGACCAGAGCAGTGAGG |
| | | TCTTAGGCTCTGGCAAAACCCTGACCATCCAAGTCAA |
| | | AGAGTTTGGAGATGCTGGCCAGTACACCTGTCACAAA |
| | | GGAGGCGAGGTTCTAAGCCATTCGCTCCTGCTGCTTCA |
| | | CAAAAAGGAAGATGGAATTTGGTCCACTGATATTTTA |
| | | AAGGACCAGAAAGAACCCAAAAATAAGACCTTTCTAA |
| | | GATGCGAGGCCAAGAATTATTCTGGACGTTTCACCTG |
| | | CTGGTGGCTGACGACAATCAGTACTGATTTGACATTCA |
| | | GTGTCAAAAGCAGCAGAGGCTCTTCTGACCCCCAAGG |
| | | GGTGACGTGCGGAGCTGCTACACTCTCTGCAGAGAGA |
| | | GTCAGAGGGGACAACAAGGAGTATGAGTACTCAGTGG |
| | | AGTGCCAGGAGGACAGTGCCTGCCCAGCTGCTGAGGA |
| | | GAGTCTGCCCATTGAGGTCATGGTGGATGCCGTTCAC |
| | | AAGCTCAAGTATGAAAACTACACCAGCAGCTTCTTCA |
| | | TCAGGGACATCATCAAACCTGACCCACCCAAGAACTT |
| | | GCAGCTGAAGCCATTAAAGAATTCTCGGCAGGTGGAG |
| | | GTCAGCTGGGAGTACCCTGACACCTGGAGTACTCCAC |
| | | ATTCCTACTTCTCCCTGACATTCTGCGTTCAGGTCCAG |
| | | GGCAAGAGCAAGAGAGAAAAGAAAGATAGAGTCTTC |
| | | ACGGACAAGACCTCAGCCACGGTCATCTGCCGCAAAA |
| | | ATGCCAGCATTAGCGTGCGGGCCCAGGACCGCTACTA |
| | | TAGCTCATCTTGGAGCGAATGGGCATCTGTGCCCTGCA |
| | | GTGTTCCTGGAGTAGGGGTACCTGGGGTGGGCGCCAG |
| | | AAACCTCCCCGTGGCCACTCCAGACCCAGGAATGTTC |
| | | CCATGCCTTCACCACTCCCAAAACCTGCTGAGGGCCGT |
| | | CAGCAACATGCTCCAGAAGGCCAGACAAACTCTAGAA |
| | | TTTTACCCTTGCACTTCTGAAGAGATTGATCATGAAGA |
| | | TATCACAAAAGATAAAACCAGCACAGTGGAGGCCTGT |
| | | TTACCATTGGAATTAACCAAGAATGAGAGTTGCCTAA |
| | | ATTCCAGAGAGACCTCTTTCATAACTAATGGGAGTTGC |
| | | CTGGCCTCCAGAAAGACCTCTTTTATGATGGCCCTGTG |
| | | CCTTAGTAGTATTTATGAAGACTTGAAGATGTACCAG |
| | | GTGGAGTTCAAGACCATGAATGCAAAGCTGCTGATGG |
| | | ATCCTAAGAGGCAGATCTTTCTAGATCAAAACATGCT |
| | | GGCAGTTATTGATGAGCTGATGCAGGCCCTGAATTTC |
| | | AACAGTGAGACTGTGCCACAAAAATCCTCCCTTGAAG |
| | | AACCGGATTTTTATAAAACTAAAATCAAGCTCTGCAT |
| | | ACTTCTTCATGCTTTCAGAATTCGGGCAGTGACTATTG |
| | | ATAGAGTGATGAGCTATCTGAATGCTTCCTAAATGTCT |
| | | GAATTTCATTTTGTTTTTTTCTATGCTATAAATGAGCAC |
| | | TGAAAGCATGATCCGGGACGTGGAGCTGGCCGAGGAG |
| | | GCGCTCCCCAAGAAGACAGGGGGGCCCCAGGGCTCCA |
| | | GGCGGTGCTTGTTCCTCAGCCTCTTCTCCTTCCTGATC |
| | | GTGGCAGGCGCCACCACGCTCTTCTGCCTGCTGCACTT |
| | | TGGAGTGATCGGCCCCCAGAGGGAAGAGTTCCCCAGG |
| | | GACCTCTCTCTAATCAGCCCTCTGGCCCAGGCAGTCAG |
| | | ATCATCTTCTCGAACCCCGAGTGACAAGCCTGTAGCCC |
| | | ATGTTGTAGCAAACCCTCAAGCTGAGGGGCAGCTCCA |
| | | GTGGCTGAACCGCCGGGCCAATGCCCTCCTGGCCAAT |
| | | GGCGTGGAGCTGAGAGATAACCAGCTGGTGGTGCCAT |
| | | CAGAGGGCCTGTACCTCATCTACTCCCAGGTCCTCTTC |
| | | AAGGGCCAAGGCTGCCCCTCCACCCATGTGCTCCTCA |
| | | CCCACACCATCAGCCGCATCGCCGTCTCCTACCAGACC |
| | | AAGGTCAACCTCCTCTCTGCCATCAAGAGCCCCTGCCA |
| | | GAGGGAGACCCCAGAGGGGGCTGAGGCCAAGCCCTG |
| | | GTATGAGCCCATCTATCTGGGAGGGGTCTTCCAGCTG |
| | | GAGAAGGGTGACCGACTCAGCGCTGAGATCAATCGGC |
| | | CCGACTATCTCGACTTTGCCGAGTCTGGGCAGGTCTAC |
| | | TTTGGGATCATTGCCCTGTGAAGCTTGAAAAATTGAA |
| | | ATTTTATTTTTTTTTTTTGGAATATAAATAATGAAGGC |
| | | CACTATCATCCTCCTTCTGCTTGCACAAGTTTCCTGGG |
| | | CTGGACCGTTTCAACAGAGAGGCTTATTTGACTTTATG |
| | | CTAGAAGATGAGGCTTCTGGGATAGGCCCAGAAGTTC |
| | | CTGATGACCGCGACTTCGAGCCCTCCCTAGGCCCAGT |
| | | GTGCCCCTTCCGCTGTCAATGCCATCTTCGAGTGGTCC |
| | | AGTGTTCTGATTTGGGTCTGGACAAAGTGCCAAAGGA |
| | | TCTTCCCCCTGACACAACTCTGCTAGACCTGCAAAACA |
| | | ACAAAATAACCGAAATCAAAGATGGAGACTTTAAGAA |
| | | CCTGAAGAACCTTCACGCATTGATTCTTGTCAACAATA |
| | | AAATTAGCAAAGTTAGTCCTGGAGCATTTACACCTTTG |
| | | GTGAAGTTGGAACGACTTTATCTGTCCAAGAATCAGC |
| | | TGAAGGAATTGCCAGAAAAAATGCCCAAAACTCTTCA |
| | | GGAGCTGCGTGCCCATGAGAATGAGATCACCAAAGTG |
| | | CGAAAAGTTACTTTCAATGGACTGAACCAGATGATTG |
| | | TCATAGAACTGGGCACCAATCCGCTGAAGAGCTCAGG |
| | | AATTGAAAATGGGGCTTTCCAGGGAATGAAGAAGCTC |
| | | TCCTACATCCGCATTGCTGATACCAATATCACCAGCAT |
| | | TCCTCAAGGTCTTCCTCCTTCCCTTACGGAATTACATC |
| | | TTGATGGCAACAAAATCAGCAGAGTTGATGCAGCTAG |
| | | CCTGAAAGGACTGAATAATTTGGCTAAGTTGGGATTG |
| | | AGTTTCAACAGCATCTCTGCTGTTGACAATGGCTCTCT |
| | | GGCCAACACGCCTCATCTGAGGGAGCTTCACTTGGAC |
| | | AACAACAAGCTTACCAGAGTACCTGGTGGGCTGGCAG |
| | | AGCATAAGTACATCCAGGTTGTCTACCTTCATAACAAC |
| | | AATATCTCTGTAGTTGGATCAAGTGACTTCTGCCCACC |
| | | TGGACACAACACCAAAAAGGCTTCTTATTCGGGTGTG |
| | | AGTCTTTTCAGCAACCCGGTCCAGTACTGGGAGATAC |
| | | AGCCATCCACCTTCAGATGTGTCTACGTGCGCTCTGCC |
| | | ATTCAACTCGGAAACTATAAGTAAAAAAATTGAAATT |
| | | TTATTTTTTTTTTTTGGAATATAAATAATGGTGAGCAA |
| | | GGGCGAGGAGCTGTTCACCGGGGTGGTGCCCATCCTG |
| | | GTCGAGCTGGACGGCGACGTAAACGGCCACAAGTTCA |
| | | GCGTGTCCGGCGAGGGCGAGGGCGATGCCACCTACGG |
| | | CAAGCTGACCCTGAAGTTCATCTGCACCACCGGCAAG |
| | | CTGCCCGTGCCCTGGCCCACCCTCGTGACCACCCTGAC |
| | | CTACGGCGTGCAGTGCTTCAGCCGCTACCCCGACCAC |
| | | ATGAAGCAGCACGACTTCTTCAAGTCCGCCATGCCCG |
| | | AAGGCTACGTCCAGGAGCGCACCATCTTCTTCAAGGA |
| | | CGACGGCAACTACAAGACCCGCGCCGAGGTGAAGTTC |
| | | GAGGGCGACACCCTGGTGAACCGCATCGAGCTGAAGG |
| | | GCATCGACTTCAAGGAGGACGGCAACATCCTGGGGCA |
| | | CAAGCTGGAGTACAACTACAACAGCCACAACGTCTAT |
| | | ATCATGGCCGACAAGCAGAAGAACGGCATCAAGGTG |
| | | AACTTCAAGATCCGCCACAACATCGAGGACGGCAGCG |
| | | TGCAGCTCGCCGACCACTACCAGCAGAACACCCCCAT |
| | | CGGCGACGGCCCCGTGCTGCTGCCCGACAACCACTAC |
| | | CTGAGCACCCAGTCCGCCCTGAGCAAAGACCCCAACG |
| | | AGAAGCGCGATCACATGGTCCTGCTGGAGTTCGTGAC |
| | | CGCCGCCGGGATCACTCTCGGCATGGACGAGCTGTAC |
| | | AAGTAATTATATAAGGTATCTCGTTTGTCTATAACAAA |
| | | GATCGTAACTGACCTTTTTTATATCGAGAAAACATACG |
| | | TTTAGTTCATCCTCAAACGTAACACCGTAACTGCCTCG |
| | | GACATCCTCCTTGTTGTCGTACACAAACATACTAATCG |
| | | GATGCGTGAAATGAGGATTCACTTTAATCGGATTGGTT |
| | | TCTAGGTTAACACATGTTACACAGGATCCTAAGATGG |
| | | TTATGGACACATCCTTGTTGTGATGTAACGAGTCGGGA |
| | | AGTTGATTGCCGTAGTTGCCCACGTCGCCCTCCGGTTC |
| | | CAGACACGTAATGGTTAGGTATATATCCGAATACTTC |
| | | GTCAACGGATGAGTCGTAAATAACATGATGGATAGCT |
| | | TGTTCCCATCTCCTGCACCAGCACTGGCCGCCACAAAT |
| | | CGTTGTACCACGTTAGTAATCGTAATGTTTATCATAAG |
| | | CCCGTACCCGGTTAATATGAGCGTGGACGTTTTATGAT |
| | | CGTATCGTTCCTTCATGTGACATTCTCCCATAACCGTT |
| | | TCGACGTACCGATTTAACCCGATGGTTAGCTCGGCGG |
| | | CTAAGTGCCAGTACTTTTTTGGATACGTCGCACATGTT |
| | | GAGGTTGCGACGAGGCAGGCGAGCACGATGATAATAT |
| | | ACCGCGCCAT |
| 22 | Full recombination | GACGAAAGGGCCTCGTGATACGCCTATTTTTATAGGTT |
| | plasmid sequence | AATGTCATGATAATAATGGTTTCTTAGACGTCAGGTGG |
| | for generation of | CACTTTTCGGGGAAATGTGCGCGGAACCCCTATTTGTT |
| | HV14 | TATTTTTCTAAATACATTCAAATATGTATCCGCTCATG |
| | | AGACAATAACCCTGATAAATGCTTCAATAATATTGAA |
| | | AAAGGAAGAGTATGAGTATTCAACATTTCCGTGTCGC |
| | | CCTTATTCCCTTTTTTGCGGCATTTTGCCTTCCTGTTTT |
| | | TGCTCACCCAGAAACGCTGGTGAAAGTAAAAGATGCT |
| | | GAAGATCAGTTGGGTGCACGAGTGGGTTACATCGAAC |
| | | TGGATCTCAACAGCGGTAAGATCCTTGAGAGTTTTCGC |
| | | CCCGAAGAACGTTTTCCAATGATGAGCACTTTTAAAGT |
| | | TCTGCTATGTGGCGCGGTATTATCCCGTATTGACGCCG |
| | | GGCAAGAGCAACTCGGTCGCCGCATACACTATTCTCA |
| | | GAATGACTTGGTTGAGTACTCACCAGTCACAGAAAAG |
| | | CATCTTACGGATGGCATGACAGTAAGAGAATTATGCA |
| | | GTGCTGCCATAACCATGAGTGATAACACTGCGGCCAA |
| | | CTTACTTCTGACAACGATCGGAGGACCGAAGGAGCTA |
| | | ACCGCTTTTTTGCACAACATGGGGGATCATGTAACTCG |
| | | CCTTGATCGTTGGGAACCGGAGCTGAATGAAGCCATA |
| | | CCAAACGACGAGCGTGACACCACGATGCCTGTAGCAA |
| | | TGGCAACAACGTTGCGCAAACTATTAACTGGCGAACT |
| | | ACTTACTCTAGCTTCCCGGCAACAATTAATAGACTGGA |
| | | TGGAGGCGGATAAAGTTGCAGGACCACTTCTGCGCTC |
| | | GGCCCTTCCGGCTGGCTGGTTTATTGCTGATAAATCTG |
| | | GAGCCGGTGAGCGTGGGTCTCGCGGTATCATTGCAGC |
| | | ACTGGGGCCAGATGGTAAGCCCTCCCGTATCGTAGTT |
| | | ATCTACACGACGGGGAGTCAGGCAACTATGGATGAAC |
| | | GAAATAGACAGATCGCTGAGATAGGTGCCTCACTGAT |
| | | TAAGCATTGGTAACTGTCAGACCAAGTTTACTCATATA |
| | | TACTTTAGATTGATTTAAAACTTCATTTTTAATTTAAA |
| | | AGGATCTAGGTGAAGATCCTTTTTGATAATCTCATGAC |
| | | CAAAATCCCTTAACGTGAGTTTTCGTTCCACTGAGCGT |
| | | CAGACCCCGTAGAAAAGATCAAAGGATCTTCTTGAGA |
| | | TCCTTTTTTTCTGCGCGTAATCTGCTGCTTGCAAACAA |
| | | AAAAACCACCGCTACCAGCGGTGGTTTGTTTGCCGGA |
| | | TCAAGAGCTACCAACTCTTTTTCCGAAGGTAACTGGCT |
| | | TCAGCAGAGCGCAGATACCAAATACTGTTCTTCTAGT |
| | | GTAGCCGTAGTTAGGCCACCACTTCAAGAACTCTGTA |
| | | GCACCGCCTACATACCTCGCTCTGCTAATCCTGTTACC |
| | | AGTGGCTGCTGCCAGTGGCGATAAGTCGTGTCTTACC |
| | | GGGTTGGACTCAAGACGATAGTTACCGGATAAGGCGC |
| | | AGCGGTCGGGCTGAACGGGGGGTTCGTGCACACAGCC |
| | | CAGCTTGGAGCGAACGACCTACACCGAACTGAGATAC |
| | | CTACAGCGTGAGCTATGAGAAAGCGCCACGCTTCCCG |
| | | AAGGGAGAAAGGCGGACAGGTATCCGGTAAGCGGCA |
| | | GGGTCGGAACAGGAGAGCGCACGAGGGAGCTTCCAG |
| | | GGGGAAACGCCTGGTATCTTTATAGTCCTGTCGGGTTT |
| | | CGCCACCTCTGACTTGAGCGTCGATTTTTGTGATGCTC |
| | | GTCAGGGGGGCGGAGCCTATGGAAAAACGCCAGCAA |
| | | CGCGGCCTTTTTACGGTTCCTGGCCTTTTGCTGGCCTTT |
| | | TGCTCACATGTTCTTTCCTGCGTTATCCCCTGATTCTGT |
| | | GGATAACCGTATTACCGCCTTTGAGTGAGCTGATACC |
| | | GCTCGCCGCAGCCGAACGACCGAGCGCAGCGAGTCAG |
| | | TGAGCGAGGAAGCGGAAGAGCGCCCAATACGCAAAC |
| | | CGCCTCTCCCCGCGCGTTGGCCGATTCATTAATGCAGC |
| | | TGGCACGACAGGTTTCCCGACTGGAAAGCGGGCAGTG |
| | | AGCGCAACGCAATTAATGTGAGTTAGCTCACTCATTA |
| | | GGCACCCCAGGCTTTACACTTTATGCTTCCGGCTCGTA |
| | | TGTTGTGTGGAATTGTGAGCGGATAACAATTTCACAC |
| | | AGGAAACAGCTATGACCATGATTACGCCAAGCTTGCA |
| | | TGCAGGCCTCTGCAGTCGACGGGCCCGGGATCCGATA |
| | | TCTAGATGCATTCGCGAGGTACCGAGCTCGAATTCGA |
| | | TGTCGTACATCGATTACACAAAGAAGTAGAGTCATAC |
| | | GACGTACGTTTCCCTATAAAATCGGTAAACCTAGACG |
| | | CGGTGTTTCTATCCATAAACGTAACACGTGTACGTCTA |
| | | CGTTGGAAGATACCCTTGACCGAACACAATCCTTATC |
| | | AGACGGCCTACGGATGTTCTAACGACAGATTATACAG |
| | | CTACAACGAGTACGCTTTTTCTCATTTAAAACAAGACC |
| | | GTGTAAAGATCATAGAACTCCCATGTGACGACGATTA |
| | | CAGCGTCGTGTTAATCACACACGATAGCCGTTCGACT |
| | | ATTACACCGGATAAAGTGACCGGGTGGCTGCGCACGA |
| | | CCCGTCTACGTTACGTAAACGTATCCCTACCCAAGGGT |
| | | TCCACGGAAACGGGACACAACGTAACGTGTCTAACTC |
| | | CCACACACGTCAATCTATGTCATCGTTGTCGTATAACG |
| | | ATTACCAAAACGGGCGTGGACGCAACCGCGTTCTCAT |
| | | GCGTCGACGGCGATACATGCACCGAACACGACACGAC |
| | | CGCGTCAACGTGTACGATTATTATAAAAACGACGGGT |
| | | CTAGACTTTTTGTTTATGGGGAAACTCTAAGAATTTCA |
| | | TTTTGTTTTTTTCTATGCTATAAATGTGTCACCAGCAGT |
| | | TGGTCATCTCTTGGTTTTCCCTGGTTTTTCTGGCATCTC |
| | | CCCTCGTGGCCATATGGGAACTGAAGAAAGATGTTTA |
| | | TGTCGTAGAATTGGATTGGTATCCGGATGCCCCTGGA |
| | | GAAATGGTGGTCCTCACCTGTGACACCCCTGAAGAAG |
| | | ATGGTATCACCTGGACCTTGGACCAGAGCAGTGAGGT |
| | | CTTAGGCTCTGGCAAAACCCTGACCATCCAAGTCAAA |
| | | GAGTTTGGAGATGCTGGCCAGTACACCTGTCACAAAG |
| | | GAGGCGAGGTTCTAAGCCATTCGCTCCTGCTGCTTCAC |
| | | AAAAAGGAAGATGGAATTTGGTCCACTGATATTTTAA |
| | | AGGACCAGAAAGAACCCAAAAATAAGACCTTTCTAAG |
| | | ATGCGAGGCCAAGAATTATTCTGGACGTTTCACCTGCT |
| | | GGTGGCTGACGACAATCAGTACTGATTTGACATTCAG |
| | | TGTCAAAAGCAGCAGAGGCTCTTCTGACCCCCAAGGG |
| | | GTGACGTGCGGAGCTGCTACACTCTCTGCAGAGAGAG |
| | | TCAGAGGGGACAACAAGGAGTATGAGTACTCAGTGGA |
| | | GTGCCAGGAGGACAGTGCCTGCCCAGCTGCTGAGGAG |
| | | AGTCTGCCCATTGAGGTCATGGTGGATGCCGTTCACA |
| | | AGCTCAAGTATGAAAACTACACCAGCAGCTTCTTCAT |
| | | CAGGGACATCATCAAACCTGACCCACCCAAGAACTTG |
| | | CAGCTGAAGCCATTAAAGAATTCTCGGCAGGTGGAGG |
| | | TCAGCTGGGAGTACCCTGACACCTGGAGTACTCCACA |
| | | TTCCTACTTCTCCCTGACATTCTGCGTTCAGGTCCAGG |
| | | GCAAGAGCAAGAGAGAAAAGAAAGATAGAGTCTTCA |
| | | CGGACAAGACCTCAGCCACGGTCATCTGCCGCAAAAA |
| | | TGCCAGCATTAGCGTGCGGGCCCAGGACCGCTACTAT |
| | | AGCTCATCTTGGAGCGAATGGGCATCTGTGCCCTGCA |
| | | GTGTTCCTGGAGTAGGGGTACCTGGGGTGGGCGCCAG |
| | | AAACCTCCCCGTGGCCACTCCAGACCCAGGAATGTTC |
| | | CCATGCCTTCACCACTCCCAAAACCTGCTGAGGGCCGT |
| | | CAGCAACATGCTCCAGAAGGCCAGACAAACTCTAGAA |
| | | TTTTACCCTTGCACTTCTGAAGAGATTGATCATGAAGA |
| | | TATCACAAAAGATAAAACCAGCACAGTGGAGGCCTGT |
| | | TTACCATTGGAATTAACCAAGAATGAGAGTTGCCTAA |
| | | ATTCCAGAGAGACCTCTTTCATAACTAATGGGAGTTGC |
| | | CTGGCCTCCAGAAAGACCTCTTTTATGATGGCCCTGTG |
| | | CCTTAGTAGTATTTATGAAGACTTGAAGATGTACCAG |
| | | GTGGAGTTCAAGACCATGAATGCAAAGCTGCTGATGG |
| | | ATCCTAAGAGGCAGATCTTTCTAGATCAAAACATGCT |
| | | GGCAGTTATTGATGAGCTGATGCAGGCCCTGAATTTC |
| | | AACAGTGAGACTGTGCCACAAAAATCCTCCCTTGAAG |
| | | AACCGGATTTTTATAAAACTAAAATCAAGCTCTGCAT |
| | | ACTTCTTCATGCTTTCAGAATTCGGGCAGTGACTATTG |
| | | ATAGAGTGATGAGCTATCTGAATGCTTCCTAAATGTCT |
| | | GAATTTCATTTTGTTTTTTTCTATGCTATAAATGAGCAC |
| | | TGAAAGCATGATCCGGGACGTGGAGCTGGCCGAGGAG |
| | | GCGCTCCCCAAGAAGACAGGGGGGCCCCAGGGCTCCA |
| | | GGCGGTGCTTGTTCCTCAGCCTCTTCTCCTTCCTGATC |
| | | GTGGCAGGCGCCACCACGCTCTTCTGCCTGCTGCACTT |
| | | TGGAGTGATCGGCCCCCAGAGGGAAGAGTTCCCCAGG |
| | | GACCTCTCTCTAATCAGCCCTCTGGCCCAGGCAGTCAG |
| | | ATCATCTTCTCGAACCCCGAGTGACAAGCCTGTAGCCC |
| | | ATGTTGTAGCAAACCCTCAAGCTGAGGGGCAGCTCCA |
| | | GTGGCTGAACCGCCGGGCCAATGCCCTCCTGGCCAAT |
| | | GGCGTGGAGCTGAGAGATAACCAGCTGGTGGTGCCAT |
| | | CAGAGGGCCTGTACCTCATCTACTCCCAGGTCCTCTTC |
| | | AAGGGCCAAGGCTGCCCCTCCACCCATGTGCTCCTCA |
| | | CCCACACCATCAGCCGCATCGCCGTCTCCTACCAGACC |
| | | AAGGTCAACCTCCTCTCTGCCATCAAGAGCCCCTGCCA |
| | | GAGGGAGACCCCAGAGGGGGCTGAGGCCAAGCCCTG |
| | | GTATGAGCCCATCTATCTGGGAGGGGTCTTCCAGCTG |
| | | GAGAAGGGTGACCGACTCAGCGCTGAGATCAATCGGC |
| | | CCGACTATCTCGACTTTGCCGAGTCTGGGCAGGTCTAC |
| | | TTTGGGATCATTGCCCTGTGAAGCTTGAAAAATTGAA |
| | | ATTTTATTTTTTTTTTTTGGAATATAAATAATGAAGGC |
| | | CACTATCATCCTCCTTCTGCTTGCACAAGTTTCCTGGG |
| | | CTGGACCGTTTCAACAGAGAGGCTTATTTGACTTTATG |
| | | CTAGAAGATGAGGCTTCTGGGATAGGCCCAGAAGTTC |
| | | CTGATGACCGCGACTTCGAGCCCTCCCTAGGCCCAGT |
| | | GTGCCCCTTCCGCTGTCAATGCCATCTTCGAGTGGTCC |
| | | AGTGTTCTGATTTGGGTCTGGACAAAGTGCCAAAGGA |
| | | TCTTCCCCCTGACACAACTCTGCTAGACCTGCAAAACA |
| | | ACAAAATAACCGAAATCAAAGATGGAGACTTTAAGAA |
| | | CCTGAAGAACCTTCACGCATTGATTCTTGTCAACAATA |
| | | AAATTAGCAAAGTTAGTCCTGGAGCATTTACACCTTTG |
| | | GTGAAGTTGGAACGACTTTATCTGTCCAAGAATCAGC |
| | | TGAAGGAATTGCCAGAAAAAATGCCCAAAACTCTTCA |
| | | GGAGCTGCGTGCCCATGAGAATGAGATCACCAAAGTG |
| | | CGAAAAGTTACTTTCAATGGACTGAACCAGATGATTG |
| | | TCATAGAACTGGGCACCAATCCGCTGAAGAGCTCAGG |
| | | AATTGAAAATGGGGCTTTCCAGGGAATGAAGAAGCTC |
| | | TCCTACATCCGCATTGCTGATACCAATATCACCAGCAT |
| | | TCCTCAAGGTCTTCCTCCTTCCCTTACGGAATTACATC |
| | | TTGATGGCAACAAAATCAGCAGAGTTGATGCAGCTAG |
| | | CCTGAAAGGACTGAATAATTTGGCTAAGTTGGGATTG |
| | | AGTTTCAACAGCATCTCTGCTGTTGACAATGGCTCTCT |
| | | GGCCAACACGCCTCATCTGAGGGAGCTTCACTTGGAC |
| | | AACAACAAGCTTACCAGAGTACCTGGTGGGCTGGCAG |
| | | AGCATAAGTACATCCAGGTTGTCTACCTTCATAACAAC |
| | | AATATCTCTGTAGTTGGATCAAGTGACTTCTGCCCACC |
| | | TGGACACAACACCAAAAAGGCTTCTTATTCGGGTGTG |
| | | AGTCTTTTCAGCAACCCGGTCCAGTACTGGGAGATAC |
| | | AGCCATCCACCTTCAGATGTGTCTACGTGCGCTCTGCC |
| | | ATTCAACTCGGAAACTATAAGTAAAAAAATTGAAATT |
| | | TTATTTTTTTTTTTTGGAATATAAATAATGGTGAGCAA |
| | | GGGCGAGGAGCTGTTCACCGGGGTGGTGCCCATCCTG |
| | | GTCGAGCTGGACGGCGACGTAAACGGCCACAAGTTCA |
| | | GCGTGTCCGGCGAGGGCGAGGGCGATGCCACCTACGG |
| | | CAAGCTGACCCTGAAGTTCATCTGCACCACCGGCAAG |
| | | CTGCCCGTGCCCTGGCCCACCCTCGTGACCACCCTGAC |
| | | CTACGGCGTGCAGTGCTTCAGCCGCTACCCCGACCAC |
| | | ATGAAGCAGCACGACTTCTTCAAGTCCGCCATGCCCG |
| | | AAGGCTACGTCCAGGAGCGCACCATCTTCTTCAAGGA |
| | | CGACGGCAACTACAAGACCCGCGCCGAGGTGAAGTTC |
| | | GAGGGCGACACCCTGGTGAACCGCATCGAGCTGAAGG |
| | | GCATCGACTTCAAGGAGGACGGCAACATCCTGGGGCA |
| | | CAAGCTGGAGTACAACTACAACAGCCACAACGTCTAT |
| | | ATCATGGCCGACAAGCAGAAGAACGGCATCAAGGTG |
| | | AACTTCAAGATCCGCCACAACATCGAGGACGGCAGCG |
| | | TGCAGCTCGCCGACCACTACCAGCAGAACACCCCCAT |
| | | CGGCGACGGCCCCGTGCTGCTGCCCGACAACCACTAC |
| | | CTGAGCACCCAGTCCGCCCTGAGCAAAGACCCCAACG |
| | | AGAAGCGCGATCACATGGTCCTGCTGGAGTTCGTGAC |
| | | CGCCGCCGGGATCACTCTCGGCATGGACGAGCTGTAC |
| | | AAGTAATTATATAAGGTATCTCGTTTGTCTATAACAAA |
| | | GATCGTAACTGACCTTTTTTATATCGAGAAAACATACG |
| | | TTTAGTTCATCCTCAAACGTAACACCGTAACTGCCTCG |
| | | GACATCCTCCTTGTTGTCGTACACAAACATACTAATCG |
| | | GATGCGTGAAATGAGGATTCACTTTAATCGGATTGGTT |
| | | TCTAGGTTAACACATGTTACACAGGATCCTAAGATGG |
| | | TTATGGACACATCCTTGTTGTGATGTAACGAGTCGGGA |
| | | AGTTGATTGCCGTAGTTGCCCACGTCGCCCTCCGGTTC |
| | | CAGACACGTAATGGTTAGGTATATATCCGAATACTTC |
| | | GTCAACGGATGAGTCGTAAATAACATGATGGATAGCT |
| | | TGTTCCCATCTCCTGCACCAGCACTGGCCGCCACAAAT |
| | | CGTTGTACCACGTTAGTAATCGTAATGTTTATCATAAG |
| | | CCCGTACCCGGTTAATATGAGCGTGGACGTTTTATGAT |
| | | CGTATCGTTCCTTCATGTGACATTCTCCCATAACCGTT |
| | | TCGACGTACCGATTTAACCCGATGGTTAGCTCGGCGG |
| | | CTAAGTGCCAGTACTTTTTTGGATACGTCGCACATGTT |
| | | GAGGTTGCGACGAGGCAGGCGAGCACGATGATAATAT |
| | | ACCGCGCCATGAATTCACTGGCCGTCGTTTTACAACGT |
| | | CGTGACTGGGAAAACCCTGGCGTTACCCAACTTAATC |
| | | GCCTTGCAGCACATCCCCCTTTCGCCAGCTGGCGTAAT |
| | | AGCGAAGAGGCCCGCACCGATCGCCCTTCCCAACAGT |
| | | TGCGCAGCCTGAATGGCGAATGGCGCCTGATGCGGTA |
| | | TTTTCTCCTTACGCATCTGTGCGGTATTTCACACCGCA |
| | | TATGGTGCACTCTCAGTACAATCTGCTCTGATGCCGCA |
| | | TAGTTAAGCCAGCCCCGACACCCGCCAACACCCGCTG |
| | | ACGCGCCCTGACGGGCTTGTCTGCTCCCGGCATCCGCT |
| | | TACAGACAAGCTGTGACCGTCTCCGGGAGCTGCATGT |
| | | GTCAGAGGTTTTCACCGTCATCACCGAAACGCGCGA |
| 23 | TNF specific | GAAGAGGACCTGGGAGTAGATG |
| | primer | |
| 24 | IRES | TATGCTAGTACGTCTCTCAAGGATAAGTAAGTAATATT |
| | | AAGGTACGGGAGGTATTGGACAGGCCGCAATAAAATA |
| | | TCTTTATTTTCATTACATCTGTGTGTTGGTTTTTTGTGT |
| | | GAATCGATAGTACTAACATACGCTCTCCATCAAAACA |
| | | AAACGAAACAAAACAAACTAGCAAAATAGGCTGTCCC |
| | | CAGTGCAAGTGCAGGTGCCAGAACATTTCTCTGGCCT |
| | | AACTGGCCGGTACCTGAGCTCTAGTTTCACTTTCCCTA |
| | | GTTTCACTTTCCCTAGTTTCACTTTCCCTAGTTTCACTT |
| | | TCCCTAGTTTCACTTTCCCCTCGAGGATATCAAGATCT |
| | | GGCCTCGGCGGCCAG |
| 25 | HV12 insert | AAAATTGAAATTTTATTTTTTTTTTTTGGAATATAAAT |
| | sequence | AATGAAGGCCACTATCATCCTCCTTCTGCTTGCACAAG |
| | | TTTCCTGGGCTGGACCGTTTCAACAGAGAGGCTTATTT |
| | | GACTTTATGCTAGAAGATGAGGCTTCTGGGATAGGCC |
| | | CAGAAGTTCCTGATGACCGCGACTTCGAGCCCTCCCTA |
| | | GGCCCAGTGTGCCCCTTCCGCTGTCAATGCCATCTTCG |
| | | AGTGGTCCAGTGTTCTGATTTGGGTCTGGACAAAGTGC |
| | | CAAAGGATCTTCCCCCTGACACAACTCTGCTAGACCTG |
| | | CAAAACAACAAAATAACCGAAATCAAAGATGGAGAC |
| | | TTTAAGAACCTGAAGAACCTTCACGCATTGATTCTTGT |
| | | CAACAATAAAATTAGCAAAGTTAGTCCTGGAGCATTT |
| | | ACACCTTTGGTGAAGTTGGAACGACTTTATCTGTCCAA |
| | | GAATCAGCTGAAGGAATTGCCAGAAAAAATGCCCAAA |
| | | ACTCTTCAGGAGCTGCGTGCCCATGAGAATGAGATCA |
| | | CCAAAGTGCGAAAAGTTACTTTCAATGGACTGAACCA |
| | | GATGATTGTCATAGAACTGGGCACCAATCCGCTGAAG |
| | | AGCTCAGGAATTGAAAATGGGGCTTTCCAGGGAATGA |
| | | AGAAGCTCTCCTACATCCGCATTGCTGATACCAATATC |
| | | ACCAGCATTCCTCAAGGTCTTCCTCCTTCCCTTACGGA |
| | | ATTACATCTcGATGGCAACAAAATCAGCAGAGTTGAT |
| | | GCAGCTAGCCTGAAAGGACTGAATAATTTGGCTAAGT |
| | | TGGGATTGAGTTTCAACAGCATCTCTGCTGTTGACAAT |
| | | GGCTCTCTGGCCAACACGCCTCATCTGAGGGAGCTTC |
| | | ACTTGGACAACAACAAGCTTACCAGAGTACCTGGTGG |
| | | GCTGGCAGAGCATAAGTACATCCAGGTTGTCTACCTTC |
| | | ATAACAACAATATCTCTGTAGTTGGATCAAGTGACTTC |
| | | TGCCCACCTGGACACAACACCAAAAAGGCTTCTTATT |
| | | CGGGTGTGAGTCTTTTCAGCAACCCGGTCCAGTACTGG |
| | | GAGATACAGCCATCCACCTTCAGATGTGTCTACGTGC |
| | | GCTCTGCCATTCAACTCGGAAACTATAAGTAAGCTTG |
| | | GACTCCTGTTGATAGATCCAGAAAAATTGAAATTTTAT |
| | | TTTTTTTTTTTGGAATATAAATAATGTGTCACCAGCAG |
| | | TTGGTCATCTCTTGGTTTTCCCTGGTTTTTCTGGCATCT |
| | | CCCCTCGTGGCCATATGGGAACTGAAGAAAGATGTTT |
| | | ATGTCGTAGAATTGGATTGGTATCCGGATGCCCCTGG |
| | | AGAAATGGTGGTCCTCACCTGTGACACCCCTGAAGAA |
| | | GATGGTATCACCTGGACCTTGGACCAGAGCAGTGAGG |
| | | TCTTAGGCTCTGGCAAAACCCTGACCATCCAAGTCAA |
| | | AGAGTTTGGAGATGCTGGCCAGTACACCTGTCACAAA |
| | | GGAGGCGAGGTTCTAAGCCATTCGCTCCTGCTGCTTCA |
| | | CAAAAAGGAAGATGGAATTTGGTCCACTGATATTTTA |
| | | AAGGACCAGAAAGAACCCAAAAATAAGACCTTTCTAA |
| | | GATGCGAGGCCAAGAATTATTCTGGACGTTTCACCTG |
| | | CTGGTGGCTGACGACAATCAGTACTGATTTGACATTCA |
| | | GTGTCAAAAGCAGCAGAGGCTCTTCTGACCCCCAAGG |
| | | GGTGACGTGCGGAGCTGCTACACTCTCTGCAGAGAGA |
| | | GTCAGAGGGGACAACAAGGAGTATGAGTACTCAGTGG |
| | | AGTGCCAGGAGGACAGTGCCTGCCCAGCTGCTGAGGA |
| | | GAGTCTGCCCATTGAGGTCATGGTGGATGCCGTTCAC |
| | | AAGCTCAAGTATGAAAACTACACCAGCAGCTTCTTCA |
| | | TCAGGGACATCATCAAACCTGACCCACCCAAGAACTT |
| | | GCAGCTGAAGCCATTAAAGAATTCTCGGCAGGTGGAG |
| | | GTCAGCTGGGAGTACCCTGACACCTGGAGTACTCCAC |
| | | ATTCCTACTTCTCCCTGACATTCTGCGTTCAGGTCCAG |
| | | GGCAAGAGCAAGAGAGAAAAGAAAGATAGAGTCTTC |
| | | ACGGACAAGACCTCAGCCACGGTCATCTGCCGCAAAA |
| | | ATGCCAGCATTAGCGTGCGGGCCCAGGACCGCTACTA |
| | | TAGCTCATCTTGGAGCGAATGGGCATCTGTGCCCTGCA |
| | | GTTAGTATGCTAGTACGTCTCTCAAGGATAAGTAAGT |
| | | AATATTAAGGTACGGGAGGTATTGGACAGGCCGCAAT |
| | | AAAATATCTTTATTTTCATTACATCTGTGTGTTGGTTTT |
| | | TTGTGTGAATCGATAGTACTAACATACGCTCTCCATCA |
| | | AAACAAAACGAAACAAAACAAACTAGCAAAATAGGC |
| | | TGTCCCCAGTGCAAGTGCAGGTGCCAGAACATTTCTCT |
| | | GGCCTAACTGGCCGGTACCTGAGCTCTAGTTTCACTTT |
| | | CCCTAGTTTCACTTTCCCTAGTTTCACTTTCCCTAGTTT |
| | | CACTTTCCCTAGTTTCACTTTCCCCTCGAGGATATCAA |
| | | GATCTGGCCTCGGCGGCCAGATGTGGCCCCCTGGGTC |
| | | AGCCTCCCAGCCACCGCCCTCACCTGCCGCGGCCACA |
| | | GGTCTGCATCCAGCGGCTCGCCCTGTGTCCCTGCAGTG |
| | | CCGGCTCAGCATGTGTCCAGCGCGCAGCCTCCTCCTTG |
| | | TGGCTACCCTGGTCCTCCTGGACCACCTCAGTTTGGCC |
| | | AGAAACCTCCCCGTGGCCACTCCAGACCCAGGAATGT |
| | | TCCCATGCCTTCACCACTCCCAAAACCTGCTGAGGGCC |
| | | GTCAGCAACATGCTCCAGAAGGCCAGACAAACTCTAG |
| | | AATTTTACCCTTGCACTTCTGAAGAGATTGATCATGAA |
| | | GATATCACAAAAGATAAAACCAGCACAGTGGAGGCCT |
| | | GTTTACCATTGGAATTAACCAAGAATGAGAGTTGCCT |
| | | AAATTCCAGAGAGACCTCTTTCATAACTAATGGGAGT |
| | | TGCCTGGCCTCCAGAAAGACCTCTTTTATGATGGCCCT |
| | | GTGCCTTAGTAGTATTTATGAAGACTTGAAGATGTACC |
| | | AGGTGGAGTTCAAGACCATGAATGCAAAGCTTCTGAT |
| | | GGATCCTAAGAGGCAGATCTTTCTAGATCAAAACATG |
| | | CTGGCAGTTATTGATGAGCTGATGCAGGCCCTGAATTT |
| | | CAACAGTGAGACTGTGCCACAAAAATCCTCCCTTGAA |
| | | GAACCGGATTTTTATAAAACTAAAATCAAGCTCTGCA |
| | | TACTTCTTCATGCTTTCAGAATTCGGGCAGTGACTATT |
| | | GATAGAGTGATGAGCTATCTGAATGCTTCCTAAGGGG |
| | | ACAACTTTGTATAATAAAGTTGCTAAAAATTGAAATTT |
| | | TATTTTTTTTTTTTGGAATATAAATAATGGTGAGCAAG |
| | | GGCGAGGAGCTGTTCACCGGGGTGGTGCCCATCCTGG |
| | | TCGAGCTGGACGGCGACGTAAACGGCCACAAGTTCAG |
| | | CGTGTCCGGCGAGGGCGAGGGCGATGCCACCTACGGC |
| | | AAGCTGACCCTGAAGTTCATCTGCACCACCGGCAAGC |
| | | TGCCCGTGCCCTGGCCCACCCTCGTGACCACCCTGACC |
| | | TACGGCGTGCAGTGCTTCAGCCGCTACCCCGACCACA |
| | | TGAAGCAGCACGACTTCTTCAAGTCCGCCATGCCCGA |
| | | AGGCTACGTCCAGGAGCGCACCATCTTCTTCAAGGAC |
| | | GACGGCAACTACAAGACCCGCGCCGAGGTGAAGTTCG |
| | | AGGGCGACACCCTGGTGAACCGCATCGAGCTGAAGGG |
| | | CATCGACTTCAAGGAGGACGGCAACATCCTGGGGCAC |
| | | AAGCTGGAGTACAACTACAACAGCCACAACGTCTATA |
| | | TCATGGCCGACAAGCAGAAGAACGGCATCAAGGTGA |
| | | ACTTCAAGATCCGCCACAACATCGAGGACGGCAGCGT |
| | | GCAGCTCGCCGACCACTACCAGCAGAACACCCCCATC |
| | | GGCGACGGCCCCGTGCTGCTGCCCGACAACCACTACC |
| | | TGAGCACCCAGTCCGCCCTGAGCAAAGACCCCAACGA |
| | | GAAGCGCGATCACATGGTCCTGCTGGAGTTCGTGACC |
| | | GCCGCCGGGATCACTCTCGGCATGGACGAGCTGTACA |
| | | AGTAA |
| 26 | HV12 insert | GATGTCGTACATCGATTACACAAAGAAGTAGAGTCAT |
| | sequence plus | ACGACGTACGTTTCCCTATAAAATCGGTAAACCTAGA |
| | recombination arms | CGCGGTGTTTCTATCCATAAACGTAACACGTGTACGTC |
| | | TACGTTGGAAGATACCCTTGACCGAACACAATCCTTAT |
| | | CAGACGGCCTACGGATGTTCTAACGACAGATTATACA |
| | | GCTACAACGAGTACGCTTTTTCTCATTTAAAACAAGAC |
| | | CGTGTAAAGATCATAGAACTCCCATGTGACGACGATT |
| | | ACAGCGTCGTGTTAATCACACACGATAGCCGTTCGAC |
| | | TATTACACCGGATAAAGTGACCGGGTGGCTGCGCACG |
| | | ACCCGTCTACGTTACGTAAACGTATCCCTACCCAAGG |
| | | GTTCCACGGAAACGGGACACAACGTAACGTGTCTAAC |
| | | TCCCACACACGTCAATCTATGTCATCGTTGTCGTATAA |
| | | CGATTACCAAAACGGGCGTGGACGCAACCGCGTTCTC |
| | | ATGCGTCGACGGCGATACATGCACCGAACACGACACG |
| | | ACCGCGTCAACGTGTACGATTATTATAAAAACGACGG |
| | | GTCTAGACTTTTTGTTTATGGGGAAACTCTAAGACAAC |
| | | TTTTCTATACAAAGTTGCCAAAATTGAAATTTTATTTT |
| | | TTTTTTTTGGAATATAAATAATGAAGGCCACTATCATC |
| | | CTCCTTCTGCTTGCACAAGTTTCCTGGGCTGGACCGTT |
| | | TCAACAGAGAGGCTTATTTGACTTTATGCTAGAAGAT |
| | | GAGGCTTCTGGGATAGGCCCAGAAGTTCCTGATGACC |
| | | GCGACTTCGAGCCCTCCCTAGGCCCAGTGTGCCCCTTC |
| | | CGCTGTCAATGCCATCTTCGAGTGGTCCAGTGTTCTGA |
| | | TTTGGGTCTGGACAAAGTGCCAAAGGATCTTCCCCCTG |
| | | ACACAACTCTGCTAGACCTGCAAAACAACAAAATAAC |
| | | CGAAATCAAAGATGGAGACTTTAAGAACCTGAAGAAC |
| | | CTTCACGCATTGATTCTTGTCAACAATAAAATTAGCAA |
| | | AGTTAGTCCTGGAGCATTTACACCTTTGGTGAAGTTGG |
| | | AACGACTTTATCTGTCCAAGAATCAGCTGAAGGAATT |
| | | GCCAGAAAAAATGCCCAAAACTCTTCAGGAGCTGCGT |
| | | GCCCATGAGAATGAGATCACCAAAGTGCGAAAAGTTA |
| | | CTTTCAATGGACTGAACCAGATGATTGTCATAGAACT |
| | | GGGCACCAATCCGCTGAAGAGCTCAGGAATTGAAAAT |
| | | GGGGCTTTCCAGGGAATGAAGAAGCTCTCCTACATCC |
| | | GCATTGCTGATACCAATATCACCAGCATTCCTCAAGGT |
| | | CTTCCTCCTTCCCTTACGGAATTACATCTcGATGGCAA |
| | | CAAAATCAGCAGAGTTGATGCAGCTAGCCTGAAAGGA |
| | | CTGAATAATTTGGCTAAGTTGGGATTGAGTTTCAACAG |
| | | CATCTCTGCTGTTGACAATGGCTCTCTGGCCAACACGC |
| | | CTCATCTGAGGGAGCTTCACTTGGACAACAACAAGCT |
| | | TACCAGAGTACCTGGTGGGCTGGCAGAGCATAAGTAC |
| | | ATCCAGGTTGTCTACCTTCATAACAACAATATCTCTGT |
| | | AGTTGGATCAAGTGACTTCTGCCCACCTGGACACAAC |
| | | ACCAAAAAGGCTTCTTATTCGGGTGTGAGTCTTTTCAG |
| | | CAACCCGGTCCAGTACTGGGAGATACAGCCATCCACC |
| | | TTCAGATGTGTCTACGTGCGCTCTGCCATTCAACTCGG |
| | | AAACTATAAGTAAGCTTGGACTCCTGTTGATAGATCC |
| | | AGAAAAATTGAAATTTTATTTTTTTTTTTTGGAATATA |
| | | AATAATGTGTCACCAGCAGTTGGTCATCTCTTGGTTTT |
| | | CCCTGGTTTTTCTGGCATCTCCCCTCGTGGCCATATGG |
| | | GAACTGAAGAAAGATGTTTATGTCGTAGAATTGGATT |
| | | GGTATCCGGATGCCCCTGGAGAAATGGTGGTCCTCAC |
| | | CTGTGACACCCCTGAAGAAGATGGTATCACCTGGACC |
| | | TTGGACCAGAGCAGTGAGGTCTTAGGCTCTGGCAAAA |
| | | CCCTGACCATCCAAGTCAAAGAGTTTGGAGATGCTGG |
| | | CCAGTACACCTGTCACAAAGGAGGCGAGGTTCTAAGC |
| | | CATTCGCTCCTGCTGCTTCACAAAAAGGAAGATGGAA |
| | | TTTGGTCCACTGATATTTTAAAGGACCAGAAAGAACC |
| | | CAAAAATAAGACCTTTCTAAGATGCGAGGCCAAGAAT |
| | | TATTCTGGACGTTTCACCTGCTGGTGGCTGACGACAAT |
| | | CAGTACTGATTTGACATTCAGTGTCAAAAGCAGCAGA |
| | | GGCTCTTCTGACCCCCAAGGGGTGACGTGCGGAGCTG |
| | | CTACACTCTCTGCAGAGAGAGTCAGAGGGGACAACAA |
| | | GGAGTATGAGTACTCAGTGGAGTGCCAGGAGGACAGT |
| | | GCCTGCCCAGCTGCTGAGGAGAGTCTGCCCATTGAGG |
| | | TCATGGTGGATGCCGTTCACAAGCTCAAGTATGAAAA |
| | | CTACACCAGCAGCTTCTTCATCAGGGACATCATCAAA |
| | | CCTGACCCACCCAAGAACTTGCAGCTGAAGCCATTAA |
| | | AGAATTCTCGGCAGGTGGAGGTCAGCTGGGAGTACCC |
| | | TGACACCTGGAGTACTCCACATTCCTACTTCTCCCTGA |
| | | CATTCTGCGTTCAGGTCCAGGGCAAGAGCAAGAGAGA |
| | | AAAGAAAGATAGAGTCTTCACGGACAAGACCTCAGCC |
| | | ACGGTCATCTGCCGCAAAAATGCCAGCATTAGCGTGC |
| | | GGGCCCAGGACCGCTACTATAGCTCATCTTGGAGCGA |
| | | ATGGGCATCTGTGCCCTGCAGTTAGTATGCTAGTACGT |
| | | CTCTCAAGGATAAGTAAGTAATATTAAGGTACGGGAG |
| | | GTATTGGACAGGCCGCAATAAAATATCTTTATTTTCAT |
| | | TACATCTGTGTGTTGGTTTTTTGTGTGAATCGATAGTA |
| | | CTAACATACGCTCTCCATCAAAACAAAACGAAACAAA |
| | | ACAAACTAGCAAAATAGGCTGTCCCCAGTGCAAGTGC |
| | | AGGTGCCAGAACATTTCTCTGGCCTAACTGGCCGGTA |
| | | CCTGAGCTCTAGTTTCACTTTCCCTAGTTTCACTTTCCC |
| | | TAGTTTCACTTTCCCTAGTTTCACTTTCCCTAGTTTCAC |
| | | TTTCCCCTCGAGGATATCAAGATCTGGCCTCGGCGGCC |
| | | AGATGTGGCCCCCTGGGTCAGCCTCCCAGCCACCGCC |
| | | CTCACCTGCCGCGGCCACAGGTCTGCATCCAGCGGCT |
| | | CGCCCTGTGTCCCTGCAGTGCCGGCTCAGCATGTGTCC |
| | | AGCGCGCAGCCTCCTCCTTGTGGCTACCCTGGTCCTCC |
| | | TGGACCACCTCAGTTTGGCCAGAAACCTCCCCGTGGC |
| | | CACTCCAGACCCAGGAATGTTCCCATGCCTTCACCACT |
| | | CCCAAAACCTGCTGAGGGCCGTCAGCAACATGCTCCA |
| | | GAAGGCCAGACAAACTCTAGAATTTTACCCTTGCACTT |
| | | CTGAAGAGATTGATCATGAAGATATCACAAAAGATAA |
| | | AACCAGCACAGTGGAGGCCTGTTTACCATTGGAATTA |
| | | ACCAAGAATGAGAGTTGCCTAAATTCCAGAGAGACCT |
| | | CTTTCATAACTAATGGGAGTTGCCTGGCCTCCAGAAA |
| | | GACCTCTTTTATGATGGCCCTGTGCCTTAGTAGTATTT |
| | | ATGAAGACTTGAAGATGTACCAGGTGGAGTTCAAGAC |
| | | CATGAATGCAAAGCTTCTGATGGATCCTAAGAGGCAG |
| | | ATCTTTCTAGATCAAAACATGCTGGCAGTTATTGATGA |
| | | GCTGATGCAGGCCCTGAATTTCAACAGTGAGACTGTG |
| | | CCACAAAAATCCTCCCTTGAAGAACCGGATTTTTATAA |
| | | AACTAAAATCAAGCTCTGCATACTTCTTCATGCTTTCA |
| | | GAATTCGGGCAGTGACTATTGATAGAGTGATGAGCTA |
| | | TCTGAATGCTTCCTAAGGGGACAACTTTGTATAATAAA |
| | | GTTGCTAAAAATTGAAATTTTATTTTTTTTTTTTGGAAT |
| | | ATAAATAATGGTGAGCAAGGGCGAGGAGCTGTTCACC |
| | | GGGGTGGTGCCCATCCTGGTCGAGCTGGACGGCGACG |
| | | TAAACGGCCACAAGTTCAGCGTGTCCGGCGAGGGCGA |
| | | GGGCGATGCCACCTACGGCAAGCTGACCCTGAAGTTC |
| | | ATCTGCACCACCGGCAAGCTGCCCGTGCCCTGGCCCA |
| | | CCCTCGTGACCACCCTGACCTACGGCGTGCAGTGCTTC |
| | | AGCCGCTACCCCGACCACATGAAGCAGCACGACTTCT |
| | | TCAAGTCCGCCATGCCCGAAGGCTACGTCCAGGAGCG |
| | | CACCATCTTCTTCAAGGACGACGGCAACTACAAGACC |
| | | CGCGCCGAGGTGAAGTTCGAGGGCGACACCCTGGTGA |
| | | ACCGCATCGAGCTGAAGGGCATCGACTTCAAGGAGGA |
| | | CGGCAACATCCTGGGGCACAAGCTGGAGTACAACTAC |
| | | AACAGCCACAACGTCTATATCATGGCCGACAAGCAGA |
| | | AGAACGGCATCAAGGTGAACTTCAAGATCCGCCACAA |
| | | CATCGAGGACGGCAGCGTGCAGCTCGCCGACCACTAC |
| | | CAGCAGAACACCCCCATCGGCGACGGCCCCGTGCTGC |
| | | TGCCCGACAACCACTACCTGAGCACCCAGTCCGCCCT |
| | | GAGCAAAGACCCCAACGAGAAGCGCGATCACATGGTC |
| | | CTGCTGGAGTTCGTGACCGCCGCCGGGATCACTCTCG |
| | | GCATGGACGAGCTGTACAAGTAATTATATAAGGTATC |
| | | TCGTTTGTCTATAACAAAGATCGTAACTGACCTTTTTT |
| | | ATATCGAGAAAACATACGTTTAGTTCATCCTCAAACGT |
| | | AACACCGTAACTGCCTCGGACATCCTCCTTGTTGTCGT |
| | | ACACAAACATACTAATCGGATGCGTGAAATGAGGATT |
| | | CACTTTAATCGGATTGGTTTCTAGGTTAACACATGTTA |
| | | CACAGGATCCTAAGATGGTTATGGACACATCCTTGTTG |
| | | TGATGTAACGAGTCGGGAAGTTGATTGCCGTAGTTGC |
| | | CCACGTCGCCCTCCGGTTCCAGACACGTAATGGTTAG |
| | | GTATATATCCGAATACTTCGTCAACGGATGAGTCGTA |
| | | AATAACATGATGGATAGCTTGTTCCCATCTCCTGCACC |
| | | AGCACTGGCCGCCACAAATCGTTGTACCACGTTAGTA |
| | | ATCGTAATGTTTATCATAAGCCCGTACCCGGTTAATAT |
| | | GAGCGTGGACGTTTTATGATCGTATCGTTCCTTCATGT |
| | | GACATTCTCCCATAACCGTTTCGACGTACCGATTTAAC |
| | | CCGATGGTTAGCTCGGCGGCTAAGTGCCAGTACTTTTT |
| | | TGGATACGTCGCACATGTTGAGGTTGCGACGAGGCAG |
| | | GCGAGCACGATGATAATATACCGCGCCAT |
| 27 | Full plasmid | CTAGTTATTAATAGTAATCAATTACGGGGTCATTAGTT |
| | sequence used to | CATAGCCCATATATGGAGTTCCGCGTTACATAACTTAC |
| | generate HV12 | GGTAAATGGCCCGCCTGGCTGACCGCCCAACGACCCC |
| | | CGCCCATTGACGTCAATAATGACGTATGTTCCCATAGT |
| | | AACGCCAATAGGGACTTTCCATTGACGTCAATGGGTG |
| | | GAGTATTTACGGTAAACTGCCCACTTGGCAGTACATC |
| | | AAGTGTATCATATGCCAAGTACGCCCCCTATTGACGTC |
| | | AATGACGGTAAATGGCCCGCCTGGCATTATGCCCAGT |
| | | ACATGACCTTATGGGACTTTCCTACTTGGCAGTACATC |
| | | TACGTATTAGTCATCGCTATTACCATGGTGATGCGGTT |
| | | TTGGCAGTACATCAATGGGCGTGGATAGCGGTTTGAC |
| | | TCACGGGGATTTCCAAGTCTCCACCCCATTGACGTCAA |
| | | TGGGAGTTTGTTTTGGCACCAAAATCAACGGGACTTTC |
| | | CAAAATGTCGTAACAACTCCGCCCCATTGACGCAAAT |
| | | GGGCGGTAGGCGTGTACGGTGGGAGGTCTATATAAGC |
| | | AGAGCTCTCTGGCTAACTAGAGAACCCACTGCTTACT |
| | | GGCTTATCGAAATTAATACGACTCACTATAGGGAGAC |
| | | CCAAGCTGGCTAGTTAAGCTATCAACAAGTTTGTACA |
| | | AAAAAGCAGGCTCGATGGATATCTTTAATCATTTAAA |
| | | TAGCATTAACCCACGCACACGTTTTTGTTTTTCTCCTGT |
| | | ATCCGTTAGCTATGCATTATCTGTATGTTGTACGGGTA |
| | | ATGTTCCGTCGGATTACGTATCCTCCACAGTCGTTGTA |
| | | AAAAACAAAGTATACATAAACGCGTTTAAACAGTCCC |
| | | CCGTACGCGGTACATCGTACGCACACTTCACTAACGA |
| | | TGTCGTACATCGATTACACAAAGAAGTAGAGTCATAC |
| | | GACGTACGTTTCCCTATAAAATCGGTAAACCTAGACG |
| | | CGGTGTTTCTATCCATAAACGTAACACGTGTACGTCTA |
| | | CGTTGGAAGATACCCTTGACCGAACACAATCCTTATC |
| | | AGACGGCCTACGGATGTTCTAACGACAGATTATACAG |
| | | CTACAACGAGTACGCTTTTTCTCATTTAAAACAAGACC |
| | | GTGTAAAGATCATAGAACTCCCATGTGACGACGATTA |
| | | CAGCGTCGTGTTAATCACACACGATAGCCGTTCGACT |
| | | ATTACACCGGATAAAGTGACCGGGTGGCTGCGCACGA |
| | | CCCGTCTACGTTACGTAAACGTATCCCTACCCAAGGGT |
| | | TCCACGGAAACGGGACACAACGTAACGTGTCTAACTC |
| | | CCACACACGTCAATCTATGTCATCGTTGTCGTATAACG |
| | | ATTACCAAAACGGGCGTGGACGCAACCGCGTTCTCAT |
| | | GCGTCGACGGCGATACATGCACCGAACACGACACGAC |
| | | CGCGTCAACGTGTACGATTATTATAAAAACGACGGGT |
| | | CTAGACTTTTTGTTTATGGGGAAACTCTAAGACAACTT |
| | | TTCTATACAAAGTTGCCAAAATTGAAATTTTATTTTTT |
| | | TTTTTTGGAATATAAATAATGAAGGCCACTATCATCCT |
| | | CCTTCTGCTTGCACAAGTTTCCTGGGCTGGACCGTTTC |
| | | AACAGAGAGGCTTATTTGACTTTATGCTAGAAGATGA |
| | | GGCTTCTGGGATAGGCCCAGAAGTTCCTGATGACCGC |
| | | GACTTCGAGCCCTCCCTAGGCCCAGTGTGCCCCTTCCG |
| | | CTGTCAATGCCATCTTCGAGTGGTCCAGTGTTCTGATT |
| | | TGGGTCTGGACAAAGTGCCAAAGGATCTTCCCCCTGA |
| | | CACAACTCTGCTAGACCTGCAAAACAACAAAATAACC |
| | | GAAATCAAAGATGGAGACTTTAAGAACCTGAAGAACC |
| | | TTCACGCATTGATTCTTGTCAACAATAAAATTAGCAAA |
| | | GTTAGTCCTGGAGCATTTACACCTTTGGTGAAGTTGGA |
| | | ACGACTTTATCTGTCCAAGAATCAGCTGAAGGAATTG |
| | | CCAGAAAAAATGCCCAAAACTCTTCAGGAGCTGCGTG |
| | | CCCATGAGAATGAGATCACCAAAGTGCGAAAAGTTAC |
| | | TTTCAATGGACTGAACCAGATGATTGTCATAGAACTG |
| | | GGCACCAATCCGCTGAAGAGCTCAGGAATTGAAAATG |
| | | GGGCTTTCCAGGGAATGAAGAAGCTCTCCTACATCCG |
| | | CATTGCTGATACCAATATCACCAGCATTCCTCAAGGTC |
| | | TTCCTCCTTCCCTTACGGAATTACATCTcGATGGCAAC |
| | | AAAATCAGCAGAGTTGATGCAGCTAGCCTGAAAGGAC |
| | | TGAATAATTTGGCTAAGTTGGGATTGAGTTTCAACAGC |
| | | ATCTCTGCTGTTGACAATGGCTCTCTGGCCAACACGCC |
| | | TCATCTGAGGGAGCTTCACTTGGACAACAACAAGCTT |
| | | ACCAGAGTACCTGGTGGGCTGGCAGAGCATAAGTACA |
| | | TCCAGGTTGTCTACCTTCATAACAACAATATCTCTGTA |
| | | GTTGGATCAAGTGACTTCTGCCCACCTGGACACAACA |
| | | CCAAAAAGGCTTCTTATTCGGGTGTGAGTCTTTTCAGC |
| | | AACCCGGTCCAGTACTGGGAGATACAGCCATCCACCT |
| | | TCAGATGTGTCTACGTGCGCTCTGCCATTCAACTCGGA |
| | | AACTATAAGTAAGCTTGGACTCCTGTTGATAGATCCA |
| | | GAAAAATTGAAATTTTATTTTTTTTTTTTGGAATATAA |
| | | ATAATGTGTCACCAGCAGTTGGTCATCTCTTGGTTTTC |
| | | CCTGGTTTTTCTGGCATCTCCCCTCGTGGCCATATGGG |
| | | AACTGAAGAAAGATGTTTATGTCGTAGAATTGGATTG |
| | | GTATCCGGATGCCCCTGGAGAAATGGTGGTCCTCACC |
| | | TGTGACACCCCTGAAGAAGATGGTATCACCTGGACCT |
| | | TGGACCAGAGCAGTGAGGTCTTAGGCTCTGGCAAAAC |
| | | CCTGACCATCCAAGTCAAAGAGTTTGGAGATGCTGGC |
| | | CAGTACACCTGTCACAAAGGAGGCGAGGTTCTAAGCC |
| | | ATTCGCTCCTGCTGCTTCACAAAAAGGAAGATGGAAT |
| | | TTGGTCCACTGATATTTTAAAGGACCAGAAAGAACCC |
| | | AAAAATAAGACCTTTCTAAGATGCGAGGCCAAGAATT |
| | | ATTCTGGACGTTTCACCTGCTGGTGGCTGACGACAATC |
| | | AGTACTGATTTGACATTCAGTGTCAAAAGCAGCAGAG |
| | | GCTCTTCTGACCCCCAAGGGGTGACGTGCGGAGCTGC |
| | | TACACTCTCTGCAGAGAGAGTCAGAGGGGACAACAAG |
| | | GAGTATGAGTACTCAGTGGAGTGCCAGGAGGACAGTG |
| | | CCTGCCCAGCTGCTGAGGAGAGTCTGCCCATTGAGGT |
| | | CATGGTGGATGCCGTTCACAAGCTCAAGTATGAAAAC |
| | | TACACCAGCAGCTTCTTCATCAGGGACATCATCAAAC |
| | | CTGACCCACCCAAGAACTTGCAGCTGAAGCCATTAAA |
| | | GAATTCTCGGCAGGTGGAGGTCAGCTGGGAGTACCCT |
| | | GACACCTGGAGTACTCCACATTCCTACTTCTCCCTGAC |
| | | ATTCTGCGTTCAGGTCCAGGGCAAGAGCAAGAGAGAA |
| | | AAGAAAGATAGAGTCTTCACGGACAAGACCTCAGCCA |
| | | CGGTCATCTGCCGCAAAAATGCCAGCATTAGCGTGCG |
| | | GGCCCAGGACCGCTACTATAGCTCATCTTGGAGCGAA |
| | | TGGGCATCTGTGCCCTGCAGTTAGTATGCTAGTACGTC |
| | | TCTCAAGGATAAGTAAGTAATATTAAGGTACGGGAGG |
| | | TATTGGACAGGCCGCAATAAAATATCTTTATTTTCATT |
| | | ACATCTGTGTGTTGGTTTTTTGTGTGAATCGATAGTAC |
| | | TAACATACGCTCTCCATCAAAACAAAACGAAACAAAA |
| | | CAAACTAGCAAAATAGGCTGTCCCCAGTGCAAGTGCA |
| | | GGTGCCAGAACATTTCTCTGGCCTAACTGGCCGGTACC |
| | | TGAGCTCTAGTTTCACTTTCCCTAGTTTCACTTTCCCTA |
| | | GTTTCACTTTCCCTAGTTTCACTTTCCCTAGTTTCACTT |
| | | TCCCCTCGAGGATATCAAGATCTGGCCTCGGCGGCCA |
| | | GATGTGGCCCCCTGGGTCAGCCTCCCAGCCACCGCCCT |
| | | CACCTGCCGCGGCCACAGGTCTGCATCCAGCGGCTCG |
| | | CCCTGTGTCCCTGCAGTGCCGGCTCAGCATGTGTCCAG |
| | | CGCGCAGCCTCCTCCTTGTGGCTACCCTGGTCCTCCTG |
| | | GACCACCTCAGTTTGGCCAGAAACCTCCCCGTGGCCA |
| | | CTCCAGACCCAGGAATGTTCCCATGCCTTCACCACTCC |
| | | CAAAACCTGCTGAGGGCCGTCAGCAACATGCTCCAGA |
| | | AGGCCAGACAAACTCTAGAATTTTACCCTTGCACTTCT |
| | | GAAGAGATTGATCATGAAGATATCACAAAAGATAAAA |
| | | CCAGCACAGTGGAGGCCTGTTTACCATTGGAATTAAC |
| | | CAAGAATGAGAGTTGCCTAAATTCCAGAGAGACCTCT |
| | | TTCATAACTAATGGGAGTTGCCTGGCCTCCAGAAAGA |
| | | CCTCTTTTATGATGGCCCTGTGCCTTAGTAGTATTTAT |
| | | GAAGACTTGAAGATGTACCAGGTGGAGTTCAAGACCA |
| | | TGAATGCAAAGCTTCTGATGGATCCTAAGAGGCAGAT |
| | | CTTTCTAGATCAAAACATGCTGGCAGTTATTGATGAGC |
| | | TGATGCAGGCCCTGAATTTCAACAGTGAGACTGTGCC |
| | | ACAAAAATCCTCCCTTGAAGAACCGGATTTTTATAAA |
| | | ACTAAAATCAAGCTCTGCATACTTCTTCATGCTTTCAG |
| | | AATTCGGGCAGTGACTATTGATAGAGTGATGAGCTAT |
| | | CTGAATGCTTCCTAAGGGGACAACTTTGTATAATAAA |
| | | GTTGCTAAAAATTGAAATTTTATTTTTTTTTTTTGGAAT |
| | | ATAAATAATGGTGAGCAAGGGCGAGGAGCTGTTCACC |
| | | GGGGTGGTGCCCATCCTGGTCGAGCTGGACGGCGACG |
| | | TAAACGGCCACAAGTTCAGCGTGTCCGGCGAGGGCGA |
| | | GGGCGATGCCACCTACGGCAAGCTGACCCTGAAGTTC |
| | | ATCTGCACCACCGGCAAGCTGCCCGTGCCCTGGCCCA |
| | | CCCTCGTGACCACCCTGACCTACGGCGTGCAGTGCTTC |
| | | AGCCGCTACCCCGACCACATGAAGCAGCACGACTTCT |
| | | TCAAGTCCGCCATGCCCGAAGGCTACGTCCAGGAGCG |
| | | CACCATCTTCTTCAAGGACGACGGCAACTACAAGACC |
| | | CGCGCCGAGGTGAAGTTCGAGGGCGACACCCTGGTGA |
| | | ACCGCATCGAGCTGAAGGGCATCGACTTCAAGGAGGA |
| | | CGGCAACATCCTGGGGCACAAGCTGGAGTACAACTAC |
| | | AACAGCCACAACGTCTATATCATGGCCGACAAGCAGA |
| | | AGAACGGCATCAAGGTGAACTTCAAGATCCGCCACAA |
| | | CATCGAGGACGGCAGCGTGCAGCTCGCCGACCACTAC |
| | | CAGCAGAACACCCCCATCGGCGACGGCCCCGTGCTGC |
| | | TGCCCGACAACCACTACCTGAGCACCCAGTCCGCCCT |
| | | GAGCAAAGACCCCAACGAGAAGCGCGATCACATGGTC |
| | | CTGCTGGAGTTCGTGACCGCCGCCGGGATCACTCTCG |
| | | GCATGGACGAGCTGTACAAGTAATTATATAAGGTATC |
| | | TCGTTTGTCTATAACAAAGATCGTAACTGACCTTTTTT |
| | | ATATCGAGAAAACATACGTTTAGTTCATCCTCAAACGT |
| | | AACACCGTAACTGCCTCGGACATCCTCCTTGTTGTCGT |
| | | ACACAAACATACTAATCGGATGCGTGAAATGAGGATT |
| | | CACTTTAATCGGATTGGTTTCTAGGTTAACACATGTTA |
| | | CACAGGATCCTAAGATGGTTATGGACACATCCTTGTTG |
| | | TGATGTAACGAGTCGGGAAGTTGATTGCCGTAGTTGC |
| | | CCACGTCGCCCTCCGGTTCCAGACACGTAATGGTTAG |
| | | GTATATATCCGAATACTTCGTCAACGGATGAGTCGTA |
| | | AATAACATGATGGATAGCTTGTTCCCATCTCCTGCACC |
| | | AGCACTGGCCGCCACAAATCGTTGTACCACGTTAGTA |
| | | ATCGTAATGTTTATCATAAGCCCGTACCCGGTTAATAT |
| | | GAGCGTGGACGTTTTATGATCGTATCGTTCCTTCATGT |
| | | GACATTCTCCCATAACCGTTTCGACGTACCGATTTAAC |
| | | CCGATGGTTAGCTCGGCGGCTAAGTGCCAGTACTTTTT |
| | | TGGATACGTCGCACATGTTGAGGTTGCGACGAGGCAG |
| | | GCGAGCACGATGATAATATACCGCGCCATTACCCAGC |
| | | TTTCTTGTACAAAGTGGTTGATCTAGAGGGCCCGCGGT |
| | | TCGAAGGTAAGCCTATCCCTAACCCTCTCCTCGGTCTC |
| | | GATTCTACGCGTACCGGTCATCATCACCATCACCATTG |
| | | AGTTTAAACCCGCTGATCAGCCTCGACTGTGCCTTCTA |
| | | GTTGCCAGCCATCTGTTGTTTGCCCCTCCCCCGTGCCT |
| | | TCCTTGACCCTGGAAGGTGCCACTCCCACTGTCCTTTC |
| | | CTAATAAAATGAGGAAATTGCATCGCATTGTCTGAGT |
| | | AGGTGTCATTCTATTCTGGGGGGTGGGGTGGGGCAGG |
| | | ACAGCAAGGGGGAGGATTGGGAAGACAATAGCAGGC |
| | | ATGCTGGGGATGCGGTGGGCTCTATGGCTTCTGAGGC |
| | | GGAAAGAACCAGCTGGGGCTCTAGGGGGTATCCCCAC |
| | | GCGCCCTGTAGCGGCGCATTAAGCGCGGCGGGTGTGG |
| | | TGGTTACGCGCAGCGTGACCGCTACACTTGCCAGCGC |
| | | CCTAGCGCCCGCTCCTTTCGCTTTCTTCCCTTCCTTTCT |
| | | CGCCACGTTCGCCGGCTTTCCCCGTCAAGCTCTAAATC |
| | | GGGGGCTCCCTTTAGGGTTCCGATTTAGTGCTTTACGG |
| | | CACCTCGACCCCAAAAAACTTGATTAGGGTGATGGTT |
| | | CACGTAGTGGGCCATCGCCCTGATAGACGGTTTTTCGC |
| | | CCTTTGACGTTGGAGTCCACGTTCTTTAATAGTGGACT |
| | | CTTGTTCCAAACTGGAACAACACTCAACCCTATCTCGG |
| | | TCTATTCTTTTGATTTATAAGGGATTTTGCCGATTTCGG |
| | | CCTATTGGTTAAAAAATGAGCTGATTTAACAAAAATTT |
| | | AACGCGAATTAATTCTGTGGAATGTGTGTCAGTTAGG |
| | | GTGTGGAAAGTCCCCAGGCTCCCCAGCAGGCAGAAGT |
| | | ATGCAAAGCATGCATCTCAATTAGTCAGCAACCAGGT |
| | | GTGGAAAGTCCCCAGGCTCCCCAGCAGGCAGAAGTAT |
| | | GCAAAGCATGCATCTCAATTAGTCAGCAACCATAGTC |
| | | CCGCCCCTAACTCCGCCCATCCCGCCCCTAACTCCGCC |
| | | CAGTTCCGCCCATTCTCCGCCCCATGGCTGACTAATTT |
| | | TTTTTATTTATGCAGAGGCCGAGGCCGCCTCTGCCTCT |
| | | GAGCTATTCCAGAAGTAGTGAGGAGGCTTTTTTGGAG |
| | | GCCTAGGCTTTTGCAAAAAGCTCCCGGGAGCTTGTAT |
| | | ATCCATTTTCGGATCTGATCAAGAGACAGGATGAGGA |
| | | TCGTTTCGCATGATTGAACAAGATGGATTGCACGCAG |
| | | GTTCTCCGGCCGCTTGGGTGGAGAGGCTATTCGGCTAT |
| | | GACTGGGCACAACAGACAATCGGCTGCTCTGATGCCG |
| | | CCGTGTTCCGGCTGTCAGCGCAGGGGCGCCCGGTTCTT |
| | | TTTGTCAAGACCGACCTGTCCGGTGCCCTGAATGAACT |
| | | GCAGGACGAGGCAGCGCGGCTATCGTGGCTGGCCACG |
| | | ACGGGCGTTCCTTGCGCAGCTGTGCTCGACGTTGTCAC |
| | | TGAAGCGGGAAGGGACTGGCTGCTATTGGGCGAAGTG |
| | | CCGGGGCAGGATCTCCTGTCATCTCACCTTGCTCCTGC |
| | | CGAGAAAGTATCCATCATGGCTGATGCAATGCGGCGG |
| | | CTGCATACGCTTGATCCGGCTACCTGCCCATTCGACCA |
| | | CCAAGCGAAACATCGCATCGAGCGAGCACGTACTCGG |
| | | ATGGAAGCCGGTCTTGTCGATCAGGATGATCTGGACG |
| | | AAGAGCATCAGGGGCTCGCGCCAGCCGAACTGTTCGC |
| | | CAGGCTCAAGGCGCGCATGCCCGACGGCGAGGATCTC |
| | | GTCGTGACCCATGGCGATGCCTGCTTGCCGAATATCAT |
| | | GGTGGAAAATGGCCGCTTTTCTGGATTCATCGACTGTG |
| | | GCCGGCTGGGTGTGGCGGACCGCTATCAGGACATAGC |
| | | GTTGGCTACCCGTGATATTGCTGAAGAGCTTGGCGGC |
| | | GAATGGGCTGACCGCTTCCTCGTGCTTTACGGTATCGC |
| | | CGCTCCCGATTCGCAGCGCATCGCCTTCTATCGCCTTC |
| | | TTGACGAGTTCTTCTGAGCGGGACTCTGGGGTTCGCGA |
| | | AATGACCGACCAAGCGACGCCCAACCTGCCATCACGA |
| | | GATTTCGATTCCACCGCCGCCTTCTATGAAAGGTTGGG |
| | | CTTCGGAATCGTTTTCCGGGACGCCGGCTGGATGATCC |
| | | TCCAGCGCGGGGATCTCATGCTGGAGTTCTTCGCCCAC |
| | | CCCAACTTGTTTATTGCAGCTTATAATGGTTACAAATA |
| | | AAGCAATAGCATCACAAATTTCACAAATAAAGCATTT |
| | | TTTTCACTGCATTCTAGTTGTGGTTTGTCCAAACTCATC |
| | | AATGTATCTTATCATGTCTGTATACCGTCGACCTCTAG |
| | | CTAGAGCTTGGCGTAATCATGGTCATAGCTGTTTCCTG |
| | | TGTGAAATTGTTATCCGCTCACAATTCCACACAACATA |
| | | CGAGCCGGAAGCATAAAGTGTAAAGCCTGGGGTGCCT |
| | | AATGAGTGAGCTAACTCACATTAATTGCGTTGCGCTCA |
| | | CTGCCCGCTTTCCAGTCGGGAAACCTGTCGTGCCAGCT |
| | | GCATTAATGAATCGGCCAACGCGCGGGGAGAGGCGGT |
| | | TTGCGTATTGGGCGCTCTTCCGCTTCCTCGCTCACTGA |
| | | CTCGCTGCGCTCGGTCGTTCGGCTGCGGCGAGCGGTAT |
| | | CAGCTCACTCAAAGGCGGTAATACGGTTATCCACAGA |
| | | ATCAGGGGATAACGCAGGAAAGAACATGTGAGCAAA |
| | | AGGCCAGCAAAAGGCCAGGAACCGTAAAAAGGCCGC |
| | | GTTGCTGGCGTTTTTCCATAGGCTCCGCCCCCCTGACG |
| | | AGCATCACAAAAATCGACGCTCAAGTCAGAGGTGGCG |
| | | AAACCCGACAGGACTATAAAGATACCAGGCGTTTCCC |
| | | CCTGGAAGCTCCCTCGTGCGCTCTCCTGTTCCGACCCT |
| | | GCCGCTTACCGGATACCTGTCCGCCTTTCTCCCTTCGG |
| | | GAAGCGTGGCGCTTTCTCATAGCTCACGCTGTAGGTAT |
| | | CTCAGTTCGGTGTAGGTCGTTCGCTCCAAGCTGGGCTG |
| | | TGTGCACGAACCCCCCGTTCAGCCCGACCGCTGCGCCT |
| | | TATCCGGTAACTATCGTCTTGAGTCCAACCCGGTAAGA |
| | | CACGACTTATCGCCACTGGCAGCAGCCACTGGTAACA |
| | | GGATTAGCAGAGCGAGGTATGTAGGCGGTGCTACAGA |
| | | GTTCTTGAAGTGGTGGCCTAACTACGGCTACACTAGA |
| | | AGAACAGTATTTGGTATCTGCGCTCTGCTGAAGCCAGT |
| | | TACCTTCGGAAAAAGAGTTGGTAGCTCTTGATCCGGC |
| | | AAACAAACCACCGCTGGTAGCGGTGGTTTTTTTGTTTG |
| | | CAAGCAGCAGATTACGCGCAGAAAAAAAGGATCTCA |
| | | AGAAGATCCTTTGATCTTTTCTACGGGGTCTGACGCTC |
| | | AGTGGAACGAAAACTCACGTTAAGGGATTTTGGTCAT |
| | | GAGATTATCAAAAAGGATCTTCACCTAGATCCTTTTAA |
| | | ATTAAAAATGAAGTTTTAAATCAATCTAAAGTATATAT |
| | | GAGTAAACTTGGTCTGACAGTTACCAATGCTTAATCA |
| | | GTGAGGCACCTATCTCAGCGATCTGTCTATTTCGTTCA |
| | | TCCATAGTTGCCTGACTCCCCGTCGTGTAGATAACTAC |
| | | GATACGGGAGGGCTTACCATCTGGCCCCAGTGCTGCA |
| | | ATGATACCGCGAGACCCACGCTCACCGGCTCCAGATT |
| | | TATCAGCAATAAACCAGCCAGCCGGAAGGGCCGAGCG |
| | | CAGAAGTGGTCCTGCAACTTTATCCGCCTCCATCCAGT |
| | | CTATTAATTGTTGCCGGGAAGCTAGAGTAAGTAGTTC |
| | | GCCAGTTAATAGTTTGCGCAACGTTGTTGCCATTGCTA |
| | | CAGGCATCGTGGTGTCACGCTCGTCGTTTGGTATGGCT |
| | | TCATTCAGCTCCGGTTCCCAACGATCAAGGCGAGTTAC |
| | | ATGATCCCCCATGTTGTGCAAAAAAGCGGTTAGCTCCT |
| | | TCGGTCCTCCGATCGTTGTCAGAAGTAAGTTGGCCGCA |
| | | GTGTTATCACTCATGGTTATGGCAGCACTGCATAATTC |
| | | TCTTACTGTCATGCCATCCGTAAGATGCTTTTCTGTGA |
| | | CTGGTGAGTACTCAACCAAGTCATTCTGAGAATAGTG |
| | | TATGCGGCGACCGAGTTGCTCTTGCCCGGCGTCAATAC |
| | | GGGATAATACCGCGCCACATAGCAGAACTTTAAAAGT |
| | | GCTCATCATTGGAAAACGTTCTTCGGGGCGAAAACTC |
| | | TCAAGGATCTTACCGCTGTTGAGATCCAGTTCGATGTA |
| | | ACCCACTCGTGCACCCAACTGATCTTCAGCATCTTTTA |
| | | CTTTCACCAGCGTTTCTGGGTGAGCAAAAACAGGAAG |
| | | GCAAAATGCCGCAAAAAAGGGAATAAGGGCGACACG |
| | | GAAATGTTGAATACTCATACTCTTCCTTTTTCAATATT |
| | | ATTGAAGCATTTATCAGGGTTATTGTCTCATGAGCGGA |
| | | TACATATTTGAATGTATTTAGAAAAATAAACAAATAG |
| | | GGGTTCCGCGCACATTTCCCCGAAAAGTGCCACCTGA |
| | | CGTCGACGGATCGGGAGATCTCCCGATCCCCTATGGT |
| | | GCACTCTCAGTACAATCTGCTCTGATGCCGCATAGTTA |
| | | AGCCAGTATCTGCTCCCTGCTTGTGTGTTGGAGGTCGC |
| | | TGAGTAGTGCGCGAGCAAAATTTAAGCTACAACAAGG |
| | | CAAGGCTTGACCGACAATTGCATGAAGAATCTGCTTA |
| | | GGGTTAGGCGTTTTGCGCTGCTTCGCGATGTACGGGCC |
| | | AGATATACGCGTTGACATTGATTATTGA |
| 28 | hu IL-12B (p40); | MCHQQLVISWFSLVFLASPLVAIWELKKDVYVVELDWY |
| | translation of SEQ | PDAPGEMVVLTCDTPEEDGITWTLDQSSEVLGSGKTLTI |
| | ID NO: 3 | QVKEFGDAGQYTCHKGGEVLSHSLLLLHKKEDGIWSTDI |
| | | LKDQKEPKNKTFLRCEAKNYSGRFTCWWLTTISTDLTFS |
| | | VKSSRGSSDPQGVTCGAATLSAERVRGDNKEYEYSVEC |
| | | QEDSACPAAEESLPIEVMVDAVHKLKYENYTSSFFIRDII |
| | | KPDPPKNLQLKPLKNSRQVEVSWEYPDTWSTPHSYFSLT |
| | | FCVQVQGKSKREKKDRVFTDKTSATVICRKNASISVRAQ |
| | | DRYYSSSWSEWASVPCS |
| 29 | hu IL-12A (p35); | MWPPGSASQPPPSPAAATGLHPAARPVSLQCRLSMCPAR |
| | translation of SEQ | SLLLVATLVLLDHLSLARNLPVATPDPGMFPCLHHSQNL |
| | ID NO: 4 | LRAVSNMLQKARQTLEFYPCTSEEIDHEDITKDKTSTVE |
| | | ACLPLELTKNESCLNSRETSFITNGSCLASRKTSFMMALC |
| | | LSSIYEDLKMYQVEFKTMNAKLLMDPKRQIFLDQNMLA |
| | | VIDELMQALNFNSETVPQKSSLEEPDFYKTKIKLCILLHA |
| | | FRIRAVTIDRVMSYLNAS |
| 30 | hu IL-12A (p35) - | ARNLPVATPDPGMFPCLHHSQNLLRAVSNMLQKARQTL |
| | truncated, lacking | EFYPCTSEEIDHEDITKDKTSTVEACLPLELTKNESCLNSR |
| | signal peptide; | ETSFITNGSCLASRKTSFMMALCLSSIYEDLKMYQVEFKT |
| | translation of SEQ | MNAKLLMDPKRQIFLDQNMLAVIDELMQALNFNSETVP |
| | ID NO: 5 | QKSSLEEPDFYKTKIKLCILLHAFRIRAVTIDRVMSYLNA |
| | | S |
| 31 | Elastin linker, | VPGVGVPGVG |
| | translation of SEQ | |
| | ID NO: 6 | |
| 32 | Human decorin, | MKATIILLLLAQVSWAGPFQQRGLFDFMLEDEASGIGPE |
| | translation of SEQ | VPDDRDFEPSLGPVCPFRCQCHLRVVQCSDLGLDKVPKD |
| | ID NO: 7 | LPPDTTLLDLQNNKITEIKDGDFKNLKNLHALILVNNKIS |
| | | KVSPGAFTPLVKLERLYLSKNQLKELPEKMPKTLQELRA |
| | | HENEITKVRKVTFNGLNQMIVIELGTNPLKSSGIENGAFQ |
| | | GMKKLSYIRIADTNITSIPQGLPPSLTELHLDGNKISRVDA |
| | | ASLKGLNNLAKLGLSFNSISAVDNGSLANTPHLRELHLD |
| | | NNKLTRVPGGLAEHKYIQVVYLHNNNISVVGSSDFCPPG |
| | | HNTKKASYSGVSLFSNPVQYWEIQPSTFRCVYVRSAIQL |
| | | GNYK |
| 33 | GFP, translation of | MVSKGEELFTGVVPILVELDGDVNGHKFSVSGEGEGDA |
| | SEQ ID NO: 8 | TYGKLTLKFICTTGKLPVPWPTLVTTLTYGVQCFSRYPD |
| | | HMKQHDFFKSAMPEGYVQERTIFFKDDGNYKTRAEVKF |
| | | EGDTLVNRIELKGIDFKEDGNILGHKLEYNYNSHNVYIM |
| | | ADKQKNGIKVNFKIRHNIEDGSVQLADHYQQNTPIGDGP |
| | | VLLPDNHYLSTQSALSKDPNEKRDHMVLLEFVTAAGITL |
| | | GMDELYK |
| 34 | IL-12B-elastin-IL- | MCHQQLVISWFSLVFLASPLVAIWELKKDVYVVELDWY |
| | 12A, translation of | PDAPGEMVVLTCDTPEEDGITWTLDQSSEVLGSGKTLTI |
| | SEQ ID NO: 9 | QVKEFGDAGQYTCHKGGEVLSHSLLLLHKKEDGIWSTDI |
| | | LKDQKEPKNKTFLRCEAKNYSGRFTCWWLTTISTDLTFS |
| | | VKSSRGSSDPQGVTCGAATLSAERVRGDNKEYEYSVEC |
| | | QEDSACPAAEESLPIEVMVDAVHKLKYENYTSSFFIRDII |
| | | KPDPPKNLQLKPLKNSRQVEVSWEYPDTWSTPHSYFSLT |
| | | FCVQVQGKSKREKKDRVFTDKTSATVICRKNASISVRAQ |
| | | DRYYSSSWSEWASVPCSVPGVGVPGVGARNLPVATPDP |
| | | GMFPCLHHSQNLLRAVSNMLQKARQTLEFYPCTSEEIDH |
| | | EDITKDKTSTVEACLPLELTKNESCLNSRETSFITNGSCLA |
| | | SRKTSFMMALCLSSIYEDLKMYQVEFKTMNAKLLMDPK |
| | | RQIFLDQNMLAVIDELMQALNFNSETVPQKSSLEEPDFY |
| | | KTKIKLCILLHAFRIRAVTIDRVMSYLNAS |
| 35 | Human TNFa, | MSTESMIRDVELAEEALPKKTGGPQGSRRCLFLSLFSFLI |
| | translation of SEQ | VAGATTLFCLLHFGVIGPQREEFPRDLSLISPLAQAVRSSS |
| | ID NO: 18 | RTPSDKPVAHVVANPQAEGQLQWLNRRANALLANGVE |
| | | LRDNQLVVPSEGLYLIYSQVLFKGQGCPSTHVLLTHTISR |
| | | IAVSYQTKVNLLSAIKSPCQRETPEGAEAKPWYEPIYLGG |
| | | VFQLEKGDRLSAEINRPDYLDFAESGQVYFGIIAL |
| 36 | Mouse IL-12 single | MCPQKLTISWFAIVLLVSPLMAMWELEKDVYVVEVDW |
| | polypeptide | TPDAPGETVNLTCDTPEEDDITWTSDQRHGVIGSGKTLTI |
| | (mIL12B-elastin | TVKEFLDAGQYTCHKGGETLSHSHLLLHKKENGIWSTEI |
| | linker-truncated | LKNFKNKTFLKCEAPNYSGRFTCSWLVQRNMDLKFNIK |
| | mIL12A) | SSSSSPDSRAVTCGMASLSAEKVTLDQRDYEKYSVSCQE |
| | | DVTCPTAEETLPIELALEARQQNKYENYSTSFFIRDIIKPD |
| | | PPKNLQMKPLKNSQVEVSWEYPDSWSTPHSYFSLKFFVR |
| | | IQRKKEKMKETEEGCNQKGAFLVEKTSTEVQCKGGNVC |
| | | VQAQDRYYNSSCSKWACVPCRVRSVPGVGVPGVGMVS |
| | | VPTASPSASSSSSQCRSSMCQSRYLLFLATLALLNHLSLA |
| | | RVIPVSGPARCLSQSRNLLKTTDDMVKTAREKLKHYSCT |
| | | AEDIDHED |
| 64 | Mouse IL-12 single | MCPQKLTISWFAIVLLVSPLMAMWELEKDVYVVEVDW |
| | polypeptide | TPDAPGETVNLTCDTPEEDDITWTSDQRHGVIGSGKTLTI |
| | (mIL12B-elastin | TVKEFLDAGQYTCHKGGETLSHSHLLLHKKENGIWSTEI |
| | linker-mIL12A) | LKNFKNKTFLKCEAPNYSGRFTCSWLVQRNMDLKFNIK |
| | | SSSSSPDSRAVTCGMASLSAEKVTLDQRDYEKYSVSCQE |
| | | DVTCPTAEETLPIELALEARQQNKYENYSTSFFIRDIIKPD |
| | | PPKNLQMKPLKNSQVEVSWEYPDSWSTPHSYFSLKFFVR |
| | | IQRKKEKMKETEEGCNQKGAFLVEKTSTEVQCKGGNVC |
| | | VQAQDRYYNSSCSKWACVPCRVRSVPGVGVPGVGMVS |
| | | VPTASPSASSSSSQCRSSMCQSRYLLFLATLALLNHLSLA |
| | | RVIPVSGPARCLSQSRNLLKTTDDMVKTAREKLKHYSCT |
| | | AEDIDHEDITRDQTSTLKTCLPLELHKNESCLATRETSSTT |
| | | RGSCLPPQKTSLMMTLCLGSIYEDLKMYQTEFQAINAAL |
| | | QNHNHQQIILDKGMLVAIDELMQSLNHNGETLRQKPPV |
| | | GEADPYRVKMKLCILLHAFSTRVVTINRVMGYLSSA |
| 37 | Mouse IL-12B | MCPQKLTISWFAIVLLVSPLMAMWELEKDVYVVEVDW |
| | (p40) | TPDAPGETVNLTCDTPEEDDITWTSDQRHGVIGSGKTLTI |
| | | TVKEFLDAGQYTCHKGGETLSHSHLLLHKKENGIWSTEI |
| | | LKNFKNKTFLKCEAPNYSGRFTCSWLVQRNMDLKFNIK |
| | | SSSSSPDSRAVTCGMASLSAEKVTLDQRDYEKYSVSCQE |
| | | DVTCPTAEETLPIELALEARQQNKYENYSTSFFIRDIIKPD |
| | | PPKNLQMKPLKNSQVEVSWEYPDSWSTPHSYFSLKFFVR |
| | | IQRKKEKMKETEEGCNQKGAFLVEKTSTEVQCKGGNVC |
| | | VQAQDRYYNSSCSKWACVPCRVRS |
| 38 | Truncated mouse | MVSVPTASPSASSSSSQCRSSMCQSRYLLFLATLALLNHL |
| | IL-12A (p35) | SLARVIPVSGPARCLSQSRNLLKTTDDMVKTAREKLKHY |
| | | SCTAEDIDHED |
| 39 | Mouse IL-12A | MVSVPTASPSASSSSSQCRSSMCQSRYLLFLATLALLNHL |
| | (p35) | SLARVIPVSGPARCLSQSRNLLKTTDDMVKTAREKLKHY |
| | | SCTAEDIDHEDITRDQTSTLKTCLPLELHKNESCLATRET |
| | | SSTTRGSCLPPQKTSLMMTLCLGSIYEDLKMYQTEFQAIN |
| | | AALQNHNHQQIILDKGMLVAIDELMQSLNHNGETLRQK |
| | | PPVGEADPYRVKMKLCILLHAFSTRVVTINRVMGYLSSA |
| 40 | Mouse Decorin | MKATLIFFLLAQVSWAGPFEQRGLFDFMLEDEASGIIPYD |
| | | PDNPLISMCPYRCQCHLRVVQCSDLGLDKVPWDFPPDTT |
| | | LLDLQNNKITEIKEGAFKNLKDLHTLILVNNKISKISPEAF |
| | | KPLVKLERLYLSKNQLKELPEKMPRTLQELRVHENEITK |
| | | LRKSDFNGLNNVLVIELGGNPLKNSGIENGAFQGLKSLS |
| | | YIRISDTNITAIPQGLPTSLTEVHLDGNKITKVDAPSLKGLI |
| | | NLSKLGLSFNSITVMENGSLANVPHLRELHLDNNKLLRV |
| | | PAGLAQHKYIQVVYLHNNNISAVGQNDFCRAGHPSRKA |
| | | SYSAVSLYGNPVRYWEIFPNTFRCVYVRSAIQLGNYK |
| 41 | Membrane bound | ATGAGCACTGAAAGCATGATCCGGGACGTGGAGCTGG |
| | hu TNF-a | CCGAGGAGGCGCTCCCCAAGAAGACAGGGGGGCCCC |
| | | AGGGCTCCAGGCGGTGCTTGTTCCTCAGCCTCTTCTCC |
| | | TTCCTGATCGTGGCAGGCGCCACCACGCTCTTCTGCCT |
| | | GCTGCACTTTGGAGTGATCGGCCCCCAGAGGGAAGAG |
| | | TTCCCCAGGGACCTCTCTCTAATCAGCCCTCTGGCCCA |
| | | GGCAGATGAGCCTGTAGCCCATGTTGTAGCAAACCCT |
| | | CAAGCTGAGGGGCAGCTCCAGTGGCTGAACCGCCGGG |
| | | CCAATGCCCTCCTGGCCAATGGCGTGGAGCTGAGAGA |
| | | TAACCAGCTGGTGGTGCCATCAGAGGGCCTGTACCTC |
| | | ATCTACTCCCAGGTCCTCTTCAAGGGCCAAGGCTGCCC |
| | | CTCCACCCATGTGCTCCTCACCCACACCATCAGCCGCA |
| | | TCGCCGTCTCCTACCAGACCAAGGTCAACCTCCTCTCT |
| | | GCCATCAAGAGCCCCTGCCAGAGGGAGACCCCAGAGG |
| | | GGGCTGAGGCCAAGCCCTGGTATGAGCCCATCTATCT |
| | | GGGAGGGGTCTTCCAGCTGGAGAAGGGTGACCGACTC |
| | | AGCGCTGAGATCAATCGGCCCGACTATCTCGACTTTGC |
| | | CGAGTCTGGGCAGGTCTACTTTGGGATCATTGCCCTGT |
| | | AG |
| 42 | Alternative IRES | CTGGCGAAAGGGGGATGTGCTGCAAGGCGATTAAGTT |
| | | GGGTAACGCCAGGGTTTTCCCAGTCACGACGTTGTAA |
| | | AACGACGGCCAGTGAATTGTAATACGACTCACTATAG |
| | | GGCGAATTAATTCCGGTTATTTTCCACCATATTGCCGT |
| | | CTTTTGGCAATGTGAGGGCCCGGAAACCTGGCCCTGT |
| | | CTTCTTGACGAGCATTCCTAGGGGTCTTTCCCCTCTCG |
| | | CCAAAGGAATGCAAGGTCTGTTGAATGTCGTGAAGGA |
| | | AGCAGTTCCTCTGGAAGCTTCTTGAAGACAAACAACG |
| | | TCTGTAGCGACCCTTTGCAGGCAGCGGAACCCCCCAC |
| | | CTGGCGACAGGTGCCTCTGCGGCCAAAAGCCACGTGT |
| | | ATAAGATACACCTGCAAAGGCGGCACAACCCCAGTGC |
| | | CACGTTGTGAGTTGGATAGTTTGTGGAAAGAGTCAAA |
| | | TGGCTCTCCTCAAGCGTATTCAACAAGGGGCTGAAGG |
| | | ATGCCCAGAAGGTACCCCATTGTATGGGATCTGATCT |
| | | GGGGCCTCGGTGCACATGCTTTACATGTGTTTAGTCGA |
| | | GGTTAAAAAAACGTCTAGGCCCCCCGAACCACGGGGA |
| | | CGTGGTTTTCCTTTGAAAAACACGATGATA |
| 43 | Membrane bound | MSTESMIRDVELAEEALPKKTGGPQGSRRCLFLSLFSFLI |
| | hu TNF-a | VAGATTLFCLLHFGVIGPQREEFPRDLSLISPLAQADEPV |
| | | AHVVANPQAEGQLQWLNRRANALLANGVELRDNQLVV |
| | | PSEGLYLIYSQVLFKGQGCPSTHVLLTHTISRIAVSYQTK |
| | | VNLLSAIKSPCQRETPEGAEAKPWYEPIYLGGVFQLEKG |
| | | DRLSAEINRPDYLDFAESGQVYFGIIAL |
| 44 | Human Decorin, | MKATIILLLLAQVSWAGPFQQRGLFDFMLEDEASGIGPE |
| | isoform B | VPDDRDFEPSLGPVCPFRCQCHLRVVQCSDLELGTNPLK |
| | | SSGIENGAFQGMKKLSYIRIADTNITSIPQGLPPSLTELHL |
| | | DGNKISRVDAASLKGLNNLAKLGLSFNSISAVDNGSLAN |
| | | TPHLRELHLDNNKLTRVPGGLAEHKYIQVVYLHNNNISV |
| | | VGSSDFCPPGHNTKKASYSGVSLFSNPVQYWEIQPSTFRC |
| | | VYVRSAIQLGNYK |
| 45 | Human Decorin, | MKATIILLLLAQVSWAGPFQQRGLFDFMLEDEASGIGPE |
| | isoform C | VPDDRDFEPSLGPVCPFRCQCHLRVVQCSDLGLPPSLTEL |
| | | HLDGNKISRVDAASLKGLNNLAKLGLSFNSISAVINGSL |
| | | ANTPHLRELHLDNNKLTRVPGGLAEHKYIQVVYLHNNN |
| | | ISVVGSSDFCPPGHNTKKASYSGVSLFSNPVQYWEIQPST |
| | | FRCVYVRSAIQLGNYK |
| 46 | Huma Decorin, | MKATIILLLLAQVSWAGPFQQRGLFDFMLEDEASGIGPE |
| | isoform D | VPDDRDFEPSLGPVCPFRCQCHLRVVQCSDLGLDKVPKD |
| | | LPPDTTLLDLQNNKITEIKDGDFKNLKNLHVVYLHNNNI |
| | | SVVGSSDFCPPGHNTKKASYSGVSLFSNPVQYWEIQPSTF |
| | | RCVYVRSAIQLGNYK |
| 47 | Human Decorin, | MKATIILLLLAQVSWAGPFQQRGLFDFMLEDEASGIGPE |
| | isoform E | VPDDRDFEPSLGPVCPFRCQCHLRVVQCSDLGCLPS |
| 48 | IL-12A isoform | MCPARSLLLVATLVLLDHLSLARNLPVATPDPGMFPCLH |
| | P29459 | HSQNLLRAVSNMLQKARQTLEFYPCTSEEIDHEDITKDK |
| | | TSTVEACLPLELTKNESCLNSRETSFITNGSCLASRKTSFM |
| | | MALCLSSIYEDLKMYQVEFKTMNAKLLMDPKRQIFLDQ |
| | | NMLAVIDELMQALNFNSETVPQKSSLEEPDFYKTKIKLCI |
| | | LLHAFRIRAVTIDRVMSYLNAS |
| 49 | IL-12A isoform | MWPPGSASQPPPSPAAATGLHPAARPVSLQCRLSMCPAR |
| | E9PGR3 | SLLLVATLVLLDHLSLARNLPVATPDPGMFPCLHHSQNL |
| | | LRAVSNMLQKARQTLEFYPCTSEEIDHEDITKDKTSTVE |
| | | ACLPLELTKNGSCLASRKTSFMMALCLSSIYEDLKMYQV |
| | | EFKTMNAKLLMDPKRQIFLDQNMLAVIDELMQALNFNS |
| | | ETVPQKSSLEEPDFYKTKIKLCILLHAFRIRAVTIDRVMSY |
| | | LNAS |
| 50 | IL-12A isoform | MWPPGSASQPPPSPAAATGLHPAARPVSLQCRLSMCPAR |
| | E7ENE1 | SLLLVATLVLLDHLSLARNLPVATPDPGMFPCLHHSQNL |
| | | LRAVSNMLQKNESCLNSRETSFITNGSCLASRKTSFMMA |
| | | LCLSSIYEDLKMYQVEFKTMNAKLLMDPKRQIFLDQNM |
| | | LAVIDELMQALNFNSETVPQKSSLEEPDFYKTKIKLCILL |
| | | HAFRIRAVTIDRVMSYLNAS |
| 51 | Linker | GGGGS |
| 52 | Linker | GGGS |
| 53 | Linker | GG |
| 54 | Linker | KESGSVSSEQLAQFRSLD |
| 55 | Linker | EGKSSGSGSESKST |
| 56 | Linker | GSAGSAAGSGEF |
| 57 | Linker | EAAAK |
| 58 | Linker | EAAAR |
| 59 | Linker | PAPAP |
| 60 | Linker | AEAAAKEAAAKA |
| 61 | alternate sE/L | AAAATTGAAATTTTATTTTTTTTTTTTGGAATATAAAT |
| | promoter | A |
| 62 | Alternate human | ATGAAGGCCACTATCATCCTCCTTCTGCTTGCACAAGT |
| | decorin | TTCCTGGGCTGGACCGTTTCAACAGAGAGGCTTATTTG |
| | | ACTTTATGCTAGAAGATGAGGCTTCTGGGATAGGCCC |
| | | AGAAGTTCCTGATGACCGCGACTTCGAGCCCTCCCTA |
| | | GGCCCAGTGTGCCCCTTCCGCTGTCAATGCCATCTTCG |
| | | AGTGGTCCAGTGTTCTGATTTGGGTCTGGACAAAGTGC |
| | | CAAAGGATCTTCCCCCTGACACAACTCTGCTAGACCTG |
| | | CAAAACAACAAAATAACCGAAATCAAAGATGGAGAC |
| | | TTTAAGAACCTGAAGAACCTTCACGCATTGATTCTTGT |
| | | CAACAATAAAATTAGCAAAGTTAGTCCTGGAGCATTT |
| | | ACACCTTTGGTGAAGTTGGAACGACTTTATCTGTCCAA |
| | | GAATCAGCTGAAGGAATTGCCAGAAAAAATGCCCAAA |
| | | ACTCTTCAGGAGCTGCGTGCCCATGAGAATGAGATCA |
| | | CCAAAGTGCGAAAAGTTACTTTCAATGGACTGAACCA |
| | | GATGATTGTCATAGAACTGGGCACCAATCCGCTGAAG |
| | | AGCTCAGGAATTGAAAATGGGGCTTTCCAGGGAATGA |
| | | AGAAGCTCTCCTACATCCGCATTGCTGATACCAATATC |
| | | ACCAGCATTCCTCAAGGTCTTCCTCCTTCCCTTACGGA |
| | | ATTACATCTcGATGGCAACAAAATCAGCAGAGTTGAT |
| | | GCAGCTAGCCTGAAAGGACTGAATAATTTGGCTAAGT |
| | | TGGGATTGAGTTTCAACAGCATCTCTGCTGTTGACAAT |
| | | GGCTCTCTGGCCAACACGCCTCATCTGAGGGAGCTTC |
| | | ACTTGGACAACAACAAGCTTACCAGAGTACCTGGTGG |
| | | GCTGGCAGAGCATAAGTACATCCAGGTTGTCTACCTTC |
| | | ATAACAACAATATCTCTGTAGTTGGATCAAGTGACTTC |
| | | TGCCCACCTGGACACAACACCAAAAAGGCTTCTTATT |
| | | CGGGTGTGAGTCTTTTCAGCAACCCGGTCCAGTACTGG |
| | | GAGATACAGCCATCCACCTTCAGATGTGTCTACGTGC |
| | | GCTCTGCCATTCAACTCGGAAACTATAAGTAA |
| 63 | Alternate HV12 | AAAAATTGAAATTTTATTTTTTTTTTTTGGAATATAAA |
| | insert sequence | TAATGAAGGCCACTATCATCCTCCTTCTGCTTGCACAA |
| | | GTTTCCTGGGCTGGACCGTTTCAACAGAGAGGCTTATT |
| | | TGACTTTATGCTAGAAGATGAGGCTTCTGGGATAGGC |
| | | CCAGAAGTTCCTGATGACCGCGACTTCGAGCCCTCCCT |
| | | AGGCCCAGTGTGCCCCTTCCGCTGTCAATGCCATCTTC |
| | | GAGTGGTCCAGTGTTCTGATTTGGGTCTGGACAAAGT |
| | | GCCAAAGGATCTTCCCCCTGACACAACTCTGCTAGAC |
| | | CTGCAAAACAACAAAATAACCGAAATCAAAGATGGA |
| | | GACTTTAAGAACCTGAAGAACCTTCACGCATTGATTCT |
| | | TGTCAACAATAAAATTAGCAAAGTTAGTCCTGGAGCA |
| | | TTTACACCTTTGGTGAAGTTGGAACGACTTTATCTGTC |
| | | CAAGAATCAGCTGAAGGAATTGCCAGAAAAAATGCCC |
| | | AAAACTCTTCAGGAGCTGCGTGCCCATGAGAATGAGA |
| | | TCACCAAAGTGCGAAAAGTTACTTTCAATGGACTGAA |
| | | CCAGATGATTGTCATAGAACTGGGCACCAATCCGCTG |
| | | AAGAGCTCAGGAATTGAAAATGGGGCTTTCCAGGGAA |
| | | TGAAGAAGCTCTCCTACATCCGCATTGCTGATACCAAT |
| | | ATCACCAGCATTCCTCAAGGTCTTCCTCCTTCCCTTAC |
| | | GGAATTACATCTTGATGGCAACAAAATCAGCAGAGTT |
| | | GATGCAGCTAGCCTGAAAGGACTGAATAATTTGGCTA |
| | | AGTTGGGATTGAGTTTCAACAGCATCTCTGCTGTTGAC |
| | | AATGGCTCTCTGGCCAACACGCCTCATCTGAGGGAGC |
| | | TTCACTTGGACAACAACAAGCTTACCAGAGTACCTGG |
| | | TGGGCTGGCAGAGCATAAGTACATCCAGGTTGTCTAC |
| | | CTTCATAACAACAATATCTCTGTAGTTGGATCAAGTGA |
| | | CTTCTGCCCACCTGGACACAACACCAAAAAGGCTTCTT |
| | | ATTCGGGTGTGAGTCTTTTCAGCAACCCGGTCCAGTAC |
| | | TGGGAGATACAGCCATCCACCTTCAGATGTGTCTACGT |
| | | GCGCTCTGCCATTCAACTCGGAAACTATAAGTAAAAA |
| | | AATTGAAATTTTATTTTTTTTTTTTGGAATATAAATAAT |
| | | GTGTCACCAGCAGTTGGTCATCTCTTGGTTTTCCCTGG |
| | | TTTTTCTGGCATCTCCCCTCGTGGCCATATGGGAACTG |
| | | AAGAAAGATGTTTATGTCGTAGAATTGGATTGGTATC |
| | | CGGATGCCCCTGGAGAAATGGTGGTCCTCACCTGTGA |
| | | CACCCCTGAAGAAGATGGTATCACCTGGACCTTGGAC |
| | | CAGAGCAGTGAGGTCTTAGGCTCTGGCAAAACCCTGA |
| | | CCATCCAAGTCAAAGAGTTTGGAGATGCTGGCCAGTA |
| | | CACCTGTCACAAAGGAGGCGAGGTTCTAAGCCATTCG |
| | | CTCCTGCTGCTTCACAAAAAGGAAGATGGAATTTGGT |
| | | CCACTGATATTTTAAAGGACCAGAAAGAACCCAAAAA |
| | | TAAGACCTTTCTAAGATGCGAGGCCAAGAATTATTCT |
| | | GGACGTTTCACCTGCTGGTGGCTGACGACAATCAGTA |
| | | CTGATTTGACATTCAGTGTCAAAAGCAGCAGAGGCTC |
| | | TTCTGACCCCCAAGGGGTGACGTGCGGAGCTGCTACA |
| | | CTCTCTGCAGAGAGAGTCAGAGGGGACAACAAGGAGT |
| | | ATGAGTACTCAGTGGAGTGCCAGGAGGACAGTGCCTG |
| | | CCCAGCTGCTGAGGAGAGTCTGCCCATTGAGGTCATG |
| | | GTGGATGCCGTTCACAAGCTCAAGTATGAAAACTACA |
| | | CCAGCAGCTTCTTCATCAGGGACATCATCAAACCTGA |
| | | CCCACCCAAGAACTTGCAGCTGAAGCCATTAAAGAAT |
| | | TCTCGGCAGGTGGAGGTCAGCTGGGAGTACCCTGACA |
| | | CCTGGAGTACTCCACATTCCTACTTCTCCCTGACATTC |
| | | TGCGTTCAGGTCCAGGGCAAGAGCAAGAGAGAAAAG |
| | | AAAGATAGAGTCTTCACGGACAAGACCTCAGCCACGG |
| | | TCATCTGCCGCAAAAATGCCAGCATTAGCGTGCGGGC |
| | | CCAGGACCGCTACTATAGCTCATCTTGGAGCGAATGG |
| | | GCATCTGTGCCCTGCAGTTAGCTGGCGAAAGGGGGAT |
| | | GTGCTGCAAGGCGATTAAGTTGGGTAACGCCAGGGTT |
| | | TTCCCAGTCACGACGTTGTAAAACGACGGCCAGTGAA |
| | | TTGTAATACGACTCACTATAGGGCGAATTAATTCCGGT |
| | | TATTTTCCACCATATTGCCGTCTTTTGGCAATGTGAGG |
| | | GCCCGGAAACCTGGCCCTGTCTTCTTGACGAGCATTCC |
| | | TAGGGGTCTTTCCCCTCTCGCCAAAGGAATGCAAGGT |
| | | CTGTTGAATGTCGTGAAGGAAGCAGTTCCTCTGGAAG |
| | | CTTCTTGAAGACAAACAACGTCTGTAGCGACCCTTTGC |
| | | AGGCAGCGGAACCCCCCACCTGGCGACAGGTGCCTCT |
| | | GCGGCCAAAAGCCACGTGTATAAGATACACCTGCAAA |
| | | GGCGGCACAACCCCAGTGCCACGTTGTGAGTTGGATA |
| | | GTTTGTGGAAAGAGTCAAATGGCTCTCCTCAAGCGTA |
| | | TTCAACAAGGGGCTGAAGGATGCCCAGAAGGTACCCC |
| | | ATTGTATGGGATCTGATCTGGGGCCTCGGTGCACATGC |
| | | TTTACATGTGTTTAGTCGAGGTTAAAAAAACGTCTAGG |
| | | CCCCCCGAACCACGGGGACGTGGTTTTCCTTTGAAAA |
| | | ACACGATGATAATGTGGCCCCCTGGGTCAGCCTCCCA |
| | | GCCACCGCCCTCACCTGCCGCGGCCACAGGTCTGCAT |
| | | CCAGCGGCTCGCCCTGTGTCCCTGCAGTGCCGGCTCAG |
| | | CATGTGTCCAGCGCGCAGCCTCCTCCTTGTGGCTACCC |
| | | TGGTCCTCCTGGACCACCTCAGTTTGGCCAGAAACCTC |
| | | CCCGTGGCCACTCCAGACCCAGGAATGTTCCCATGCCT |
| | | TCACCACTCCCAAAACCTGCTGAGGGCCGTCAGCAAC |
| | | ATGCTCCAGAAGGCCAGACAAACTCTAGAATTTTACC |
| | | CTTGCACTTCTGAAGAGATTGATCATGAAGATATCAC |
| | | AAAAGATAAAACCAGCACAGTGGAGGCCTGTTTACCA |
| | | TTGGAATTAACCAAGAATGAGAGTTGCCTAAATTCCA |
| | | GAGAGACCTCTTTCATAACTAATGGGAGTTGCCTGGC |
| | | CTCCAGAAAGACCTCTTTTATGATGGCCCTGTGCCTTA |
| | | GTAGTATTTATGAAGACTTGAAGATGTACCAGGTGGA |
| | | GTTCAAGACCATGAATGCAAAGCTTCTGATGGATCCT |
| | | AAGAGGCAGATCTTTCTAGATCAAAACATGCTGGCAG |
| | | TTATTGATGAGCTGATGCAGGCCCTGAATTTCAACAGT |
| | | GAGACTGTGCCACAAAAATCCTCCCTTGAAGAACCGG |
| | | ATTTTTATAAAACTAAAATCAAGCTCTGCATACTTCTT |
| | | CATGCTTTCAGAATTCGGGCAGTGACTATTGATAGAGT |
| | | GATGAGCTATCTGAATGCTTCCTAAAAAAATTGAAAT |
| | | TTTATTTTTTTTTTTTGGAATATAAATAATGGTGAGCA |
| | | AGGGCGAGGAGCTGTTCACCGGGGTGGTGCCCATCCT |
| | | GGTCGAGCTGGACGGCGACGTAAACGGCCACAAGTTC |
| | | AGCGTGTCCGGCGAGGGCGAGGGCGATGCCACCTACG |
| | | GCAAGCTGACCCTGAAGTTCATCTGCACCACCGGCAA |
| | | GCTGCCCGTGCCCTGGCCCACCCTCGTGACCACCCTGA |
| | | CCTACGGCGTGCAGTGCTTCAGCCGCTACCCCGACCA |
| | | CATGAAGCAGCACGACTTCTTCAAGTCCGCCATGCCC |
| | | GAAGGCTACGTCCAGGAGCGCACCATCTTCTTCAAGG |
| | | ACGACGGCAACTACAAGACCCGCGCCGAGGTGAAGTT |
| | | CGAGGGCGACACCCTGGTGAACCGCATCGAGCTGAAG |
| | | GGCATCGACTTCAAGGAGGACGGCAACATCCTGGGGC |
| | | ACAAGCTGGAGTACAACTACAACAGCCACAACGTCTA |
| | | TATCATGGCCGACAAGCAGAAGAACGGCATCAAGGTG |
| | | AACTTCAAGATCCGCCACAACATCGAGGACGGCAGCG |
| | | TGCAGCTCGCCGACCACTACCAGCAGAACACCCCCAT |
| | | CGGCGACGGCCCCGTGCTGCTGCCCGACAACCACTAC |
| | | CTGAGCACCCAGTCCGCCCTGAGCAAAGACCCCAACG |
| | | AGAAGCGCGATCACATGGTCCTGCTGGAGTTCGTGAC |
| | | CGCCGCCGGGATCACTCTCGGCATGGACGAGCTGTAC |
| | | AAGTAA |
| 65 | 5′ recombination | GATGTCGTACATCGATTACACAAAGAAGTAGAGTCAT |
| | arm | ACGACGTACGTTTCCCTATAAAATCGGTAAACCTAGA |
| | | CGCGGTGTTTCTATCCATAAACGTAACACGTGTACGTC |
| | | TACGTTGGAAGATACCCTTGACCGAACACAATCCTTAT |
| | | CAGACGGCCTACGGATGTTCTAACGACAGATTATACA |
| | | GCTACAACGAGTACGCTTTTTCTCATTTAAAACAAGAC |
| | | CGTGTAAAGATCATAGAACTCCCATGTGACGACGATT |
| | | ACAGCGTCGTGTTAATCACACACGATAGCCGTTCGAC |
| | | TATTACACCGGATAAAGTGACCGGGTGGCTGCGCACG |
| | | ACCCGTCTACGTTACGTAAACGTATCCCTACCCAAGG |
| | | GTTCCACGGAAACGGGACACAACGTAACGTGTCTAAC |
| | | TCCCACACACGTCAATCTATGTCATCGTTGTCGTATAA |
| | | CGATTACCAAAACGGGCGTGGACGCAACCGCGTTCTC |
| | | ATGCGTCGACGGCGATACATGCACCGAACACGACACG |
| | | ACCGCGTCAACGTGTACGATTATTATAAAAACGACGG |
| | | GTCTAGACTTTTTGTTTATGGGGAAACTCTAAGA |
| 66 | 3′ recombination | TATATAAGGTATCTCGTTTGTCTATAACAAAGATCGTA |
| | arm | ACTGACCTTTTTTATATCGAGAAAACATACGTTTAGTT |
| | | CATCCTCAAACGTAACACCGTAACTGCCTCGGACATC |
| | | CTCCTTGTTGTCGTACACAAACATACTAATCGGATGCG |
| | | TGAAATGAGGATTCACTTTAATCGGATTGGTTTCTAGG |
| | | TTAACACATGTTACACAGGATCCTAAGATGGTTATGG |
| | | ACACATCCTTGTTGTGATGTAACGAGTCGGGAAGTTG |
| | | ATTGCCGTAGTTGCCCACGTCGCCCTCCGGTTCCAGAC |
| | | ACGTAATGGTTAGGTATATATCCGAATACTTCGTCAAC |
| | | GGATGAGTCGTAAATAACATGATGGATAGCTTGTTCC |
| | | CATCTCCTGCACCAGCACTGGCCGCCACAAATCGTTGT |
| | | ACCACGTTAGTAATCGTAATGTTTATCATAAGCCCGTA |
| | | CCCGGTTAATATGAGCGTGGACGTTTTATGATCGTATC |
| | | GTTCCTTCATGTGACATTCTCCCATAACCGTTTCGACG |
| | | TACCGATTTAACCCGATGGTTAGCTCGGCGGCTAAGT |
| | | GCCAGTACTTTTTTGGATACGTCGCACATGTTGAGGTT |
| | | GCGACGAGGCAGGCGAGCACGATGATAATATACCGCG |
| | | CCAT |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A recombinant nucleic acid comprising:
at least a portion of myxoma virus (MYXV) genome and a first nucleic acid encoding interleukin-12 subunit beta (IL-12β);
wherein the first nucleic acid is inserted at the MYXV genome to reduce or disrupt the expression of M153 gene of the MYXV genome; and
wherein expression of the IL-12β is driven by a first poxvirus P11 late promoter.

2. The recombinant nucleic acid of claim 1, wherein the IL-12β is human IL-12β.

3. The recombinant nucleic acid of claim 1, further comprising a second nucleic acid encoding interleukin-12 subunit alpha (IL-12α).

4. The recombinant nucleic acid of claim 3, wherein the IL-12α is human IL-12α.

5. The recombinant nucleic acid of claim 3, wherein the 5′ end of the second nucleic acid is coupled to the 3′-end of the first nucleic acid.

6. The recombinant nucleic acid of claim 5, wherein the first and second nucleic acids are coupled via a third nucleic acid encoding an elastin linker.

7. The recombinant nucleic acid of claim 6, further comprising a fourth nucleic acid encoding decorin.

8. The recombinant nucleic acid of claim 7, wherein the decorin is human decorin.

9. The recombinant nucleic acid of claim 7, wherein expression of the decorin is driven by a first sE/L promoter.

10. The recombinant nucleic acid of claim 7, wherein the 5′ end of the fourth nucleic acid is coupled to the 3′-end of the second nucleic acid.

11. The recombinant nucleic acid of claim 9, wherein the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; and (f) the fourth nucleic acid encoding the decorin.

12. The recombinant nucleic acid of claim 1, wherein the recombinant nucleic acid comprises, consists essentially of, or consists of a vMyx-P11 late promoter-hIL-12β-elastin linker-hIL-12α-sE/L promoter-hdecorin expression cassette.

13. The recombinant nucleic acid of claim 1, wherein the recombinant nucleic acid comprises, consists essentially of, or consists of a nucleotide sequence with at least 80%, at least 85%, at least 90%, at least 95%, or at least 98% sequence identity to nucleotides 1-2762 of SEQ ID NO: 10.

14. The recombinant nucleic acid of claim 1, wherein the recombinant nucleic acid comprises, consists essentially of, or consists of a nucleotide sequence that is nucleotides 1-2762 of SEQ ID NO: 10.

15. The recombinant nucleic acid of claim 9, further comprising a fifth nucleic acid encoding a reporter tag.

16. The recombinant nucleic acid of claim 15, wherein the reporter tag comprises a green fluorescent protein (GFP).

17. The recombinant nucleic acid of claim 15, wherein expression of the reporter tag is driven by a second sE/L promoter.

18. The recombinant nucleic acid of claim 17, wherein the recombinant nucleic acid comprises, consists essentially of, or consists of, from 5′ to 3′: (a) the first poxvirus P11 late promoter; (b) the first nucleic acid encoding the IL-12β; (c) the third nucleic acid encoding the elastin linker; (d) the second nucleic acid encoding the IL-12α; (e) the first sE/L promoter; (f) the fourth nucleic acid encoding the decorin; (g) the second sE/L promoter; and (h) the fifth nucleic acid encoding the reporter tag.

19-33. (canceled)

34. A recombinant nucleic acid comprising at least a portion of myxoma virus (MYXV) genome, and a nucleic acid expression cassette inserted at the MYXV genome to reduce or disrupt expression of M153 gene of the MYXV genome, wherein nucleic acid expression cassette comprises, from 5′ to 3′: sE/L promoter-hdecorin-sE/L promoter-hIL-12β-IRES-hIL-12α-sE/L promoter-GFP.

35-40. (canceled)

41. A genetically engineered MYXV comprising a nucleic acid that encodes a cytokine, wherein expression of the cytokine is driven by a poxvirus p11 late promoter, wherein the MYXV is genetically engineered to attenuate expression or activity of M153.

42-72. (canceled)
